(19) United States
(12) Patent Application Publication
(10) Pub. No.: US 2010/0247601 A1
(43) Pub. Date: Sep. 30, 2010

(54) REPAIR AND TREATMENT OF BONE DEFECT USING CELLS INDUCED BY AGENT PRODUCED BY CHONDROCYTES CAPABLE OF HYPERTROPHICATION AND SCAFFOLD

(75) Inventor: Hiroyuki Okihana, Tokyo (JP)

Correspondence Address: SEED INTELLECTUAL PROPERTY LAW GROUP PLLC, 701 FIFTH AVE, SUITE 5400, SEATTLE, WA, 98104, US

(73) Assignee: HOYA CORPORATION, Tokyo (JP)

(21) Appl. No.: 12/665,317

(22) PCT Filed: Jun. 20, 2008
(86) PCT No.: PCT/JP2008/061691

§ 371 Date: Jun. 9, 2010

(30) Foreign Application Priority Data
Jun. 20, 2007 (JP) 2007-163224

(51) Int. Cl. A61K 9/14; C12N 5/071; C12N 5/077; A61K 35/12; A61K 9/00; A61P 19/00

(52) U.S. Cl. 424/423; 435/377; 435/353; 424/93.7; 424/489

(57) ABSTRACT

The present invention provides a method of inducing undifferentiated cells into induced osteoblasts. The method includes: A) a step of providing an induced osteoblast differentiation inducing agent obtained by culturing chondrocytes capable of hypertrophication in a differentiation agent producing medium containing dexamethasone, β-glycerophosphate, ascorbic acid and a serum component; and B) a step of culturing the undifferentiated cells in an undifferentiated cell culture medium containing the induced osteoblast differentiation inducing agent and a medium component, to differentiate the undifferentiated cells into the induced osteoblasts.

### TECHNICAL FIELD

The present invention is directed to a method of inducing undifferentiated cells into osteoblasts by an agent produced by chondrocytes capable of hypertrophication (chondrocytes with an ability of hypertrophication), osteoblasts induced (produced) using the method, a medicine or medical material containing the osteoblasts, a composition containing the osteoblasts and an extracellular matrix, a composite material containing the osteoblasts and a scaffold, and a composite material containing the osteoblasts, an extracellular matrix and a scaffold, and a method of producing them and a method of utilizing them.

### BACKGROUND ART

Osteogenesis is a preferred method of treating diseases associated with decrease of osteogenesis, bone injuries or bone defects. In the case where a bone tissue sustains injury such as a bone fracture or abscission due to a bone tumor, osteoblasts which are bone forming cells proliferate and differentiate to form bone, so that the bone fracture or a bone defective region is cured. In the case of mild injury, by immobilizing bone at an affected area, the osteoblasts can be activated so that the affected area is cured.

In the case where osteoblasts cannot be effectively activated in circumstances such as a complex fracture, large injury of ostectomy and injury in combination with osteomyelitis, autologous bone implantation is generally considered as a standard treatment for repairing such injury or defect. Further, in the case where the bone defective region is too large to repair with an autologous bone, an artificial bone may be used in partial combination with the autologous bone.

However, in the case of human, sources of the autologous bone are limited to a patient itself and an amount thereof which can be collected from the patient is limited. Further, an additional operation is required for collecting the autologous bone, and the collection thereof becomes high costs and causes pain to the patient. In addition, the use of the autologous bone causes a new bone defect to a region (normal bone region) from which the bone is collected.

Therefore, various surgical treatments such as use of an artificial bone implant and use of another bone supply material have been conducted. It is also possible to repair a defective region of a biological organism such as bone, which is generated due to trauma and surgical removal of a bone tumor, by implanting a biological tissue supply material such as a bone supply material into the defective region. Generally, hydroxyapatite (HAP) or tricalcium phosphate (TCP) is known as the bone supply material.

However, as compared with the autologous bone, each of the conventional artificial bone implant and bone supply material also has disadvantages in that it hardly forms bone due to a poor osteogenic ability thereof and is easily broken on impact due to low rigidity thereof. Therefore, prognosis after these surgical treatments is not always so good, this often requires multiple operations. For these reasons, although the percentage of the use of the artificial bone has increased, it remains at about 30% while the autologous bone is used at the remaining 60 to 70%.

In the United States, an allogeneic bone is often used. On the other hand, most Japanese people dislike use of cadaveric tissues, and thus the cadaveric tissues are not used so often. Although bone banks are an alternative way of providing the autologous bone, so far, development thereof is insufficient.

In order to eliminate the above-described disadvantages of the conventional artificial bone, attempts have been made to utilize regenerative medicine using a regenerative ability of cells to apply a treatment for a bone fractural region or a bone defective region. In addition, these attempts also have been applied to increase a postoperative repairing rate of the bone defective region.

Stem cells derived from bone marrow are generally used in such regenerative medicine. It has been proposed to use a biological tissue supply material such as a cultured bone which is produced by culturing bone marrow stem cells or differentiated osteoblasts collected from a patient together with a bone supply material. In such a biological tissue supply material, many bone marrow stem cells or differentiated osteoblasts are proliferated on the bone supply material as a scaffold for culturing them.

In the case where the biological tissue supply material is implanted into a bone defective region, the cells are also implanted thereinto together with the bone supply material. This makes it possible to compensate the above-described disadvantages of the artificial bone and to reduce a period of osteogenesis, as compared with a method in which the bone supply material alone is implanted into the bone defective region.

Conventionally, in order to differentiate mesenchymal stem cells derived from bone marrow into osteoblasts with the use of the regenerative medicine, a method proposed by Maniatopoulos et al. in which three compounds of dexamethasone, β-glycerophosphate and ascorbic acid are used, and a method in which concentrations of the three compounds are modified are utilized.

However, these methods are artificial, but are not natural (non-patent document 1, that is, Maniatopoulos et al.: Bone formation in vitro by stromal cells obtained from bone marrow of young adult rats. Cell Tissue Res, 254: 317-330, 1988.). There still exist cells which are not differentiated by these three compounds among the stem cells. As a result, there is an anxiety for a property and function of differentiated osteoblasts.

Therefore, there is a need to provide osteoblasts used for treating diseases associated with decrease of osteogenesis, bone injuries or bone defects safely, economically and stably.

It is believed that BMP (Bone Morphogenetic Protein)-2, BMP-4, and BMP-7 play important role in osteogenesis by inducing osteoblasts. The BMP-2, the BMP-4 and the BMP-7 are believed to induce into the osteoblasts. Although there are many family members of a BMP family, molecules other than the BMP-2, the BMP-4 and the BMP-7 are homologs obtained based on a previously identified sequence of the BMP-2 with disregard to their function. Therefore, the homologs do not always have an ability of inducing differentiation into the osteoblasts.

It is reported that the BMP-2, the BMP-4 and the BMP-7 induce the osteoblasts effectively in mouse and rat, but an efficiency is only a thousandth of that in human (non-patent documents 2 to 5, that is, Wozney, J. M. et al.: Novel Regulators of Bone Formation: Molecular Clones and Activities. Science, 242: 1528-1534, 1988.; Wuerzler K K et al.: Radiation-Induced Impairment of Bone Healing Can Be overcome by Recombinant Human Bone Morphogenetic Protein-2. J. Craniofacial Surg., 9:131-137, 1998.; Govender S et al.: Recombinant Human Bone Morphogenetic Protein-2 for treatment of Open Tibial Fractures. J. Bone Joint Surg., 84A: 2123-2134, 2002.; Johnsson R et al.: Randomized Radiostereometric Study Comparing Osteogenic Protein-1 (BMP-7) and Autograft Bone in Human Noninstrumented Posterolateral Lumber Fusion. Spine, 27: 2654-2661, 2002.).

The present inventor observed that osteogenesis due to intracartilaginous ossification was induced by implanting BMP into heterotopias. Wozney et al. who cloned BMP used the term “cartilage-inducing activity” upon measuring an activity of the BMP (non-patent document 2, that is, Wozney, J. M. et al.: Novel Regulators of Bone Formation: Molecular Clones and Activities. Science, 242: 1528-1534, 1988.).

Through this observation, the present inventor considered that the osteogenesis is not directly induced by the BMP-2, the BMP-4 and the BMP-7, but is induced by osteoblasts differentiated by an agent produced by chondrocytes capable of hypertrophication, which are induced by the BMP-2, the BMP-4 and the BMP-7 (non-patent documents 6 and 7, that is, Hiroyuki Okihana: seichonankotsu no seisansuru kotsukeiseiinshi [an osteogenesic agent produced by growth cartilage], igaku no ayumi [Journal of Clinical and Experimental Medicine], 165: 419, 1993.; Okihana, H. & Shimomura, Y: Osteogenic Activity of Growth Cartilage Examined by Implanting Decalcified and Devitalized Ribs and Costal Cartilage Zone, and Living Growth Cartilage Cells. Bone, 13: 387-393, 1992.).

However, a peptidergic agent or agent derived from a biological organism having a molecular weight of 50,000 or higher, which directly affects induction, chemotaxis and activation of the osteoblasts, has not been known.

A patent document 1 (Japanese Patent Application Laid-open No. 2004-305259) discloses a method of producing a biological tissue supply material. The method comprises allowing stem cells to adhere to a biological tissue supply material, inducing differentiation of the stem cells, to generate an effect of formation of a biological tissue using the biological tissue supply material as a scaffold, and then subjecting the formed tissue cells to an extinction treatment. Namely, the patent document 1 discloses that the stem cells adhere to the biological tissue supply material, and then are differentiated into osteoblasts.

The patent document 1 also discloses that differentiation inducing agents such as a minimum essential medium, fetal bovine serum (FBS), dexamethasone and β-glycerophosphate and nutritional supplements such as ascorbic acid may be mixed with a medium to be used in this culture, and that a medium with which the minimum essential medium, the fetal bovine serum (FBS) and the dexamethasone are mixed is used in a cell culture.

However, the patent document 1 does not disclose a method of inducing undifferentiated cells into osteoblasts using an agent produced by chondrocytes capable of hypertrophication, osteoblasts induced (produced) using the method, a composition containing the osteoblasts, a composite material containing the osteoblasts, an extracellular matrix and a scaffold, and a fact that the composite material promotes and induces osteogenesis in a biological organism.

A patent document 2 (Japanese Patent Application Laid-open No. 2004-305260) discloses a method of producing a biological tissue supply material. The method comprises allowing stem cells to adhere to a biological tissue supply material, inducing differentiation of the stem cells, to generate an effect of formation of a biological tissue using the biological tissue supply material as a scaffold, and then subjecting the formed tissue cells to an extinction treatment, wherein the extinction treatment includes freezing the biological tissue supply material, and then drying it. Namely, the patent document 2 discloses that the stem cells adhere to the biological tissue supply material, and then are differentiated into osteoblasts.

The patent document 2 also discloses that differentiation inducing agents such as a minimum essential medium, fetal bovine serum (FBS), dexamethasone and β-glycerophosphate and nutritional supplements such as ascorbic acid are mixed with a medium to be used in this culture, and that a medium with which the minimum essential medium, the fetal bovine serum (FBS) and the dexamethasone are mixed is used in a cell culture.

However, the patent document 2 does not disclose a method of inducing undifferentiated cells into osteoblasts using an agent produced by chondrocytes capable of hypertrophication, osteoblasts induced (produced) using the method, a composition containing the osteoblasts and an extracellular matrix, a composite material containing the osteoblasts, an extracellular matrix and a scaffold, and a fact that the composite material promotes and induces osteogenesis in a biological organism.

A patent document 3 (Japanese Patent Application Laid-open No. 2004-49142) discloses a method of producing a cultured bone. The method comprises: a primary culturing step of obtaining mesenchymal stem cells by culturing bone marrow cells collected from a patient in a predetermined culture medium; a secondary culturing step of inducing differentiation of the cultured mesenchymal stem cells into osteoblasts by culturing them in a predetermined bone forming culture medium; a collecting step of collecting the differentiated osteoblasts and a produced bone substrate; and a mixing step of mixing the collected osteoblasts and the bone substrate with granules of a bone supply material.

The patent document 3 also discloses that the mesenchymal stem cells are differentiated into the osteoblasts using a medium with which differentiation inducing agents such as a minimum essential medium, fetal bovine serum (FBS), dexamethasone and β-glycerophosphate and nutritional supplements such as ascorbic acid are mixed.

However, the patent document 3 does not disclose a method of inducing undifferentiated cells into osteoblasts using an agent produced by chondrocytes capable of hypertrophication, osteoblasts induced (produced) using the method, a composition containing the osteoblasts and an extracellular matrix, a composite material containing the osteoblasts, an extracellular matrix and a scaffold, and a fact that the composite material promotes and induces osteogenesis in a biological organism.

A patent document 4 (Japanese Patent Application Laid-open No. 2005-205074) discloses a method of producing a cultured bone by carrying mesenchymal stem cells obtained by culturing cells collected from a patient on a bone supply material, and then culturing the mesenchymal stem cells carried on the bone supply material to differentiate them into osteoblasts, or a method of producing a cultured bone by culturing mesenchymal stem cells obtained from cells collected from a patient to differentiate them into osteoblasts, and then carrying the osteoblasts on a bone supply material.

The patent document 4 also discloses that the mesenchymal stem cells are differentiated into the osteoblasts using a medium with which differentiation inducing agents such as a minimum essential medium, fetal bovine serum (FBS), dexamethasone and β-glycerophosphate and nutritional supplements such as ascorbic acid are mixed. In this method, platelet-rich plasma needs to be added to a culture liquid for culturing the cells collected from the patient, a culture liquid for culturing the mesenchymal stem cells, or a culture liquid after inducing differentiation of the mesenchymal stem cells into the osteoblasts.

However, the patent document 4 does not disclose a method of inducing undifferentiated cells into osteoblasts using an agent produced by chondrocytes capable of hypertrophication, osteoblasts induced (produced) using the method, a composition containing the osteoblasts and an extracellular matrix, a composite material containing the osteoblasts, an extracellular matrix and a scaffold, and a fact that the composite material promotes and induces osteogenesis in a biological organism.

A patent document 5 (Japanese National Phase PCT Laid-Open No. 2003-531604) discloses a method of isolating mesenchymal stem cells from a human tissue after birth such as a human prepuce tissue after birth, and a method of inducing differentiation of the isolated mesenchymal stem cells into various cell lineages such as osteogenesis, adipogenesis and a cartilage formation lineage.

The patent document 5 also discloses that the mesenchymal stem cells are differentiated into osteoblasts using a medium containing fetal bovine serum (FBS), an antibiotic and osteogenic complementary substances such as dexamethasone, β-glycerophosphate and ascorbic acid-2-phosphoric acid.

However, the patent document 5 does not disclose a method of inducing undifferentiated cells into osteoblasts using an agent produced by chondrocytes capable of hypertrophication, osteoblasts induced (produced) using the method, a composition containing osteoblasts and an extracellular matrix, a composite material containing osteoblasts, an extracellular matrix and a scaffold, and a fact that the composite material promotes and induces osteogenesis in a biological organism.

A patent document 6 (Japanese Patent No. 2984176) discloses a method of culturing bone marrow cells, a mixture for culturing and a material to be implanted into a hard tissue defective region. In the patent document 6, it is described that both hormones such as dexamethasone and serums need not to be added to a medium to be used for culturing osteoblasts, and that the medium preferably contains ascorbic acid. Further, in the patent document 6, a mixture of L-ascorbic acid, a HEPES buffer solution and a minimum essential medium α (αMEM) is used for inducing differentiation.

However, the patent document 6 does not disclose a method of inducing undifferentiated cells into osteoblasts using an agent produced by chondrocytes capable of hypertrophication, osteoblasts induced (produced) using the method, a composition containing the osteoblasts and an extracellular matrix, a composite material containing the osteoblasts, an extracellular matrix and a scaffold, and a fact that the composite material promotes and induces osteogenesis in a biological organism.

A patent document 7 (Japanese Patent No. 3808900) discloses a physiological substance, a process of preparing the same and a method of applying the same to implantation. In the patent document 7, it is described that, for osteogenesis, mesenchymal stem cells are differentiated using a medium containing fetal bovine serum (FBS), L-glutamine, penicillin/streptomycin, ascorbic acid and dexamethasone.

However, the patent document 7 does not disclose a method of inducing undifferentiated cells into osteoblasts using an agent produced by chondrocytes capable of hypertrophication, osteoblasts induced (produced) using the method, a composition containing the osteoblasts and an extracellular matrix, a composite material containing the osteoblasts, an extracellular matrix and a scaffold, and a fact that the composite material promotes and induces osteogenesis in a biological organism.

In each of the patent documents 1 to 7, it is only described that differentiation of undifferentiated cells into osteoblasts is induced using a medium itself containing dexamethasone, β-glycerophosphate and ascorbic acid which are commonly used as components capable of inducing differentiation into the osteoblasts.

A patent document 8 (Japanese Patent Application Laid-open No. 2006-289062) discloses a bone supply material produced using chondrocytes capable of hypertrophication and a scaffold. However, the patent document 8 does not disclose a method of inducing undifferentiated cells into osteoblasts using an agent produced by chondrocytes capable of hypertrophication, osteoblasts induced (produced) using the method, a composition containing the osteoblasts and an extracellular matrix, a composite material containing the osteoblasts, an extracellular matrix and a scaffold, and a fact that the composite material promotes and induces osteogenesis in a biological organism.

### DISCLOSURE OF THE INVENTION

### Problems to be Resolved by the Invention

An object of the present invention is to massively and stably provide osteoblasts which can be used for treating diseases associated with decrease of osteogenesis, bone injuries or bone defects, especially, for treating bone tumors, complex fractures and the like.

Heretofore, osteogenesis did not occur, even in the case where osteoblasts alone had been implanted into a biological organism. Therefore, the osteoblasts alone could not treat the diseases associated with the decrease of osteogenesis, the bone injuries or the bone defects, especially, could not treat the bone tumors, the complex fractures and the like. Accordingly, an object of the present invention is to provide osteoblasts having an ability of independently treating diseases or injuries for which the osteogenesis is required.

Further, an object of the present invention is to provide osteoblasts which can be used for forming bone in a region where the bone does not exist in the vicinity thereof.

### Means of Solving the Problems

In the present invention, a method of inducing undifferentiated cells into osteoblasts has been found by using an induced osteoblast differentiation inducing agent produced by chondrocytes capable of hypertrophication. This makes it possible to massively and stably provide osteoblasts which could not be mass-produced heretofore.

The present invention have, first, achieved promotion and induction of osteogenesis even in a region where bone does not exist in the vicinity thereof within a biological organism without using a scaffold, by independently implanting osteoblasts which are induced from undifferentiated cells using the inducing method of the present invention.

In order to achieve the above objects, the present invention provides the following means.

(Item 1)

A method of inducing undifferentiated cells into induced osteoblasts, comprising:

A) a step of providing a supernatant obtained by culturing chondrocytes capable of hypertrophication in a differentiation agent producing medium containing at least one selected from the group comprising glucocorticoid, β-glycerophosphate and ascorbic acid, or an induced osteoblast differentiation inducing agent existing in the supernatant; and

B) a step of culturing the undifferentiated cells in an undifferentiated cell culture medium containing the supernatant or the induced osteoblast differentiation inducing agent and a medium component at a sufficient condition that the undifferentiated cells are induced into the induced osteoblasts.

(Item 1A)

The method as described above, wherein in the step A), the induced osteoblast differentiation inducing agent is obtained by culturing the chondrocytes capable of hypertrophication in the differentiation agent producing medium containing dexamethasone, the β-glycerophosphate, the ascorbic acid and a serum component, and

wherein in the step B), the undifferentiated cells are induced into the induced osteoblasts by culturing the undifferentiated cells in the undifferentiated cell culture medium containing the induced osteoblast differentiation inducing agent and the medium component.

(Item 1B)

The method as described above, wherein the induced osteoblast differentiation inducing agent exists (1) in the medium in which the chondrocytes capable of hypertrophication are cultured, or (2) in a fraction with a molecular weight of 50,000 or higher obtained by subjecting the medium in which the chondrocytes capable of hypertrophication are cultured to ultrafiltration using a filter having a molecular cutoff of 50,000.

(Item 1C)

The method as described above, wherein the step A) includes:

culturing the chondrocytes capable of hypertrophication in the differentiation agent producing medium containing dexamethasone, the β-glycerophosphate, the ascorbic acid and a serum component; and

collecting the supernatant of the differentiation agent producing medium after the culture.

(Item 1D)

The method as described above, wherein the step A) includes subjecting the differentiation agent producing medium in which the chondrocytes capable of hypertrophication are cultured to ultrafiltration to separate it into a fraction with a molecular weight of 50,000 or higher.

(Item 2)

The method as described above, wherein the differentiation agent producing medium used for culturing the chondrocytes capable of hypertrophication contains both the β-glycerophosphate and the ascorbic acid.

(Item 3)

The method as described above, wherein the undifferentiated cells comprise cells derived from a mammal.

(Item 4)

The method as described above, wherein the undifferentiated cells comprise cells derived from human, mouse, rat or rabbit.

(Item 5)

The method as described above, wherein the undifferentiated cells comprise cells selected from the group comprising mesenchymal stem cells, hematopoietic stem cells, vascular stem cells, hepatic stem cells, pancreatic stem cells and neural stem cells.

(Item 5A)

The method as described above, wherein the undifferentiated cells comprise the mesenchymal stem cells.

(Item 5B)

The method as described above, wherein the mesenchymal stem cells comprise mesenchymal stem cells derived from bone marrow.

(Item 5C)

The method as described above, wherein the mesenchymal stem cells comprise rat mesenchymal stem cells or human mesenchymal stem cells.

(Item 6)

The method as described above, wherein the undifferentiated cells comprise cells selected from the group comprising C3H10T1/2 cells, ATDC5 cells, 3T3-Swiss albino cells, BALB/3T3 cells, NIH3T3 cells, C2C12 cells, PT-2501 cells and stem cells derived from primary rat bone marrow.

(Item 6A)

The method as described above, wherein the undifferentiated cells comprise cells selected from the group comprising the C3H10T1/2 cells, the 3T3-Swiss albino cells, the BALB/3T3 cells, the NIH3T3 cells, the PT-2501 cells and the stem cells derived from primary rat bone marrow.

(Item 6B)

The method as described above, wherein the undifferentiated cells comprise the C3H10T1/2 cells.

(Item 7)

The method as described above, wherein the undifferentiated cell culture medium contains an eagle' s basal medium (BME), a minimum essential medium (MEM), a dulbecco's modified eagle medium (DMEM), a Ham's F12 medium (HAM), a minimum essential medium α (αMEM) or a mixed medium thereof.

(Item 7A)

The method as described above, wherein the undifferentiated cell culture medium contains the eagle's basal medium (BME), the minimum essential medium (MEM), the Ham's F12 medium (HAM) or the dulbecco's modified eagle medium (DMEM).

(Item 8)

The method as described above, further comprising a step of forming the undifferentiated cells into a pellet.

(Item 9)

The method as described above, wherein the pellet forming step is performed by being centrifuged at 170 to 200×g for 3 to 5 minutes.

(Item 10)

The method as described above, wherein the sufficient condition that the undifferentiated cells are induced into the induced osteoblasts includes a culture period of 3 days to 3 weeks.

(Item 11)

The method as described above, wherein the chondrocytes capable of hypertrophication are cultured in the differentiation agent producing medium containing the glucocorticoid, the β-glycerophosphate and the ascorbic acid,

wherein the undifferentiated cells are selected from the group comprising mesenchymal stem cells, hematopoietic stem cells, vascular stem cells, hepatic stem cells, pancreatic stem cells and neural stem cells,

wherein the undifferentiated cell culture medium contains an eagle's basal medium (BME), a minimum essential medium (MEM), a minimum essential medium α (αMEM) or a dulbecco's modified eagle medium (DMEM), and

wherein the sufficient condition that the undifferentiated cells are induced into the induced osteoblasts includes a culture period of 3 days to 3 weeks.

(Item 11A)

The method as described above, wherein the undifferentiated cells comprise mesenchymal stem cells derived from bone marrow, and

wherein the step A) includes: (1) culturing the chondrocytes capable of hypertrophication in the differentiation agent producing medium containing dexamethasone, the β-glycerophosphate, the ascorbic acid and a serum component, and then collecting the supernatant of the differentiation agent producing medium after the culture; and (2) subjecting the supernatant to ultrafiltration to separate it into a fraction with a molecular weight of 50,000 or higher.

(Item 12)

The method as described above, wherein the chondrocytes capable of hypertrophication are cultured in the differentiation agent producing medium containing the glucocorticoid, the β-glycerophosphate and the ascorbic acid,

wherein the undifferentiated cells are selected from the group comprising C3H10T1/2 cells, ATDC5 cells, 3T3-Swiss albino cells, BALB/3T3 cells, NIH3T3 cells, C2C12 cells, PT-2501 cells and stem cells derived from primary rat bone marrow,

wherein the undifferentiated cell culture medium contains an eagle's basal medium (BME), a minimum essential medium (MEM), a minimum essential medium α (αMEM) or a dulbecco's modified eagle medium (DMEM), and

wherein the sufficient condition that the undifferentiated cells are induced into the induced osteoblasts includes a culture period of 3 days to 3 weeks.

(Item 12A)

The method as described above, wherein the undifferentiated cells comprise the C3H10T1/2 cells, the PT-2501 cells or the stem cells derived from primary rat bone marrow, and

wherein the step A) includes: (1) culturing the chondrocytes capable of hypertrophication in the differentiation agent producing medium containing dexamethasone, the β-glycerophosphate, the ascorbic acid and a serum component, and then collecting the supernatant of the differentiation agent producing medium after the culture; and (2) subjecting the supernatant to ultrafiltration to separate it into a fraction with a molecular weight of 50,000 or higher.

(Item 13)

The method as described above, wherein the chondrocytes capable of hypertrophication are cultured in the differentiation agent producing medium containing the dexamethasone, the β-glycerophosphate and the ascorbic acid,

wherein the undifferentiated cells are selected from the group comprising mesenchymal stem cells, hematopoietic stem cells, vascular stem cells, hepatic stem cells, pancreatic stem cells and neural stem cells,

wherein the undifferentiated cells are formed into a pellet by being centrifuged at 170 to 200×g for 3 to 5 minutes,

wherein the undifferentiated cell culture medium contains an eagle's basal medium (BME), a minimum essential medium (MEM), a minimum essential medium α (αMEM) or a dulbecco's modified eagle medium (DMEM), and

wherein the sufficient condition that the undifferentiated cells are induced into the induced osteoblasts includes a culture period of 3 days to 3 weeks.

(Item 14)

The method as described above, wherein the chondrocytes capable of hypertrophication are cultured in the differentiation agent producing medium containing the glucocorticoid, the β-glycerophosphate and the ascorbic acid,

wherein the undifferentiated cells are selected from the group comprising C3H10T1/2 cells, ATDC5 cells, 3T3-Swiss albino cells, BALB/3T3 cells, NIH3T3 cells, C2C12 cells, PT-2501 cells and stem cells derived from primary rat bone marrow,

wherein the undifferentiated cells are formed into a pellet by being centrifuged at 170 to 200×g for 3 to 5 minutes,

wherein the undifferentiated cell culture medium contains an eagle's basal medium (BME), a minimum essential medium (MEM), a minimum essential medium α (αMEM) or a dulbecco's modified eagle medium (DMEM), and

wherein the sufficient condition that the undifferentiated cells are induced into the induced osteoblasts includes a culture period of 3 days to 3 weeks.

(Item 15)

Induced osteoblasts produced using the method as described above.

(Item 16)

A composition for promoting or inducing osteogenesis in a biological organism, comprising:

A) an extracellular matrix; and

B) induced osteoblasts.

(Item 17)

The composition as described above, wherein the extracellular matrix is derived from the induced osteoblasts.

(Item 18)

The composition as described above, wherein the extracellular matrix is selected from the group comprising type I collagen, bone proteoglycan, osteocalcin, matrix Gla protein, osteoglycin, osteopontin and bone sialic acid protein.

(Item 19)

The composition as described above, wherein the extracellular matrix and induced osteoblasts exist in a mixed state.

(Item 20)

The composition as described above, wherein the induced osteoblasts are derived from cells selected from the group comprising mesenchymal stem cells, hematopoietic stem cells, vascular stem cells, hepatic stem cells, pancreatic stem cells and neural stem cells.

(Item 20A)

The composition as described above, wherein the induced osteoblasts are derived from the mesenchymal stem cells.

(Item 20B)

The composition as described above, wherein the mesenchymal stem cells comprise mesenchymal stem cells derived from bone marrow.

(Item 20C)

The composition as described above, wherein the mesenchymal stem cells comprise rat mesenchymal stem cells or human mesenchymal stem cells.

(Item 21)

The composition as described above, wherein the induced osteoblasts are derived from cells selected from the group comprising C3H10T1/2 cells, ATDC5 cells, 3T3-Swiss albino cells, BALB/3T3 cells, NIH3T3 cells, C2C12 cells, PT-2501 cells and stem cells derived from primary rat bone marrow.

(Item 22)

The composition as described above, wherein the induced osteoblasts are derived from cells selected from the group comprising mesenchymal stem cells, hematopoietic stem cells, vascular stem cells, hepatic stem cells, pancreatic stem cells and neural stem cells, and

wherein the induced osteoblasts excrete the extracellular matrix.

(Item 23)

The composition as described above, wherein the induced osteoblasts are derived from cells selected from the group comprising C3H10T1/2 cells, ATDC5 cells, 3T3-Swiss albino cells, BALB/3T3 cells, NIH3T3 cells, C2C12 cells, PT-2501 cells and stem cells derived from primary rat bone marrow, and

wherein the induced osteoblasts excrete the extracellular matrix.

(Item 24)

The composition as described above, wherein the osteogenesis is utilized for repairing or treating a bone defect.

(Item 25)

The composition as described above, wherein the bone defect has a size that cannot be repaired only by immobilizing bone.

(Item 26)

The composition as described above, wherein the osteogenesis is utilized for forming bone in a region where the bone does not exist in the vicinity thereof.

(Item 27)

A composite material for promoting or inducing osteogenesis in a biological organism, comprising:

A) an extracellular matrix;

B) induced osteoblasts; and

C) a biocompatible scaffold.

(Item 28)

The composite material as described above, wherein the extracellular matrix is derived from the induced osteoblasts.

(Item 29)

The composite material as described above, wherein the extracellular matrix is selected from the group comprising type I collagen, bone proteoglycan, osteocalcin, matrix Gla protein, osteoglycin, osteopontin and bone sialic acid protein.

(Item 30)

The composite material as described above, wherein the biocompatible scaffold contains a material selected from the group comprising calcium phosphate, calcium carbonate, alumina, zirconia, apatite-wollastonite deposited glass, gelatin, collagen, chitin, fibrin, hyaluronic acid, an extracellular matrix mixture, silk, cellulose, dextran, agarose, agar, synthetic polypeptide, polylactic acid, polyleucine, alginic acid, polyglycolic acid, polymethyl methacrylate, polycyanoacrylate, polyacrylonitrile, polyurethane, polypropylene, polyethylene, polyvinyl chloride, an ethylene-vinyl acetate copolymer, nylon and a combination thereof.

(Item 31)

The composite material as described above, wherein the biocompatible scaffold contains a material selected from the group comprising porous hydroxyapatite, super porous hydroxyapatite, an apatite-collagen mixture, an apatite-collagen complex, collagen gel, collagen sponge, gelatin sponge, fibrin gel, synthetic peptide, an extracellular matrix mixture, alginate, agarose, polyglycolic acid, polylactic acid, a polyglycolic acid/polylactic acid copolymer and a combination thereof.

(Item 32)

The composite material as described above, wherein the induced osteoblasts adhere to the biocompatible scaffold via the extracellular matrix or directly.

(Item 33)

A method of promoting or inducing osteogenesis in a biological organism, comprising:

a step of implanting a composition containing an extracellular matrix and induced osteoblasts or a composite material containing an extracellular matrix, induced osteoblasts and a biocompatible scaffold, into a region where the osteogenesis is required to be promoted or induced in the biological organism.

(Item 34)

The method as described above, being utilized for repairing or treating a bone defect.

(Item 35)

The method as described above, wherein the bone defect has a size that cannot be repaired only by immobilizing bone.

(Item 36)

The method as described above, being utilized for forming bone in a region where the bone does not exist in the vicinity thereof.

(Item 37)

A method of producing a composite material for promoting or inducing osteogenesis in a biological organism, comprising:

A) a step of providing induced osteoblasts induced using an agent produced by chondrocytes capable of hypertrophication;

B) a step of culturing the induced osteoblasts on a biocompatible scaffold.

(Item 38)

A method of producing a composite material for promoting or inducing osteogenesis in a biological organism, comprising:

A) a step of providing induced osteoblasts produced using the method as described above;

B) a step of culturing the induced osteoblasts on a biocompatible scaffold.

(Item 39)

The method as described above, wherein the induced osteoblasts are induced from undifferentiated cells on the biocompatible scaffold.

(Item 40)

A medicine or medical material for promoting or inducing osteogenesis in a biological organism, comprising:

induced osteoblasts produced using the method as described above.

(Item 41)

The medicine or medical material as described above, wherein the undifferentiated cells comprise mesenchymal stem cells.

(Item 42)

The medicine or medical material as described above, wherein the undifferentiated cells comprise mesenchymal stem cells derived from bone marrow.

(Item 43)

The medicine or medical material as described above, wherein the undifferentiated cells comprise C3H10T1/2 cells, 3T3-Swiss albino cells, BALB/3T3 cells, NIH3T3 cells, PT-2501 cells or stem cells derived from primary rat bone marrow.

(Item 44)

The medicine or medical material as described above, wherein the undifferentiated cells comprise the C3H10T1/2 cells, the PT-2501 cells or the stem cells derived from primary rat bone marrow.

(Item 45)

The medicine or medical material as described above, wherein the undifferentiated cells are formed into a pellet.

(Item 46)

The medicine or medical material as described above, wherein the C3H10T1/2 cells are formed into a pellet.

(Item 47)

The medicine or medical material as described above, wherein the mesenchymal stem cells are encapsulated with a gel.

(Item 48)

A method of promoting or inducing osteogenesis in a biological organism, comprising:

a step of implanting induced osteoblasts produced using the method as described above, into a region where the osteogenesis is required to be promoted or induced in the biological organism.

(Item 49)

Use of induced osteoblasts for producing a medicine or medical material for promoting or inducing osteogenesis in a biological organism, wherein the induced osteoblasts are produced using the method as described above.

According to the present invention, it is possible to provide a method of massively and stably producing osteoblasts which can promote or induce osteogenesis in a biological organism.

According to the producing method of the present invention, it is possible to provide osteoblasts which can be used for treating diseases associated with decrease of osteogenesis, bone injuries or bone defects, especially, for treating bone tumors, complex fractures and the like. The osteoblasts according to the present invention can independently promote or induce the osteogenesis in the biological organism without using a scaffold. This is an unexpected advantageous effect which has been first achieved by the present invention.

By utilizing the osteoblasts according to the present invention, it is possible to provide a medicine or medical material containing the osteoblasts, a composition containing the osteoblasts and an extracellular matrix and a composite material containing the osteoblasts, an extracellular matrix and a scaffold, each of which can be used for treating diseases associated with decrease of osteogenesis, bone injuries or bone defects, especially, for treating bone tumors, complex fractures and the like. Further, it is also possible to provide a method of producing them and a method of utilizing them.

Such osteoblasts, medicine or medical material, composition and composite material can promote or induce the osteogenesis in the biological organism. By using them, the osteogenesis came to be capable of being induced even in a region where bone did not exist in the vicinity thereof. These osteoblasts, medicine or medical material, composition and composite material have not been provided using the prior art, but is, first, provided using the present invention.

### BRIEF DESCRIPTION OF THE DRAWINGS

FIG. 1A shows a result of alkaline phosphatase staining of chondrocytes capable of hypertrophication inoculated on hydroxyapatite by diluting them to prepare a cell suspension, and then applying the cell suspension to the hydroxyapatite.

The chondrocytes capable of hypertrophication were inoculated on the hydroxyapatite at a density of 1×10⁶ cells/mL, and then cultured in a 5% CO₂ incubator at 37° C. for 1 week to obtain a sample. Thereafter, the sample was subjected to the alkaline phosphatase staining. The sample (hydroxyapatite) was stained red with the alkaline phosphatase staining. In FIG. 1A, the length of the bar shown in the lower left thereof is 300.00 μm.

FIG. 1B shows a result of toluidine blue staining of the sample subjected to the alkaline phosphatase staining shown in FIG. 1A. It was confirmed that the same areas of the sample (hydroxyapatite) shown in FIG. 1A were stained blue with the toluidine blue staining, and therefore cells were present in the sample. In FIG. 1B, the length of the bar shown in the lower left thereof is 300.00 μm.

FIG. 1C shows a result of alkaline phosphatase staining of resting cartilage cells inoculated on hydroxyapatite by diluting them to prepare a cell suspension, and then applying the cell suspension to the hydroxyapatite.

The resting cartilage cells were inoculated on the hydroxyapatite at a density of 1×10⁶ cells/mL, and then cultured in a 5% CO₂ incubator at 37° C. for 1 week to obtain a sample. Thereafter, the sample was subjected to the alkaline phosphatase staining. The sample (hydroxyapatite) was not stained with the alkaline phosphatase staining. In FIG. 1C, the length of the bar shown in the lower left thereof is 300.00 μm.

FIG. 1D shows a result of toluidine blue staining of the sample subjected to the alkaline phosphatase staining shown in FIG. 1C. It was confirmed that the sample (hydroxyapatite) was stained blue with the toluidine blue staining, and therefore cells were present in the sample. In FIG. 1D, the length of the bar shown in the lower left thereof is 300.00 μm.

FIG. 1E shows a result of alkaline phosphatase staining of chondrocytes derived from articular cartilage on hydroxyapatite by diluting them to prepare a cell suspension, and then applying the cell suspension to the hydroxyapatite.

The chondrocytes derived from articular cartilage were inoculated on the hydroxyapatite at a density of 1×10⁶ cells/mL, and then cultured in a 5% CO₂ incubator at 37° C. for 1 week to obtain a sample. Thereafter, the sample was subjected to the alkaline phosphatase staining. The sample (hydroxyapatite) was not stained with the alkaline phosphatase staining. In FIG. 1E, the length of the bar shown in the lower left thereof is 300.00 μm.

FIG. 1F shows a result of toluidine blue staining of the sample subjected to the alkaline phosphatase staining shown in FIG. 1E. It was confirmed that spotted areas of the sample (hydroxyapatite) were stained blue with the toluidine blue staining, and therefore cells were present in the sample. In FIG. 1F, the length of the bar shown in the lower left thereof is 300.00 μm.

FIG. 2 shows alkaline phosphatase activities of mouse C3H10T1/2 cells each measured by culturing chondrocytes capable of hypertrophication derived from costa/costal cartilage in an MEM differentiation agent producing medium and an MEM growth medium, respectively, collecting a supernatant of each of the mediums (culture supernatant) on a time course (4 days, 1 week, 2 weeks, 3 weeks) to obtain fractional supernatants, adding each of the fractional supernatants to the mouse C3H10T1/2 cells, and then culturing them.

In the case where the supernatant of the MEM differentiation agent producing medium (culture of cells) was added to the mouse C3H10T1/2 cells, when a value of the alkaline phosphatase activity of the mouse C3H10T1/2 cells cultured by adding only the MEM differentiation agent producing medium was defined as “1”, in a 4 week-old rat group: a relative value thereof increased to about 4.1 times by adding the fractional supernatant collected 4 days after the culture; to about 5.1 times by adding the fractional supernatant collected 1 week after the culture; to about 5.4 times by adding the fractional culture supernatant collected 2 weeks after the culture; and to about 4.9 times by adding the fractional culture supernatant collected 3 weeks after the culture.

In the above same case, in a 8 week-old rat group: a relative value thereof increased to about 2.9 times by adding the fractional supernatant collected 4 days after the culture; to about 3.1 times by adding the fractional supernatant collected 1 week after the culture; to about 3.8 times by adding the fractional supernatant collected 2 weeks after the culture; and to about 4.2 times by adding the fractional supernatant collected 3 weeks after the culture.

In each of the 4 and 8 week-old rat groups, there was little difference between a value of the alkaline phosphatase activity of the mouse C3H10T1/2 cells cultured by adding the supernatant of the MEM growth medium in which the chondrocytes capable of hypertrophication were cultured (culture of cells) and a value of the alkaline phosphatase activity thereof cultured by adding only the MEM growth medium.

The following abbreviations show the added supernatants. 4 week-old differentiation supernatant: supernatant of MEM differentiation agent producing medium in which chondrocytes capable of hypertrophication derived from 4 week-old rat were cultured; 8 week-old differentiation supernatant: supernatant of MEM differentiation agent producing medium in which chondrocytes capable of hypertrophication derived from 8 week-old rat were cultured; 4 week-old growth supernatant: supernatant of MEM growth medium in which chondrocytes capable of hypertrophication derived from 4 week-old rat were cultured; 8 week-old growth supernatant: supernatant of MEM growth medium in which chondrocytes capable of hypertrophication derived from 8 week-old rat were cultured.

FIG. 3A shows a result of alkaline phosphatase staining of mouse C3H10T1/2 cells cultured by adding a supernatant of each of an MEM differentiation agent producing medium and an MEM growth medium in which chondrocytes capable of hypertrophication derived from costa/costal cartilage were cultured (culture supernatant).

The mouse C3H10T1/2 cells were inoculated in a 24-well plate (in a BME medium). Eighteen hours after the inoculation, the supernatant was added to the plate, and after 72 hours, the alkaline phosphatase staining was performed. Upper column: it was confirmed that samples, to which the supernatant of the MEM differentiation agent producing medium was added, were stained red and had alkaline phosphatase activities. Lower column: it was confirmed that samples, to which the supernatant of the MEM growth medium was added, were not stained and did not have the alkaline phosphatase activities.

FIG. 3B shows a result of alkaline phosphatase staining of mouse C3H10T1/2 cells cultured by adding a supernatant of an MEM differentiation agent producing medium in which chondrocytes capable of hypertrophication derived from costa/costal cartilage were cultured (culture supernatant).

The mouse C3H10T1/2 cells were inoculated on hydroxyapatite (in a BME medium). Eighteen hours after the inoculation, the supernatant was added to the hydroxyapatite, and after 72 hours, the alkaline phosphatase staining was performed. It was confirmed that a sample, to which the supernatant of the MEM differentiation agent producing medium was added, was stained red and had an alkaline phosphatase activity. In FIG. 3B, the length of the bar shown in the lower left thereof is 500.00 μm.

FIG. 3C shows a result of toluidine blue staining of mouse C3H10T1/2 cells cultured by adding a supernatant of an MEM differentiation agent producing medium in which chondrocytes capable of hypertrophication derived from costa/costal cartilage were cultured (culture supernatant).

The mouse C3H10T1/2 cells were inoculated on hydroxyapatite (in a BME medium). Eighteen hours after the inoculation, the supernatant was added to the hydroxyapatite, and after 72 hours, the toluidine blue staining was performed. It was confirmed that a sample was stained blue with the toluidine blue staining, and therefore cells were present in the sample. In FIG. 3C, the length of the bar shown in the lower left thereof is 500.00 μm.

FIG. 3D shows a result of alkaline phosphatase staining of mouse C3H10T1/2 cells cultured by adding a supernatant of an MEM growth medium in which chondrocytes capable of hypertrophication derived from costa/costal cartilage were cultured (culture supernatant).

The mouse C3H10T1/2 cells were inoculated on hydroxyapatite (in a BME medium). Eighteen hours after the inoculation, the supernatant was added to the hydroxyapatite, and after 72 hours, the alkaline phosphatase staining was performed. It was confirmed that a sample, to which the supernatant of the MEM growth medium was added, was not stained and did not have an alkaline phosphatase activity. In FIG. 3D, the length of the bar shown in the lower left thereof is 500.00 μm.

FIG. 3E shows a result of toluidine blue staining of mouse C3H10T1/2 cells cultured by adding a supernatant of an MEM growth medium in which chondrocytes capable of hypertrophication derived from costa/costal cartilage were cultured (culture supernatant).

The mouse C3H10T1/2 cells were inoculated on hydroxyapatite (in a BME medium). Eighteen hours after the inoculation, the supernatant was added to the hydroxyapatite, and after 72 hours, the toluidine blue staining was performed. It was confirmed that a sample was stained blue with the toluidine blue staining, and therefore cells were present in the sample. In FIG. 3E, the length of the bar shown in the lower left thereof is 500.00 μm.

FIG. 4 shows alkaline phosphatase activities of mouse C3H10T1/2 cells each measured by culturing resting cartilage cells derived from costal cartilage in an MEM differentiation agent producing medium and an MEM growth medium, respectively, collecting a supernatant of each of the mediums (culture supernatant) on a time course (4 days, 1 week, 2 weeks, 3 weeks) to obtain fractional supernatants, adding each of the fractional supernatants to the mouse C3H10T1/2 cells, and then culturing them.

There was little difference between a value of the alkaline phosphatase activity of the mouse C3H10T1/2 cells cultured by adding the supernatant of each of the MEM differentiation agent producing medium and the MEM growth medium (cultures of cells) and a value of the alkaline phosphatase activity thereof cultured by adding only each of the MEM differentiation agent producing medium and the MEM growth medium.

The following abbreviations show the added supernatants. 8 week-old differentiation supernatant: supernatant of MEM differentiation agent producing medium in which resting cartilage cells derived from 8 week-old rat were cultured; 8 week-old growth supernatant: supernatant of MEM growth medium in which resting cartilage cells derived from 8 week-old rat were cultured.

Each value is indicated by defining the value of the alkaline phosphatase activity of the mouse C3H10T1/2 cells cultured by adding only each of the MEM differentiation agent producing medium and the MEM growth medium as “1”.

FIG. 5A shows alkaline phosphatase activities of mouse C3H10T1/2 cells each measured by culturing chondrocytes derived from articular cartilage were cultured in an MEM differentiation agent producing medium and an MEM growth medium, respectively, collecting a supernatant of each of the mediums (culture supernatant) on a time course (4 days, 1 week, 2 weeks, 3 weeks) to obtain fractional supernatants, adding each of the fractional supernatants to the mouse C3H10T1/2 cells, and then culturing them.

There was little difference between a value of the alkaline phosphatase activity of the mouse C3H10T1/2 cells cultured by adding the supernatant of each of the MEM differentiation agent producing medium and the MEM growth medium in which the chondrocytes derived from articular cartilage were cultured (cultures of cells) and a value of the alkaline phosphatase activity thereof cultured by adding only each of the MEM differentiation agent producing medium and the MEM growth medium.

The following abbreviations show the added supernatants. 8 week-old differentiation supernatant: supernatant of MEM differentiation agent producing medium in which chondrocytes derived from 8 week-old rat articular cartilage were cultured; 8 week-old growth supernatant: supernatant of MEM growth medium in which chondrocytes derived from 8 week-old rat articular cartilage were cultured.

Each value is indicated by defining the value of the alkaline phosphatase activity of the mouse C3H10T1/2 cells cultured by adding only each of the MEM differentiation agent producing medium and the MEM growth medium as “1”.

FIG. 5B shows alkaline phosphatase activities of mouse C3H10T1/2 cells each measured by culturing chondrocytes capable of hypertrophication derived from costa/costal cartilage in a HAM differentiation agent producing medium, collecting a supernatant of the medium (culture supernatant) on a time course (4 days, 7 days, 14 days, 21 days) to obtain fractional supernatants, adding each of the fractional supernatants to the mouse C3H10T1/2 cells, and then culturing them.

Each value is indicated by defining a value of the alkaline phosphatase activity of the mouse C3H10T1/2 cells cultured by adding only the HAM differentiation agent producing medium as “1”. The alkaline phosphatase activity of the mouse C3H10T1/2 cells cultured by adding the supernatant of the HAM differentiation agent producing medium, in which the chondrocytes capable of hypertrophication derived from costa/costal cartilage were cultured, increased.

FIG. 5C shows alkaline phosphatase activities of mouse C3H10T1/2 cells each measured by culturing chondrocytes capable of hypertrophication derived from costa/costal cartilage in a HAM growth medium, collecting a supernatant of the medium (culture supernatant) on a time course (4 days, 7 days, 14 days, 21 days) to obtain fractional supernatants, adding each of the fractional supernatants to the mouse C3H10T1/2 cells, and then culturing them.

Each value is indicated by defining a value of the alkaline phosphatase activity of the mouse C3H10T1/2 cells cultured by adding only the HAM growth medium as “1”. The alkaline phosphatase activity of the mouse C3H10T1/2 cells cultured by adding the supernatant of the HAM growth medium, in which the chondrocytes capable of hypertrophication derived from costa/costal cartilage were cultured, did not increase.

FIG. 6A shows the presence of an agent capable of increasing an alkaline phosphatase activity of each kind of 3T3-Swiss albino cells and BALB/3T3 cells, and capable of inducing differentiation of each kind of these undifferentiated cells into induced osteoblasts, in a supernatant (culture supernatant) obtained by culturing chondrocytes capable of hypertrophication in an MEM differentiation agent producing medium.

On the other hand, FIG. 6A also shows the absence of the above agent in a supernatant (culture supernatant) obtained by culturing the chondrocytes capable of hypertrophication in an MEM growth medium. In addition, FIG. 6A also shows the absence of the above agent in a supernatant (culture supernatant) obtained by culturing chondrocytes incapable of hypertrophication in the MEM differentiation agent producing medium or the MEM growth medium.

FIG. 6B shows alkaline phosphatase activities of mouse C3H10T1/2 cells each measured by culturing chondrocytes capable of hypertrophication in a medium to which dexamethasone, β-glycerophosphate, ascorbic acid or a combination thereof was added as a conventional osteoblast differentiation inducing component, collecting a supernatant of the medium (culture supernatant), adding the supernatant to the mouse C3H10T1/2 cells, and then culturing them. Dex: dexamethasone, BGP: β-glycerophosphate, Asc: ascorbic acid.

FIG. 7A shows a result of alkaline phosphatase staining of mouse C3H10T1/2 cells inoculated in a 24-well plate and cultured by adding a fraction with a molecular weight of 50,000 or higher separated from a supernatant of an MEM differentiation agent producing medium, in which chondrocytes capable of hypertrophication derived from costa/costal cartilage were cultured, to the plate.

Samples each containing the mouse C3H10T1/2 cells were stained red with the alkaline phosphatase staining. Therefore, it was confirmed that an agent capable of increasing an alkaline phosphatase activity of the mouse C3H10T1/2 cells was present in the fraction with the molecular weight of 50,000 or higher.

FIG. 7B shows a result of alkaline phosphatase staining of mouse C3H10T1/2 cells inoculated on hydroxyapatite and cultured by adding a fraction with a molecular weight of 50,000 or higher separated from a supernatant of an MEM differentiation agent producing medium, in which chondrocytes capable of hypertrophication derived from costa/costal cartilage were cultured, to the hydroxyapatite.

A sample (hydroxyapatite) containing the mouse C3H10T1/2 cells was stained red. Therefore, it was confirmed that an agent capable of increasing an alkaline phosphatase activity of the mouse C3H10T1/2 cells was present in the fraction with the molecular weight of 50,000 or higher.

FIG. 7C shows a result of alkaline phosphatase staining of mouse C3H10T1/2 cells inoculated in a 24-well plate and cultured by adding a fraction with a molecular weight of lower than 50,000 separated from a supernatant of an MEM differentiation agent producing medium, in which chondrocytes capable of hypertrophication derived from costa/costal cartilage were cultured, to the plate.

An agent capable of increasing an alkaline phosphatase activity of the mouse C3H10T1/2 cells was not observed in the fraction with the molecular weight of lower than 50,000. In FIG. 7C, the length of the bar shown in the lower left thereof is 500.00 μm.

FIG. 7D shows a result of alkaline phosphatase staining of mouse C3H10T1/2 cells inoculated on hydroxyapatite and cultured by adding a fraction with a molecular weight of lower than 50,000 separated from a supernatant of an MEM differentiation agent producing medium, in which chondrocytes capable of hypertrophication derived from costa/costal cartilage were cultured, to the hydroxyapatite.

An agent capable of increasing an alkaline phosphatase activity of the mouse C3H10T1/2 cells was not observed in the fraction with the molecular weight of lower than 50,000. In FIG. 7D, the length of the bar shown in the lower left thereof is 500.00 μm.

FIG. 8 shows alkaline phosphatase activities of mouse C3H10T1/2 cells each measured by culturing each kind of chondrocytes capable of hypertrophication collected from mouse costa/costal cartilage and resting cartilage cells collected from costal cartilage in an MEM differentiation agent producing medium and an MEM growth medium, respectively, collecting a supernatant of each of the mediums (culture supernatant), adding the supernatant to the mouse C3H10T1/2 cells, and then culturing them.

A relative value of an alkaline phosphatase activity of the mouse C3H10T1/2 cells cultured by adding the supernatant of the MEM differentiation agent producing medium, in which the chondrocytes capable of hypertrophication were cultured, increased to 3.1 times.

There was little difference between a value of the alkaline phosphatase activity of the mouse C3H10T1/2 cells cultured by adding the supernatant of each of the MEM growth medium, in which the chondrocytes capable of hypertrophication were cultured, and the MEM differentiation agent producing and MEM growth mediums, in which the resting cartilage cells derived from costal cartilage were cultured, and a value of the alkaline phosphatase activity thereof cultured by adding only each of the MEM growth medium and the MEM differentiation agent producing medium.

The following abbreviations show the added supernatants. GC differentiation supernatant: supernatant of MEM differentiation agent producing medium in which chondrocytes capable of hypertrophication were cultured; GC growth supernatant: supernatant of MEM growth medium in which chondrocytes capable of hypertrophication were cultured; RC differentiation supernatant: supernatant of MEM differentiation agent producing medium in which resting cartilage cells were cultured; RC growth supernatant: supernatant of MEM growth medium in which resting cartilage cells were cultured.

Each value is indicated by defining the value of the alkaline phosphatase activity of the mouse C3H10T1/2 cells cultured by adding only each of the MEM differentiation agent producing medium and the MEM growth medium as “1”.

FIG. 9 shows effect of a medium for culturing undifferentiated cells (undifferentiated cell culture medium) on induction of differentiation of the undifferentiated cells into induced osteoblasts. Each kind of chondrocytes capable of hypertrophication, resting cartilage cells and articular cartilage cells were cultured in an MEM differentiation agent producing medium and an MEM growth medium, respectively. A supernatant of each of the mediums (culture supernatant) was added to mouse C3H10T1/2 cells, they were cultured, and then an alkaline phosphatase activity thereof was measured. A HAM medium or an MEM medium was used as a medium for culturing the mouse C3H10T1/2 cells.

In the case where the HAM medium was used for culturing the mouse C3H10T1/2 cells, it was observed that the alkaline phosphatase activity thereof cultured only by adding the supernatant of the MEM differentiation agent producing medium, in which the chondrocytes capable of hypertrophication were cultured, increased. In the case where the MEM medium was also used for culturing the mouse C3H10T1/2 cells, the same result was observed.

The following abbreviations show the added supernatants. GC differentiation supernatant: supernatant of MEM differentiation agent producing medium in which chondrocytes capable of hypertrophication were cultured; GC growth supernatant: supernatant of MEM growth medium in which chondrocytes capable of hypertrophication were cultured; RC differentiation supernatant: supernatant of MEM differentiation agent producing medium in which resting cartilage cells were cultured; RC growth supernatant: supernatant of MEM growth medium in which resting cartilage cells were cultured; AC differentiation supernatant: supernatant of MEM differentiation agent producing medium in which articular cartilage cells were cultured; AC growth supernatant: supernatant of MEM growth medium in which articular cartilage cells were cultured.

Each value is indicated by defining a value of the alkaline phosphatase activity of the mouse C3H10T1/2 cells cultured by adding only each of the MEM differentiation agent producing medium and the MEM growth medium as “1”.

FIG. 10 shows an alkaline phosphatase activity of mouse C3H10T1/2 cells cultured by adding an agent capable of inducing differentiation of undifferentiated cells into induced osteoblasts, which was produced by chondrocytes capable of hypertrophication, after being heated. A supernatant of an MEM differentiation agent producing medium, in which the chondrocytes capable of hypertrophication were cultured, (culture supernatant) was subjected to a heat treatment for 3 minutes in boiling water.

Each of the supernatant not subjected to the heat treatment, the supernatant subjected to the heat treatment and the MEM differentiation agent producing medium alone was added to the mouse C3H10T1/2 cells, and after 72 hours, the alkaline phosphatase activity thereof was measured. The alkaline phosphatase activity of the mouse C3H10T1/2 cells cultured by adding the supernatant subjected to the heat treatment did not increase. Therefore, it was confirmed that the agent capable of inducing differentiation of undifferentiated cells into induced osteoblasts was degenerated (inactivated) by the heat treatment.

The following abbreviations show the added supernatants. GC heat treatment: supernatant of MEM differentiation agent producing medium, in which chondrocytes capable of hypertrophication were cultured, subjected to heat treatment; GC differentiation supernatant: supernatant of MEM differentiation agent producing medium in which chondrocytes capable of hypertrophication were cultured; Only differentiation supernatant: MEM differentiation agent producing medium alone.

Each value is indicated by defining a value of the alkaline phosphatase activity of the mouse C3H10T1/2 cells cultured by adding only the MEM differentiation agent producing medium as “1”.

FIG. 11A shows a TGF activity in a supernatant of an MEM differentiation agent producing medium containing an induced osteoblast differentiation inducing agent.

FIG. 11B shows a BMP activity in a supernatant of an MEM differentiation agent producing medium containing an induced osteoblasts differentiation inducing agent.

FIG. 12A shows a roentgenogram (left side) and a micro-computerized tomography image (right side) of a pellet of C3H10T1/2 cells. The pellet was formed from 5×10⁵ C3H10T1/2 cells, cultured in a medium containing a supernatant of a differentiation agent producing medium, in which chondrocytes capable of hypertrophication were cultured, for 1 week, and then implanted under the dorsal skin of C3H mouse (individual number

Four weeks after the implantation, the pellet was surgically removed, and then roentgenographed and imaged using a micro-computerized tomography. In this regard, the imaging was performed because a shadow had been observed in the roentgenogram of the pellet.

FIG. 12B shows a roentgenogram (left side) and a micro-computerized tomography image (right side) of a pellet of the C3H10T1/2 cells implanted in another recipient mouse (individual number 2) in the same condition as FIG. 12A.

FIG. 12C shows a roentgenogram (left side) and a micro-computerized tomography image (right side) of a pellet of the C3H10T1/2 cells implanted in another recipient mouse (individual number 3) in the same condition as FIG. 12A.

FIG. 12D shows a roentgenogram (left side) and a micro-computerized tomography image (right side) of a pellet of C3H10T1/2 cells. The pellet was formed from 5×10⁵ C3H10T1/2 cells, cultured in a medium containing a supernatant of a differentiation agent producing medium, in which chondrocytes incapable of hypertrophication were cultured, for 1 week, and then implanted under the dorsal skin of the C3H mouse (individual number 1 used for obtaining FIG. 12A).

Four weeks after the implantation, the pellet was surgically removed, and then roentgenographed and imaged using a micro-computerized tomography. In this regard, the imaging was performed because a shadow had been observed in the roentgenogram of the pellet.

FIG. 12E shows roentgenograms of pellets of the C3H10T1/2 cells implanted in the other recipient mice (individual numbers 2 and 3 used for obtaining FIGS. 12B and 12C), respectively, in the same condition as FIG. 12D. In this regard, the micro-computerized tomography imaging was not performed because shadows had not been observed in the roentgenograms of the pellets.

FIGS. 13A to 13D show preimplantation composite materials each produced using collagen gel and chondrocytes capable of hypertrophication. FIG. 13A shows a sample subjected to HE staining (20-fold visual field in magnification of ocular). FIG. 13B shows a sample subjected to TB staining (20-fold visual field in magnification of ocular). FIG. 13C shows a sample subjected to AB staining (20-fold visual field in magnification of ocular). FIG. 13D shows a sample subjected to SO staining (20-fold visual field in magnification of ocular).

FIG. 13E shows a roentgenogram of an implanted region obtained by implanting a composite material produced using collagen gel and chondrocytes capable of hypertrophication under the dorsal skin of rat, and then surgically removing it 4 weeks after the implantation. In this regard, a circular image is an image of a silicone ring embedded in the rat for determining the implanted region. Calcification is confirmed at an inside central portion of the ring.

FIG. 13F shows a micro-computerized tomography image obtained by imaging the same sample used for obtaining FIG. 13E. In this regard, a circular image is an image of the silicone ring embedded in the rat for determining the implanted region. Calcification is confirmed at an inside central portion of the ring.

FIGS. 14A to 14D show whole images of tissues each obtained by implanting a composite material produced using collagen gel and chondrocytes capable of hypertrophication under the dorsal skin of rat, surgically removing an implanted region 4 weeks after the implantation, and then staining it.

FIG. 14A shows the implanted region subjected to HE staining (35-fold visual field in magnification of magnifier). FIG. 14B shows the implanted region subjected to TB staining (35-fold visual field in magnification of magnifier). FIG. 14C shows the implanted region subjected to AB staining (35-fold visual field in magnification of magnifier). FIG. 14D shows the implanted region subjected to SO staining (35-fold visual field in magnification of magnifier).

FIGS. 15A to 15D show enlarged views (4-fold visual field in magnification of ocular) of histologies each obtained by implanting the composite material produced using the collagen gel and the chondrocytes capable of hypertrophication and shown in each of FIGS. 13A to 13D under the dorsal skin of rat, surgically removing an implanted region 4 weeks after the implantation, and then staining it. FIGS. 15A to 15D correspond to FIGS. 14A to 14D.

FIGS. 16A to 16D show enlarged views (10-fold visual field in magnification of ocular) of histologies each obtained by implanting the composite material produced using the collagen gel and the chondrocytes capable of hypertrophication and shown in each of FIGS. 13A to 13D under the dorsal skin of rat, surgically removing an implanted region 4 weeks after the implantation, and then staining it. FIGS. 16A to 16D correspond to FIGS. 14A to 14D.

FIGS. 17A to 17D show preimplantation composite materials each produced using alginic acid and chondrocytes capable of hypertrophication. FIG. 17A shows a sample subjected to HE staining (20-fold visual field in magnification of ocular). FIG. 17B shows a sample subjected to TB staining (20-fold visual field in magnification of ocular). FIG. 17C shows a sample subjected to AB staining (20-fold visual field in magnification of ocular). FIG. 17D shows a sample subjected to SO staining (20-fold visual field in magnification of ocular).

FIG. 17E shows a roentgenogram of an implanted region obtained by implanting a composite material produced using alginic acid and chondrocytes capable of hypertrophication under the dorsal skin of rat, and then surgically removing it 4 weeks after the implantation. In this regard, a circular image is an image of a silicone ring and calcification is confirmed at an inside central portion of the ring.

FIG. 17F shows a micro-computerized tomography image obtained by imaging the same sample used for obtaining FIG. 17E. In this regard, a circular image is an image of the silicone ring and calcification is confirmed at an inside central portion of the ring.

FIGS. 18A to 18D show whole images of tissues each obtained by implanting a composite material produced using alginic acid and chondrocytes capable of hypertrophication under the dorsal skin of rat, surgically removing an implanted region 4 weeks after the implantation, and then staining it.

FIG. 18A shows the implanted region subjected to HE staining (35-fold visual field in magnification of magnifier). FIG. 18B shows the implanted region subjected to TB staining (35-fold visual field in magnification of magnifier). FIG. 18C shows the implanted region subjected to AB staining (35-fold visual field in magnification of magnifier). FIG. 18D shows the implanted region subjected to SO staining (35-fold visual field in magnification of magnifier.

FIGS. 19A to 19D show enlarged views (4-fold visual field in magnification of ocular) of histologies each obtained by implanting the composite material produced using the alginic acid and the chondrocytes capable of hypertrophication and shown in each of FIGS. 17A to 17D under the dorsal skin of rat, surgically removing an implanted region 4 weeks after the implantation, and then staining it. FIGS. 19A to 19D correspond to FIGS. 18A to 18D.

FIGS. 20A to 20D show enlarged views (10-fold visual field in magnification of ocular) of histologies each obtained by implanting the composite material produced using the alginic acid and the chondrocytes capable of hypertrophication and shown in each of FIGS. 17A to 17D under the dorsal skin of rat, surgically removing an implanted region 4 weeks after the implantation, and then staining it. FIGS. 20A to 20D correspond to FIGS. 18A to 18D.

FIGS. 21A to 21D show preimplantation composite materials each produced using Matrigel and chondrocytes capable of hypertrophication. FIG. 21A shows a sample subjected to HE staining (20-fold visual field in magnification of ocular). FIG. 21B shows a sample subjected to TB staining (20-fold visual field in magnification of ocular). FIG. 21C shows a sample subjected to AB staining (20-fold visual field in magnification of ocular). FIG. 21D shows a sample subjected to SO staining (20-fold visual field in magnification of ocular).

FIG. 21E shows a roentgenogram of an implanted region obtained by implanting a composite material produced using Matrigel and chondrocytes capable of hypertrophication under the dorsal skin of rat, and then surgically removing it 4 weeks after the implantation. In this regard, a circular image is an image of a silicone ring and calcification is confirmed at an inside central portion of the ring.

FIG. 21F shows a micro-computerized tomography image obtained by imaging the same sample used for obtaining FIG. 21E. In this regard, a circular image is an image of the silicone ring and calcification is confirmed at an inside central portion of the ring.

FIGS. 22A to 22D show whole images of tissues each obtained by implanting a composite material produced using Matrigel and chondrocytes capable of hypertrophication under the dorsal skin of rat, surgically removing an implanted region 4 weeks after the implantation, and then staining it.

FIG. 22A shows the implanted region subjected to HE staining (35-fold visual field in magnification of magnifier). FIG. 22B shows the implanted region subjected to TB staining (35-fold visual field in magnification of magnifier). FIG. 22C shows the implanted region subjected to AB staining (35-fold visual field in magnification of magnifier). FIG. 22D shows the implanted region subjected to SO staining (35-fold visual field in magnification of magnifier).

FIGS. 23A to 23D show enlarged views (4-fold visual field in magnification of ocular) of histologies each obtained by implanting the composite material produced using the Matrigel and the chondrocytes capable of hypertrophication and shown in each of FIGS. 22A to 22D under the dorsal skin of rat, surgically removing an implanted region 4 weeks after the implantation, and then staining it. FIGS. 23A to 23D correspond to FIGS. 22A to 22D.

FIGS. 24A to 24D show enlarged views (10-fold visual field in magnification of ocular) of histologies each obtained by implanting the composite material produced using the Matrigel and the chondrocytes capable of hypertrophication and shown in each of FIGS. 22A to 22D under the dorsal skin of rat, surgically removing an implanted region 4 weeks after the implantation, and then staining it. FIGS. 24A to 24D correspond to FIGS. 22A to 22D.

FIGS. 25A to 25D show preimplantation composite materials each produced using collagen gel and chondrocytes incapable of hypertrophication. FIG. 25A shows a sample subjected to HE staining (20-fold visual field in magnification of ocular). FIG. 25B shows a sample subjected to TB staining (20-fold visual field in magnification of ocular). FIG. 25C shows a sample subjected to AB staining (20-fold visual field in magnification of ocular). FIG. 25D shows a sample subjected to SO staining (20-fold visual field in magnification of ocular).

FIG. 25E shows a roentgenogram of an implanted region obtained by implanting a composite material produced using collagen gel and chondrocytes incapable of hypertrophication under the dorsal skin of rat, and then surgically removing it 4 weeks after the implantation. In this regard, a circular image is an image of a silicone ring and calcification is not confirmed at an inside central portion of the ring.

FIG. 25F shows a micro-computerized tomography image obtained by imaging the same sample used for obtaining FIG. 25E. In this regard, a circular image is an image of the silicone ring and calcification is not confirmed at an inside central portion of the ring.

FIGS. 26A to 26D show whole images of tissues each obtained by implanting a composite material produced using collagen gel and chondrocytes incapable of hypertrophication under the dorsal skin of rat, surgically removing an implanted region 4 weeks after the implantation, and then staining it.

FIG. 26A shows the implanted region subjected to HE staining (35-fold visual field in magnification of magnifier). FIG. 26B shows the implanted region subjected to TB staining (35-fold visual field in magnification of magnifier). FIG. 26C shows the implanted region subjected to AB staining (35-fold visual field in magnification of magnifier). FIG. 26D shows the implanted region subjected to SO staining (35-fold visual field in magnification of magnifier).

FIGS. 27A to 27D show enlarged views (4-fold visual field in magnification of ocular) of histologies each obtained by implanting the composite material produced using the collagen gel and the chondrocytes incapable of hypertrophication and shown in each of FIGS. 26A to 26D under the dorsal skin of rat, surgically removing an implanted region 4 weeks after the implantation, and then staining it. FIGS. 27A to 27D correspond to FIGS. 26A to 26D.

FIGS. 28A to 28D show preimplantation composite materials each produced using alginic acid and chondrocytes incapable of hypertrophication. FIG. 28A shows a sample subjected to HE staining (20-fold visual field in magnification of ocular). FIG. 28B shows a sample subjected to TB staining (20-fold visual field in magnification of ocular). FIG. 28C shows a sample subjected to AB staining (20-fold visual field in magnification of ocular). FIG. 28D shows a sample subjected to SO staining (20-fold visual field in magnification of ocular).

FIG. 28E shows a roentgenogram of an implanted region obtained by implanting a composite material produced using alginic acid and chondrocytes incapable of hypertrophication under the dorsal skin of rat, and then surgically removing it 4 weeks after the implantation. In this regard, a circular image is an image of a silicone ring and calcification is not confirmed at an inside central portion of the ring.

FIG. 28F shows a micro-computerized tomography image obtained by imaging the same sample used for obtaining FIG. 28E. In this regard, a circular image is an image of the silicone ring and calcification is not confirmed at an inside central portion of the ring.

FIGS. 29A to 29D show whole images of tissues each obtained by implanting a composite material produced using alginic acid and chondrocytes incapable of hypertrophication under the dorsal skin of rat, surgically removing an implanted region 4 weeks after the implantation, and then staining it.

FIG. 29A shows the implanted region subjected to HE staining (35-fold visual field in magnification of magnifier). FIG. 29B shows the implanted region subjected to TB staining (35-fold visual field in magnification of magnifier). FIG. 29C shows the implanted region subjected to AB staining (35-fold visual field in magnification of magnifier). FIG. 29D shows the implanted region subjected to SO staining (35-fold visual field in magnification of magnifier).

FIGS. 30A to 30D show enlarged views (4-fold visual field in magnification of ocular) of histologies each obtained by implanting the composite material produced using the alginic acid and the chondrocytes incapable of hypertrophication and shown in each of FIGS. 28A to 28D under the dorsal skin of rat, surgically removing an implanted region 4 weeks after the implantation, and then staining it. FIGS. 30A to 30D correspond to FIGS. 29A to 29D.

FIGS. 31A to 31D show preimplantation composite materials each produced using Matrigel and chondrocytes incapable of hypertrophication. FIG. 31A shows a sample subjected to HE staining (20-fold visual field in magnification of ocular). FIG. 31B shows a sample subjected to TB staining (20-fold visual field in magnification of ocular). FIG. 31C shows a sample subjected to AB staining (20-fold visual field in magnification of ocular). FIG. 31D shows a sample subjected to SO staining (20-fold visual field in magnification of ocular).

FIG. 31E shows a roentgenogram of an implanted region obtained by implanting a composite material produced using Matrigel and chondrocytes incapable of hypertrophication under the dorsal skin of rat, and then surgically removing it 4 weeks after the implantation. In this regard, a circular image is an image of a silicone ring and calcification is not confirmed at an inside central portion of the ring.

FIG. 31F shows a micro-computerized tomography image obtained by imaging the same sample used for obtaining FIG. 31E. In this regard, a circular image is an image of the silicone ring and calcification is not confirmed at an inside central portion of the ring.

FIGS. 32A to 32D show whole images of tissues each obtained by implanting a composite material produced using Matrigel and chondrocytes incapable of hypertrophication under the dorsal skin of rat, surgically removing an implanted region 4 weeks after the implantation, and then staining it.

FIG. 32A shows the implanted region subjected to HE staining (35-fold visual field in magnification of magnifier). FIG. 32B shows the implanted region subjected to TB staining (35-fold visual field in magnification of magnifier. FIG. 32C shows the implanted region subjected to AB staining (35-fold visual field in magnification of magnifier). FIG. 32D shows the implanted region subjected to SO staining (35-fold visual field in magnification of magnifier).

FIGS. 33A to 33D show enlarged views (4-fold visual field in magnification of ocular) of histologies each obtained by implanting the composite material produced using the Matrigel and the chondrocytes incapable of hypertrophication and shown in each of FIGS. 31A to 31D under the dorsal skin of rat, surgically removing an implanted region 4 weeks after the implantation, and then staining it. FIGS. 33A to 33D correspond to FIGS. 32A to 32D.

FIG. 34A shows a roentgenogram of an implanted region obtained by implanting only hydroxyapatite under the dorsal skin of rat, and then surgically removing it 4 weeks after the implantation. In FIG. 34A, the length of the bar shown in the upper left thereof is 100.00 μm. FIG. 34B shows an enlarged view (20-fold visual field in magnification of ocular) of FIG. 34A.

FIG. 34C shows a roentgenogram of an implanted region obtained by implanting only collagen gel under the dorsal skin of rat, and then surgically removing it 4 weeks after the implantation. FIG. 34D shows a micro-computerized tomography image obtained by imaging the same sample used for obtaining FIG. 34C.

FIG. 34E shows a roentgenogram of an implanted region obtained by implanting only alginic acid under the dorsal skin of rat, and then surgically removing it 4 weeks after the implantation. FIG. 34F shows a micro-computerized tomography image obtained by imaging the same sample used for obtaining FIG. 34E.

FIG. 34G shows a roentgenogram of an implanted region obtained by implanting only Matrigel under the dorsal skin of rat, and then surgically removing it 4 weeks after the implantation. FIG. 34H shows a micro-computerized tomography image obtained by imaging the same sample used for obtaining FIG. 34G.

In this regard, in each of FIGS. 34C to 34H, a circular image is an image of the silicone ring. Calcification is not confirmed in all scaffolds.

FIG. 35A shows chondrocytes capable of hypertrophication derived from rat costa formed into a pellet and cultured (35-fold visual field in magnification of magnifier). In FIG. 35A, an enlarged tissue is observed. FIG. 35B shows chondrocytes incapable of hypertrophication derived from rat costa formed into a pellet and cultured (35-fold visual field in magnification of magnifier). In FIG. 35B, it is confirmed that the chondrocytes incapable of hypertrophication are not enlarged.

FIG. 35C shows a roentgenogram of an implanted region obtained by forming a pellet from chondrocytes capable of hypertrophication derived from rat costa, implanting it under the dorsal skin of rat, and then surgically removing it 4 weeks after the implantation. In this regard, a circular image is an image of a silicone ring and calcification is confirmed at an inside central portion of the ring.

FIG. 35D shows a micro-computerized tomography image obtained by imaging the same sample used for obtaining FIG. 35C. In this regard, a circular image is an image of the silicone ring and calcification is confirmed at an inside central portion of the ring.

FIG. 35E shows a roentgenogram an implanted region obtained by forming a pellet from chondrocytes incapable of hypertrophication derived from rat costa, implanting it under the dorsal skin of rat, surgically removing it 4 weeks after the implantation. In this regard, a circular image is an image of a silicone ring and calcification is not confirmed at an inside central portion of the ring.

FIG. 35F shows a micro-computerized tomography image obtained by imaging the same sample used for obtaining FIG. 35E. In this regard, a circular image is an image of the silicone ring and calcification is not confirmed at an inside central portion of the ring.

FIGS. 36A to 36D show histologies each obtained by forming a pellet from chondrocytes capable of hypertrophication derived from rat costa, implanting it under the dorsal skin of rat, surgically removing an implanted region 4 weeks after the implantation, and then staining it.

FIG. 36A shows the implanted region subjected to HE staining (4-fold visual field in magnification of ocular). FIG. 36B shows the implanted region subjected to TB staining (4-fold visual field in magnification of ocular). FIG. 36C shows the implanted region subjected to AB staining (4-fold visual field in magnification of ocular). FIG. 36D shows the implanted region subjected to SO staining (4-fold visual field in magnification of ocular).

FIGS. 37A to 37D show enlarged views (10-fold visual field in magnification of ocular) of histologies each obtained by implanting the pellet formed from the chondrocytes capable of hypertrophication derived from rat costa and shown in FIG. 35A under the dorsal skin of rat, surgically removing an implanted region 4 weeks after the implantation, and then staining it. FIGS. 37A to 37D correspond to FIGS. 36A to 36D.

FIGS. 38A to 38D show histologies each obtained by forming a pellet from chondrocytes incapable of hypertrophication derived from rat costa, implanting it under the dorsal skin of rat, surgically removing an implanted region 4 weeks after the implantation, and then staining it.

FIG. 38A shows the implanted region subjected to HE staining (4-fold visual field in magnification of ocular). FIG. 38B shows the implanted region subjected to TB staining (4-fold visual field in magnification of ocular). FIG. 38C shows the implanted region subjected to AB staining (4-fold visual field in magnification of ocular). FIG. 38D shows the implanted region subjected to SO staining (4-fold visual field in magnification of ocular).

FIG. 39(1) shows a photograph of alkaline phosphatase staining of human undifferentiated mesenchymal stem cells cultured by adding a supernatant of an agent producing medium in which chondrocytes capable of hypertrophication were cultured (differentiation medium containing an agent). It was confirmed that the human undifferentiated mesenchymal stem cells were stained red.

FIG. 39(2) shows a photograph of alkaline phosphatase staining of human undifferentiated mesenchymal stem cells cultured by adding only an MEM differentiation agent producing medium, that is, a medium containing no agent according to the present invention but containing dexamethasone (Maniatopoulos's osteoblast differentiation medium). It was confirmed that the human undifferentiated mesenchymal stem cells were slightly stained red.

FIG. 39(3) shows a photograph of alkaline phosphatase staining of human undifferentiated mesenchymal stem cells cultured by adding only an MEM growth medium (containing no agent and no dexamethasone). It was confirmed that the human undifferentiated mesenchymal stem cells were hardly stained. In this regard, each of the above operations was performed three times.

### DETAILED DESCRIPTION OF THE INVENTION

The present invention is described hereinafter. It is to be understood that, unless otherwise described, singular representations throughout the present specification include the concept of plural thereof. Therefore, it is to be understood that, unless particularly described, singular articles (for example, in the case of the English language, “a”, “an”, “the” and the like) include the concept of plural.

It should be also understood that terms used herein have the definitions ordinarily used in the art unless otherwise mentioned. Therefore, unless otherwise defined, all technical and scientific terms used herein have the same meaning as that commonly understood by those skilled in the art. Otherwise, the present application (including definitions) takes precedence.

(Definition of Term)

The definitions of the terms particularly used herein are listed below.

A “composite material” used herein refers to a material containing cells and a scaffold.

Examples of a “bone defect” used herein include lesions such as bone tumors, osteoporosis, rheumatoid arthritis, osteoarthritis, osteomyelitis and osteonecrosis; correction such as immobilization of bone, foraminotomy and osteotomy; trauma such as complex fracture; bone defects derived from collecting ilium; and the like, but are not limited thereto.

“Promotion” of osteogenesis used herein means that in the case where a desired change was applied to a region where the osteogenesis has been induced, a rate thereof increases. “Induction” of the osteogenesis means that in the case where a desired change was applied to a region where the osteogenesis has not been induced, the osteogenesis is induced.

“Repair” of a bone defective region means that the bone defective region becomes a normal state or comes close to such a state.

A “size that is not repaired only by immobilization” used herein refers to a size that needs to use an implant or a bone supply material.

(Cells)

“Growth cartilage cells” or “growth chondrocytes” interchangeably used herein refers to cells in a tissue (i.e., growth cartilage) which forms bone during a developmental or growth stage, and a period of bone recovery or proliferation. The growth cartilage generally refers to a tissue which forms the bone during the growth stage, while it herein means a tissue which forms the bone during the developmental or growth stage, and the period of bone proliferation or recovery.

The growth cartilage cells (growth chondrocytes) are also referred to as chondrocytes capable of hypertrophication, chondrocytes capable of calcification or epiphysial (line) chondrocytes. In the case of using the growth cartilage cells in human, cells derived from human are preferred, but it is also possible to use non-human cells since problems such as immunological rejection can be avoided using techniques well-known in the art.

The growth cartilage cells according to the present invention are derived from a mammal, preferably, derived from human, mouse, rat or rabbit.

The growth cartilage cells according to the present invention may be collected from a chondro-osseous junction of costa, an epiphysial line of long bone (e.g., femur, tibia, fibula, humerus, ulna, radius), an epiphysial line of vertebra, a zone of proliferating cartilage of hand bone, foot bone, breast bone and others, perichondrium, bone primordium formed from cartilage of fetus, a callus region of a healing bone-fracture, and a cartilaginous part of a bone proliferation phase. These growth cartilage cells may be prepared, for example, using methods described in Examples of the present specification.

“Chondrocytes capable of hypertrophication” used herein refer to cells which can undergo hypertrophic growth in the future. The chondrocytes capable of hypertrophication include any other cells capable of hypertrophication determined by a method of determining an “ability of hypertrophication” defined hereinafter in addition to “growth cartilage cells” collected from a biological organism.

The chondrocytes capable of hypertrophication according to the present invention are derived from a mammal, preferably, derived from human, mouse, rat or rabbit. In the case of using the chondrocytes capable of hypertrophication in human, cells derived from human are preferred, but it is also possible to use non-human cells since problems such as immunological rejection can be avoided using techniques well-known in the art.

The chondrocytes capable of hypertrophication according to the present invention may be obtained, for example, from a chondro-osseous junction of costa, an epiphysial line of long bone (e.g., femur, tibia, fibula, humerus, ulna, radius), an epiphysial line of vertebra, a zone of proliferating cartilage of hand bone, foot bone, breastbone and others, perichondrium, bone primordium formed from cartilage of fetus, a callus region of a healing bone-fracture, and a cartilaginous part of a bone proliferation phase.

The chondrocytes capable of hypertrophication according to the present invention also can be obtained by inducing differentiation of undifferentiated cells.

The chondrocytes capable of hypertrophication according to the present invention may be chondrocytes collected from any regions other than the above-described regions. Because bone formed by endochondral ossification (enchondral ossification) is formed by the same mechanism irrespective of the regions of the body. In other words, cartilage is formed and substituted with the bone.

A major part of bone other than cranium and clavicle is formed by the endochondral ossification (enchondral ossification). Therefore, the chondrocytes capable of hypertrophication exist in the major part of the bone other than the cranium and the clavicle of the body. The chondrocytes capable of hypertrophication have abilities capable of inducing osteogenesis.

The chondrocytes capable of hypertrophication may be morphologically characterized by hypertrophy.

“Hypertrophy” used herein may be determined morphologically under a microscope. In the case where cells are arranged in a columnar manner to form a growth layer, the hypertrophy of cells is observed next to the growth layer. On the other hand, in the case where cells are not arranged in a columnar manner, the hypertrophy of cells means a state that they have larger sizes than those of marginal cells.

The ability of hypertrophication is identified by centrifuging a HAM's F12 culture solution containing 5×10⁵ cells to form a pellet of the cells, culturing the pellet for a pre-determined period, and then comparing sizes of the cells before and after the culture under a microscope. In this comparison, in the case where a significant increase in sizes thereof is observed, the cells are determined to have the ability of hypertrophication.

“Resting cartilage cells” or “resting chondrocytes” interchangeably used herein refer to cartilage cells or chondrocytes located in a region apart from a chondro-osseous junction of costa (zone of proliferating cartilage). The region is a tissue that exists as cartilage throughout an entire lifetime thereof. Cells located in resting cartilage are referred to as the resting cartilage cells. “Articular cartilage cells” used herein refer to cells in a cartilaginous tissue (articular cartilage) located on an articular surface.

The chondrocytes used herein are determined by identifying expression of at least one marker selected from the group consisting of type II collagen, cartilage proteoglycan (aglycan) or components thereof, hyaluronic acid, type IX collagen, type XI collagen, and chondromodulin. Among the chondrocytes, cells capable of hypertrophication are further determined by identifying expression of at least one marker selected from the group consisting of type X collagen, alkaline phosphatase, and osteonectin. Chondrocytes, which do not express any of the type X collagen, the alkaline phosphatase or the osteonectin, are determined to have no ability of hypertrophication.

Therefore, the chondrocytes capable of hypertrophication used herein also may be determined by identifying expression of at least one selected from markers for chondrocytes and at least one selected from markers for chondrocytes capable of hypertrophication, instead of the identification of the morphological hypertrophy. Localization or expression of these markers is identified by any method of analyzing proteins or RNAs extracted from cultured cells, such as a specific staining method, an immunohistochemical method, an in situ hybridization method, a Western blotting method or a PCR method.

A “chondrocyte marker (marker for chondrocytes)” used herein refers to any substance whose localization or expression in the chondrocytes aids in the identification thereof. Preferably, it refers to any substance which can be used for identifying the chondrocytes by its localization or expression (for example, localization or expression of type II collagen, cartilage proteoglycan (aglycan) or components thereof, hyaluronic acid, type IX collagen, type XI collagen, or chondromodulin).

A “marker for chondrocytes capable of hypertrophication” used herein refers to any substance whose localization or expression in the chondrocytes capable of hypertrophication aids in the identification thereof. Preferably, it refers to any substance which can be used for identifying the chondrocytes capable of hypertrophication by its localization or expression (for example, localization or expression of type X collagen, alkaline phosphatase or osteonectin).

“Cartilage proteoglycan” used herein refers to a macromolecule composed of a core protein and a plurality of glucosaminoglycans bonded to the core protein. Examples of the glucosaminoglycans include chondroitin tetrasulfate, chondroitin hexasulfate, keratan sulfate, O-linked oligosaccharide, N-linked oligosaccharide and the like. The cartilage proteoglycan is further bonded to hyaluronic acid via a linkage protein to form a cartilage proteoglycan aggregate. In a cartilaginous tissue, the glucosaminoglycan is rich and occupies 20 to 40% of a dry weight of the tissue. The cartilage proteoglycan is also referred to as aglycan.

“Bone proteoglycan” used herein refers to a macromolecule having a smaller molecular weight than that of the cartilage proteoglycan and composed of a core protein and glucosaminoglycans bonded to the core protein. Examples of the glucosaminoglycans include chondroitin sulfate, dermatan sulfate, O-linked oligosaccharide, N-linked oligosaccharide and the like. In a bone tissue, the glucosaminoglycan occupies 1% or less of a dry weight of decalcified bone. Examples of the bone proteoglycan include decorin and biglycan.

“Osteoblasts” used herein refer to cells which locate on a bone matrix, and form and calcifie the bone matrix. The osteoblasts are cells each having a size of 20 to 30 μm and being of a cubic or columnar form. As used herein, the osteoblasts may include “preosteoblasts” which are precursor cells of the osteoblasts.

The osteoblasts are determined by identifying expression of at least one marker selected from the group consisting of type I collagen, bone proteoglycan (e.g., decorin, biglycan), alkaline phosphatase, osteocalcin, matrix Gla protein, osteoglycin, osteopontin, bone sialic acid protein, osteonectin and pleiotrophin. In addition, the osteoblasts may be determined by identifying no expression of the chondrocyte marker such as the type II collagen, the cartilage proteoglycan (aglycan) or the components thereof, the hyaluronic acid, the type IX collagen, the type XI collagen, or the chondromodulin.

Localization or expression of these markers is identified by any method of analyzing proteins or RNAs extracted from cultured cells, such as a specific staining method, an immunohistochemical method, an in situ hybridization method, a Western blotting method or a PCR method.

An “osteoblast marker (marker for osteoblasts)” used herein refers to any substance whose localization or expression in the osteoblasts aids in the identification thereof. Preferably, it refers to any substance which can be used for identifying the osteoblasts by its localization or expression (for example, localization or expression of type I collagen, bone proteoglycan (e.g., decorin, biglycan), alkaline phosphatase, osteocalcin, matrix Gla protein, osteoglycin, osteopontin, bone sialic acid protein, osteonectin or pleiotrophin).

The osteoglycin is referred to as an osteoinductive factor (OIF). The osteopontin is referred to as BSP-I or 2ar. The bone sialic acid protein is referred to as BSP-II. The pleiotrophin is referred to as osteoblast specific protein or an osteoblast specific factor-1 (OSF-1). The osteonectin is referred to as SPARC or BM-40.

The osteoblasts maybe identified, for example, by: determining cells to be positive for a marker that identifies only the osteoblasts; determining cells to be positive for a marker that identifies the osteoblasts and the chondrocytes capable of hypertrophication and does not identify the chondrocytes, and to be positive for a marker that identifies the osteoblasts and the chondrocytes and does not identify the chondrocytes capable of hypertrophication; determining cells to be positive for a marker that identifies the osteoblasts and the chondrocytes capable of hypertrophication, but to be negative for a marker that does not identify the osteoblasts and identify the chondrocytes capable of hypertrophication; or determining cells to be positive for a marker that identifies the osteoblasts and the chondrocytes, but to be negative for a marker that does not identify the osteoblasts and identifies the chondrocytes.

The chondrocytes capable of hypertrophication may be identified, for example, by: determining cells to be positive for a marker that identifies only the chondrocytes capable of hypertrophication; determining cells to be positive for a marker that identifies the chondrocytes capable of hypertrophication and the osteoblasts and does not identify the chondrocytes, and to be positive for a marker that identifies the chondrocytes capable of hypertrophication and the chondrocytes and does not identify the osteoblasts; determining cells to be positive for a marker that identify the chondrocytes capable of hypertrophication and the osteoblasts, but to be negative for a marker that does not identify the chondrocytes capable of hypertrophication and identifies the osteoblasts; or determining cells to be positive for a marker that identifies the chondrocytes capable of hypertrophication and the chondrocytes, but to be negative for a marker that does not identify the chondrocytes capable of hypertrophication and identifies the chondrocytes.

Chondrocytes (incapable of hypertrophication) may be identified, for example, by: determining cells to be positive for a marker that identifies only the chondrocytes; determining cells to be positive for a marker that identifies the chondrocytes and the osteoblasts and does not identify the chondrocytes capable of hypertrophication, and to be positive for a marker that identifies the chondrocytes and the chondrocytes capable of hypertrophication and does not identify the osteoblasts; determining cells to be positive for a marker that identify the chondrocytes and the osteoblasts, but to be negative for a marker that does not identify the chondrocytes and identifies the osteoblasts; or determining cells to be positive for a marker that identifies the chondrocytes and the chondrocytes capable of hypertrophication, but to be negative for a marker that does not identify the chondrocytes and identifies the chondrocytes capable of hypertrophication.

In the present specification, the chondrocytes, the chondrocytes capable of hypertrophication, the osteoblasts and the induced osteoblasts may be identified, for example, using combinations of markers listed below.

| | | Chondrocytes | Osteoblasts, |
|---|---|---|---|
| | | capable of hyper- | induced |
| | Chondrocytes | trophication | osteoblasts |
| Type II collagen, | +: Expression | +: Expression | −: No expression |
| Cartilage proteoglycan | | | |
| (aglycan), Hyaluronic | | | |
| acid, Type IX collagen, | | | |
| Type XI collagen, | | | |
| Chondromodulin | | | |
| Type X collagen | −: No expression | +: Expression | −: No expression |
| Alkaline phosphatase, | −: No expression | +: Expression | +: Expression |
| Osteonectin | | | |
| Type I collagen, Bone | −: No expression | −: No expression | +: Expression |
| proteoglycan (e.g., | | | |
| decorin, biglycan), | | | |
| Osteocalcin, Matrix Gla | | | |
| protein, Osteoglycin, | | | |
| Osteopontin, Bone sialic | | | |
| acid protein, | | | |
| Pleiotrophin | | | |

Further, in the present specification, the chondrocytes, the chondrocytes capable of hypertrophication and the osteoblasts also may be identified by observing morphologies thereof or various stained states thereof in addition to the above detection of the markers.

The chondrocytes are cells whose aggregating state is observed under a microscopy. The cells show metachromasia with acid toluidine blue staining, and they are stained blue with alcian blue staining, are strained red with safranine O staining, and are not strained with alkaline phosphatase staining.

In the case where cells are arranged in a columnar manner to form a growth layer, the chondrocytes capable of hypertrophication are observed as cells next to the growth layer having larger sizes than those of the cells forming the growth layer under a microscopy. On the other hand, in the case where cells are not arranged in a columnar manner, the chondrocytes capable of hypertrophication are observed as cells having sizes larger than those of marginal cells under a microscopy.

The cells show metachromasia with acid toluidine blue staining, and are stained blue with alcian blue staining, are strained red with safranine O staining, and are strained with alkaline phosphatase staining.

The osteoblasts are cells each having a size of 20 to 30 μm, being of a cubic or columnar form, and having an alkaline phosphatase activity.

The alkaline phosphatase activity is determined by: A) a step of measuring two absorbances at 405 nm of a sample, wherein one absorbance is measured by adding 50 μL of a 4 mg/mL p-nitrophenyl phosphate solution and 50 μL of an alkali buffer (“A9226” produced by Sigma) to 100 μL of the sample, being reacted at 37° C. for 15 minutes, and then adding 50 μL of 1N NaOH to the sample to terminate the reaction, and the other absorbance is measured by further adding 20 μL of concentrated hydrochloric acid to the sample whose one absorbance has been measured; and B) a step of calculating a difference between the absorbances before and after the addition of the concentrated hydrochloric acid.

In this regard, the difference between the absorbances is an indicator of the alkaline phosphatase activity. Specifically, in the case where an absolute value of the difference between the absorbances increased, the osteoblasts are determined to have the alkaline phosphatase activity.

This alkaline phosphatase activity is also determined by: A) a step of measuring two absorbances at 405 nm of a sample, wherein one absorbance is measured by adding 50 μL of a 4 mg/mL p-nitrophenyl phosphate solution and 50 μL of an alkali buffer (“A9226” produced by Sigma) to 100 μL of the sample, being reacted at 37° C. for 15 minutes, and then adding 50 μL of 1N NaOH to the sample to terminate the reaction, and the other absorbance is measured by further adding 20 μL of concentrated hydrochloric acid to the sample whose one absorbance has been measured; and B) a step of calculating a difference between the absorbances before and after the addition of the concentrated hydrochloric acid.

In this regard, the difference between the absorbances is an indicator of the alkaline phosphatase activity. Specifically, in the case where a relative value of the difference between the absorbances increased by more than about one times, the osteoblasts are determined to have the alkaline phosphatase activity.

Zero to ten mM of p-nitro phenol solutions are prepared in every experiments, absorbances are measured using the p-nitro phenol solutions, and then measured values are plotted by defining X axis as concentration and Y axis as absorbance to make a linear calibration curve of the values. The absolute value of the alkaline phosphatase activity can be calculated from the absorbance based on this linear calibration curve.

“Induced osteoblasts” used herein refer to cells induced from undifferentiated cells by an induced osteoblast differentiation inducing agent according to the present invention. These induced osteoblasts may be produced using a method including: A) a step of providing a supernatant obtained by culturing chondrocytes capable of hypertrophication in a differentiation agent producing medium containing at least one selected from the group comprising glucocorticoid, β-glycerophosphate and ascorbic acid, or an induced osteoblast differentiation inducing agent existing in the supernatant; and B) a step of culturing undifferentiated cells in an undifferentiated cell culture medium containing the supernatant or the induced osteoblast differentiation inducing agent and a medium component at a sufficient condition that the undifferentiated cells are induced into the induced osteoblasts.

The above induced osteoblasts also may be induced by a method including: A) a step of providing an induced osteoblast differentiation inducing agent obtained by culturing chondrocytes capable of hypertrophication in a differentiation agent producing medium containing dexamethasone, β-glycerophosphate, ascorbic acid and a serum component; and B) a step of culturing undifferentiated cells in an undifferentiated cell culture medium containing the induced osteoblast differentiation inducing agent and a medium component, to differentiate the undifferentiated cells into the induced osteoblasts.

The induced osteoblasts of the present invention do not show metachromasia with acid toluidine blue staining, and may show negative with safranine O staining.

An “induced osteoblast marker (marker for induced osteoblasts)” used herein refers to any substance whose localization or expression in the induced osteoblasts aids in the identification thereof, for example, any substance which can be used for identifying the induced osteoblasts by its localization or expression. The induced osteoblasts as well as natural osteoblasts can be identified by localization or expression of the following markers (for example, localization or expression of type I collagen, bone proteoglycan (e.g., decorin, biglycan), alkaline phosphatase, osteocalcin, matrix Gla protein, osteoglycin, osteopontin, bone sialic acid protein, osteonectin or pleiotrophin).

“Induction of differentiation” used herein refers to a development process of parts in a biological organism such as cells, tissues and organs, which is a process of inducing formation of tissues or organs each having a specific feature. The terms “differentiation” and “induction of differentiation” are mainly used in embryology, development biology and the like.

The tissues and the organs in the biological organism are formed by divisions of a fertilized ovum consisting of a single cell until one reaches adulthood. It is difficult to distinguish between cells and cell populations in the early development of the biological organism which is before differentiation or is not well differentiated, because the cells and the cell populations do not have any morphological or functional feature at all. Such a condition is referred to as “undifferentiation”.

Furthermore, “differentiation” occurs in an organ, and thereby various cells constituting the organ develop into specific cells and cell populations. This is referred to as differentiation within the organ in organogenesis. Such induction of development is also referred to as the induction of differentiation.

An “ability of inducing differentiation into induced osteoblasts” used herein refers to an ability of inducing differentiation of undifferentiated cells, preferably embryonic stem (ES) cells, embryonic germ (EG) stem cells or tissue stem cells, and more preferably mesenchymal stem cells into the induced osteoblasts of the present invention. As one indicator for the ability of inducing differentiation into induced osteoblasts, the induced osteoblast marker (e.g., alkaline phosphatase) may be used.

Specifically, in the case where the alkaline phosphatase (ALP) activity of C3H10T1/2 cells exposed to an agent used in the present invention in an eagle's basal medium or the alkaline phosphatase activity of mesenchymal stem cells exposed to the agent in a minimum essential medium (MEM) increases by more than about 1 times that of each kind of the cells cultured in the eagle's basal or minimum essential medium containing no agent (e.g., the alkaline phosphatase activity of whole the cells), the agent is determined to have the ability of inducing differentiation into induced osteoblasts.

The alkaline phosphatase activity is determined by: A) a step of measuring two absorbances at 405 nm of a sample, wherein one absorbance is measured by adding 50 μL of a 4 mg/mL p-nitrophenyl phosphate solution and 50 μL of an alkali buffer (“A9226” produced by Sigma) to 100 μL of the sample containing the agent or no agent, being reacted at 37° C. for 15 minutes, and then adding 50 μL of 1N NaOH to the sample to terminate the reaction, and the other absorbance is measured by further adding 20 μL of concentrated hydrochloric acid to the sample whose one absorbance has been measured; and B) a step of calculating a difference between the absorbances before and after the addition of the concentrated hydrochloric acid. In this regard, the difference between the absorbances is an indicator of the alkaline phosphatase activity.

Further, in the case where the alkaline phosphatase (ALP) activity of C3H10T1/2 cells exposed to an agent used in the present invention in an eagle's basal medium or the alkaline phosphatase activity of mesenchymal stem cells exposed to the agent in a minimum essential medium (MEM) increases as compared with that of each kind of the cells cultured in the eagle's basal or minimum essential medium containing no agent (e.g., the alkaline phosphatase activity of whole the cells), the agent is determined to have the ability of inducing differentiation into induced osteoblasts.

This alkaline phosphatase activity is determined by: A) a step of measuring two absorbances at 405 nm of a sample, wherein one absorbance is measured by adding 50 μL of a 4 mg/mL p-nitrophenyl phosphate solution and 50 μL of an alkali buffer (“A9226” produced by Sigma) to 100 μL of the sample containing the agent or no agent, being reacted at 37° C. for 15 minutes, and then adding 50 μL of 1N NaOH to the sample to terminate the reaction, and the other absorbance is measured by further adding 20 μL of concentrated hydrochloric acid to the sample whose one absorbance has been measured; and B) a step of calculating a difference between the absorbances before and after the addition of the concentrated hydrochloric acid. In this regard, the difference between the absorbances is an indicator of the alkaline phosphatase activity.

Zero to ten mM of p-nitro phenol solutions are prepared in every experiments, absorbances are measured using the p-nitro phenol solutions, and then measured values are plotted by defining X axis as concentration and Y axis as absorbance to make a linear calibration curve of the values. The absolute value of the alkaline phosphatase activity can be calculated from the absorbance based on this linear calibration curve.

Heretofore, this alkaline phosphatase activity is used as an indicator of osteogenesis. In the case where the alkaline phosphatase activity increases, the osteogenesis is determined to be promoted (Suda Tatsuo edit, “kotsukeisei to kotsukyushu oyobi sorera no tyosetsuinshi 1 [osteogenesis, osteoclasis and regulators thereof 1” Kabushiki Kaisya Hirokawa Shoten [Hirokawa Shoten Co.], 1995, March 30, p. 39-44.).

In the present specification, an “ability of inducing differentiation into induced osteoblasts” for undifferentiated cells (e.g., embryonic stem cells, embryonic germ stem cells, mesenchymal stem cells, hematopoietic stem cells, vascular stem cells, hepatic stem cells, pancreatic (common) stem cells, neural stem cells) refers to an ability of inducing differentiation of the undifferentiated cells into the induced osteoblasts.

For example, the ability of inducing differentiation into induced osteoblasts may include an ability of inducing differentiation of undifferentiated cells, whose differentiation has not been induced by glucocorticoid, β-glycerophosphate and ascorbic acid, into the induced osteoblasts.

The ability of inducing differentiation into induced osteoblasts may be determined by evenly inoculating subject cells in a 24-well plate (produced by Becton, Dickinson and Company) at a density of 1.25×10⁴ cells/cm² (i.e., 2.5×10⁴ cells/well), culturing the cells in a 5% CO₂ incubator at 37° C. for 72 hours, and then measuring a degree of induced or increased expression of at least one of the induced osteoblast markers.

“undifferentiated cells” used herein refer to cells which have not reached terminal differentiation or cells which can be differentiated. In the present specification, the undifferentiated cells may be stem cells (e.g., embryonic stem cells, embryonic germ stem cells or tissue stem cells). For example, the stem cells may be mesenchymal stem cells (e.g., mesenchymal stem cells derived from bone marrow), hematopoietic stem cells, vascular stem cells, hepatic stem cells, pancreatic (common) stem cells, or neural stem cells. The undifferentiated cells further include all cells on the way of differentiation. Such undifferentiated cells may be C3H10T1/2 cells, ATDC5 cells, 3T3-Swiss albino cells, BALB/3T3 cells, NIH3T3 cells, PT-2501 cells, or stem cells derived from primary rat bone marrow.

These cells can be available from domestic and foreign sales companies such as Sanko Junyaku Co., Ltd., Cosmo Bio Co., Ltd., Takara Bio Inc., Toyobo Co., Ltd., Summit Pharma Biomedical, Cambrex Corporation, Stem Cell Technology, Invitrogen Corporation, a cell bank and the like in addition to Dainippon Sumitomo Pharma Co. Ltd. The undifferentiated cells used in the present invention may be any cells which can be differentiated into the induced osteoblasts. Further, the undifferentiated cells used in the present invention may be cells derived from a mammal (e.g., human, rat, mouse, rabbit). Examples of the cells may include mesenchymal stem cells collected from rat bone marrow.

“Stem cells” used herein refer to cells having a self-replicating ability and a pluripotency (i.e., multipotency). Typically, the stem cells can regenerate an injured tissue. The stem cells used herein may be embryonic stem (ES) cells, embryonic germ (EG) stem cells or tissue stem cells (also referred to as tissular stem cells or tissue-specific stem cells), but are not limited thereto. Further, the stem cells maybe artificially produced cells (e.g., fusion cells or reprogrammed cells as described in the present specification) as long as they can have the above-described abilities.

The embryonic stem cells refer to pluripotent stem cells derived from early embryo. The embryonic stem cells were, first, established in 1981 and have been applied to production of a knockout mouse since 1989. In 1998, human embryonic stem cells were established and are currently becoming available for regenerative medicine. It is believed that the embryonic germ cells are formed due to dedifferentiation of primordial germ cells by exposing them to a specific surrounding agent. While the embryonic germ cells have properties as embryonic stem cells, the embryonic germ cells hold a part of properties of the primordial germ cells from which they are derived.

The tissue stem cells are present in a tissue, have lower levels of pluripotency than those of the embryonic stem cells, and have relatively limited levels of differentiation, unlike the embryonic stem cells. Generally, the stem cells have undifferentiated intracellular structures, high nucleus/cytoplasm ratios, and few intracellular organelles. The stem cells used herein may be preferably mesenchymal stem cells, but the tissue stem cells, the embryonic germ cells or the embryonic stem cells may also be used as the stem cells depending on the circumstances.

The tissue stem cells are separated into categories of sites from which they are derived, such as dermal system, digestive system, bone marrow system and nervous system. Examples of the tissue stem cells in the dermal system include epidermal stem cells, hair follicle stem cells and the like. Examples of the tissue stem cells in the digestive system include pancreatic stem cells, hepatic stem cells and the like. Examples of the tissue stem cells in the bone marrow system include hematopoietic stem cells, mesenchymal stem cells and the like. Examples of the tissue stem cells in the nervous system include neural stem cells, retinal stem cells and the like.

The stem cells may be categorized based on origins thereof, and specifically, are categorized into stem cells derived from ectoderm, stem cells derived from endoderm, or stem cells derived from mesoderm. The stem cells derived from ectoderm are mostly present in brain, and include neural stem cells and the like. The stem cells derived from endoderm are mostly present in bone marrow, and include vascular stem cells, hematopoietic stem cells, mesenchymal stem cells and the like. The stem cells derived from mesoderm are mostly present in viscus, and include hepatic stem cells, pancreas stem cells and the like.

“Mesenchymal stem cells” used herein refer to stem cells observed in a mesenchymal tissue. Examples of the mesenchymal tissue include bone marrow, adipose tissue, vascular endothelium, smooth muscle, cardiac muscle, skeletal muscle, cartilage, bone and ligament, but are not limited thereto. The mesenchymal stem cells may be cells typically derived from bone marrow, adipose tissue, synovial tissue, muscular tissue, peripheral blood, placental tissue, menstrual blood, or cord blood (preferably, bone marrow).

A “growth medium” used herein refers to a medium containing a basal medium, antibiotics (e.g., penicillin and streptomycin), an antibacterial agent (e.g., amphotericin B) and a serum component (e.g., human serum, bovine serum, fetal bovine serum). Typically, the serum component maybe contained in the growth medium up to about 20% thereof. Furthermore, in the case where the basal medium is a minimum essential medium (MEM), the growth medium is referred to as an “MEM growth medium”, whereas in the case where the basal medium is a Ham's F12 medium (HAM), the growth medium is referred to as a “HAM growth medium”.

A “differentiation agent producing medium” used herein refers to a medium containing a basal medium, and at least one conventional osteoblast differentiation inducing component selected from the group consisting of glucocorticoid, p-glycerophosphate and ascorbic acid. The differentiation agent producing medium may contain at least one conventional osteoblast differentiation inducing component selected from the group consisting of the β-glycerophosphate and the ascorbic acid. The differentiation agent producing medium may contain all of the glucocorticoid, the β-glycerophosphate and the ascorbic acid as the conventional osteoblast differentiation inducing components.

Preferably, the differentiation agent producing medium contains the minimum essential medium (MEM) as the basal medium (base component), and the β-glycerophosphate and the ascorbic acid as the conventional osteoblast differentiation inducing components. Preferably, the “differentiation agent producing medium” may further contain a serum component (e.g., human serum, bovine serum, fetal bovine serum). Typically, the serum component may contain in the differentiation agent producing medium up to about 20% thereof. More preferably, the differentiation agent producing medium may contain the glucocorticoid, the β-glycerophosphate, the ascorbic acid and the serum component.

Furthermore, in the case where the basal medium is the minimum essential medium (MEM), the differentiation agent producing medium is referred to as an “MEM differentiation agent producing medium”, whereas in the case where the basal medium is a Ham's F12 medium (HAM), the differentiation agent producing medium is referred to as a “HAM differentiation agent producing medium”.

In this regard, it has not been shown that the differentiation agent producing medium itself has an ability of inducing differentiation of C3H10T1/2 cells, 3T3-Swiss albino cells, Balb 3T3 cells or NIH3T3 cells into osteoblasts. Therefore, it is believed that the agent used in the present invention is different from the components contained in the differentiation agent producing medium.

A “conventional osteoblast differentiation inducing component” used herein has been proposed by Maniatopoulos et al. (Maniatopoulos, C et. al.: Bone formation in vitro by stromal cells obtained from bone marrow of young adult rats. Cell Tissue Res., 254: 317-330, 1988.). Therefore, the conventional osteoblast differentiation inducing component is a component used for inducing differentiation of bone marrow cells into osteoblasts, and refers to a combination of glucocorticoid, β-glycerophosphate and ascorbic acid.

“Glucocorticoid” used herein is an adrenal cortex hormone, and is a generic name of a steroid hormone associated with saccharometabolism. The glucocorticoid is known as a component capable of inducing differentiation of bone marrow cells into osteoblasts (Maniatopoulos, C. et. al.: Bone formation in vitro by stromal cells obtained from bone marrow of young adult rats. Cell Tissue Res., 254: 317-330, 1988.). However, it is not known that the glucocorticoid has an ability of inducing the differentiation of the above-described cells into the osteoblasts.

The glucocorticoid is also referred to as carbohydrate corticoid. Typically, examples of the glucocorticoid include dexamethasone, betamethasone, predonisolone, predonisone, cortisone, cortisol, corticosterone, and the like, but are not limited thereto. The dexamethasone is preferably used as the glucocorticoid. Examples of the glucocorticoid also may include a chemically synthesized substance having the same effect as native glucocorticoid.

In the case where these typical glucocorticoids are used in culture of chondrocytes capable of hypertrophication together with β-glycerophosphate and ascorbic acid, an agent having an activity capable of inducing differentiation of C3H10T1/2 cells into osteoblasts is produced. Therefore, in the present invention, these typical glucocorticoids can be contained in the differentiation agent producing medium. The glucocorticoid may be contained in the differentiation agent producing medium at a concentration of 0.1 nM to 10 mM, and preferably a concentration of 10 to 100 nM.

“β-glycerophosphate” used herein is a generic name of a salt in which a phosphate group is bonded to at a p-position of glycerophosphoric acid (C₃H₅(OH)₂OPO₃H₂). Examples of the salt include a calcium salt, a sodium salt and the like. The β-glycerophosphate is known as a component capable of inducing differentiation of bone marrow cells into osteoblasts (Maniatopoulos, C. et. al.: Bone formation in vitro by stromal cells obtained from bone marrow of young adult rats. Cell Tissue Res., 254: 317-330, 1988.). However, it is not known that the β-glycerophosphate has an ability of inducing the differentiation of the above-described cells into the osteoblasts.

In the case where the β-glycerophosphate is used in culture of chondrocytes capable of hypertrophication together with glucocorticoid and ascorbic acid, an agent having an activity of inducing differentiation of C3H10T1/2 cells into osteoblasts is produced. Therefore, in the present invention, the β-glycerophosphate can be contained in the differentiation agent producing medium. The β-glycerophosphate may be contained in the differentiation agent producing medium at a concentration of 0.1 mM to 1 M, and preferably at a concentration of 10 mM.

“Ascorbic acid” used herein is a white water-soluble vitamin crystalline. The ascorbic acid is contained in a majority of plants, especially, citrus fruits. The ascorbic acid is also referred to as vitamin C. The ascorbic acid is known as a component capable of inducing differentiation of bone marrow cells into osteoblasts (Maniatopoulos, C. et. al.: Bone formation in vitro by stromal cells obtained from bone marrow of young adult rats. Cell Tissue Res., 254: 317-330, 1988.). However, it is not known that the ascorbic acid has an ability of inducing the differentiation of the above-described cells into the osteoblasts.

In the present invention, the ascorbic acid may include ascorbic acid and derivatives thereof. Examples of the ascorbic acid include L-ascorbic acid, L-ascorbic acid sodium, L-ascorbyl palmitate, L-ascorbyl stearate, L-ascorbic acid 2-glucoside, ascorbic acid phosphate magnesium, and ascorbic acid glucoside, but are not limited thereto. Examples of the ascorbic acid may include a chemically synthesized substance having the same effect as native ascorbic acid.

In the case where these typical ascorbyl acids are used in culture of chondrocytes capable of hypertrophication together with glucocorticoid and β-glycerophosphate, an agent having an activity capable of inducing differentiation of C3H10T1/2 cells into osteoblasts. Therefore, in the present invention, these typical ascorbic acids can be contained in the differentiation agent producing medium. The ascorbic acid may be contained in the differentiation agent producing medium at a concentration of 0.1 μg/mL to 5 mg/mL, and preferably at a concentration of 10 to 50 μg/mL.

An “undifferentiated cell culture medium” used herein refers to a medium containing an induced osteoblast differentiation inducing agent of the present invention and a medium component. The undifferentiated cell culture medium may include, but not limited to, an eagle's basal medium (BME), a minimum essential medium (MEM), a dulbecco's modified eagle medium (DMEM), Ham's F12 medium (HAM), a minimum essential medium α (αMEM) and a mixed medium thereof.

This medium may further contain a serum component (e.g., human serum, bovine serum, fetal bovine serum). Typically, the serum component may be contained in the undifferentiated cell culture medium up to about 20% thereof (preferably in the range of 10 to 15% thereof, and more preferably about 10% thereof).

### DESCRIPTION OF THE PREFERRED EMBODIMENTS

The best modes of the present invention are described below. It is appreciated that the embodiments provided below are be provided for the purpose of a better understanding of the present invention. The scope of the present invention should not be limited to the following descriptions. Therefore, it is apparent that those skilled in the art can read the descriptions herein and modify them appropriately within the scope of the present invention.

(Inducing Method of Induced Osteoblasts)

In one aspect, the present invention provides a method of inducing undifferentiated cells into induced osteoblasts according to the present invention. This method may include: A) a step of providing a supernatant obtained by culturing chondrocytes capable of hypertrophication in a differentiation agent producing medium containing at least one selected from the group comprising glucocorticoid, β-glycerophosphate and ascorbic acid, or an induced osteoblast differentiation inducing agent existing in the supernatant; and B) a step of culturing the undifferentiated cells in an undifferentiated cell culture medium containing the supernatant or the induced osteoblast differentiation inducing agent and a medium component at a sufficient condition that the undifferentiated cells are induced into the induced osteoblasts.

Although, heretofore, osteoblasts could not be massively collected or produced, by using the inducing method of the present invention, it is possible to provide the osteoblasts massively and stably. The induced osteoblasts according to the present invention are used for treating diseases associated with decrease of osteogenesis, bone injuries or bone defects, especially, for treating bone tumors, complex fractures and the like.

In one embodiment, the method of inducing the undifferentiated cells into the induced osteoblasts may include: A) a step of providing an induced osteoblast differentiation inducing agent obtained by culturing chondrocytes capable of hypertrophication in a differentiation agent producing medium containing dexamethasone, β-glycerophosphate, ascorbic acid and a serum component; and B) a step of culturing the undifferentiated cells in an undifferentiated cell culture medium containing the induced osteoblast differentiation inducing agent and a medium component, to differentiate the undifferentiated cells into the induced osteoblasts.

In one embodiment, the medium used for culturing the chondrocytes capable of hypertrophication in the inducting method of the present invention (in the present specification, referred to as a “differentiation agent producing medium”) may contain at least one of glucocorticoid (e.g., dexamethasone, predonisolone, predonisone, cortisone, betamethasone, cortisol, corticosterone), β-glycerophosphate, ascorbic acid and the like. Preferably, this medium may contain both the β-glycerophosphate and the ascorbic acid. More preferably, this medium contains all of the glucocorticoid, the β-glycerophosphate and the ascorbic acid.

In addition, this medium may further contain other components such as transforming growth factor-β (TGF-β), bone morphogenetic factor (BMP), leukemia inhibitory factor (LIF), colony stimulating factor (CSF), insulin-like growth factor (IGF), fibroblast growth factor (FGF), platelet-rich plasma (PRP), platelet-derived growth factor (PDGF), and vascular endothelial growth factor (VEGF). It may be useful that this medium further contains a serum component (e.g., human serum, bovine serum, fetal bovine serum). Typically, the serum component maybe contained in the medium up to about 20% thereof.

Further, examples of the medium used for culturing the chondrocytes capable of hypertrophication in the inducing method of the present invention may include a Ham's F12 (HamF12 or HAM), a dulbecco's modified eagle medium (DMEM), a minimum essential medium (MEM), a minimum essential medium α (αMEM), an eagle's basal medium (BME), a fitton-jackson modified medium (BGJb), but are not limited thereto. This medium may contain any substance capable of promoting proliferation and induction of differentiation of cells. In this regard, it has not been shown that this medium has an ability of inducing differentiation of C3H10T1/2 cells, 3T3-Swiss albino cells, Balb 3T3 cells or N1H3T3 cells into osteoblasts.

In one embodiment, undifferentiated cells used in the inducing method of the present invention may be cells derived from a mammal, preferably, cells derived from human, mouse, rat or rabbit, but are not limited thereto.

In one embodiment, the undifferentiated cells used in the producing method of the present invention may be stem cells (e.g., embryonic stem cells, embryonic germ stem cells, tissue stem cells). For example, the stem cells may be mesenchymal stem cells, hematopoietic stem cells, vascular stem cells, hepatic stem cells, pancreatic (common) stem cells or neural stem cells. Preferably, the undifferentiated cells may be the mesenchymal stem cells. The undifferentiated cells further include all cells on the way of differentiation.

Preferably, these undifferentiated cells may be C3H10T1/2 cells, ATDC5 cells, 3T3-Swiss albino cells, BALB/3T3 cells, NIH3T3 cells, C2C12 cells, PT-2501 cells, stem cells derived from primary rat bone marrow and the like (preferably the C3H10T1/2 cells, the 3T3-Swiss albino cells, the BALB/3T3 cells, the NIH3T3 cells, the PT-2501 cells and the stem cells derived from primary rat bone marrow, and more preferably the C3H10T1/2 cells, the PT-2501 cells and the stem cells derived from primary rat bone marrow). In this regard, the undifferentiated cells used in the present invention may be any cells capable of be differentiated into induced osteoblasts.

In another embodiment, a medium used for culturing the undifferentiated cells in the inducing method of the present invention (in the present specification, referred to as an “undifferentiated cell culture medium”) may include, but not limited to, an eagle's basal medium (BME), a minimum essential medium (MEM), a dulbecco's modified eagle medium (DMEM), a Ham's F12 (HAM), a minimum essential medium α (αMEM) or a mixed medium thereof.

In the case where a basal medium contained in the medium is one which can be generally used for culturing cells, the basal medium does not affect the induced osteoblast differentiation inducing agent according to the present invention. Therefore, if the induced osteoblast differentiation inducing agent can make contact with the undifferentiated cells in the culture, the undifferentiated cells can be differentiated into the induced osteoblasts.

Those skilled in the art can appropriately select a medium suitable for culturing the undifferentiated cells based on the disclosure of the present specification and the common technical knowledge. Preferably, the undifferentiated cell culture medium may be the eagle's basal medium (BME), the minimum essential medium (MEM), the Ham's F12 (HAM) or the minimum essential medium α (αMEM).

In one embodiment, the induced osteoblast differentiation inducing agent used in the present invention may exist (1) in the medium in which the chondrocytes capable of hypertrophication are cultured, or (2) in a fraction with a molecular weight of 50,000 or higher obtained by subjecting the medium in which the chondrocytes capable of hypertrophication are cultured to ultrafiltration using a filter having a molecular cutoff of 50,000.

In one embodiment, in the inducing method of the present invention, the step A) may include: culturing the chondrocytes capable of hypertrophication in the differentiation agent producing medium containing the dexamethasone, the β-glycerophosphate, the ascorbic acid and the serum component; and collecting the supernatant of the differentiation agent producing medium after the culture.

In one embodiment, in the inducing method of the present invention, the step A) may include subjecting the medium in which the chondrocytes capable of hypertrophication are cultured to ultrafiltration to separate it into a fraction with a molecular weight of 50,000 or higher.

In another embodiment, the inducing method of the present invention may further include a step of forming the undifferentiated cells into a pellet. This pellet forming step can be performed by being centrifuged at 170 to 200×g for 3 to 5 minutes, but is not limited thereto.

In a different embodiment, in the inducing method of the present invention, the sufficient condition that the undifferentiated cells are induced into the induced osteoblasts may include a culture period of 3 days to 3 weeks.

In a preferred embodiment, in the inducing method of the present invention, the chondrocytes capable of hypertrophication may be cultured in the differentiation agent producing medium containing the glucocorticoid, the β-glycerophosphate and the ascorbic acid; the undifferentiated cells may be selected from the group comprising the C3H10T1/2 cells, the ATDC5 cells, the 3T3-Swiss albino cells, the BALB/3T3 cells, the NIH3T3 cells, the C2C12 cells, the PT-2501 cells and the stem cells derived from primary rat bone marrow; the undifferentiated cell culture medium may contain the eagle's basal medium (BME), the minimum essential medium (MEM), the minimum essential medium a (aMEM) or the dulbecco's modified eagle medium (DMEM); and the sufficient condition that the undifferentiated cells are induced into the induced osteoblasts may include the culture period of 3 days to 3 weeks. This inducing method may further include the step of forming the undifferentiated cells into the pellet by being centrifuged at 170 to 200×g for 3 to 5 minutes.

In another preferred embodiment, the undifferentiated cells used in the present inducing method may be the mesenchymal stem cells (e.g., the mesenchymal stem cells derived from bone marrow); and the step A) may include (1) culturing the chondrocytes capable of hypertrophication in the differentiation agent producing medium containing the dexamethasone, the β-glycerophosphate, the ascorbic acid and the serum compomnent, and then collecting the supernatant of the differentiation agent producing medium after the culture, and (2) subjecting the supernatant to the ultrafiltration to separate it into the fraction with the molecular weight of 50,000 or higher.

In a preferred embodiment, the undifferentiated cells used in the present inducing method may be the C3H10T1/2 cells, the PT-2501 cells or the stem cells derived from primary rat bone marrow; and the step A) may include (1) culturing the chondrocytes capable of hypertrophication in the differentiation agent producing medium containing the dexamethasone, the β-glycerophosphate, the ascorbic acid and the serum compomnent, and then collecting the supernatant of the differentiation agent producing medium after the culture, and (2) subjecting the supernatant to the ultrafiltration to separate it into the fraction with the molecular weight of 50,000 or higher.

The induced osteoblast differentiation inducing agent used in the present invention has an ability of increasing the alkaline phosphatase (ALP) activity of the C3H10T1/2 cells exposed thereto in the eagle's basal medium or the alkaline phosphatase activity of the mesenchymal stem cells exposed thereto in the minimum essential medium (MEM) by more than about one times that of each kind of the cells cultured in the eagle's basal or minimum essential medium containing no agent (e.g., the alkaline phosphatase activity of whole the cells).

The alkaline phosphatase activity is determined by: A) a step of measuring two absorbances at 405 nm of a sample, wherein one absorbance is measured by adding 50 μL of a 4 mg/mL p-nitrophenyl phosphate solution and 50 μL of an alkali buffer (“A9226” produced by Sigma) to 100 μL of the sample containing the agent or no agent, being reacted at 37° C. for 15 minutes, and then adding 50 μL of 1N NaOH to the sample to terminate the reaction, and the other absorbance is measured by further adding 20 μL of concentrated hydrochloric acid to the sample whose one absorbance has been measured; and B) a step of calculating a difference between the absorbances before and after the addition of the concentrated hydrochloric acid. In this regard, the difference between the absorbances is an indicator of the alkaline phosphatase activity.

The alkaline phosphatase activity preferably shows increase by at least 2 times, at least 3 times, at least 4 times, at least 5 times, at least 6 times, at least 7 times, at least 8 times, at least 9 times, at least 10 times, at least 11 times, at least 12 times, or at least 13 times.

The induced osteoblast differentiation inducing agent used in the present invention has an ability of increasing the alkaline phosphatase (ALP) activity of the C3H10T1/2 cells exposed thereto in the eagle's basal medium or the alkaline phosphatase activity of the mesenchymal stem cells exposed thereto in the minimum essential medium (MEM) as compared with that of each kind of the cells cultured in the eagle's basal or minimum essential medium containing no agent (e.g., the alkaline phosphatase activity of whole the cells).

The alkaline phosphatase activity is determined by: A) a step of measuring two absorbances at 405 nm of a sample, wherein one absorbance is measured by adding 50 μL of a 4 mg/mL p-nitrophenyl phosphate solution and 50 μL of an alkali buffer (“A9226” produced by Sigma) to 100 μL of the sample containing the agent or no agent, being reacted at 37° C. for 15 minutes, and then adding 50 μL of 1N NaOH to the sample to terminate the reaction, and the other absorbance is measured by further adding 20 μL of concentrated hydrochloric acid to the sample whose one absorbance has been measured; and B) a step of calculating a difference between the absorbances before and after the addition of the concentrated hydrochloric acid. In this regard, the difference between the absorbances is an indicator of the alkaline phosphatase activity.

The alkaline phosphatase activity preferably shows increase by at least 2 times, at least 3 times, at least 4 times, at least 5 times, at least 6 times, at least 7 times, at least 8 times, at least 9 times, at least 10 times, at least 11 times, at least 12 times, or at least 13 times.

An “agent” or a “factor” interchangeably used herein may be any substance or component as long as it achieves the purpose intended. The induced osteoblast differentiation inducing agent used in the present invention may be, for example, a protein, a polypeptide, an oligopeptide, a peptide, an amino acid, a nucleic acid, a polysaccharide, a lipid, an organic low molecular weight molecule or a complex thereof.

An “induced osteoblast differentiation inducing agent” used herein refers to an agent capable of inducing differentiation of undifferentiated cells into induced osteoblasts, and may be a simplex or a complex as long as it can hold an activity itself. The induced osteoblast differentiation inducing agent can be obtained by culturing chondrocytes capable of hypertrophication in a differentiation agent producing medium containing at least one selected from the group comprising glucocorticoid, β-glycerophosphate and ascorbic acid.

It is understood that an agent obtained by another method or an agent of a distinct formation is exchangeably used in the present invention as the agent capable of inducing differentiation of undifferentiated cells into induced osteoblasts, as long as the agent has the same activity of the induced osteoblast differentiation inducing agent used in the present invention. Such an agent can be identified using the common technical knowledge in the art, based on the disclosure of the present specification, in addition to agents basically identified in Examples.

The induced osteoblast differentiation inducing agent used in the present invention has an ability of increasing expression of a specific substance for the induced osteoblasts, which is selected from the group comprising type I collagen, bone proteoglycan (e.g., decorin, biglycan), alkaline phosphatase, osteocalcin, matrix Gla protein, osteoglycin, osteopontin, bone sialic acid protein, osteonectin and pleiotrophin.

Therefore, the induced osteoblast differentiation inducing agent used herein is characterized by increasing an alkaline phosphatase activity of undifferentiated cells in an enzyme activity. Further, the induced osteoblast differentiation inducing agent is an agent having an ability of expressing at least one of osteoblast markers in the undifferentiated cells at the level of gene expression or protein expression.

In a preferred embodiment, the induced osteoblast differentiation inducing agent used in the present invention may be identified by detecting increase of the alkaline phosphatase activity and expression or localization of induced osteoblast markers in the undifferentiated cells.

In another embodiment, the induced osteoblast differentiation inducing agent used in the present invention loses the ability of inducing differentiation of undifferentiated cells into induced osteoblasts by heating it for 3 minutes in boiling water (generally, including about 96 to 100° C., e.g., about 96° C., about 97° C., about 98° C., about 99° C. and about 100° C.). The boiling is confirmed by observation. The lose of the ability of inducing differentiation of undifferentiated cells into induced osteoblasts means a state that does not substantially increase the localization or expression of the induced osteoblast markers.

In a different embodiment, the induced osteoblast differentiation inducing agent used in the present invention loses the ability of inducing alkaline phosphatase activity of undifferentiated cells by heating it for 3 minutes in boiling water. The lose of the ability of inducing alkaline phosphatase activity of undifferentiated cells means a state that does not substantially increase the alkaline phosphatase activity therein.

The terms “protein”, “polypeptide”, “oligopeptide” and “peptide” used herein have the same meaning and refer to an amino acid polymer having any length. This polymer may have a linear chemical structure, a branched chemical structure or a cyclic chemical structure. The amino acid may be a natural or non-natural amino acid, or a modified amino acid. Since the amino acid polymer used herein preferably has a form translated based on a nucleic acid molecule, it has the linear chemical structure and is composed of only the natural amino acids, but is not limited thereto. The term may include those assembled into a complex of a plurality of polypeptide chains.

The term also includes a naturally or artificially modified amino acid polymer. Examples of such modification include disulfide bond formation, glycosylation, lipidation, acetylation, phosphorylation, or any other manipulation or modification (e.g., conjugation with a labeling moiety). This definition encompasses a polypeptide containing at least one amino acid analog (e.g., a non-natural amino acid), a peptide-like compound (e.g., peptoid), and compounds modified using other methods known in the art.

It should be understood that, as particularly mentioned herein, a “protein” refers to an amino acid polymer having a relatively large molecular weight or a modified polymer thereof, and a “peptide” refers to an amino acid polymer having a relatively low molecular weight or a modified polymer thereof.

(Chondrocytes Capable of Hypertrophication)

Chondrocytes capable of hypertrophication used in the present invention are derived from a mammal, preferably, derived from human, mouse, rat, or rabbit. There are two manners of ossification including membranous ossification and chondral ossification which are common to the mammal.

The membranous ossification is a manner which functions at formation of flat bone (e.g., majority of skull, clavicle) in nearby surface. In the membranous ossification, membranous bone is directly formed intra connective tissue without passing cartilage. The membranous ossification is also referred to as intramembraous ossification or connective tissue ossification.

On the other hand, the chondral ossification is a manner which functions at formation of endoskeleton (e.g., vertebra, costa, limb bone) in interior body. In the chondral ossification, cartilage is first formed, blood vessels infiltrate into cadre of the cartilage, and then the cartilage is calcified to form calcified cartilage. The formed calcified cartilage is crashed momentarily, and then ossification is induced to form bone and primordial bone marrow.

In this process, cartilage primordium is formed intra cartilage, and then the cartilage primordium is affected by a growth hormone and the like. As a result, the cartilage elongates and increases in a size thereof towards long axis and minor axis. Thereafter, the blood vessels infiltrate in epiphysis to induce ossification.

The chondral ossification is also referred to as endochondral ossification or enchondral ossification (see, Fujita Hisao, Fujita Tsuneo, “hone no hassei” hyojun soshikigaku souron, page 127 [“the development of bone” standard histological review, page 127]; kososhiki no kigen to shinka-josetsu-, Suda Tateo, The BONE, 18 kan, pages 421-426, 2004 [The origin and evolution of the hard tissue-introduction-, Suda Tateo, The BONE, 18th volume, pages 421-426, 2004; nainankotsusei kotsukeisei no katei, Suzuki Fujio, “hone wa donoyonishite dekiruka” Osaka Daigaku Shuppankai, page 21, 2004 [The process of endochondral ossification, Suzuki Fujio, “How does bone formation?” Osaka University Press, page 21, 2004]; Suzuki Takao et al. edit, “Hone no jiten”, Asakura shoten [Suzuki Takao et al. edit, “The dictionary of bone”, Asakura shoten]).

Therefore, the chondrocytes capable of hypertrophication, which can produce an agent capable of inducing differentiation of undifferentiated cells into induced osteoblasts, exist evenly in a mammal including rat, mouse, rabbit, human and the like. The agent acts as an important role in the ossification. Thus, the agent of the present invention can be produced form the chondrocytes capable of hypertrophication by using the same procedure, in a mammal and the like in which the endochondral ossification is induced, in spite of species.

Using molecular biology methods, it is demonstrated that osteogenesis is induced by implanting a human recombinant BMP protein into rat, and therefore BMP derived from human functions in the same manner as BMP protein derived from rat (see, Wozney, J. M. et al., Science, 242: 1528-1534, 1988., and Wuerzler K K. et al., J. Craniofacial Surg., 9: 131-137, 1998.). It is also proven that the agent associated with the ossification can be interchangeably used in between human and rat. It is known that these BMPs are different from each other at the level of amino acid sequences thereof, but are substantively identical to each other in their properties as a protein (i.e., solid state properties on conditions of induction and the like).

The chondrocytes capable of hypertrophication according to the present invention may be isolated or induced from, for example, a region such as a chondro-osseous junction of costa, an epiphysial line of long bone (e.g., femur, tibia, fibula, humerus, ulna, radius), an epiphysial line of vertebra, a zone of proliferating cartilage of ossicle (e.g., hand bone, foot bone and sterna), perichondrium, bone primordium formed from cartilage of fetus, a callus region of a healing bone-fracture and a cartilaginous part of a bone proliferation phase.

The chondrocytes capable of hypertrophication used in the present invention maybe chondrocytes obtained from any regions as long as they have an ability of hypertrophication. The chondrocytes capable of hypertrophication also may be obtained by induction of differentiation.

In the present invention, in the case where the induced osteoblast differentiation inducing agent is produced by the chondrocytes capable of hypertrophication, the chondrocytes capable of hypertrophication may be typically adjusted to a cell density of 4×10⁴ cells/cm². The cell density is normally adjusted to a value in the range of 10⁴ to 10⁶ cells/cm², but may be adjusted to less than 10⁴ cells/cm², or be adjusted to more than 10⁶ cells/cm².

In the present invention, culture of the chondrocytes capable of hypertrophication is performed using cells isolated or induced by the above-described methods.

The chondrocytes capable of hypertrophication used in the present invention may be cells cultured in any medium containing a Ham's F12 (HamF12), a dulbecco's modified eagle medium (DMEM), a minimum essential medium (MEM), a minimum essential medium α (αMEM), an eagle's basal medium (BME), a fitton-jackson modified medium (BGJb), but are not limited thereto. The chondrocytes capable of hypertrophication may be cells cultured in a medium containing any substance capable of promoting proliferation and induction of differentiation of cells.

In the present invention, a differentiation agent producing medium may contain at least one conventional osteoblast differentiation inducing component selected from the group comprising glucocorticoid (e.g., dexamethasone, predonisolone, predonisone, cortisone, betamethasone, cortisol, corticosterone), β-glycerophosphate and ascorbic acid. The agent used in the present invention is also produced using the differentiation agent producing medium containing only the β-glycerophosphate and the ascorbic acid. Preferably, the differentiation agent producing medium contains all of the glucocorticoid, the β-glycerophosphate and the ascorbic acid.

In the present invention, the differentiation agent producing medium may further contain other components such as transforming growth factor-β (TGF-β), bone morphogenetic factor (BMP), leukemia inhibitory factor (LIF), colony stimulating factor (CSF), insulin-like growth factor (IGF), fibroblast growth factor (FGF), platelet-rich plasma (PRP), platelet-derived growth factor (PDGF), and vascular endothelial growth factor (VEGF). It may be useful that the differentiation agent producing medium further contains a serum component (e.g., human serum, bovine serum, fetal bovine serum). Typically, the serum component may be contained in the differentiation agent producing medium up to about 20% thereof.

In the present specification, a period for culturing the chondrocytes capable of hypertrophication may be a period in which the agent can be produced in a sufficient amount (e.g., several months to half year, or 3 days to 3 weeks (e.g., 3 days, 4 days, 5 days, 6 days, 7 days, 8 days, 9 days, 10 days, 20 days, more than 1 month, half year, 5 months, 4 months, 3 months, 2 months, 1 month, 3 weeks or less, and possible combinations thereof within any range). When the period of the culture is progressed, and cells are confluent in a culture vessel, it is preferred that the cells are passaged.

(Induced Osteoblasts)

The present invention provides induced osteoblasts induced from undifferentiated cells using an induced osteoblast differentiation inducing agent produced by chondrocytes capable of hypertrophication. In the induction of the induced osteoblasts, any that is described above in (Inducing method of induced osteoblasts), (Chondrocytes capable of hypertrophication) and the like in the present specification may be used.

The induced osteoblasts produced using the inducing method of the present invention can be used in the same manner as natural osteoblasts. Therefore, the induced osteoblasts of the present invention can be independently used for treating bone defects and the like, be used as a composition together with an extracellular matrix and the like, or be used as a composite material together with a scaffold.

(Medicine and Medical material)

In one aspect, the present invention provides a medicine or medical material for promoting or inducing osteogenesis in a biological organism containing induced osteoblasts. The medicine according to the present invention can be used for treating diseases associated with decrease of osteogenesis, bone injuries or bone defects, especially, for treating bone tumors, complex fractures and the like. The medicine according to the present invention can promote or induce the osteogenesis in the biological organism, amazingly, also can induce the osteogenesis even in a region where bone does not exist in the vicinity thereof.

In one embodiment, undifferentiated cells used in the present invention may be C3H10T1/2 cells, ATDC5 cells, 3T3-Swiss albino cells, BALB/3T3 cells, NIH3T3 cells, C2C12 cells, PT-2501 cells, stem cells derived from primary rat bone marrow and the like (preferably, the C3H10T1/2 cells, the PT-2501 cells, and the stem cells derived from primary rat bone marrow), but are not limited thereto.

In one embodiment, the undifferentiated cells used in the present invention may be mesenchymal stem cells (e.g., mesenchymal stem cells derived from bone marrow). The undifferentiated cells used in the present invention may be cells derived from a mammal (e.g., human, rat, mouse, rabbit).

Examples of such undifferentiated cells may include, for example, stem cells derived from rat bone marrow. As the undifferentiated cells, a commercial cell strain (e.g., human mesenchymal stem cells (hMSC): “PT-2501” produced by Cambrex Corporation) also can be used.

In one embodiment, the undifferentiated cells used in the present invention may be formed into a pellet. For example, the above undifferentiated cells may be C3H10T1/2 cells formed into the pellet. The induced osteoblasts produced by the inducing method of the present invention can be used in the same manner as natural osteoblasts. Therefore, the medicine of the present invention can be used for treating bone defects and the like.

In the production of the induced osteoblasts, any that is described above in (Inducing method of induced osteoblasts), (Chondrocytes capable of hypertrophication) and the like in the present specification may be used.

(Composition)

In another aspect, the present invention provides a composition for promoting or inducing osteogenesis in a biological organism containing A) an extracellular matrix and B) induced osteoblasts. The composition according to the present invention can be used for treating diseases associated with decrease of osteogenesis, bone injuries or bone defects, especially, for treating bone tumors, complex fractures and the like. The composition according to the present invention can promote or induce the osteogenesis in the biological organism, amazingly, also can induce the osteogenesis even in a region where bone does not exist in the vicinity thereof.

In one embodiment, the extracellular matrix maybe an extracellular matrix derived from the induced osteoblasts, but is not limited thereto.

In a preferred embodiment, this extracellular matrix maybe type I collagen, bone proteoglycan, osteocalcin, matrix Gla protein, osteoglycin, osteopontin, bone sialic acid protein and the like, but is not limited thereto.

In one embodiment, in the composition of the present invention, the induced osteoblasts and the extracellular matrix may exist in a mixed state.

In one embodiment, undifferentiated cells from which the induced osteoblasts are derived and which is contained in the composition of the present invention may be stem cells (e.g., embryonic stem cells, embryonic germ stem cells or tissue stem cells). For example, the stem cells may be mesenchymal stem cells (e.g., mesenchymal stem cells derived from bone marrow), hematopoietic stem cells, vascular stem cells, hepatic stem cells, pancreatic (common) stem cells, or neural stem cells.

The undifferentiated cells further include all cells on the way of differentiation. Preferably, these undifferentiated cells may be C3H10T1/2 cells, ATDC5 cells, 3T3-Swiss albino cells, BALB/3T3 cells, NIH3T3 cells, C2C12 cells, PT-2501 cells, stem cells derived from primary rat bone marrow, and the like (more preferably, the C3H10T1/2 cells, the PT-2501 cells and the stem cells derived from primary rat bone marrow).

The undifferentiated cells used in the present invention may be any cells which can be differentiated into the induced osteoblasts. The undifferentiated cells used in the present invention maybe cells derived from a mammal (e.g., human, rat, mouse, rabbit). Examples of the cells may include mesenchymal stem cells collected from rat bone marrow. As the undifferentiated cells, a commercial cell strain (e.g., human mesenchymal stem cells (hMSC): “PT-2501” produced by Cambrex Corporation) also can be used.

In another embodiment, in the composition of the present invention, the induced osteoblasts may contain cells secreting the extracellular matrix. In a preferred embodiment, in the composition of the present invention, the induced osteoblasts may be cells derived from C3H10T1/2 cells, ATDC5 cells, 3T3-Swiss albino cells, BALB/3T3 cells, NIH3T3 cells, C2C12 cells, PT-2501 cells, stem cells derived from primary rat bone marrow and the like (preferably, the C3H10T1/2 cells, the PT-2501 cells and the stem cells derived from primary rat bone marrow). In addition, the induced osteoblasts are secreting the extracellular matrix.

In another embodiment, the composition of the present invention maybe used in osteogenesis for repairing or treating bone defects. Each of the bone defects may have a size that cannot be repaired only by immobilizing bone.

In a different embodiment, the composition of the present invention maybe used in osteogenesis for forming bone in a region where the bone does not exist in the vicinity thereof. In the composition of the present invention, any that is described above in (Inducing method of induced osteoblasts), (Chondrocytes capable of hypertrophication) and the like in the present specification may be used.

(Composite Material)

In one aspect, the present invention provides a composite material for promoting or inducing osteogenesis in a biological organism. The composite material may contain A) an extracellular matrix, B) induced osteoblasts and C) a biocompatible scaffold. The composite material according to the present invention can be used for treating diseases associated with decrease of osteogenesis, bone injuries or bone defects, especially, for treating bone tumors, complex fractures and the like. The composite material according to the present invention can promote or induce the osteogenesis in the biological organism, amazingly, also can induce the osteogenesis even in a region where bone does not exist in the vicinity thereof.

In one embodiment, the extracellular matrix maybe an extracellular matrix derived from the induced osteoblasts, but is not limited thereto.

In a preferred embodiment, this extracellular matrix may be type I collagen, bone proteoglycan, osteocalcin, matrix Gla protein, osteoglycin, osteopontin, bone sialic acid protein, and the like, but is not limited thereto.

In one embodiment, the biocompatible scaffold used in the composite material of the present invention may be calcium phosphate, calcium carbonate, alumina, zirconia, apatite-wollastonite deposited glass, gelatin, collagen, chitin, fibrin, hyaluronic acid, an extracellular matrix mixture, silk, cellulose, dextran, agarose, agar, synthetic polypeptide, polylactic acid, polyleucine, alginic acid, polyglycolic acid, polymethyl methacrylate, polycyanoacrylate, polyacrylonitrile, polyurethane, polypropylene, polyethylene, polyvinyl chloride, an ethylene-vinyl acetate copolymer, nylon, a combination thereof, and the like, but is not limited thereto. This is because any biocompatible scaffold can be used as long as the present agent adheres thereto or is dispersed thereinto, or can adhere thereto or be dispersed thereinto.

Preferably, the biocompatible scaffold may be, for example, porous hydroxyapatite (e.g., “APACERAM porosity of 50%” produced by HOYA CORPORATION), super porous hydroxyapatite (e.g., “APACERAM porosity of 85%” produced by HOYA CORPORATION, “3D Scaffold” produced by BD Corporation), an apatite-collagen mixture (e.g., a mixture of “APACERAM GRANULE” produced by HOYA CORPORATION and “Collagen Gel” produced by Nitta Gelatin Inc.), a apatite-collagen complex (e.g., “APACOLLA” produced by HOYA CORPORATION), collagen gel (e.g., “Collagen Gel” produced by Nitta Gelatin Inc.), collagen sponge (e.g., “Collagen Sponge” produced by Nitta Gelatin Inc.), gelatin sponge (e.g., “Hemostatic Gelatin Sponge” produced by Yamanouchi Pharmaceutical Co., Ltd.), fibrin gel (“Beriplast P” produced by Nipro), synthetic peptide (e.g., “Pramax” produced by 3D Matrix Corporation), an extracellular matrix mixture (e.g., “Matrigel” produced by BD Corporation), alginate (“Kelton LVCR” produced by Kelco Corporation), agarose (“Agarose” produced by Wako Pure Chemical Industries, Ltd.), polyglycolic acid, polylactic acid, a polyglycolic acid-polylactic acid copolymer and a combination thereof. More preferably, the biocompatible scaffold may be the hydroxyapatite, the collagen gel and the extracellular matrix mixture.

In another embodiment, the induced osteoblasts may adhere to the biocompatible scaffold via the extracellular matrix or directly.

In one embodiment, the composite material of the present invention may be used in osteogenesis for repairing or treating bone defects. Examples of such bone defects include lesions such as bone tumors, osteoporosis, rheumatoid arthritis, osteoarthritis, osteomyelitis, and osteonecrosis; correction such as immobilization of bone, foraminotomy and osteotomy; trauma such as complex fracture; bone defects derived from collecting ilium; and the like, but are not limited thereto. Each of the bone defects may have a size that cannot be repaired only by immobilizing bone.

In another embodiment, the composite material of the present invention may be used in osteogenesis for forming bone in a region where the bone does not exist in the vicinity thereof. Such a region where the bone does not exist in the vicinity thereof may include soft tissues such as a subcutaneous tissue, a muscle tissue and a fat tissue, a digestive organ, a respiratory organ, an urinary organ, a genital organ, an endocrine organ, a vascular system, a nervous system and a sense organ, but is not limited thereto.

In the composite material of the present invention, as the induced osteoblasts, any that is described above in (Inducing method of induced osteoblasts), (Chondrocytes capable of hypertrophication), (Composition) and the like in the present specification may be used.

(Producing Method)

In one aspect, the present invention provides a method of producing a composite material for promoting or inducing osteogenesis in a biological organism. The method includes: A) a step of providing induced osteoblasts induced using an agent produced by chondrocytes capable of hypertrophication; B) a step of culturing the induced osteoblasts on a biocompatible scaffold. The producing method of the present invention can provide the composite material for promoting or inducing the osteogenesis in the biological organism massively and stably. The composite material also can induce the osteogenesis even in a region where bone does not exist in the vicinity thereof.

In one embodiment, the producing method of the present invention may include: A) a step of providing the induced osteoblasts in which they are produced using the method described above in (Inducing method of induced osteoblasts) in the present specification; and B) a step of culturing the induced osteoblasts on the biocompatible scaffold.

In one embodiment, in the present producing method, the induced osteoblasts may be induced from undifferentiated cells on the biocompatible scaffold.

In a different embodiment, the composite material produced using this producing method may contain an extracellular matrix. In one embodiment, the extracellular matrix can be produced by culturing the induced osteoblasts on the biocompatible scaffold. In a different embodiment, the extracellular matrix may be externally added to the biocompatible scaffold. The extracellular matrix may be added to the biocompatible scaffold before, after or in process of inoculating the induced osteoblasts on the biocompatible scaffold.

In the producing method of the composite material of the present invention, as the induced osteoblasts and the composite material, respectively, any that is described above in (Inducing method of induced osteoblasts), (Chondrocytes capable of hypertrophication), (Composition), (Composite material) and the like in the present specification maybe used.

(Scaffold)

A “scaffold” used herein refers to a material for supporting cells. The scaffold has constant strength and biocompatibility. The scaffold used herein is produced from biological materials, naturally supplied materials, or naturally occurring materials or synthetically supplied materials.

In a specially described case, the scaffold is formed of materials other than organisms such as tissues or cells (i.e., non-cellular materials). The scaffold used herein is a composition formed of materials other than organisms such as tissues or cells, including materials derived from a biological organism such as collagen and hydroxyapatite. An “organism” used herein refers to a material-system organized so as to have a living function. That is, the term “organism” distinguishes living beings from other material-systems. The concept of the organism includes cells, tissues or others, but does not include materials derived from living beings and extracted from the organism.

Examples of a region of the scaffold on which cells are fixed include a surface of the scaffold, and an internal pore of the scaffold (in the case where it has such an internal pore that can place cells). For example, a scaffold made of hydroxyapatite includes many pores which can normally place cells sufficiently.

A material constituting the scaffold may be calcium phosphate, calcium carbonate, alumina, zirconia, apatite-wollastonite deposited glass, gelatin, collagen, chitin, fibrin, hyaluronic acid, an extracellular matrix mixture, silk, cellulose, dextran, agarose, agar, synthetic polypeptide, polylactic acid, polyleucine, alginic acid, polyglycolic acid, polymethyl methacrylate, polycyanoacrylate, polyacrylonitrile, polyurethane, polypropylene, polyethylene, polyvinyl chloride, an ethylene-vinyl acetate copolymer, nylon, a combination thereof, and the like, but is not limited thereto. This is because any biocompatible scaffold can be used as long as the present agent adheres thereto or is dispersed thereinto, or can adhere thereto or be dispersed thereinto.

Preferably, the biocompatible scaffold may be, for example, porous hydroxyapatite (e.g., “APACERAM porosity of 50%” produced by HOYA CORPORATION), super porous hydroxyapatite (e.g., “APACERAM porosity of 85%” produced by HOYA CORPORATION, “3D Scaffold” produced by BD Corporation), an apatite-collagen mixture (e.g., a mixture of “APACERAM GRANULE” produced by HOYA CORPORATION and “Collagen Gel” produced by Nitta Gelatin Inc.), a apatite-collagen complex (e.g., “APACOLLA” produced by HOYA CORPORATION), collagen gel (e.g., “Collagen Gel” produced by Nitta Gelatin Inc.), collagen sponge (e.g., “Collagen Sponge” produced by Nitta Gelatin Inc.), gelatin sponge (e.g., “Hemostatic Gelatin Sponge” produced by Yamanouchi Pharmaceutical Co., Ltd.), fibrin gel (“Beriplast P” produced by Nipro), synthetic peptide (e.g., “Pramax” produced by 3D Matrix Corporation), an extracellular matrix mixture (e.g., “Matrigel” produced by BD Corporation), alginate (“Kelton LVCR” produced by Kelco Corporation), agarose (“Agarose” produced by Wako Pure Chemical Industries, Ltd.), polyglycolic acid, polylactic acid, a polyglycolic acid-polylactic acid copolymer and a combination thereof. More preferably, the biocompatible scaffold may be the hydroxyapatite, the collagen gel and the extracellular matrix mixture.

These scaffolds may be provided in any form such as a granular form, a block form, or a sponge form. These scaffolds may be porous or non-porous. As such scaffolds, those commercially available from, for example, HOYA CORPORATION, Olympus Corporation, Kyocera Corporation, Mitsubishi Pharma Corporation, Dainippon Sumitomo Pharma Co. Ltd., Kobayashi Pharmaceuticals Co. Ltd., Zimmer Inc. may be used. Standard procedures for preparation and characterization of the scaffolds are known in the art, which only require the routine experimentation and the common technical knowledge in the art. For example, see U.S. Pat. No. 4,975,526; No. 5,011,691; No. 5,171,574; No. 5,266,683; No. 5,354,557; and No. 5,468,845, which are incorporated herein as references.

Other scaffolds are also described, for example, in the following documents: articles for biocompatible materials such as LeGeros and Daculsi, Handbook of Bioactive Ceramics, II pp. 17-28 (1990, CRC Press); other published descriptions such as Yang Cao, JieWeng, Biomaterials17 (1996) pp. 419-424; LeGeros, Adv. Dent. Res. 2, 164 (1988); Johnson et al., J. Orthopaedic Research, 1996, vol. 14, pp. 351-369; and Piattelli et al., Biomaterials 1996, vol. 17, pp. 1767-1770, which are incorporated herein as references.

“Calcium phosphate” used herein is a generic name for phosphates of calcium. Examples of the calcium phosphate include compounds represented by the following chemical formulas such as CaHPO₄, Ca₃(PO₄)₂, Ca₄O(PO₄)₂, Ca₁₀(PO₄)₆(OH)₂, CaP₄O₁₁, Ca(PO₃)₂, Ca₂P₂O₇, and Ca(H₂PO₄)₂.H₂O, but are not limited thereto.

“Hydroxyapatite” used herein refers to a compound whose general composition is Ca₁₀(PO₄)₆(OH)₂. This hydroxyapatite is a main component of mammalian hard tissues (bone and teeth), like collagen. Although the hydroxyapatite contains a series of the above-described calcium phosphates, the PO₄ and OH components within the apatite in the hard tissues of a biological organism are often substituted with a CO₃ component in body fluids.

Furthermore, the hydroxyapatite is a material having safety approval by the Ministry of Health, Labour and Welfare of Japan, and the FDA (U.S. Food and Drug Administration). Although many commercially available hydroxyapatites are non-absorbable by the biological organism and remain hardly absorbed in the biological organism, some are absorbable by the biological organism.

An “extracellular matrix mixture” used herein refers to a mixture of an extracellular matrix and a growth factor. Examples of the extracellular matrix include laminin, collagen and the like, but are not limited thereto. The extracellular matrix may be derived from a biological organism or synthesized.

(Method of Promoting or Inducing Osteogenesis in Biological Organism)

In one aspect, the present invention provides a method of promoting or inducing osteogenesis in a biological organism. The method may include a step of implanting induced osteoblasts, a medicine, a composition or a composite material according to the present invention into a region where the promotion and induction of the osteogenesis in the biological organism are required.

As the induced osteoblasts, the medicine, the composition or the composite material according to the present invention, any that is described above in (Inducing method of induced osteoblasts), (Chondrocytes capable of hypertrophication), (Medicine and medical material), (Composition), (Composite material) and the like in the present specification maybe used. The induced osteoblasts according to the present invention can be used in the same manner as natural osteoblasts. Therefore, they can be used for promoting or inducing the osteogenesis in the biological organism.

In one embodiment, in the method of the present invention, the osteogenesis may be used for repairing or treating bone defects. Each of the bone defects may have a size that cannot be repaired only by immobilizing bone.

In another embodiment, the osteogenesis also may be used for forming bone in a region where the bone does not exist in the vicinity thereof.

A “subject” used herein refers to a living being to which a treatment according to the present invention is applied. It is also referred to as a “patient”. The subject or the patient may be dog, cat or horse, preferably human.

A subcutaneous test for osteogenesis is a test for evaluating an osteogenic ability of forming bone in a region where the bone does not originally exist. This osteogenic ability is also referred to as prosthesis. Since this test can be performed easily, it is broadly used in the art. In the case of a bone treatment, a bone defect test may be used as a method of testing.

In this test, the osteogenesis occurs under an environment in which conditions capable of inducing it have been completed. Further, the osteogenesis is induced by osteoblasts already existing near a bone defective region or osteoblasts induced/migrated thereto. Thus, it is normally believed that a rate of the osteogenesis in the bone defect test is better than that in the subcutaneous test.

It is well-known that a result of the subcutaneous test is consistent with a rate of osteogenesis in an actual bone defect (see, e.g., Urist, M. R., Science, 150: 893-899 (1965); Wozney, J. M. et al., Science, 242: 1528-1532 (1988); Johnson, E. E. et al., Clin. Orthop., 230: 257-265 (1988); Ekelund, A. et al., Clin. Orthop., 263: 102-112 (1991); and Riley, E. H. et al., Clin. Orthop., 324: 39-46 (1996)). Therefore, if the osteogenesis is observed as a result of the subcutaneous test, those skilled in the art understand that osteogenesis should be also induced in the bone defect test.

In the case where mouse C3H10T1/2 cells are formed into pellets (cell pellets), are then the pellets are cultured in a medium to which a supernatant containing the induced osteoblast differentiation inducing agent of the present invention is added, the mouse C3H10T1/2 cells of the pellets are induced into induced osteoblasts. Thereafter, in the case where the pellets of the induced osteoblasts are implanted under the skins of and into bone defective regions of syngenic animals or immunodeficient animals, osteogenesis is induced.

On the other hand, in the case where the pellets of the mouse C3H10T1/2 cells are cultured in a medium to which a supernatant containing no induced osteoblast differentiation inducing agent (a supernatant of a differentiation agent producing medium in which chondrocytes incapable of hypertrophication are cultured, or a supernatant of a growth medium in which chondrocytes capable of hypertrophication are cultured) is added, a medium to which only the differentiation agent producing medium is added, or a medium to which only the differentiation agent producing medium is added, the mouse C3H10T1/2 cells of the pellets are not induced into the induced osteoblasts in the cell pellet. Even in the case where the pellets are implanted under the skins and into bone defective regions, it is predicted that osteogenesis is not induced.

In the case where mesenchymal stem cells (e.g., undifferentiated cells derived from bone marrow) are formed into pellets (cell pellets), are then the pellets are cultured in a medium to which a supernatant containing the induced osteoblast differentiation inducing agent of the present invention is added, it is predicted that the mesenchymal stem cells of the pellets are induced into induced osteoblasts. Further, in the case where the pellets of the induced osteoblasts are implanted under the skins and into bone defective regions of syngenic animals or immunodeficient animals, it is predicted that osteogenesis is induced.

In the case where the pellets of the mesenchymal stem cells (e.g., the undifferentiated cells derived from bone marrow) are cultured in a medium to which a supernatant containing no induced osteoblast differentiation inducing agent (a supernatant of a differentiation agent producing medium in which chondrocytes incapable of hypertrophication are cultured) is added, or a medium to which only the differentiation agent producing medium is added, it is predicted that the mesenchymal stem cells of the pellets are slightly induced into the induced osteoblasts.

This is because the differentiation agent producing medium contains components used for inducing differentiation of bone marrow cells into osteoblasts (glucocorticoid, β-glycerophosphate and ascorbic acid). In the case where the pellets of the mesenchymal stem cells are implanted under the skins and into bone defective regions, it is predicted that osteogenesis is slightly induced in a small amount thereof.

In the case where the pellets of the mesenchymal stem cells (e.g., the undifferentiated cells derived from bone marrow) are cultured in a medium to which a supernatant containing no induced osteoblast differentiation inducing agent (a supernatant of a growth medium in which the chondrocytes capable of hypertrophication are cultured), or a medium to which only the differentiation agent producing medium is added, the mesenchymal stem cells are not induced into the induced osteoblasts. Even in the case where the pellets of the mesenchymal stem cells are implanted under the skins and into bone defective regions, it is predicted that osteogenesis is not induced.

In order to produce the composite material of the present invention, in the case where mouse C3H10T1/2 cells are inoculated on a scaffold, and then cultured in a medium to which a supernatant containing the induced osteoblast differentiation inducing agent of the present invention is added, the mouse C3H10T1/2 cells are induced into induced osteoblasts on the scaffold. In the case where this composite material is implanted under the skin of a syngenic animal or an immunodeficient animal, it is predicted that osteogenesis is induced. Further, in the case where the composite material is implanted into a bone defective region thereof, it is predicted that good osteogenesis is induced.

On the other hand, even in the case where mouse C3H10T1/2 cells are inoculated on a scaffold, and then cultured in a medium to which a supernatant containing no induced osteoblast differentiation inducing agent (a supernatant of a differentiation agent producing medium in which chondrocytes incapable of hypertrophication are cultured, or a supernatant of a growth medium in which the chondrocytes capable of hypertrophication are cultured) is added, a medium to which only the differentiation agent producing medium is added, or a medium to which only the differentiation agent producing medium is added, the mouse C3H10T1/2 cells are not induced into induced osteoblasts on the scaffold.

Even in the case where this composite material is implanted under the skin, it is predicted that osteogenesis is not induced. In the case where the composite material is implanted into a bone defective region, it is predicted that osteogenesis is slightly induced (on par with a case that the scaffold is independently implanted thereinto), even if the induced osteoblasts have not been induced on the scaffold.

In order to produce the composite material of the present invention, in the case where mesenchymal stem cells (e.g., undifferentiated cells derived from bone marrow) are inoculated on a scaffold, and then cultured in a medium to which a supernatant containing the induced osteoblast differentiation inducing agent of the present invention is added, the mesenchymal stem cells are induced into induced osteoblasts on the scaffold. In the case where this composite material is implanted under the skin of a syngenic animal or an immunodeficient animal, it is predicted that osteogenesis is induced. Further, in the case where the composite material is implanted into a bone defective region thereof, it is predicted that good osteogenesis is induced.

On the other hand, even in the case where the mesenchymal stem cells (e.g., the undifferentiated cells derived from bone marrow) are inoculated on a scaffold, and then cultured in a medium to which a supernatant containing no induced osteoblast differentiation inducing agent (a supernatant of a differentiation agent producing medium in which the chondrocytes incapable of hypertrophication are cultured is added, or a medium to which only the differentiation agent producing medium is added, it is predicted that the mesenchymal stem cells are slightly induced into the induced osteoblasts.

This is because the differentiation agent producing medium contains components used for inducing differentiation of bone marrow cells into osteoblasts (glucocorticoid, β-glycerophosphate and ascorbic acid). In the case where the composite material is implanted under the skin of a syngenic animal or an immunodeficient animal, it is predicted that osteogenesis is slightly induced. In the case where the composite material is implanted into a bone defective region thereof, it is predicted that osteogenesis is induced at a slightly large degree as compared with a case that the scaffold is independently implanted thereinto.

Further, in the case where the mesenchymal stem cells (e.g., the undifferentiated cells derived from bone marrow) are inoculated on a scaffold, and then cultured in a medium to which a supernatant containing no induced osteoblast differentiation inducing agent (a supernatant of a growth medium in which the chondrocytes capable of hypertrophication are cultured) is added, or a medium to which only the growth medium is added, it is predicted that the mesenchymal stem cells are not induced into the induced osteoblasts on the scaffold.

Even if this composite material is implanted under the skin of a syngenic animals or an immunodeficient animal, It is predicted that osteogenesis is not induced. In the case where the composite material is implanted into a bone defective region thereof, it is predicted that osteogenesis is induced slightly (on par with a case that the scaffold is independently inoculated thereinto), even if the induced osteoblasts have not been induced on the scaffold.

The composite material of the present invention can be used for repairing and reconstructing bone by being implanted. Examples of a region where the composite material is implanted include, but not limited to, bone defective regions formed due to lesions, excision of bone tumors or the like, to which repair and reconstitution of bone is normally required. The composite material of the present invention may be used for forming bone in a region where the bone does not exist in the vicinity thereof. The implantation can be performed in the same manner as known implantation using stem cells derived from bone marrow. An amount of the composite material to be implanted is properly selected depending on sizes of bone defective regions and symptoms and the like.

The present invention also can be optionally used together with a physiologically active substance such as cytokine.

A “cellular physiologically active substance” or a “physiologically active substance” interchangeably used herein refers to a substance which affects cells or tissues. Examples of such effects include control or modification of the cells or the tissues, but are not limited thereto. The physiologically active substance includes cytokine or a growth factor. The physiologically active substance maybe a natural or a synthesized substance.

Preferably, the physiologically active substance may be one produced by cells, or one having a function similar to, but modified from those produced in the cells. The physiologically active substance used herein may be in the form of a protein including a peptide, in the form of a nucleic acid, or in another form.

“Cytokine” used herein is defined as the same meaning as the broadest meaning used in the art. It refers to a physiologically active substance produced in cells that affects the same or different cells. Generally, the cytokine is a protein or a polypeptide, and has activities that control immune response, modulate endocrine system, modulate nervous system, affect anti-tumor action, affect anti-viral action, modulate cell growth, modulate cell differentiation, modulate cellular function, and the like. The cytokine used herein may be in the form of the protein, in the form of the nucleic acid, or in the other form. However, at the time of actually affecting cells, the cytokine is often in the form of the protein including the peptide.

A “growth factor” or a “cellular growth factor” interchangeably used herein refers to a substance which promotes or controls proliferation and induction of differentiation of cells. The growth factor is also referred to as a proliferation factor or a development factor. In cell culture or tissue culture, the growth factor may be substituted for a function of a serum macromolecule when being added to a medium. It is proved that, many growth factors function as factors that regulate a differentiation state in addition to cell growth.

Typical examples of cytokine associated with osteogenesis include factors such as transforming growth factor-β (TGF-β), bone morphogenetic factor (BMP), leukemia inhibitory factor (LIF), colony stimulating factor (CSF), insulin-like growth factor (IGF), fibroblast growth factor (FGF), platelet-rich plasma (PRP), platelet-derived growth factor (PDGF), and vascular endothelial growth factor (VEGF); and compounds such as ascorbic acid, glucocorticoid and glycerophosphoric acid.

Since the physiologically active substance such as the cytokine or the growth factor has generally redundancy, cytokines or growth factors known by another name and function (such as cell adhesion activity or cell-matrix adhesion activity) also may be used in the present invention, as long as they have the activity of the physiologically active substance used in the present invention.

Cytokines or growth factors can be used in the implementation of the present invention, as long as they have preferred activities such as an activity of growing stem cells, an activity of inducing differentiation into the osteoblasts, an activity of promoting production of the induced osteoblast differentiation inducing agent to the chondrocytes capable of hypertrophication.

The induced osteoblast differentiation inducing agent used in the present invention may be derived from cells originated from an individual being in a syngenic relation to a biological organism, an individual being in an allogenic relation to a biological organism, or an individual being in a heterologous relation to a biological organism.

“Originated from an individual being in a syngenic relation to a biological organism” used herein means originated from an autologous, pure line, or inbred line.

“Originated from an individual being in an allogenic relation to a biological organism” used herein means originated from another individual of the same species that are genetically different.

“Originated from an individual being in a heterologous relation to a biological organism” used herein means originated from a heterologous individual. Thus, for example, in the case where a recipient is human, cells derived from rat are “originated from an individual being in a heterologous relation to a biological organism”.

(Use)

In one aspect, the present invention provides use of induced osteoblasts for producing a medicine or medical material capable of promoting or inducing osteogenesis in a biological organism. The induced osteoblasts used in the present invention maybe produced by any that is described above in (Inducing method of induced osteoblasts) in the present specification.

As the induced osteoblasts, any that is described above in (Inducing method of induced osteoblasts), (Chondrocytes capable of hypertrophication) and the like in the present specification maybe used. The induced osteoblasts can be used in the same manner as natural osteoblasts. Therefore, the induced osteoblasts of the present invention can be used for producing the medicine or medical material capable of promoting or inducing the osteogenesis in the biological organism.

Hereinafter, the present invention will be described by various Examples. Examples described below are provided only for illustrative purposes. Accordingly, the scope of the present invention is not limited by the above-described embodiments or the examples below, and instead is limited only by the appended claims.

### Examples

In Examples described below, reagents which were marketed from Wako Pure Chemical Industries Ltd., Invitrogen Corporation, Cambrex Corporation, Aldrich-Sigma Corporation and the like were used with few exceptions.

(Preparation of Medium)

In Examples of the present specification, the following mediums were used except for a specially described case.

(Medium used for Cells)

| Kind of cells | | Medium used |
|---|---|---|
| Chondrocytes | | HAM medium |
| C3H10T1/2 cells | | BME medium |
| 3T3 | Swiss albino cells | D-MEM medium |
| | BALB cells | D-MEM medium |
| | NIH cells | D-MEM medium |

| | |
|---|---|
| Bone marrow cells | MEM growth medium |
| Human mesenchymal stem cells (h-MSC) | MSCGM (growth medium) |

A HAM medium, a BME medium, a D-MEM medium, an MEM growth medium and a MSCGM (growth medium) were prepared so as to become compositions thereof indicated in the following table.

* HAM medium, BME medium, D-MEM medium . . . Common

| | |
|---|---|
| Medium (HAM, BME, D-MEM) | 88.9% |
| Fetal bovine serum | 10% |
| Penicillin•Streptomycin | 1% |
| Fungizone | 0.1% |
| Total | 100% |

* MEM growth medium

| | |
|---|---|
| Minimum essential medium | 83.9% |
| Fetal bovine serum | 15% |
| Penicillin•Streptomycin | 1% |
| Fungizone | 0.1% |
| Total | 100% |

* MEM differentiation agent producing medium

| | |
|---|---|
| Minimum essential medium | 80.9% |
| Fetal bovine serum | 15% |
| Penicillin•Streptomycin | 1% |
| Fungizone | 0.1% |
| β-glycerophosphate | 1% |
| Dexamethasone | 1% |
| Ascorbic acid | 1% |
| Total | 100% |

* MSCGM (growth medium)

| | |
|---|---|
| MSCBM | 88% |
| MSCGS | 10% |
| L-glutamine | 2% |
| Penicillin•Streptomycin | 0.1% |
| Total | 100% |

Medium: basal medium used for preparing each of mediums
HAM: HAM's F12 medium
BME: eagle's basal medium
D-MEM: dulbecco's modified eagle medium
Fungizone: 250 μg/mL Amphotericin B (“15290-018” produced by Invitrogen Corporation)
MSCBM: “PT-3238” produced by Cambrex Corporation
MSCGS: “PT-3001” produced by Cambrex Corporation

### Example 1

### Preparation and Detection of Cellular Function Regulating Agent Produced by Culturing Chondrocytes Capable of Hypertrophication Derived from Costa/Costal Cartilage in MEM Differentiation Agent Producing Medium

(Preparation of Chondrocytes Capable of Hypertrophication from Costa/Costal Cartilages)

Four week-old male rats (Wistar) and 8 week-old male rats (Wistar) were, respectively, divided into groups, and examined in this Example. These rats were sacrificed using chloroform. The rats' chests were shaved using a razor and their whole bodies were immersed into a Hibitane solution (10-fold dilution) to be disinfected. The rats' chests were incised and costa/costal cartilages were collected aseptically.

Translucent growth cartilage regions were collected from boundary regions of the costa/costal cartilages. The growth cartilage regions were sectioned and stirred in a 0.25% trypsin-EDTA/dulbecco's phosphate buffered saline (D-PBS) at 37° C. for 1 hour. Next, the sections were rinsed by centrifugation (at 170×g for 3 minutes), and then stirred together with a 0.2% collagenase (produced by Invitrogen Corporation)/D-PBS at 37° C. for 2.5 hours.

Thereafter, the sections were rinsed by centrifugation (at 170×g for 3minutes), and then stirred together with a 0.2% dispase (produced by Invitrogen Corporation)/(HAM+10% FBS) in a stirring flask overnight at 37° C. In the following day, cells were filtered and rinsed by centrifugation (at 170×g for 3 minutes). The cells were stained with trypan blue and the number thereof was counted under a microscope.

The cells evaluated as cells not stained were considered to be living cells, and those stained blue were considered to be dead cells.

(Identification of Chondrocytes Capable of Hypertrophication)

Since the cells obtained in Example 1 were impaired by the enzymes used in the separation thereof (e.g., the trypsin, the collagenase, and the dispase), they were cultured to recover. Chondrocytes capable of hypertrophication were identified using localization and expression of chondrocyte markers and their morphological hypertrophies under a microscope.

(Expression of Specific Marker for Chondrocytes Capable of Hypertrophication)

A lysate prepared using the method as described above is treated with sodium dodecyl sulfate (SDS) to obtain a SDS-treated solution. The SDS-treated solution is subjected to SDS polyacrylamide gel electrophoresis. Thereafter, a gel used in the SDS polyacrylamide gel electrophoresis is blotted onto a transfer membrane (Western blotting), reacted with a primary antibody to a chondrocyte marker, and then detected with a secondary antibody labeled with an enzyme such as peroxidase, alkaline phosphatase or glucosidase, or a fluorescent tag such as fluorescein isothiocyanate (FITC), phycoerythrin (PE), Texas Red, 7-amino-4-methyl coumarin-3-acetate (AMCA) or rhodamine.

(Expression of Marker Gene for Chondrocytes Capable of Hypertrophication)

Expression of the marker also can be detected by extracting RNAs from the cells obtained using the method as described above, and then assaying the RNAs using a PCR method. In this Example, expression amounts of alkaline phosphatase, type II collagen, aglycan and osteocalcin were measured using a real-time PCR method. GAPDH was used as an integral control gene.

Samples (Gp1 and Gp2) was produced by centrifuging 5×10⁵ chondrocytes capable of hypertrophication prepared in this Example at 170 to 200×g for 3 to 5 minutes to form into pellets, and then culturing the pellets in a 5% CO₂ incubator at 37° C. for 1 week, and used. As a medium, a HAM medium+10% FBS or a HEM medium+15% FBS was used.

(Extraction of Whole RNAs)

1 mL of ISOGEN (produced by Wako Pure Chemical Industries Ltd.) was added to a culture (culture of cells) having a culture area of 12 cm². Cells were removed using a cell scraper, collected into a 2 mL tube, and placed at room temperature for 10 minutes. 0.2 mL of chloroform was added into the tube, acutely vortexed (stirred), and then left at 4° C. for 5 minutes. The tube was centrifuged at 12,000×g and at 4° C. for 15 minutes, and then a supernatant which was a liquid phase was collected into a 1.5 mL tube.

0.5 mL of isopropanol was added into the tube, and the tube was acutely vortexed and left at room temperature for 10 minutes. The tube was centrifuged at 12,000×g and at 4° C. for 15 minutes, a supernatant was completely removed, 1 mL of 70% ethanol was added into the tube, and then the tube was vortexed to obtain whole RNAs. The obtained whole RNAs were dissolved into about 20 μL of a RNase-free water, and then stored at −80° C.

cDNAs were synthesized based on the whole RNAs using a High-Capacity cDNA Archive Kit (produced by Applied Biosystems, Inc.). Expressions of alkaline phosphatase, type II collagen, cartilage proteoglycan (aglycan), osteocalcin and GAPDH were assayed using the cDNAs as templates by a Taqman assay method (“Taqman (registered trademark) Gene Expression Assays” produced by Applied Biosystems, Inc.).

Next, expression amounts of the alkaline phosphatase, the type II collagen, the cartilage proteoglycan (aglycan), the osteocalcin and the GAPDH were measured using a real-time PCR apparatus (“PRISM 7900HT” produced by Applied Biosystems, Inc.). Specifically, a real-time PCR reaction liquid (containing 25 μL of 2×TaqMan Universal PCR Master Mix, 2.5 μL of 20×Taqman (registered trademark) Gene Expression Assay Mix, 21.5 μL of a RNase-free water, and 1 μL of template cDNAs) was prepared, and then dispensed in a 96-well reaction plate.

After the real-time PCR reaction liquid was heated at 50° C. for 2 minutes and at 95° C. for 10 minutes, a PCR was performed through 40 cycles of heating treatments each including heating at 95° C. for 15 seconds and heating at 60° C. for 1 minute. After complication of the PCR, setting of threshold values and calculation of attainment cycles were performed using an analysis software incorporated in the apparatus (“PRISM 7900HT”).

Expression amounts of each cell marker were divided by an expression amount of the GAPDH to calculate correction values thereof, and then an average expression amount thereof was obtained by averaging the correction values. As a result, the chondrocytes capable of hypertrophication expressed the alkaline phosphatase, the type II collagen and the aglycan, but did not express the osteocalcin (see, Table I).

TABLE I

Amount (correction value by GAPDH)
average value

| | | | | |
|---|---|---|---|---|
| Sample | 1 | 2 | 3 | Average |

Alkaline phosphatase

| | | | | |
|---|---|---|---|---|
| Gp1 | 0.0455 | 0.0490 | 0.0596 | 0.0514 |
| Gp2 | 0.0656 | 0.0571 | 0.0650 | 0.0626 |

Type II collagen

| | | | | |
|---|---|---|---|---|
| Gp1 | 0.2574 | 0.2576 | 0.2628 | 0.2593 |
| Gp2 | 0.3724 | 0.4158 | 0.5251 | 0.4378 |

Aglycan

| | | | | |
|---|---|---|---|---|
| Gp1 | 0.6254 | 0.6284 | 0.6227 | 0.6255 |
| Gp2 | 0.9471 | 0.9735 | 1.0005 | 0.9737 |

Osteocalcin

| | | | | |
|---|---|---|---|---|
| Gp1 | 0.0006 | 0.0007 | 0.0005 | 0.0006 |
| Gp2 | 0.0065 | 0.0062 | 0.0087 | 0.0071 |

Gp1 and Gp2: pellets of chondrocytes capable of hypertrophication cultured for 1 week

It can be confirmed whether type X collagen, type I collagen, matrix Gla protein, pleiotrophin, decorin and biglycan are also expressed in the same manner as this Example.

(Localization of Specific Marker for Chondrocytes Capable of Hypertrophication)

The culture obtained using the above-described manipulation is fixed with a 10% neutral formalin buffer, reacted with a primary antibody to a chondrocyte marker, and then detected with a secondary antibody labeled with an enzyme such as peroxidase, alkaline phosphatase or glucosidase, or a fluorescent tag such as FITC, PE, Texas Red, AMCA or rhodamine.

An alkaline phosphatase also can be detected using a staining method. The culture obtained using the above-described manipulation was stained by fixing it with a 60% acetone/citric acid buffer, rinsing it with distilled water, and then immersing it into a mixture of First Violet B and Naphthol AS-MX at room temperature in the dark for 30 minutes to react with each other.

In order to determine whether chondrocytes capable of hypertrophication were present in a cell suspension in which the chondrocytes capable of hypertrophication were diluted, the following experiment was performed. The chondrocytes capable of hypertrophication were inoculated on hydroxyapatite at a density of 1×10⁶ cells/mL, and cultured in a 5% CO₂ incubator at 37° C. for 1 week. Next, this sample (the hydroxyapatite on which the cells were inoculated) was subjected to alkaline phosphatase staining, and then subjected to toluidine blue staining.

The alkaline phosphatase staining was preformed by immersing the sample in a 60% acetone/citric acid buffer for 30 seconds to fix it, rinsing it with water, and then incubating it together with an alkaline phosphatase staining solution (2 mL of a 0.25% naphthol AS-MX alkaline phosphate (Sigma-Aldrich Corporation)+48 mL of a 25% First Violet B salt solution (Sigma-Aldrich Corporaition)) at room temperature in the dark for 30 minutes.

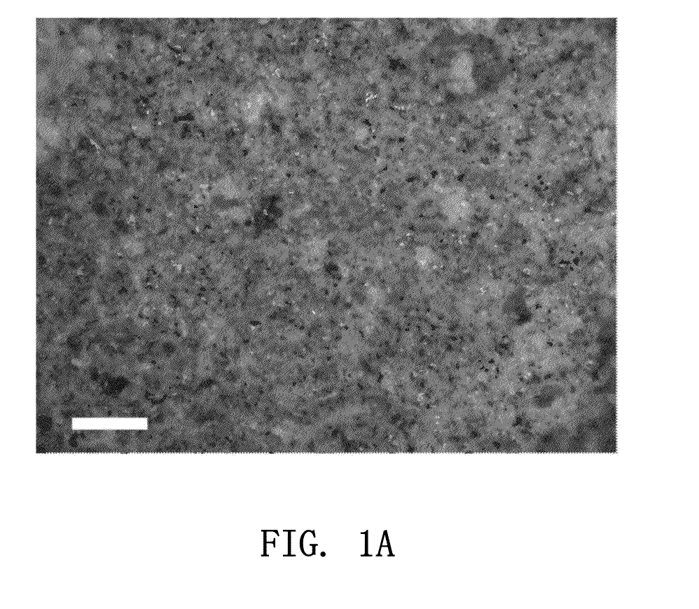

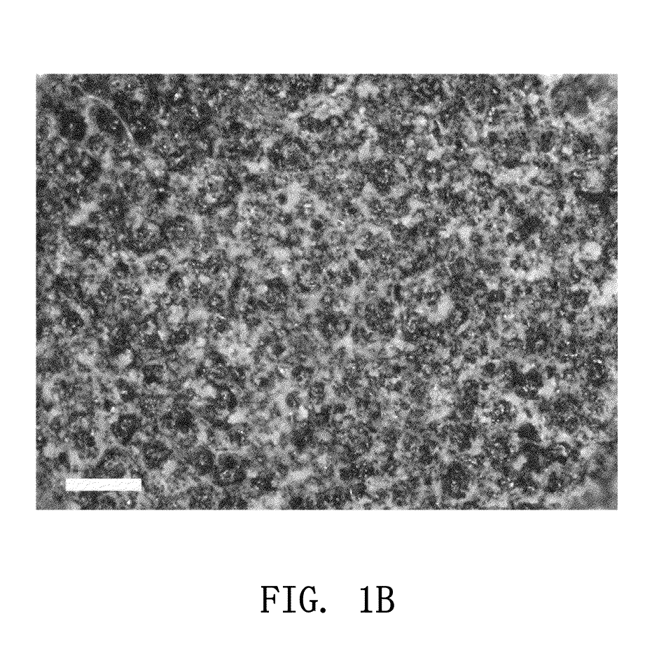

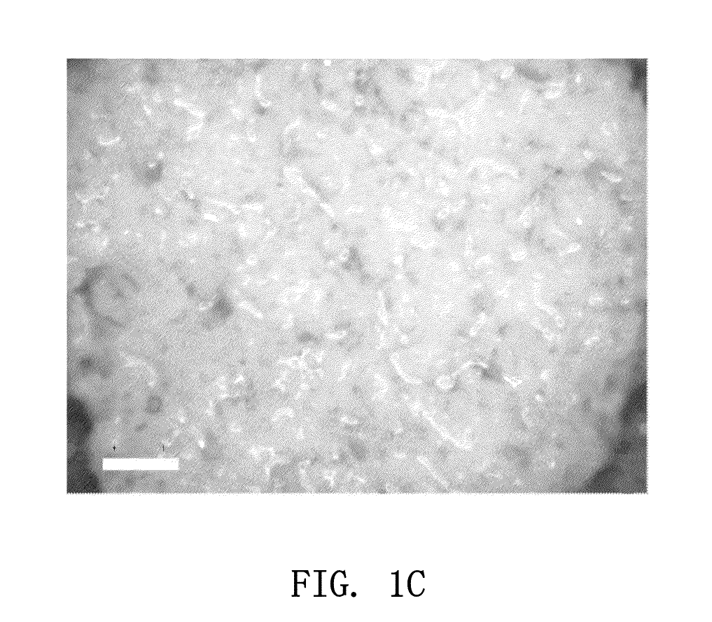

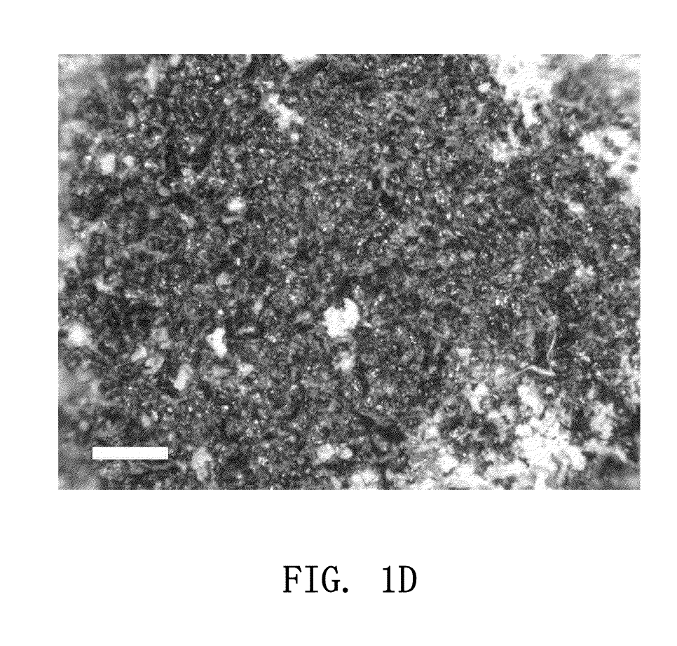

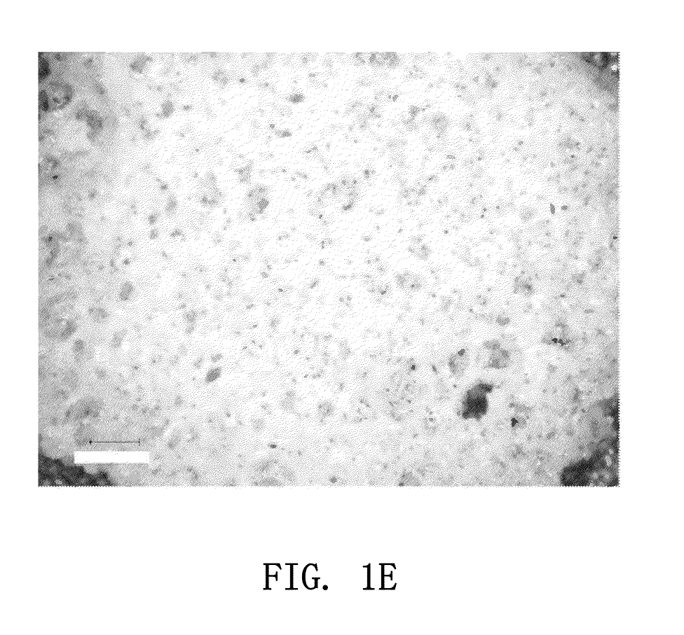

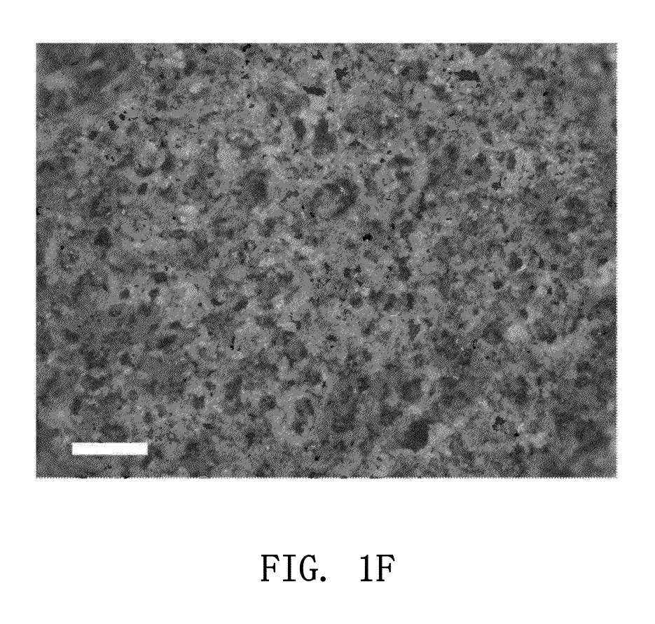

On the other hand, the toluidine blue staining was performed by incubating the sample with a toluidine blue staining solution (“0.25% toluidine blue solution: pH 7.0” produced by Wako Pure Chemical Industries Ltd.) at room temperature for 5 minutes. Spotted areas of the sample were stained red with the alkaline phosphate staining (see FIG. 1A). The same areas of the sample were stained blue with the toluidine blue staining, thereby showing the presence of cells (see FIG. 1B). Thus, it was observed that cells existing on the hydroxyapatite have an alkaline phosphatase activity.

(Morphological Assessment of Ability of Hypertrophication in Chondrocytes)

A HAM's F12 medium containing 5×10⁵ cells was centrifuged to form a pellet of the cells. The pellet (cell pellet) was cultured for a predetermined period. Cell sizes before and after the culture were compared under a microscope. In the case where a significant increase in size was observed, the cells were determined to be capable of hypertrophication.

(Results)

The cells obtained in Example 1 expressed a chondrocyte marker, and were determined to be morphologically-hypertrophic. This shows that the cells obtained in Example 1 were chondrocytes capable of hypertrophication. These cells were used in the following experiments.

(Detection of Agent Produced by Chondrocytes Capable of Hypertrophication Collected from Costa/Costal Cartilage)

Chondrocytes capable of hypertrophication were obtained in the same manner as Example 1. An MEM differentiation agent producing medium (containing a minimum essential medium (MEM), 15% FBS (fetal bovine serum), 10 nM dexamethasone, 10 mM β-glycerophosphate, 50 μg/mL ascorbic acid, 100 U/mL penicillin, 0.1 mg/mL streptomycin and 0.25 μg/mL amphotericin B) was added to the chondrocytes capable of hypertrophication so that they were diluted so as to become a density of 4×10⁴ cells/cm² to prepare a cell suspension.

The cell suspension was inoculated evenly on a dish (produced by Becton, Dickinson and Company), the chondrocytes capable of hypertrophication were cultured in a 5% CO₂ incubator at 37° C., and then a supernatant (culture supernatant) of the medium was collected on a time course (4 days, 7 days, 11 days, 14 days, 18 days, 21 days) to obtain fractional supernatants.

(Study on Whether Supernatant Collected has Ability of Inducing Differentiation of Undifferentiated Cells into Induced Osteoblasts)

Mouse C3H10T1/2 cells (“CCL-226” produced by Dainippon Sumitomo Pharma Co. Ltd.) were inoculated evenly in a 24-well plate (produced by Becton, Dickinson and Company) at a density of 1.25×10⁴ cells/cm² (i.e., 2.5×10⁴ cells/well).

These cells are available from domestic and foreign sales companies such as Sanko Junyaku Co., Ltd., Cosmo Bio Co., Ltd., Takara Bio Inc., Toyobo Co., Ltd., Summit Pharma Biomedical, Stem Cell Sciences, Cambrex Corporation, Stem Cell Technologies, Invitrogen Corporation, and Osiris Therapeutic Inc. or resource exploitation organizations (tissue cell banks) such as domestic organizations (e.g., Health Science Research Resources Bank; Cell Bank, RIKEN BioResource Center; Cell Bank, National Institute of Health Sciences; Institute of Development, and Aging, Cancer at Tohoku University) and foreign organizations (e.g., IIAM, ATCC) in addition to Dainippon Sumitomo Pharma Co. Ltd.

Eighteen hours after the inoculation, 1 mL of each of the fractional supernatants and the medium was added to the plate, and then the mouse C3H10T1/2 cells were cultured in a 5% CO₂ incubator at 37° C. to obtain samples and a control sample. After 72 hours, an alkaline phosphatase activity thereof was measured using the following procedures.

(Measurement of Alkaline Phosphatase Activity)

In order to measure an alkaline phosphatase activity, 50 μL of a 4 mg/mL p-nitrophenyl phosphate solution and 50 μL of an alkali buffer (“A9226” produced by Sigma Corporation) were added to 100 μL of each of the samples containing the agents or the control sample containing no agent, and then reacted at 37° C. for 15 minutes. Thereafter, the reaction was terminated by adding 50 μL of a 1N NaOH to the same, and then absorbance (at 405 nm) thereof was measured. Next, 20 μL of concentrated hydrochloric acid was further added to the same, and then absorbance (at 405 nm) thereof was measured.

A difference between these absorbances was referred to as an “absolute active value” (indicated as an “absolute value” in Table), and was used as an indicator of the alkaline phosphatase activity of the mouse C3H10T1/2 cells. In a 4 week-old group, five experiments were performed, and three trials were carried out per 1 experiment. In a 8 week-old group, three experiments were performed, and two trials in the first experiment, two trials in the second experiment, and one trial in the third experiment were performed.

The absolute value in each of the samples is divided by the absolute value in the control sample in which only the medium was added (that is, the absolute active value in the control sample obtained by adding only the medium to the mouse C3H10T1/2 cells) is referred to as a “relative active value” (indicated as a “relative value” in Table) in the present specification, and was used as another indicator of the alkaline phosphatase activity of the mouse C3H10T1/2 cells.

In this Example, in the case where a value of the alkaline phosphatase (ALP) activity of the mouse C3H10T1/2 cells (whole the cells) cultured by adding the supernatant containing the present agent increased by more than 1.5 times that of the mouse C3H10T1/2 cells cultured by adding the medium containing no present agent, the present agent was determined to have an ability of increasing the alkaline phosphatase activity.

In the case where the alkaline phosphatase activity was evaluated using the relative active value, when a value of the alkaline phosphatase activity of the mouse C3H10T1/2 cells cultured by adding only the MEM differentiation agent producing medium was defined as “1”, in the 4 week-old rat group: the relative active value thereof increased about 4.1 times by adding the fractional supernatant collected 4 days after the culture; to about 5.1 times by adding the fractional supernatant collected 1 week after the culture; to about 5.4 times by adding the fractional supernatant collected 2 weeks after the culture; and to about 4.9 times by adding the fractional supernatant collected 3 weeks after the culture.

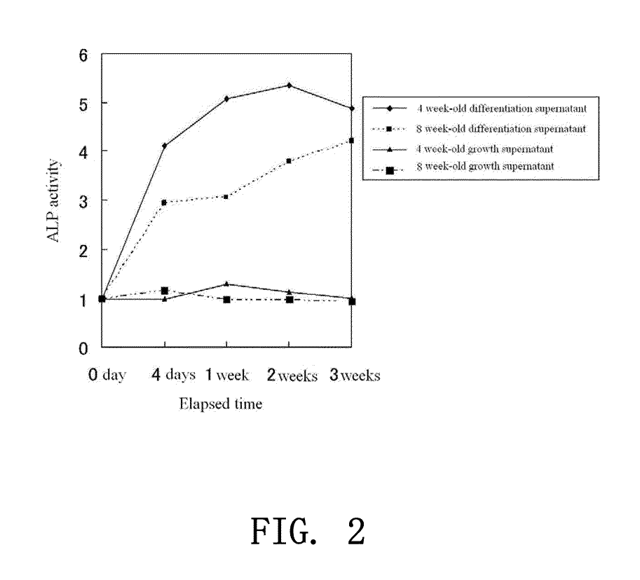

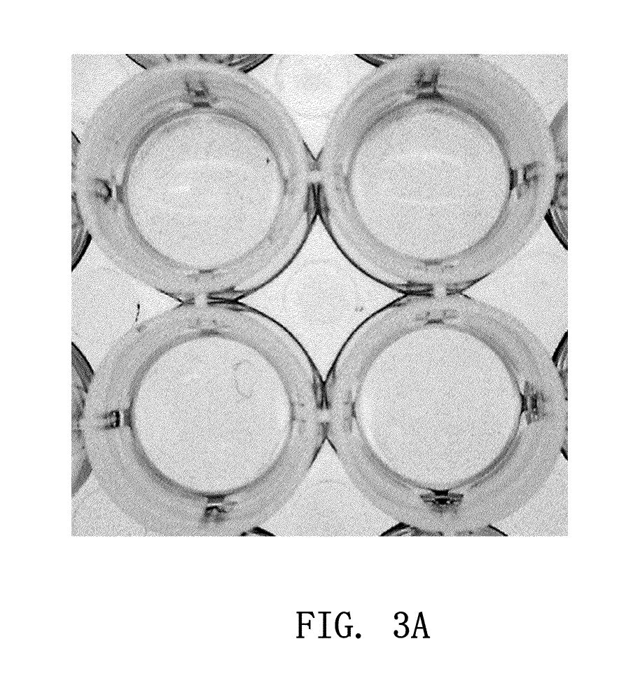

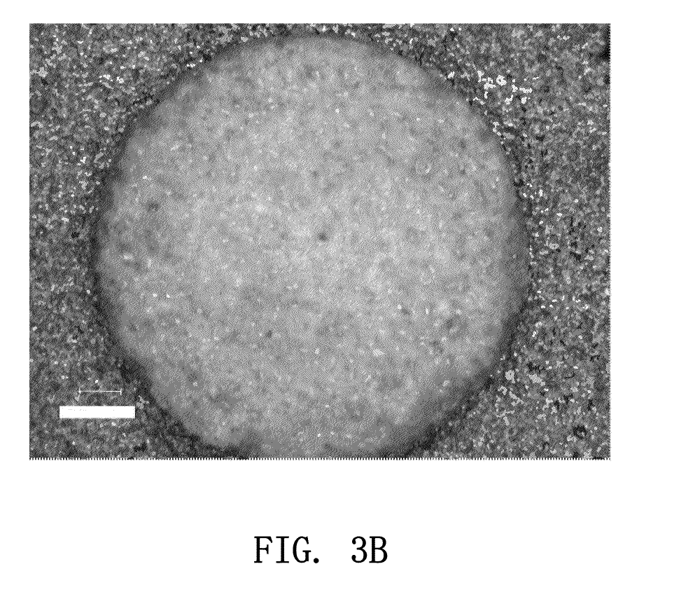

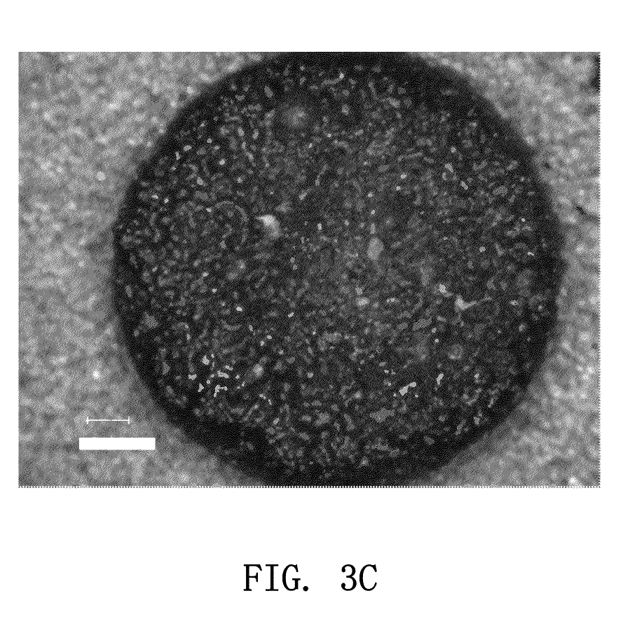

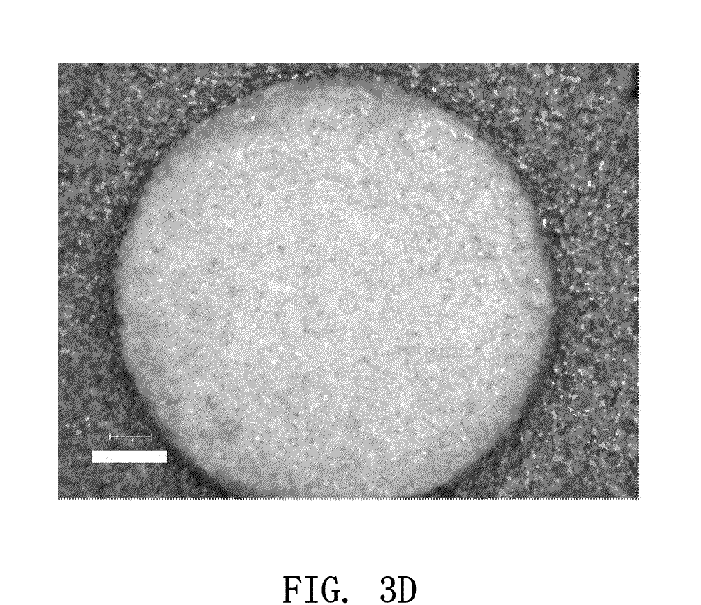

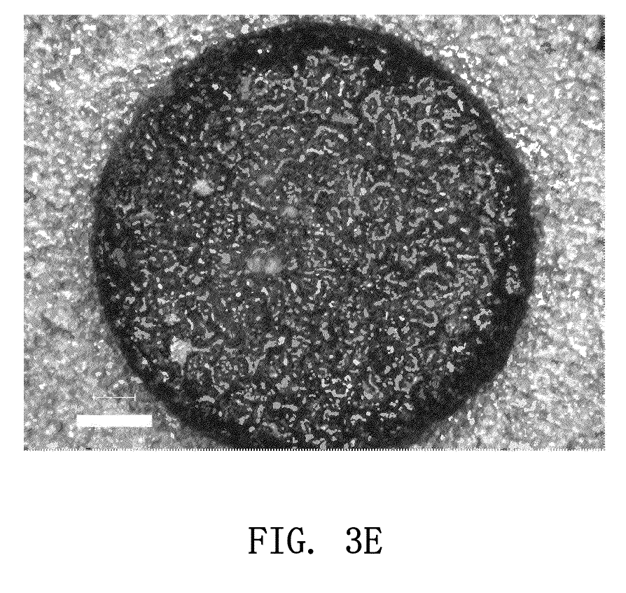

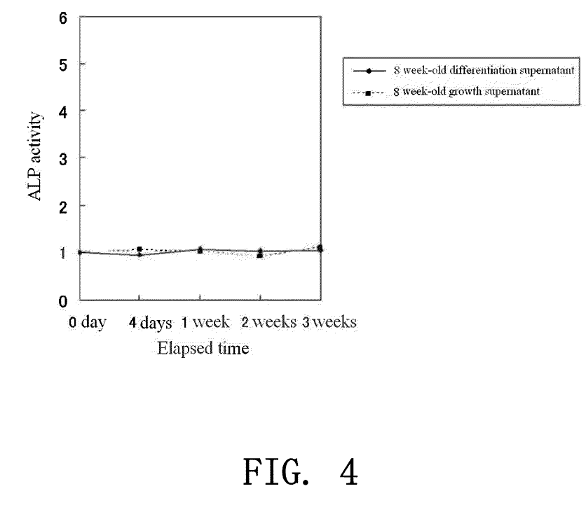

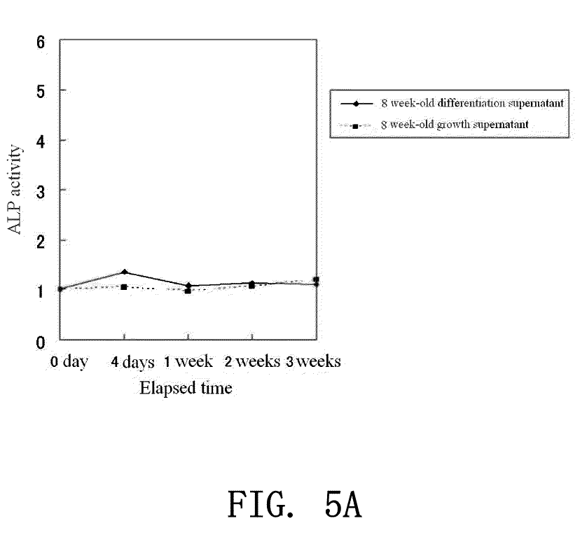

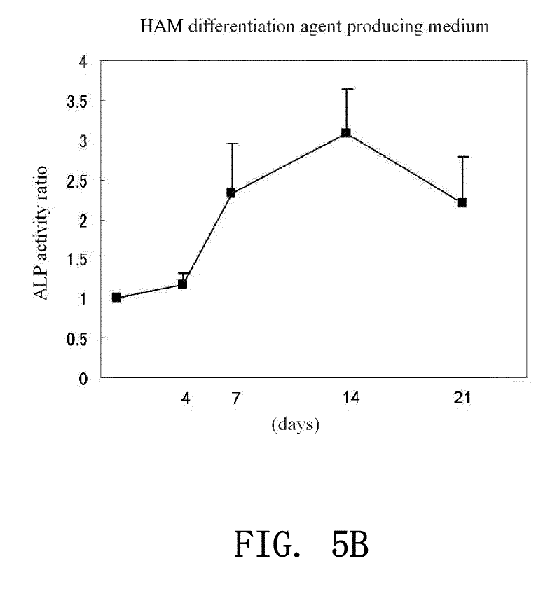

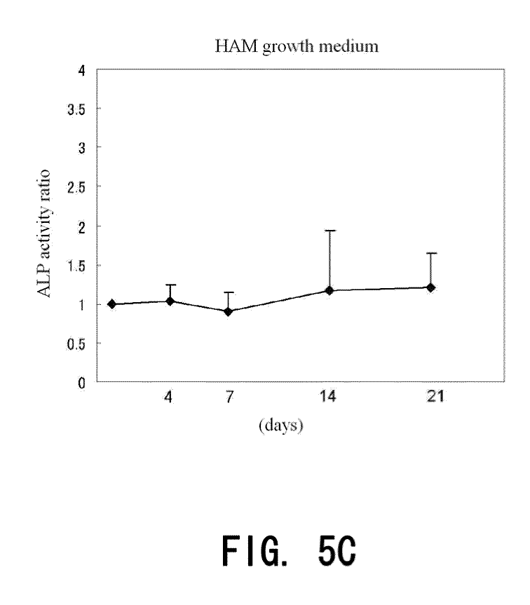

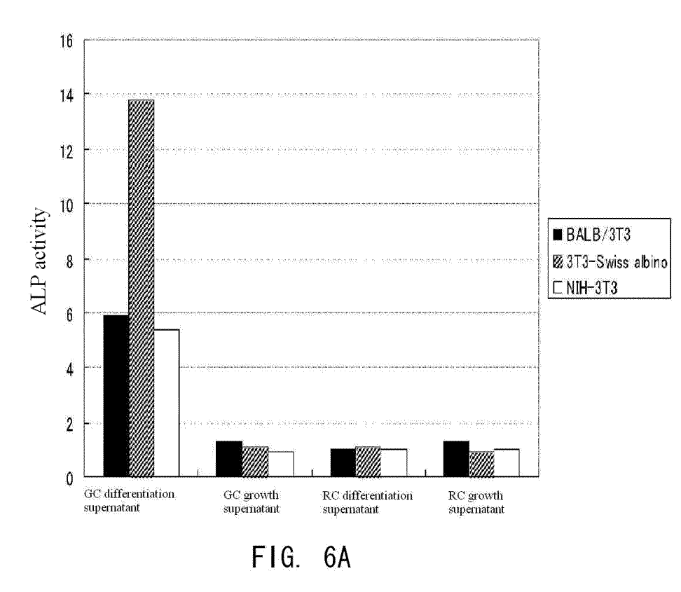

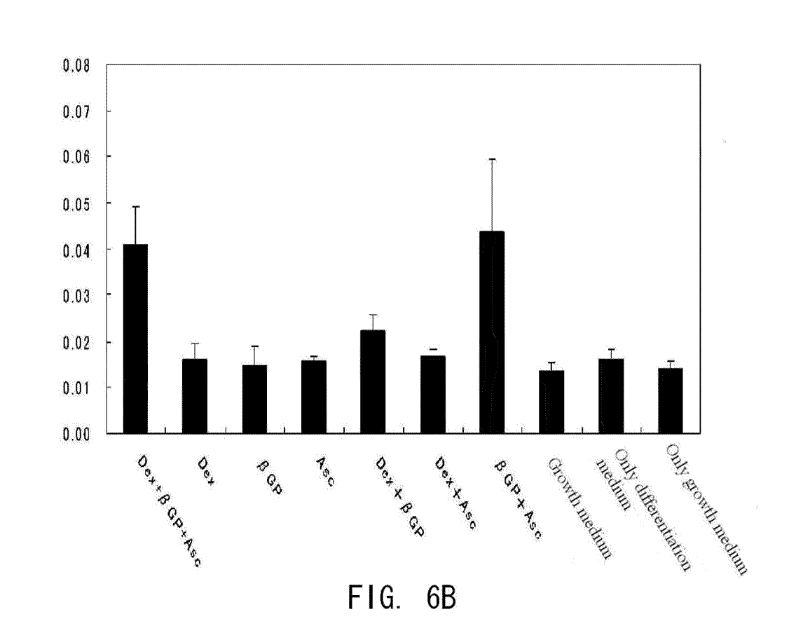

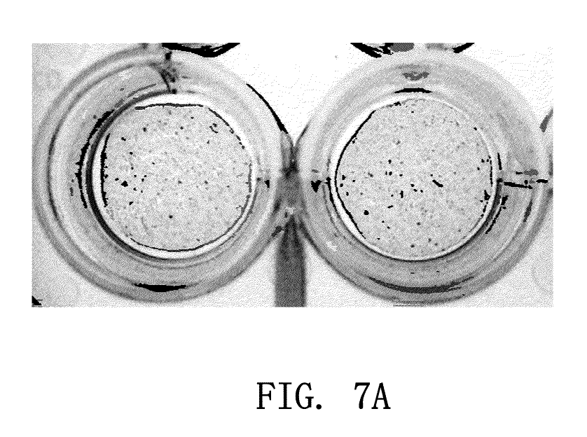

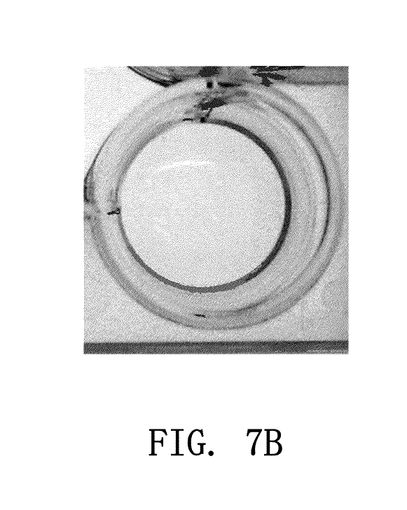

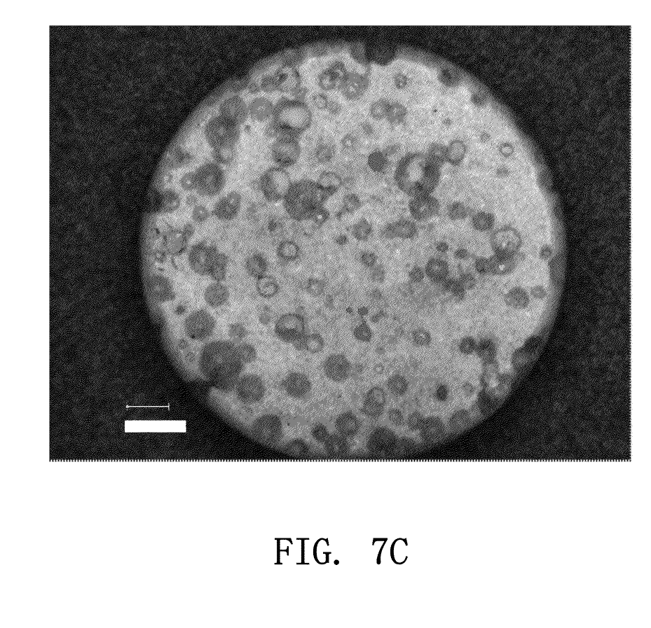

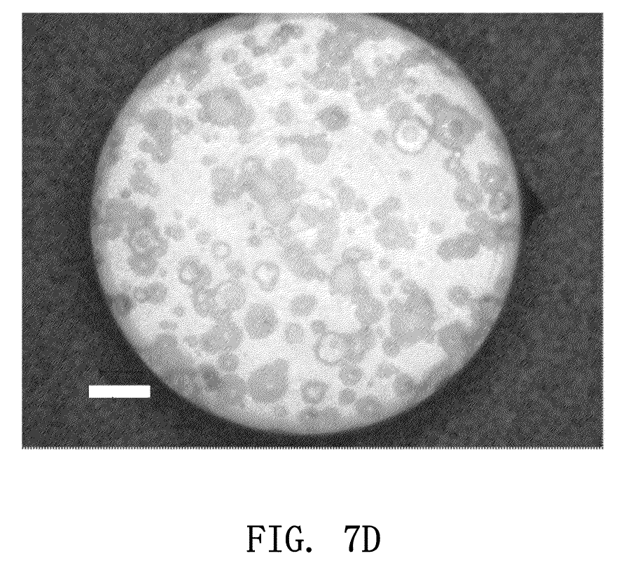

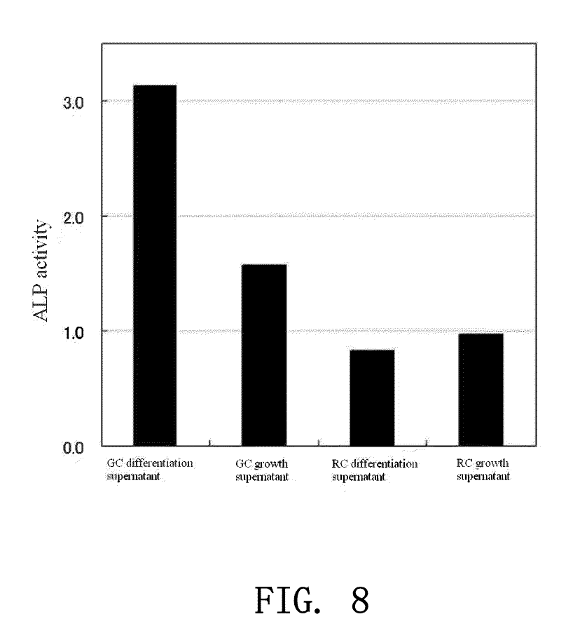

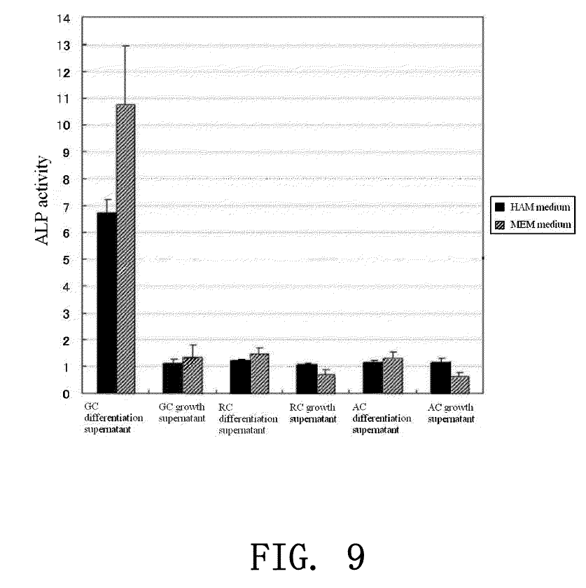

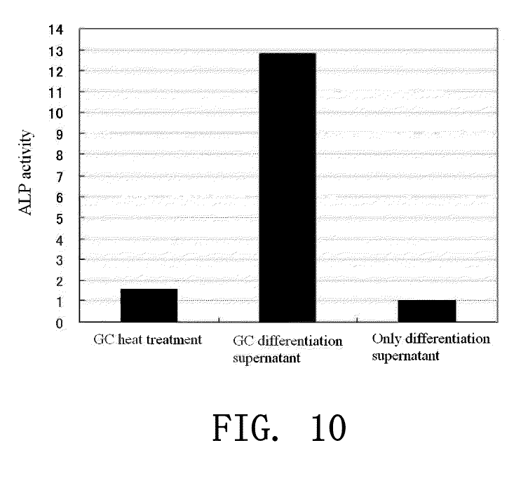

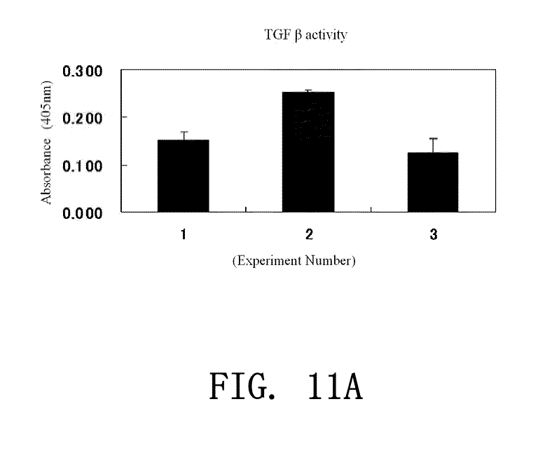

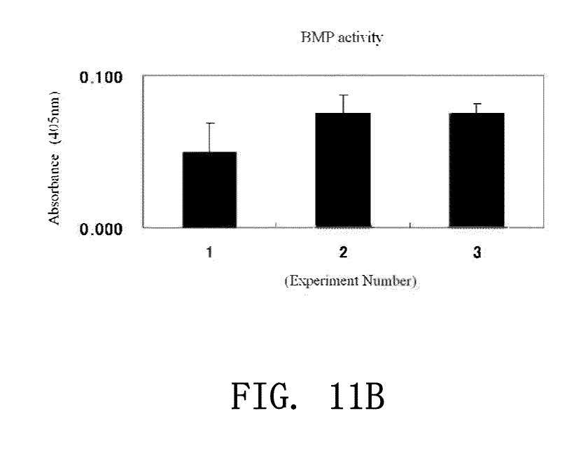

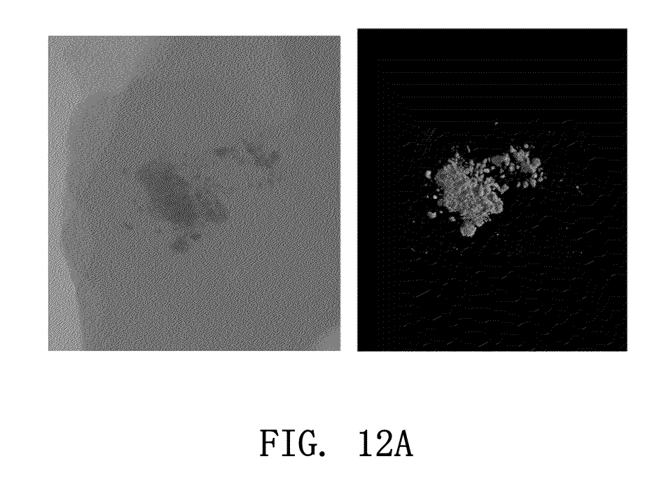

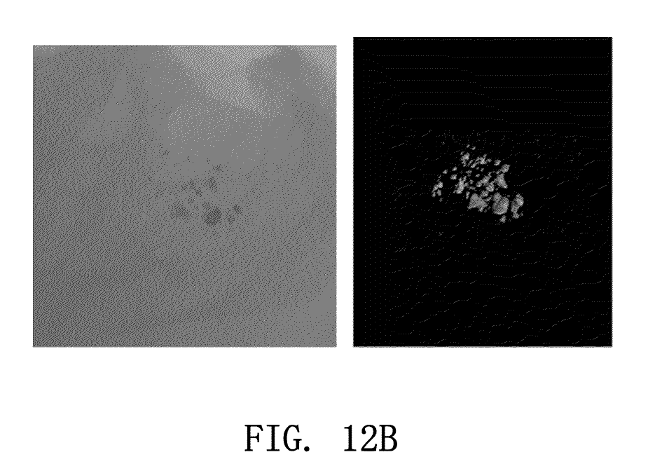

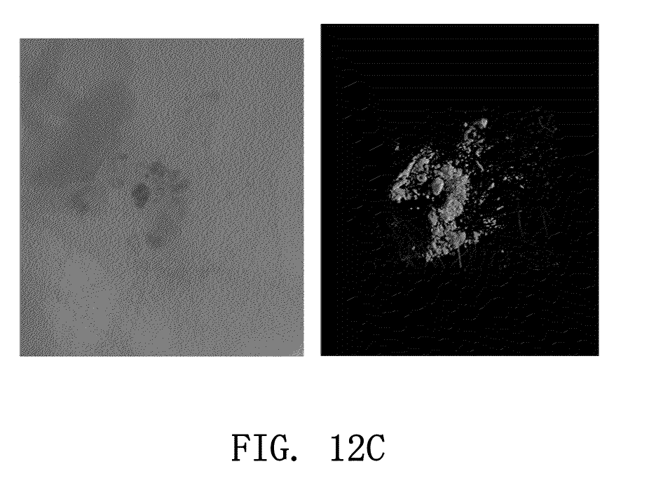

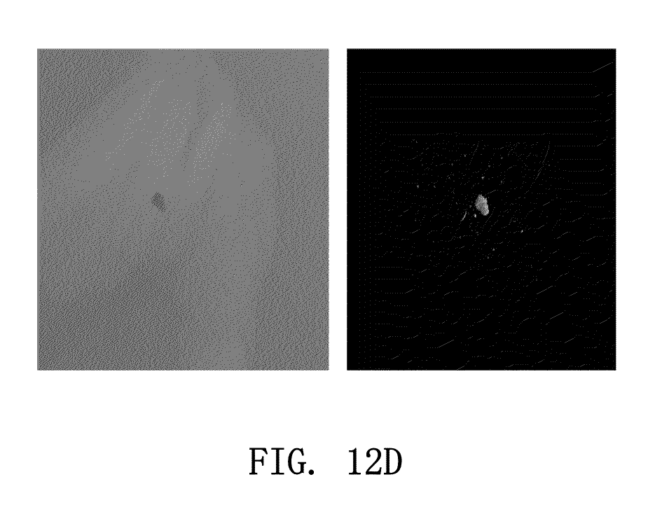

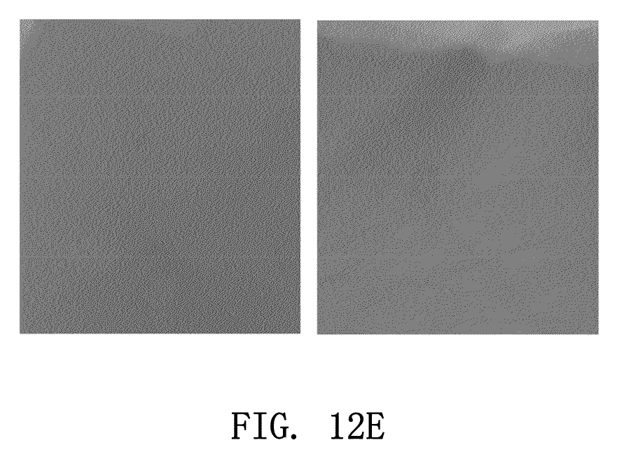

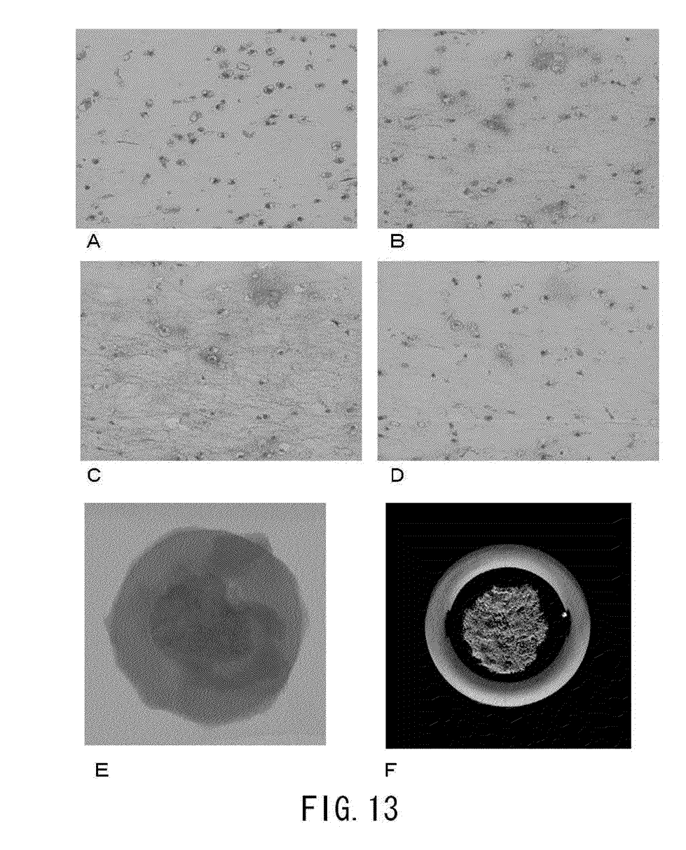

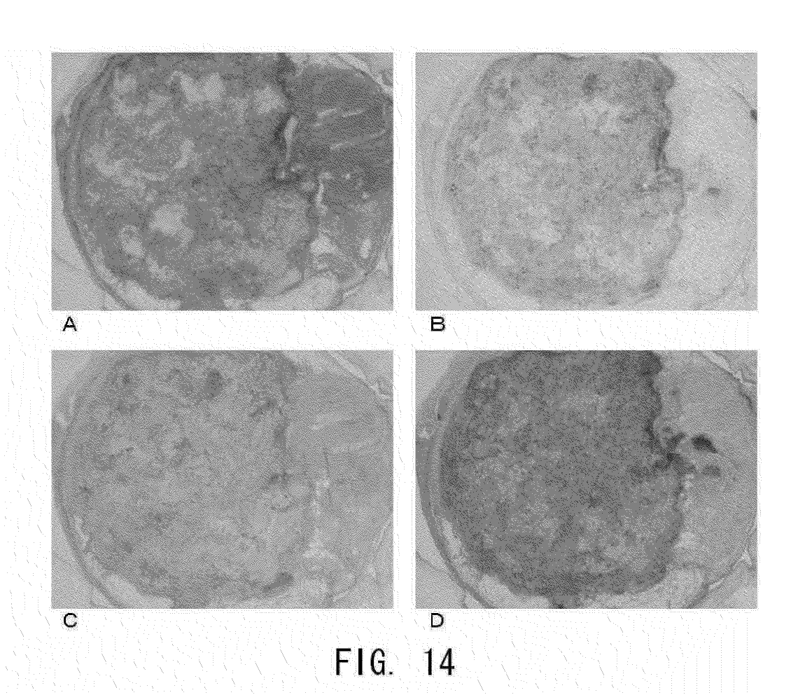

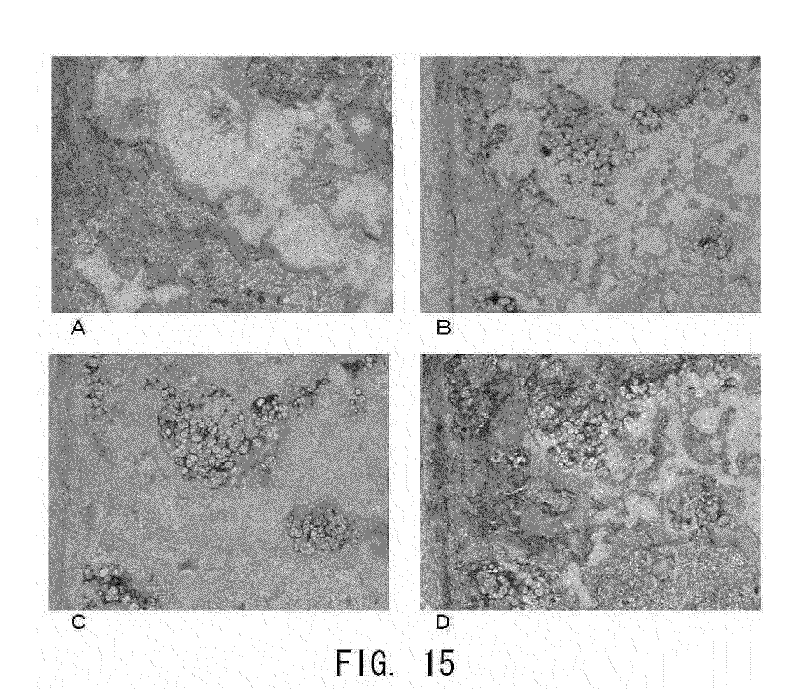

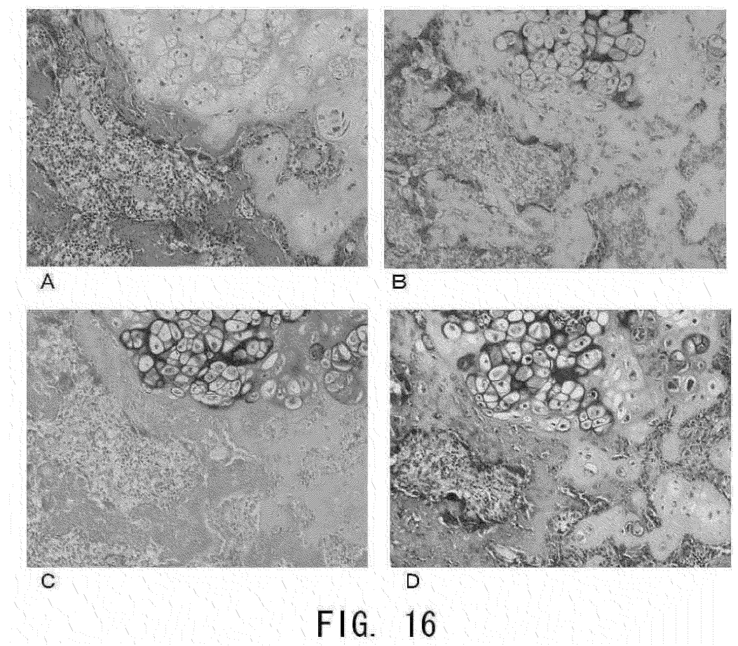

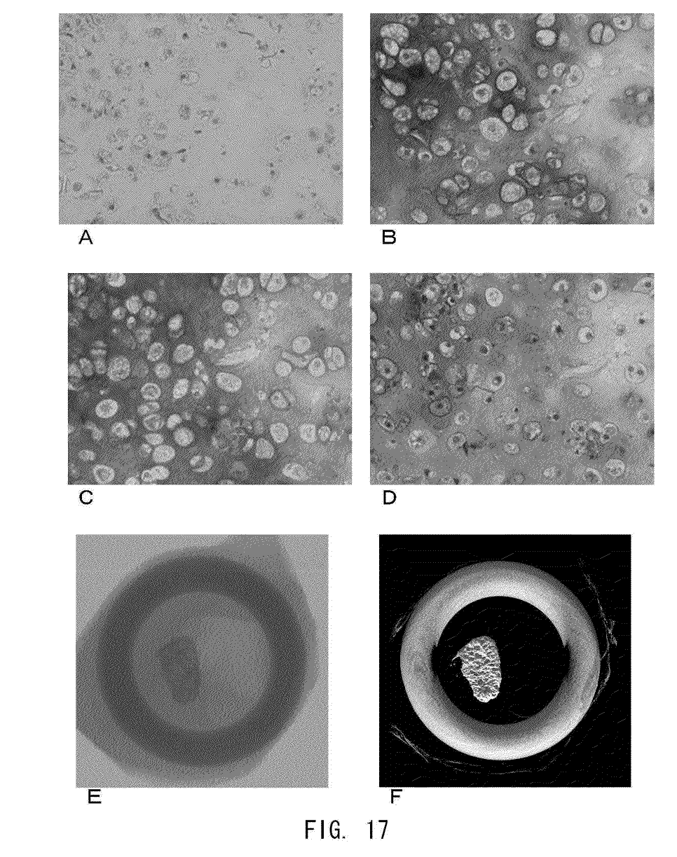

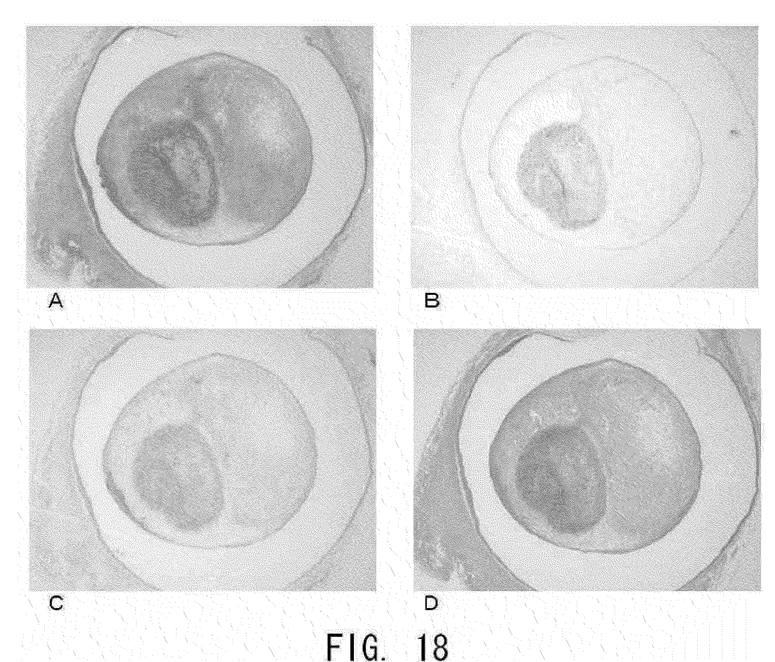

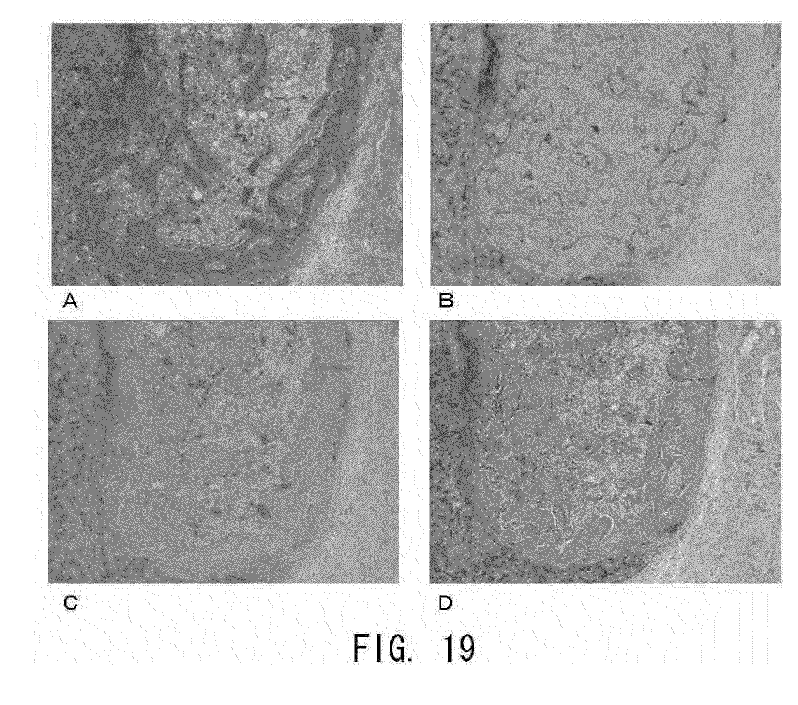

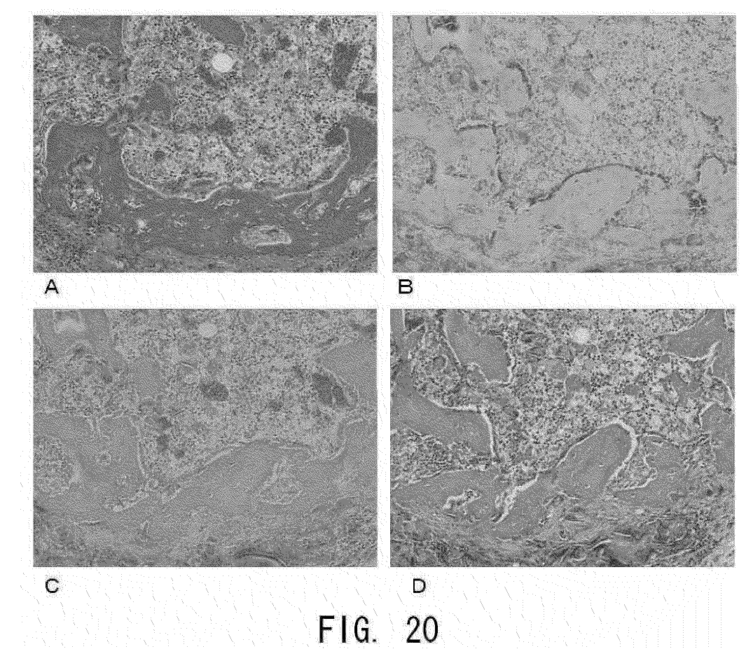

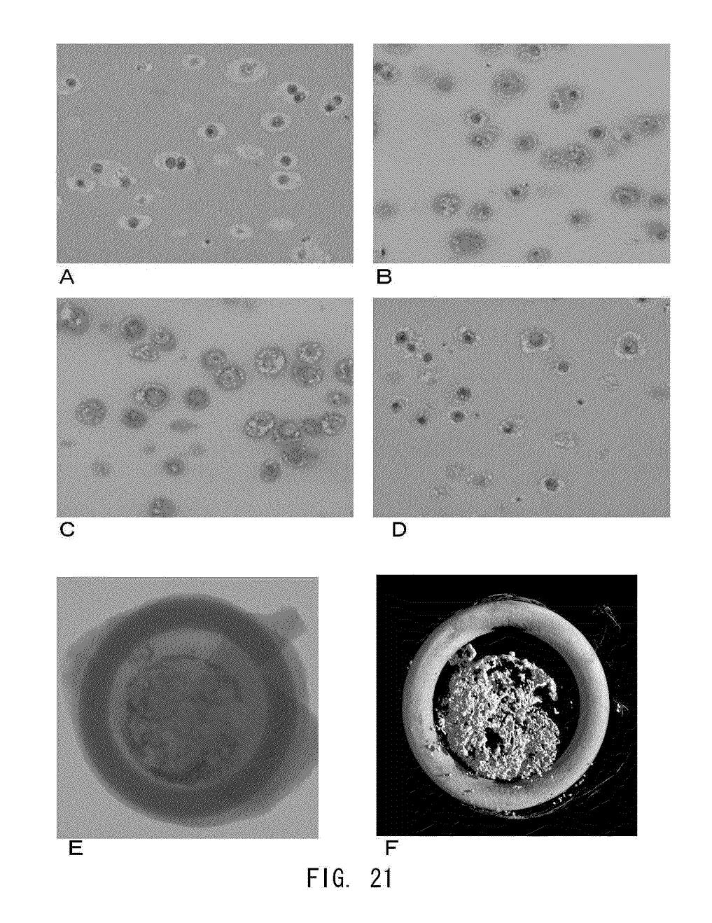

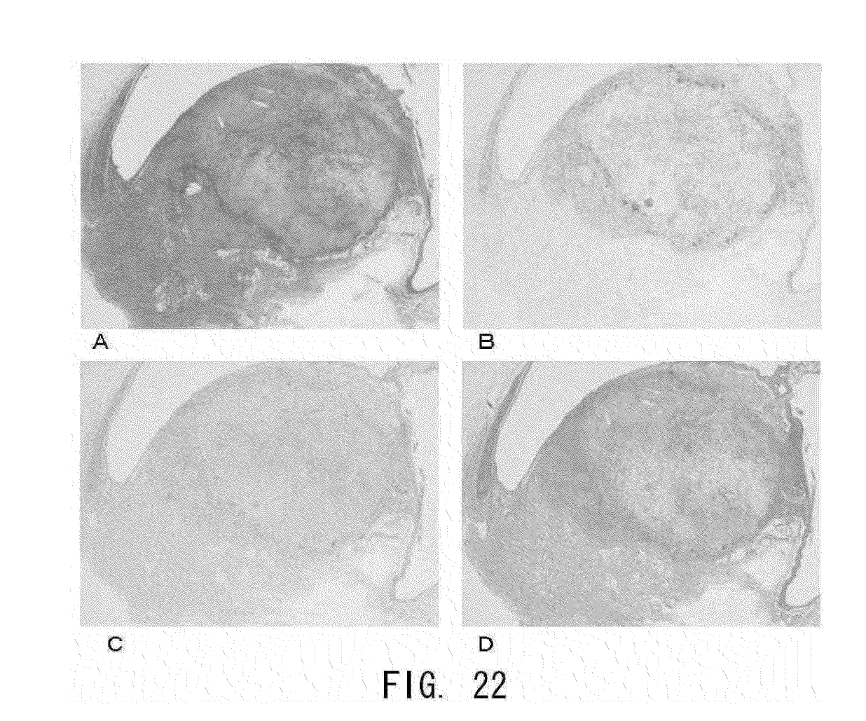

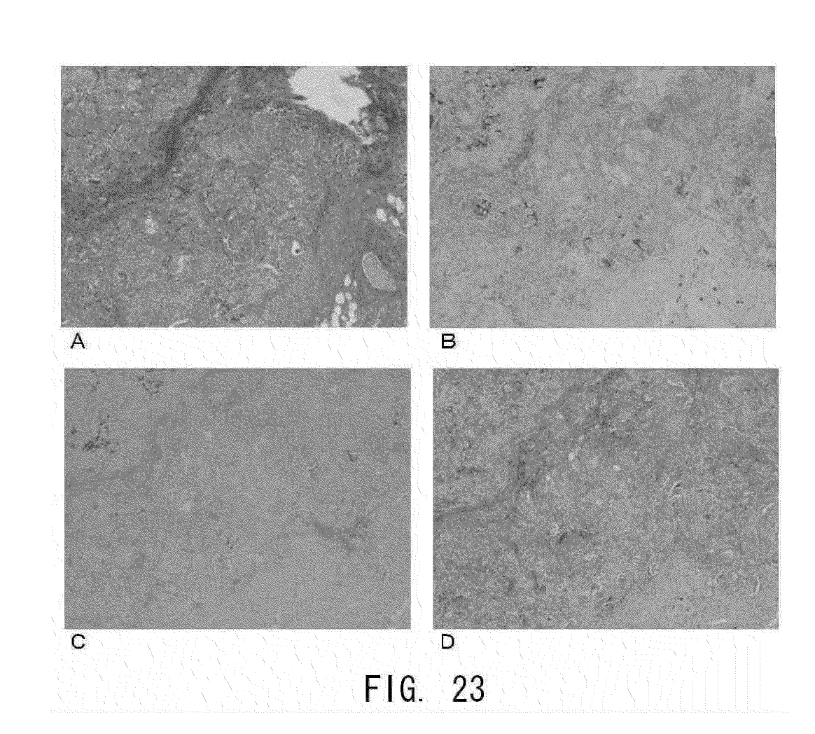

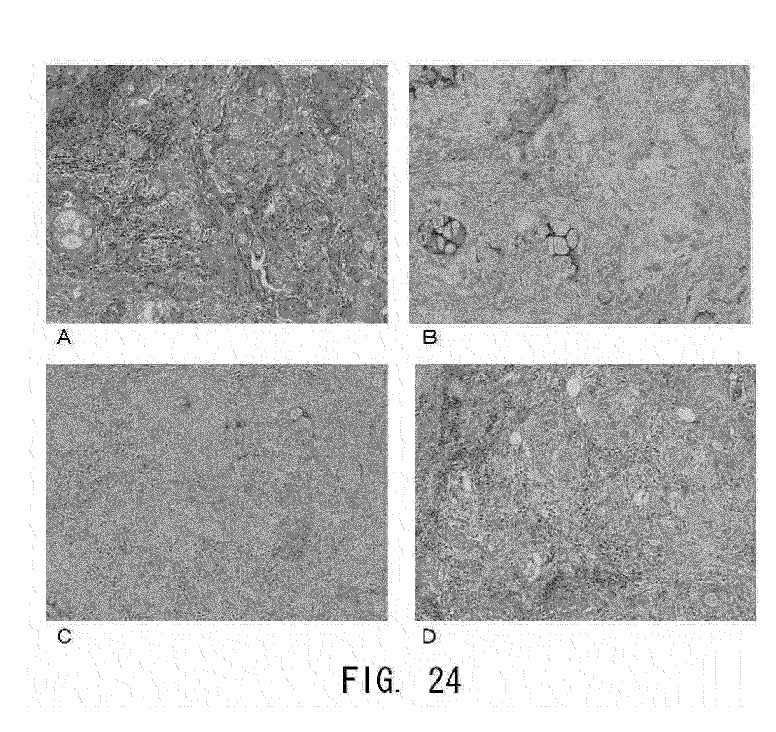

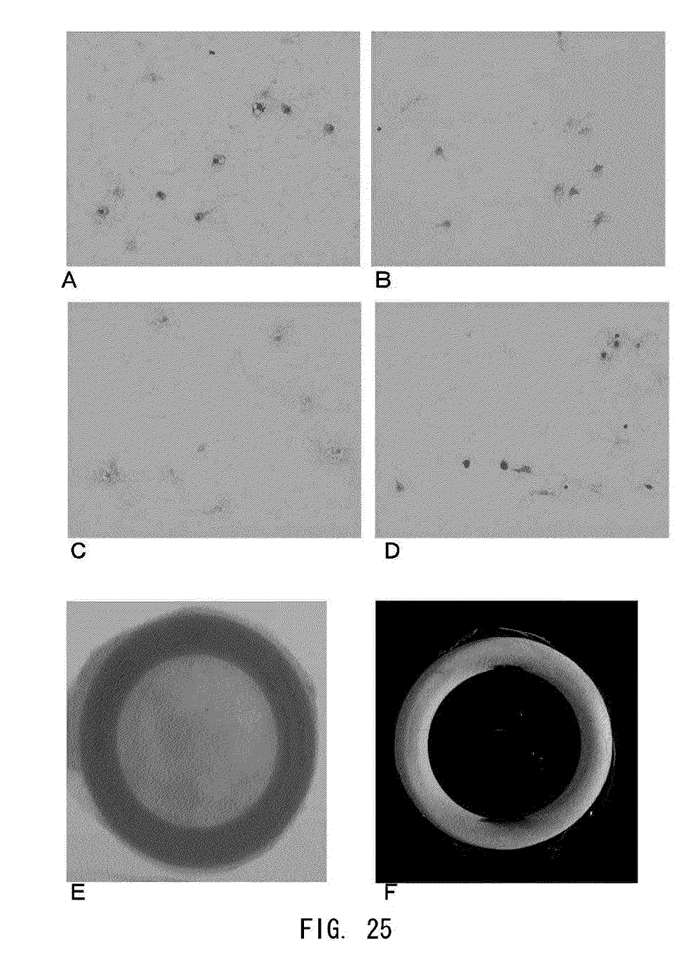

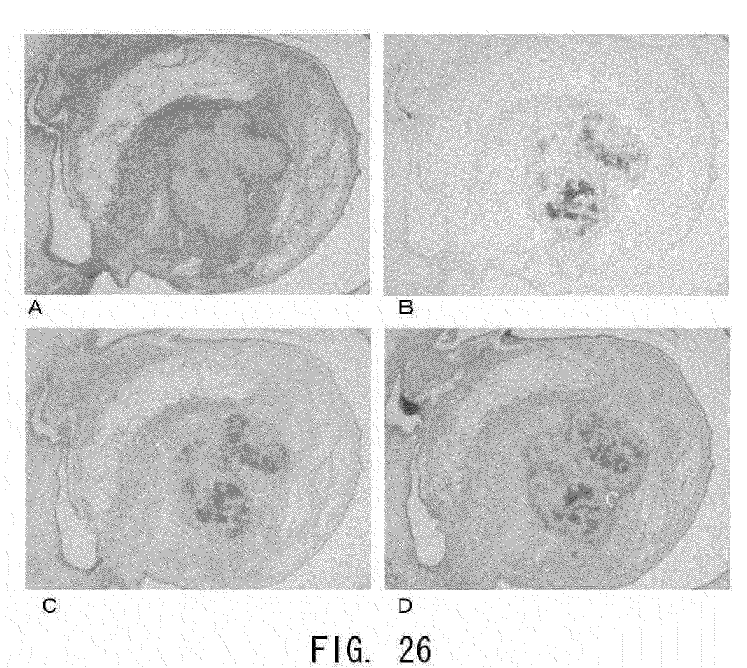

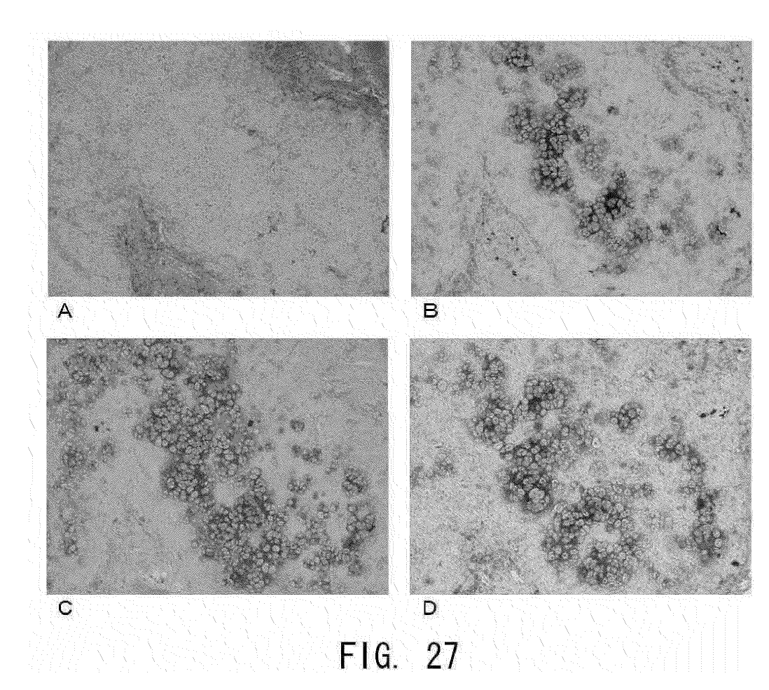

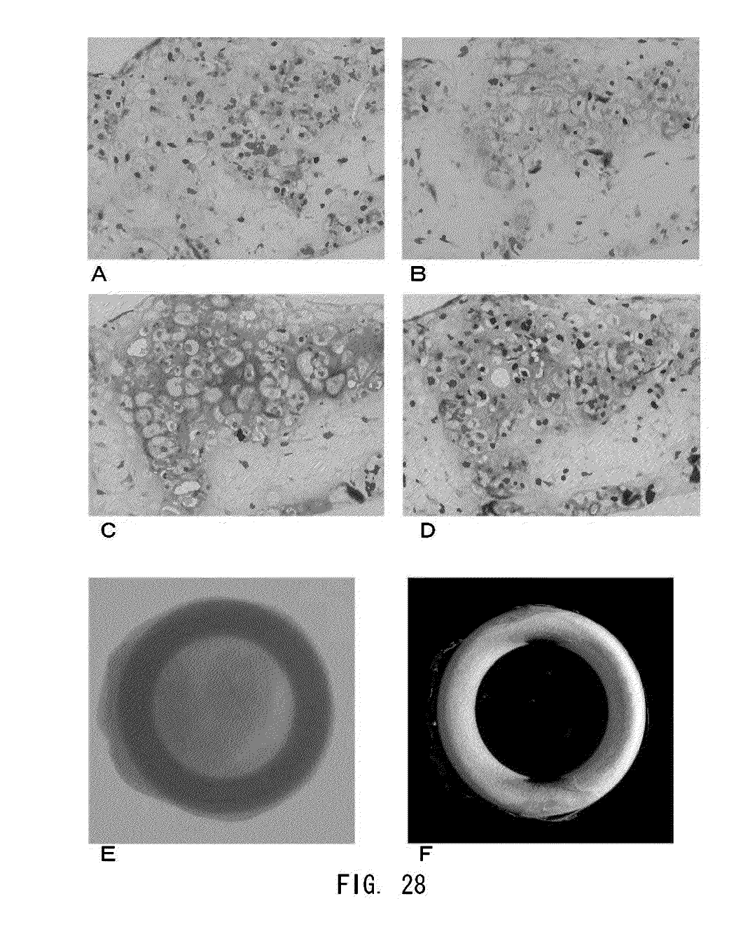

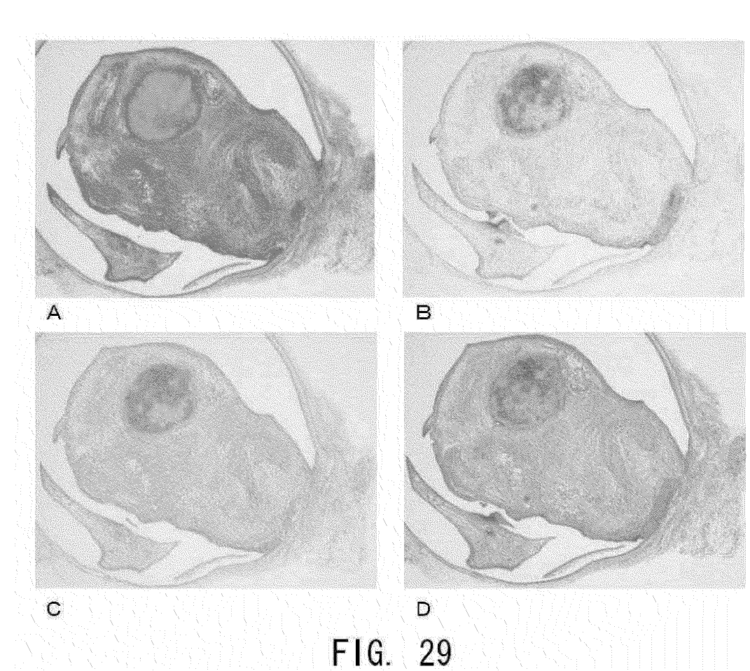

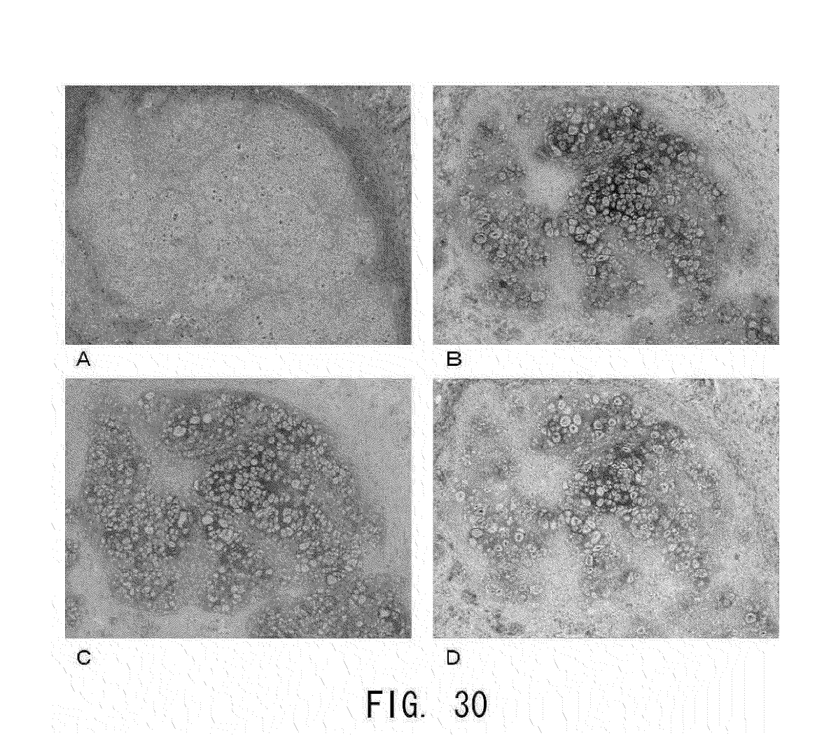

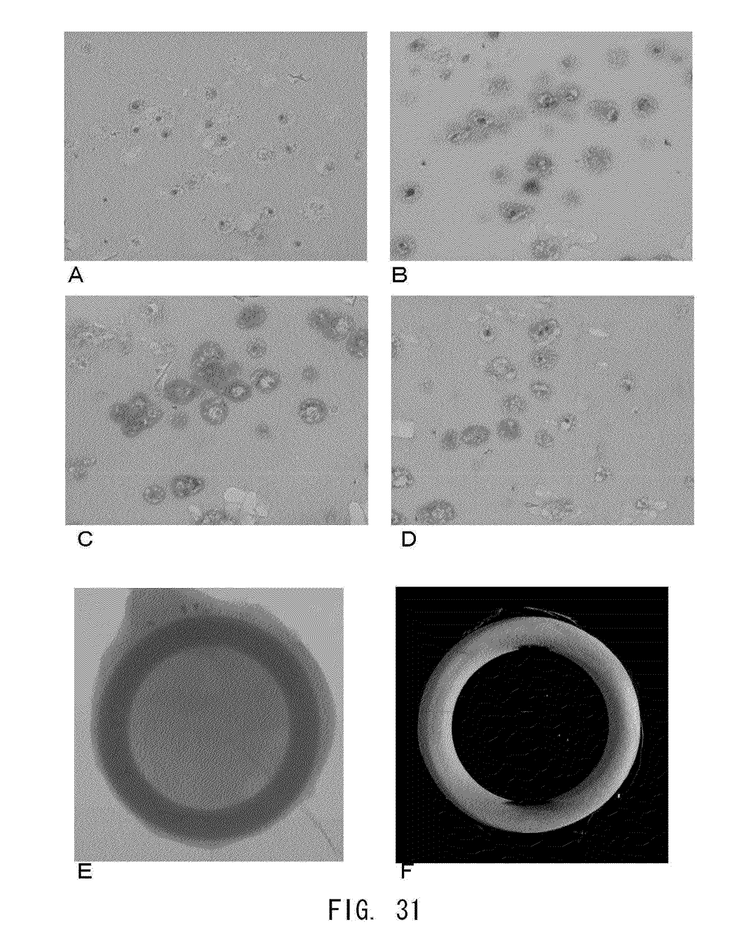

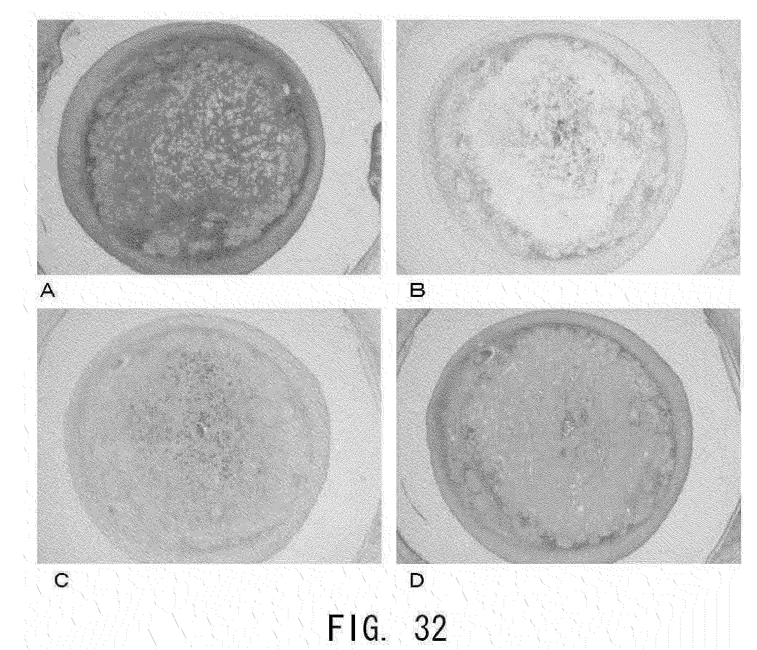

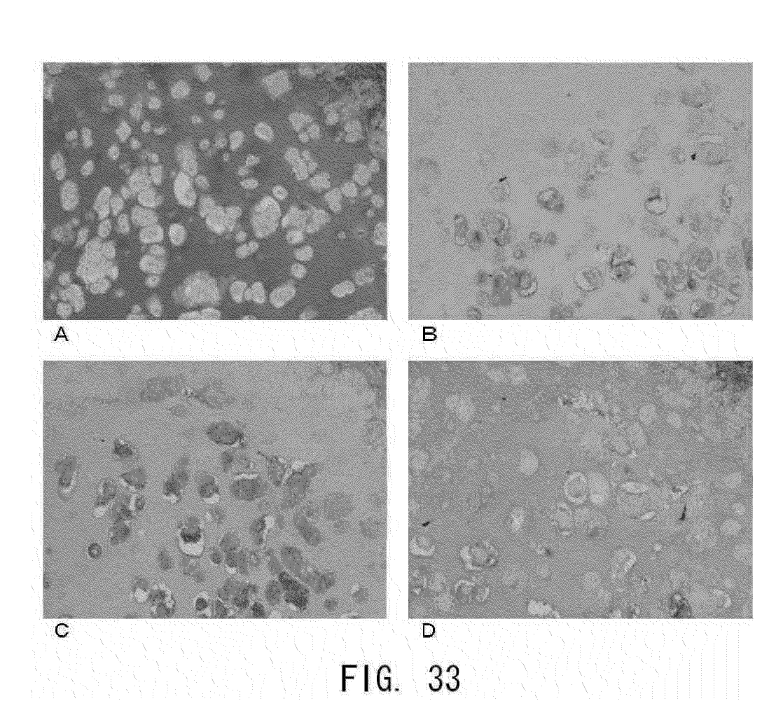

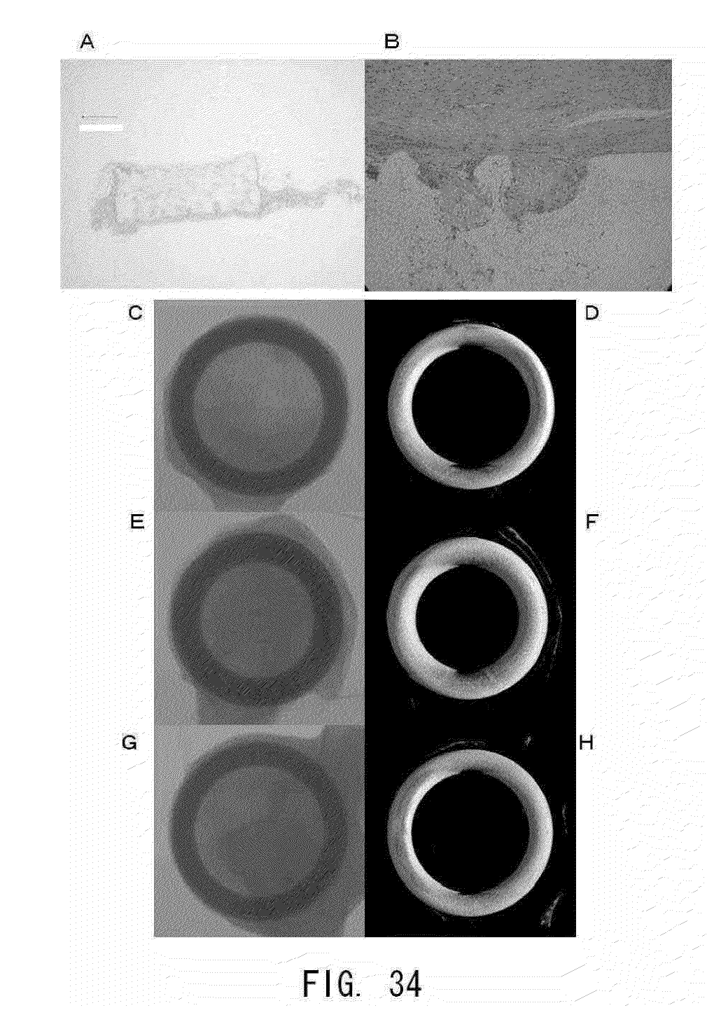

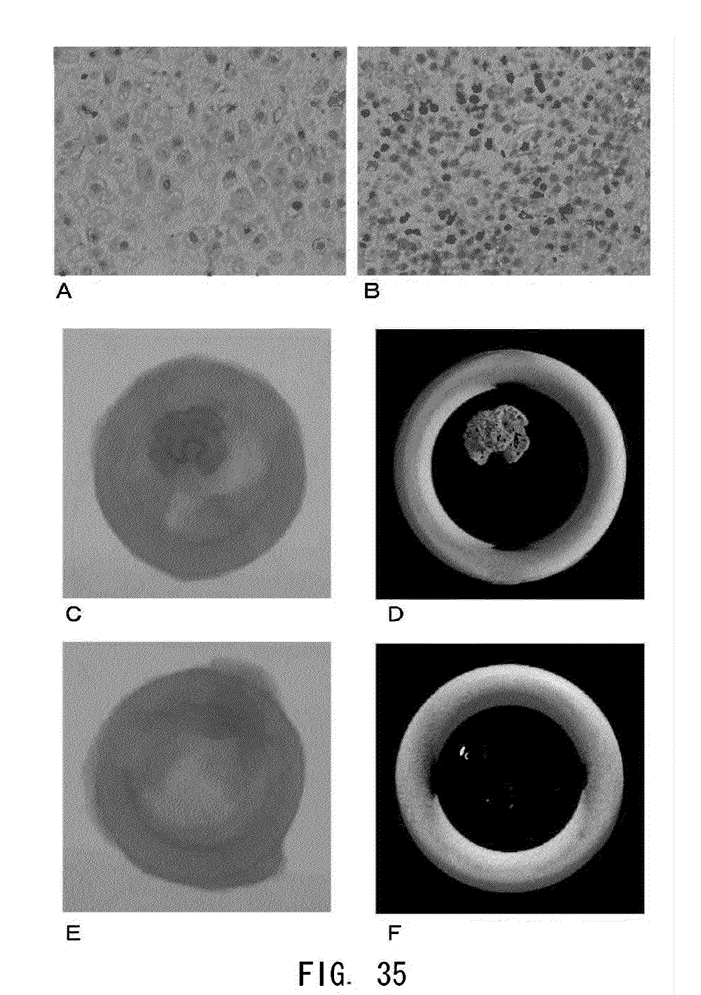

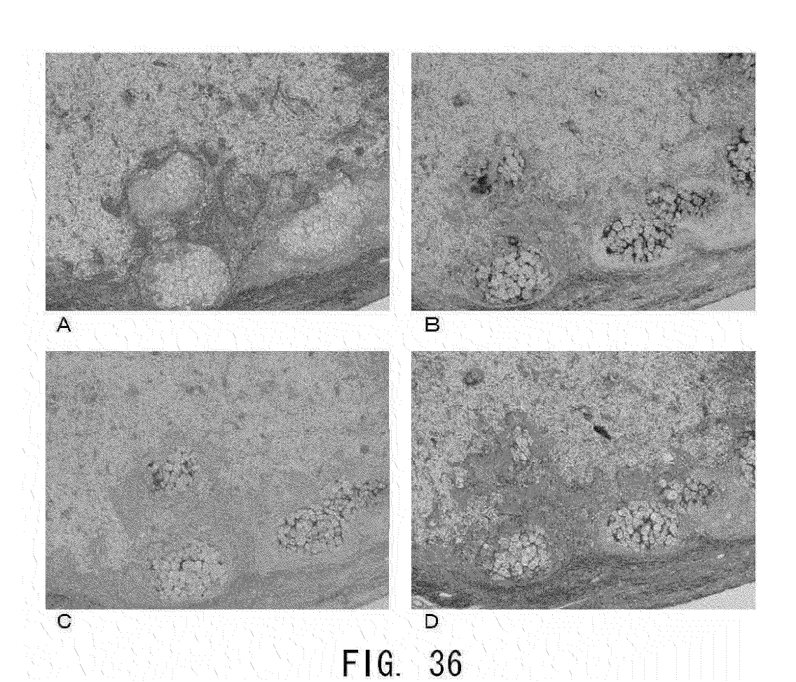

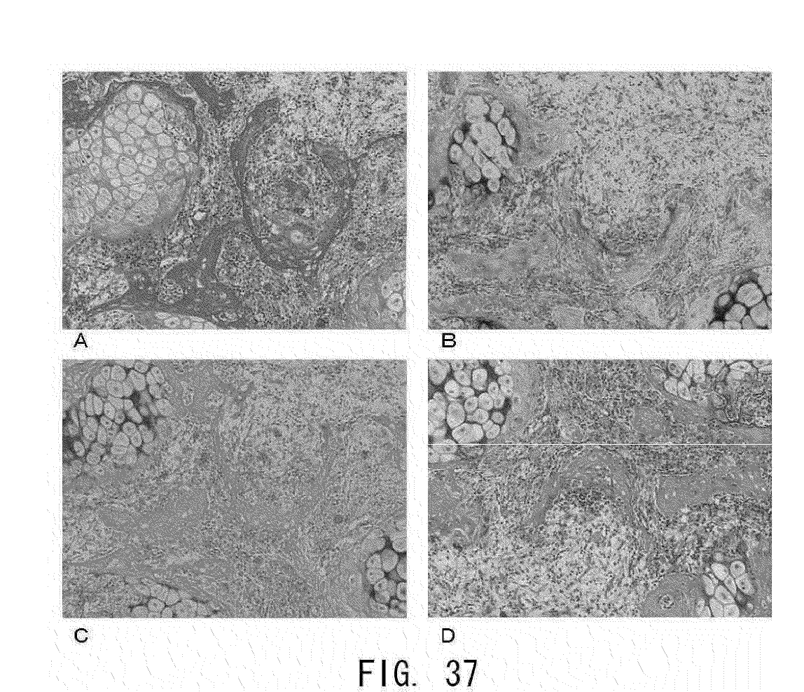

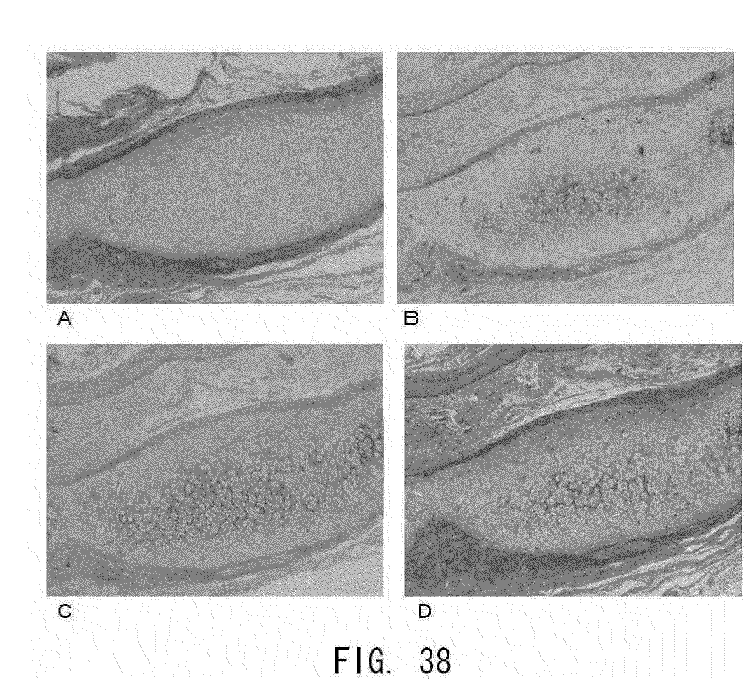

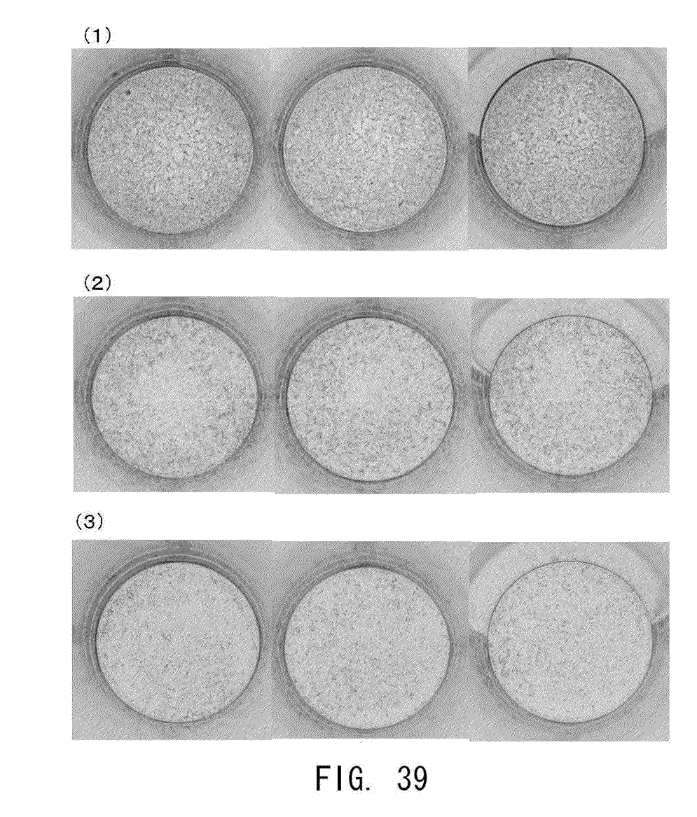

In the above same case, in the 8 week-old rat group: the relative value thereof increased to about 2.9 times by adding the fractional supernatant collected 4 days after the culture; to about 3.1 times by adding the fractional supernatant collected 1 week after the culture; to about 3.8 times by adding the fractional supernatant collected 2 weeks after the culture; and to about 4.2 times by adding the fractional supernatant collected 3 weeks after the culture (see upper column in Table 1, and FIG. 2).

(Identification of Induced Osteoblasts)

(Alkaline Phosphatase Staining)

(In the Case of Addition of Supernatant of MEM Differentiation Agent Producing Medium in which Chondrocytes Capable of Hypertrophication were Cultured)

Mouse C3H10T1/2 cells (“CCL-226” produced by Dainippon Sumitomo Pharma Co. Ltd.) were inoculated evenly in a 24-well plate (produced by Becton, Dickinson and Company) at a density of 1.25×10⁴ cells/cm² (i.e., 2.5×10⁴ cells/well) and on hydroxyapatite at a density of 1×10⁶ cells/mL. Eighteen hours after the inoculation, 1 mL of a supernatant (culture supernatant) of an MEM differentiation agent producing medium, in which chondrocytes capable of hypertrophication were cultured, was added to the plate and the hydroxyapatite, and then the cells were cultured in a 5% CO₂ incubator at 37° C. to obtain cultures (cultures of cells).

The cultures were stained by fixing them with a 60% acetone/citric acid buffer, rinsing them with distilled water, and immersing them into a mixture of First Violet B and Naphthol AS-MX at room temperature in the dark for 30 minutes.

(In the Case of Addition of Supernatant of MEM Growth Medium in which Chondrocytes Capable of Hypertrophication were Cultured)

Mouse C3H10T1/2 cells (“CCL-226” produced by Dainippon Sumitomo Pharma Co. Ltd.) were inoculated evenly in a 24-well plate (produced by Becton, Dickinson and Company) at a density of 1.25×10⁴ cells/cm² (i.e., 2.5×10⁴ cells/well) and on hydroxyapatite at a density of 1×10⁶ cells/mL. Eighteen hours after the inoculation, 1 mL of a supernatant (culture supernatant) of an MEM growth medium (containing a minimum essential medium (MEM), 15% FBS, 100 U/mL penicillin, 0.1 mg/mL streptomycin and 0.25 μg/mL amphotericin B), in which chondrocytes capable of hypertrophication were cultured, was added to the plate and the hydroxyapatite, and then the mouse C3H10T1/2 cells were cultured in a 5% CO₂ incubator at 37° C. to obtain cultures (cultures of cells).

The cultures were stained by fixed them with a 60% acetone/citric acid buffer, rinsing them with distilled water, and immersing them into a mixture of First Violet B and Naphthol AS-MX at room temperature in the dark for 30 minutes.

As described above, it was shown that the alkaline phosphatase (ALP) activity, which was one of the induced osteoblast markers, of the mouse C3H10T1/2 cells increased by an agent capable of inducing differentiation into induced osteoblasts. Furthermore, it was also shown that the mouse C3H10T1/2 cells cultured by adding the agent capable of inducing differentiation into induced osteoblasts were stained red with the alkaline phosphatase staining.

Therefore, expression of the alkaline phosphatase was also indicated using the staining method. As a result, it was confirmed that the mouse C3H10T1/2 cells were differentiated into the induced osteoblasts (see upper column in Table 1, FIG. 2, upper column in FIG. 3A, and FIG. 3B).

Furthermore, a pellet of the differentiated cells was prepared in the same manner as described above, and then stained with acid toluidine blue and safranine O. As a result, no metachromasia was shown and the safranine staining was negative. Thus, it was confirmed that these differentiated cells were not chondrocytes. Therefore, it could be confirmed that the differentiated cells were not the chondrocytes capable of hypertrophication.

### Comparative Example 1A

### Preparation and Detection of Agent Produced by Culturing Chondrocytes Capable of Hypertrophication Derived from Costa/Costal Cartilage in MEM Growth Medium

Chondrocytes capable of hypertrophication were collected from costa/costal cartilages in the same manner as Example 1. An MEM growth medium (containing a minimum essential medium (MEM), 15% FBS, 100 U/mL penicillin, 0.1 mg/mL streptomycin and 0.25 μg/mL amphotericin B) was added to the chondrocytes capable of hypertrophication so that they were diluted so as to become a density of 4×10⁴ cells/cm². The chondrocytes capable of hypertrophication were cultured, and then a supernatant of the medium was collected on a time course (4 days, 7 days, 11 days, 14 days, 18 days, 21 days) to obtain fractional supernatants.

Mouse C3H10T1/2 cells (“CCL-226” produced by Dainippon Sumitomo Pharma Co. Ltd.) were inoculated evenly in a 24-well plate (produced by Becton, Dickinson and Company) at a density of 1.25×10⁴ cells/cm² (i.e., 2.5×10⁴ cells/well). Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

In the case where the alkaline phosphatase activity was evaluated using a relative active value, when a value of the alkaline phosphatase activity of the mouse C3H10T1/2 cells cultured by adding only the MEM growth medium was defined as “1”, in a 4 week-old rat group: the relative active value thereof was about 1.0 time by adding the fractural supernatant collected 4 days after the culture; about 1.3 times by adding the fractural supernatant collected 1 week after the culture; about 1.1 times by adding the fractural supernatant collected 2 weeks after the culture; and about 1.0 time by adding the fractural supernatant collected 3 weeks after the culture.

In the above same case, in a 8 week-old rat group: the relative active value thereof was about 1.2 times by adding the fractural supernatant collected 4 days after the culture; about 1.0 time by adding the fractural supernatant collected 1 week after the culture; about 1.0 time by adding the fractural supernatant collected 2 weeks after the culture; and about 0.9 time by adding the fractural supernatant collected 3 weeks after the culture (see lower column in Table 1, and FIG. 2).

In each of the 4 and 8 week-old rat groups, there was little difference between a value of the alkaline phosphatase activity of the mouse C3H10T1/2 cells cultured by adding the supernatant of the MEM growth medium in which the chondrocytes capable of hypertrophication were cultured (culture of cells) and a value of the alkaline phosphatase activity thereof cultured by adding only the MEM growth medium.

(Identification of Induced Osteoblasts)

(Alkaline Phosphatase Staining)

Mouse C3H10T1/2 cells were inoculated in a 24-well plate and hydroxyapatite (in a BME medium), and cultured for 18 hours to obtain cultures (cultures of cells). Next, a supernatant (culture supernatant) of an MEM growth medium, in which chondrocytes capable of hypertrophication were cultured, was added to the cultures, and then alkaline phosphatase staining was performed after 72 hours. It was confirmed that the mouse C3H10T1/2 cells cultured by adding the supernatant were not stained with the alkaline phosphatase staining, and therefore they did not have the alkaline phosphatase activity (see lower column in FIG. 3A, and FIG. 3D).

TABLE 1
(alkaline phosphatase activity in the case
of addition of supernatant of MEM differentiation agent
producing medium or MEM growth medium in which chondrocytes
capable of hypertrophication were cultured)

| | | | | |
|---|---|---|---|---|
| 0 day | 4 days | 1 week | 2 weeks | 3 weeks |

MEM differentiation agent producing medium (mean value)

| | | | | | | |
|---|---|---|---|---|---|---|
| 4 | Relative value | 1 | 4.1 | 5.1 | 5.4 | 4.9 |
| week-old | Absolute value | | 0.077 | 0.098 | 0.103 | 0.095 |
| | (addition of supernatant) | | | | | |
| | Absolute value | 0.023 | 0.023 | 0.024 | 0.023 | 0.021 |
| | (only addition of medium) | | | | | |
| 8 | Relative value | 1 | 2.9 | 3.1 | 3.8 | 4.2 |
| week- | Absolute value | | 0.065 | 0.066 | 0.077 | 0.079 |
| old | (addition of supernatant) | | | | | |
| | Absolute value | 0.021 | 0.021 | 0.021 | 0.019 | 0.019 |
| | (only addition of medium) | | | | | |

MEM growth medium (mean value)

| | | | | | | |
|---|---|---|---|---|---|---|
| 4 | Relative value | 1 | 1.0 | 1.3 | 1.1 | 1.0 |
| week- | Absolute value | | 0.020 | 0.023 | 0.027 | 0.024 |
| old | (addition of supernatant) | | | | | |
| | Absolute value | 0.022 | 0.022 | 0.020 | 0.024 | 0.024 |
| | (only addition of medium) | | | | | |
| 8 | Relative value | 1 | 1.2 | 1.0 | 1.0 | 0.9 |
| week- | Absolute value | | 0.023 | 0.021 | 0.019 | 0.017 |
| old | (addition of supernatant) | | | | | |
| | Absolute value | 0.020 | 0.020 | 0.021 | 0.019 | 0.019 |
| | (only addition of medium) | | | | | |

4 week-old: five experiments were performed, and 3 trials were carried out per 1 experiment.
8 week-old: three experiments were performed. Two trials in the first experiment, two trials in the second experiment, and one trial in the third experiment were performed.

In the same manner as Example 1, it can be confirmed whether a supernatant (culture supernatant) of an MEM growth medium in which the chondrocytes capable of hypertrophication derived from costa/costal cartilage obtained using the above-described manipulation were cultured expresses osteoblast markers in the mouse C3H10T1/2 cells.

### Conclusion of Example 1 and Comparative Example 1A

In the case where the chondrocytes capable of hypertrophication were cultured in the MEM differentiation agent producing medium, it was confirmed that there was an agent capable of increasing the alkaline phosphatase activity of the mouse C3H10T1/2 cells which were undifferentiated cells, and capable of inducing differentiation thereof into induced osteoblasts in the supernatant of the medium (culture supernatant). On the other hand, in the case where the chondrocytes capable of hypertrophication were cultured in the MEM growth medium, it was confirmed that there was not the agent in the supernatant of the medium.

Therefore, it was found that the chondrocytes capable of hypertrophication produced an agent capable of inducing differentiation of undifferentiated cells into induced osteoblasts by culturing them in the MEM differentiation agent producing medium. Such an agent was not known hitherto. Therefore, it is believed that the existence of the agent itself is unexpected. Furthermore, BMP known hitherto would not have an effect of directly inducing the differentiation of the undifferentiated cells into the induced osteoblasts as described in other parts.

### Comparative Example 1B

### Preparation and Detection of Agent Produced by Culturing Resting Cartilage Cells Derived from Costal Cartilage in MEM Differentiation Agent Producing Medium

(Preparation of Resting Cartilage Cells from Costal Cartilages

Four week-old Male rats (Wistar) and 8 week-old Male rats (Wistar) were sacrificed using chloroform. The rats' chests were shaved using a razor and their whole bodies were immersed into a Hibitane solution (10-fold dilution) to be disinfected. The rats' chests were incised and costal cartilages were collected aseptically. Opaque resting cartilage regions were collected from the costal cartilages.

The resting cartilage regions were sectioned and stirred in a 0.25% trypsin-EDTA/D-PBS (dulbecco's phosphate buffered saline) at 37° C. for 1 hour. Next, the sections were rinsed by centrifugation (at 170×g for 3 minutes), and then stirred together with a 0.2% collagenase (produced by Invitrogen Corporation)/D-PBS at 37° C. for 2.5 hours. Thereafter, the sections were rinsed by centrifugation (at 170×g for 3 minutes), and then stirred together with a 0.2% dispase (produced by Invitrogen Corporation)/(HAM+10% FBS) in a stirring flask overnight at 37° C. Optionally, the overnight treatment with the 0.2% dispase was omitted. In the following day, cells were filtered and rinsed by centrifugation (at 170×g for 3 minutes). The cells were stained with trypan blue and the number thereof was counted under a microscope.

The cells evaluated as cells not stained were considered to be living cells, and those stained blue were considered to be dead cells.

(Identification of Chondrocytes Incapable of Hypertrophication Derived from Costal Cartilage)

It was determined whether chondrocytes capable of hypertrophication were present in a cell suspension obtained by diluting resting cartilage cells derived from costal cartilage in the same manner as Example 1. A hydroxyapatite was not stained with alkaline phosphatase staining (see, FIG. 1C). Spotted areas of the hydroxyapatite were stained blue with toluidine blue staining, thereby showing the existence of cells (see, FIG. 1D).

Thus, it was confirmed that the cells existing on the hydroxyapatite did not have an alkaline phosphate activity, thereby indicating that the chondrocytes incapable of hypertrophication (chondrocytes without an ability of hypertrophication) were present in the cell suspension used in this Comparative Example.

(Identification of Expression of Marker Gene for Chondrocytes Capable of Hypertrophication

In this Comparative Example, expression amounts of alkaline phosphatase, type II collagen, aglycan and osteocalcin were measured by a real-time PCR method in the same manner as Example 1. GAPDH was used as an integral control gene.

Samples (Rp1 and Rp2) were produced by centrifuging 5×10⁵ chondrocytes incapable of hypertrophication prepared in this Comparative Example at 170 to 200×g for 3 to 5 minutes to form into pellets, and then culturing the pellets in a 5% CO₂ incubator at 37° C. for 1 week, and used. As a medium, a HAM medium+10% FBS or a HEM medium+15% FBS was used.

A real-time PCR was performed and expression amounts of each cell marker were measured using the real-time PCR apparatus (“PRISM 7900HT” produced by Applied Biosystems, Inc.) in the same manner as Example 1. After complication of the PCR, setting of the threshold values and calculation of attainment cycles were performed using the analysis software incorporated in the apparatus (“PRISM 7900HT”).

Expression amounts of each cell marker were divided by an expression amount of the GAPDH to calculate correction values thereof, and then an average expression amount thereof was obtained by averaging the correction values. As a result, the chondrocyte incapable of hypertrophication expressed the type II collagen and the aglycan, but did not express the alkaline phosphatase and the osteocalcin (see, Table II).

TABLE II

Amount (correction value by GAPDH)
average value

| | | | | |
|---|---|---|---|---|
| Sample | 1 | 2 | 3 | Average |

Alkaline phosphatase

| | | | | |
|---|---|---|---|---|
| Rp1 | 0.0002 | 0.0003 | 0.0003 | 0.0002 |
| Rp2 | 0.0001 | 0.0001 | 0.0001 | 0.0001 |

Type II Collagen

| | | | | |
|---|---|---|---|---|
| Rp1 | 0.3448 | 0.4111 | 0.4168 | 0.3909 |
| Rp2 | 0.2838 | 0.2762 | 0.2877 | 0.2826 |

Aglycan

| | | | | |
|---|---|---|---|---|
| Rp1 | 1.0586 | 1.1427 | 1.1478 | 1.1164 |
| Rp2 | 1.0437 | 0.8835 | 0.9133 | 0.9468 |

Osteocalcin

| | | | | |
|---|---|---|---|---|
| Rp1 | 0.0001 | 0.0001 | 0.0002 | 0.0001 |
| Rp2 | 0.0000 | 0.0000 | 0.0000 | 0.0000 |

Rp1 and Rp2: pellets of chondrocytes incapable of hypertrophication cultured for 1 week.

By detecting localization or expression of the chondrocyte markers in the same method as Example 1, and assessing cells morphologically, it was determined that the cells obtained were the chondrocytes incapable of hypertrophication.

(Detection of Agent Produced by Culturing Resting Cartilage Cells Collected from Costal Cartilage in MEM Differentiation Agent Producing Medium)

An MEM differentiation agent producing medium (containing a minimum essential medium (MEM), 15% FBS (fetal bovine serum), 10 nM dexamethasone, 10 mM β-glycerophosphate, 50 μg/mL ascorbic acid, 100 U/mL penicillin, 0.1 mg/mL streptomycin and 0.25 μg/mL amphotericin B) was added to the resting cartilage cells collected from costal cartilage so that they were diluted so as to become a density of 4×10⁴ cells/cm². The resting cartilage cells were cultured, and then a supernatant of the medium was collected on a time course (4 days, 7 days, 11 days, 14 days, 18 days, 21 days) to obtain fractional supernatants.

Mouse C3H10T1/2 cells (“CCL-226” produced by Dainippon Sumitomo Pharma Co. Ltd.) were inoculated in a 24-well plate. Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

In the case where the alkaline phosphatase activity was evaluated using a relative active value, when a value of the alkaline phosphatase activity of the mouse C3H10T1/2 cells cultured by adding only the MEM differentiation agent producing medium was defined as “1”, the relative active value thereof was about 0.9 time by adding the fractural supernatant collected 4 days after the culture; about 1.1 times by adding the fractural supernatant collected 1 week after the culture; about 1.0 time by adding the fractural supernatant collected 2 weeks after the culture; and about 1.1 times by adding the fractural supernatant collected 3 weeks after the culture (see upper column in Table 2, and FIG. 4).

There was little difference between a value of the alkaline phosphatase activity of the mouse C3H10T1/2 cells cultured by adding the supernatant of the MEM differentiation agent producing medium in which the resting cartilage cells were cultured (culture of cells) and a value of the alkaline phosphatase activity thereof cultured by adding only the MEM differentiation agent producing medium.

In the same method and criteria as Example 1, it can be confirmed whether the supernatant of the medium (culture of cells) obtained using the above-described manipulation expresses induced osteoblast markers in the mouse C3H10T1/2 cells.

### Comparative Example 1C

### Preparation and Detection of Agent Produced by Culturing Resting Cartilage Cells Derived from Costal Cartilage in MEM Growth Medium

Resting cartilage cells were collected from costal cartilages in the same manner as Comparative Example 1B. An MEM growth medium (containing a minimum essential medium (MEM), 15% FBS, 100 U/mL penicillin, 0.1 mg/mL streptomycin and 0.25 μg/mL amphotericin B) was added to the resting cartilage cells so that they were diluted so as to become a density of 4×10⁴ cells/cm². The resting cartilage cells were cultured, and then a supernatant of the medium was collected on a time course (4 days, 7 days, 11 days, 14 days, 18 days, 21 days) to obtain fractional supernatants.

Mouse C3H10T1/2 cells (“CCL-226” produced by Dainippon Sumitomo Pharma Co. Ltd.) were inoculated evenly in a 24-well plate. Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

In the case where the alkaline phosphatase activity was evaluated using a relative active value, when a value of the alkaline phosphatase activity of the mouse C3H10T1/2 cells cultured by adding only the MEM growth medium was defined as “1”, the relative active value thereof was about 1.0 time by adding the fractural supernatant collected 4 days after the culture; about 1.0 time by adding the fractural supernatant collected 1 week after the culture; about 0.9 time by adding the fractural supernatant collected 2 weeks after the culture; and about 1.1 times by adding the fractural supernatant collected 3 weeks after the culture (see lower column in Table 2, and FIG. 4).

There was little difference between a value of the alkaline phosphatase activity of the mouse C3H10T1/2 cells cultured by adding the supernatant of the MEM growth medium in which the resting cartilage cells were cultured (culture of cells) and a value of the alkaline phosphatase activity thereof cultured by adding only the MEM growth medium (see lower column in Table 2, and FIG. 4).

In the same method and criteria as Example 1, it can be confirmed whether the supernatant of the medium (culture of cells) obtained using the above-described manipulation expresses induced osteoblast markers in the mouse C3H10T1/2 cells.

TABLE 2
(alkaline phosphatase activity in the case
of addition of supernatant of MEM differentiation agent
producing medium or MEM growth medium in which resting cartilage
cells derived from costal cartilage were cultured)

| | | | | |
|---|---|---|---|---|
| 0 day | 4 days | 1 week | 2 weeks | 3 weeks |

MEM differentiation agent producing medium (mean value)

| | | | | | | |
|---|---|---|---|---|---|---|
| 8 | Relative value | 1 | 0.9 | 1.1 | 1.0 | 1.1 |
| week-old | Absolute value | | 0.014 | 0.015 | 0.015 | 0.014 |
| | (addition of supernatant) | | | | | |
| | Absolute value | 0.015 | 0.015 | 0.014 | 0.014 | 0.014 |
| | (only addition of medium) | | | | | |

MEM growth medium (mean value)

| | | | | | | |
|---|---|---|---|---|---|---|
| 8 | Relative value | 1 | 1.0 | 1.0 | 0.9 | 1.1 |
| week- | Absolute value | | 0.014 | 0.012 | 0.012 | 0.012 |
| old | (addition of supernatant) | | | | | |
| | Absolute value | 0.013 | 0.013 | 0.012 | 0.011 | 0.011 |
| | (only addition of medium) | | | | | |

8 week-old: three experiments were performed. Three trials in the first experiment, one trial in the second experiment, and three trials in the third experiment were performed.

### Conclusion of Comparative Example 1B and Comparative Example 1C

Even in the case where the resting cartilage cells incapable of hypertrophication collected from costal cartilage were cultured in the MEM differentiation agent producing medium or the MEM growth medium, it was confirmed that they did not produce an agent capable of inducing differentiation of undifferentiated cells into induced osteoblasts.

### Comparative Example 1D

### Preparation and Detection of Agent Produced by Culturing Chondrocytes Derived from Articular Cartilage in MEM Differentiation Agent Producing Medium

(Preparation of Chondrocytes from Articular Cartilages)

Eight week-old male rats (Wistar) were sacrificed using chloroform. The rats were shaved around their knee joint regions using a razor and their whole bodies were immersed into a Hibitane solution (10-fold dilution) to be disinfected. The rats were incised at their knee joint regions and articular cartilages were collected aseptically.

The articular cartilages were sectioned and stirred in a 0.25% trypsin-EDTA/D-PBS at 37° C. for 1 hour. Next, the sections were rinsed by centrifugation (at 170×g for 3 minutes), and then stirred together with a 0.2% collagenase/D-PBS at 37° C. for 2.5 hours. Thereafter, the sections were rinsed by centrifugation (at 170×g for 3 minutes), and then stirred together with a 0.2% dispase/(HAM+10% FBS) in a stirring flask overnight at 37° C.

Optionally, the overnight treatment with the 0.2% dispase was omitted. In the following day, cells were filtered and rinsed by centrifugation (at 170×g for 3 minutes). The cells were stained with trypan blue and the number thereof was counted under a microscope.

The cells evaluated as cells not stained were considered to be living cells, and those stained blue were considered to be dead cells.

(Identification of Chondrocytes Incapable of Hypertrophication Derived from Articular Cartilage)

It was determined whether chondrocytes capable of hypertrophication were present in a cell suspension obtained by diluting chondrocytes derived from articular cartilage in the same manner as Example 1. A hydroxyapatite was not stained with alkaline phosphatase staining (see FIG. 1E). Spotted areas of the hydroxyapatite was stained blue with toluidine blue staining, thereby showing the existence of cells (see FIG. 1F).

Thus, it was confirmed that the cells existing on the hydroxyapatite did not have an alkaline phosphate activity, thereby indicating that the chondrocytes incapable of hypertrophication were present in the cell suspension used in this Comparative Example.

By detecting localization or expression of chondrocyte markers in the same method and criteria as Example 1, and assessing cells morphologically, it is determined whether the cells obtained are the chondrocytes incapable of hypertrophication.

(Detection of Agent Produced by Culturing Chondrocytes Collected from Articular Cartilage in MEM Differentiation Agent Producing Medium)

An MEM differentiation agent producing medium (containing a minimum essential medium (MEM), 15% FBS (fetal bovine serum), 10 nM dexamethasone, 10 mM β-glycerophosphate, 50 μg/mL ascorbic acid, 100 U/mL penicillin, 0.1 mg/mL streptomycin and 0.25 μg/mL amphotericin B) was added to the chondrocytes collected from articular cartilage so that they were diluted so as to become a density of 4×10⁴ cells/cm². The chondrocytes were cultured, and then a supernatant of the medium was collected on a time course (4 days, 7 days, 11 days, 14 days, 18 days, 21 days) to obtain fractional supernatants.

Mouse C3H10T1/2 cells (“CCL-226” produced by Dainippon Sumitomo Pharma Co. Ltd.) were inoculated in a 24-well plate. Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

In the case where the alkaline phosphatase activity was evaluated using a relative active value, when a value of the alkaline phosphatase activity of the mouse C3H10T1/2 cells cultured by adding only the MEM differentiation agent producing medium was defined as “1”, the relative active value thereof was about 1.4 times by adding the fractural supernatant collected 4 days after the culture; about 1.1 times by adding the fractural supernatant collected 1 week after the culture; about 1.1 times by adding the fractural supernatant collected 2 weeks after the culture; and about 1.1 times by adding the fractural supernatant collected 3 weeks after the culture (see upper column in Table 3, and FIG. 5A).

There was little difference between a value of the alkaline phosphatase activity of the mouse C3H10T1/2 cells cultured by adding the supernatant of the MEM differentiation agent producing medium in which the the chondrocytes were cultured (culture of cells) and a value of the alkaline phosphatase activity thereof cultured by adding only the MEM differentiation agent producing medium.

In the same method and criteria as Example 1, it can be determined whether the supernatant of the medium (culture of cells) obtained using the above-described manipulation expresses induced osteoblast markers in the mouse C3H10T1/2 cells.

### Comparative Example 1E

### Preparation and Detection of Agent Produced by Culturing Chondrocytes Collected from Articular Cartilage in MEM Growth Medium

Chondrocytes were collected from articular cartilages in the same manner as Comparative Example 1D. An MEM growth medium (containing a minimum essential medium (MEM), 15% FBS, 100 U/mL penicillin, 0.1 mg/mL streptomycin and 0.25 μg/mL amphotericin B) was added to the chondrocytes so that they were diluted so as to become a density of 4×10⁴ cells/cm². The chondrocytes were cultured, and then a supernatant of the medium (culture supernatant) was collected on a time course (4 days, 7 days, 11 days, 14 days, 18 days, 21 days) to obtain fractional supernatants.

Mouse C3H10T1/2 cells (“CCL-226” produced by Dainippon Sumitomo Pharma Co. Ltd.) were inoculated in a 24-well plate. Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

In the case where the alkaline phosphatase activity was evaluated using a relative active value, when a value of the alkaline phosphatase activity of the mouse C3H10T1/2 cells cultured by adding only the MEM growth medium was defined as “1”, the relative active value thereof was about 1.1 times by adding the fractural supernatant collected 4 days after the culture; about 1.0 time by adding the fractural supernatant collected 1 week after the culture; about 1.1 times by adding the fractural supernatant collected 2 weeks after the culture; and about 1.2 times by adding the fractural supernatant collected 3 weeks after the culture (see lower column in Table 3, and FIG. 5A).

There was little difference between a value of the alkaline phosphatase activity of the mouse C3H10T1/2 cells cultured by adding the supernatant of the MEM growth medium in which the chondrocytes derived from articular cartilage were cultured and a value of the alkaline phosphatase activity thereof cultured by adding only the MEM growth medium (see lower column in Table 3, and FIG. 5A).

In the same method and criteria as Example 1, it can be determined whether the supernatant of the medium (culture of cells) obtained using the above-described manipulation expresses induced osteoblast markers in the mouse C3H10T1/2 cells.

TABLE 3
(alkaline phosphatase activity in the case
of addition of supernatant of MEM differentiation agent
producing medium or MEM growth medium in which chondrocytes
derived from articular cartilage were cultured)

| | | | | |
|---|---|---|---|---|
| 0 day | 4 days | 1 week | 2 weeks | 3 weeks |

MEM differentiation agent producing medium (mean value)

| | | | | | | |
|---|---|---|---|---|---|---|
| 8 week-old | Relative value | 1 | 1.4 | 1.1 | 1.1 | 1.1 |
| | Absolute value | | 0.020 | 0.019 | 0.019 | 0.020 |
| | (addition of supernatant) | | | | | |
| | Absolute value | 0.016 | 0.016 | 0.017 | 0.016 | 0.017 |
| | (only addition of medium) | | | | | |

8 week-old: six experiments were performed. One trial in the first experiment,
one trial in the second experiment, three trials in the third experiment,
two trials in the fourth experiment, one trial in the fifth experiment,
and one trial in the sixth experiment were performed.

MEM growth medium (mean value)

| | | | | | | |
|---|---|---|---|---|---|---|
| 8 week-old | Relative value | 1 | 1.1 | 1.0 | 1.1 | 1.2 |
| | Absolute value | | 0.019 | 0.017 | 0.017 | 0.019 |
| | (addition of supernatant) | | | | | |
| | Absolute value | 0.018 | 0.018 | 0.018 | 0.014 | 0.017 |
| | (only addition of medium) | | | | | |

8 week-old: five experiments were performed. Two trials in the first experiment,
two trials in the second experiment, three trials in the third experiment,
one trial in the fourth experiment, and one trial in the fifth experiment
were performed.

### Conclusion of Comparative Example 1D and Comparative Example 1E

Even in the case where the chondrocytes incapable of hypertrophication derived from articular cartilage were cultured in the MEM differentiation agent producing medium or the MEM growth medium, it was confirmed that they did not produce an agent capable of inducting differentiation of undifferentiated cells into induced osteoblasts.

### Example 2

### Preparation and Detection of Cellular Function Regulating Agent Produced by Culturing Chondrocytes Capable of Hypertrophication Derived from Sternal Cartilage in MEM Differentiation Agent Producing Medium

(Preparation of Chondrocytes Capable of Hypertrophication from Sternal Cartilages)

Eight week-old male rats (Wistar) are sacrificed using chloroform. The rats' chests are shaved using a razor and their whole bodies are immersed into a Hibitane solution (10-fold dilution) to be disinfected. The rats' chests are incised and inferior portions of sternal cartilages and processus xiphoideus are collected aseptically. Translucent growth cartilage regions are collected from the inferior portions of sternal cartilages and the processus xiphoideus.

The growth cartilage regions are sectioned and stirred in a 0.25% trypsin-EDTA/dulbecco's phosphate buffered saline (D-PBS) at 37° C. for 1 hour. Next, the sections are rinsed by centrifugation (at 170×g for 3 minutes), and then stirred together with a 0.2% collagenase (produced by Invitrogen Corporation)/D-PBS at 37° C. for 2.5 hours. Thereafter, the sections were rinsed by centrifugation (at 170×g for 3 minutes), and then stirred together with a 0.2% dispase (produced by Invitrogen Corporation)/(HAM+10% FBS) in a stirring flask overnight at 37° C. Optionally, the overnight treatment with the 0.2% dispase is omitted. In the following day, cells are filtered and rinsed by centrifugation (at 170×g for 3 minutes). The cells are stained with trypan blue and the number thereof is counted under a microscope.

The cells evaluated as cells not stained are considered to be living cells, and those stained blue are considered to be dead cells.

(Identification of Chondrocytes Capable of Hypertrophication)

It is determined whether cells collected are chondrocytes capable of hypertrophication in the same method and criteria as Example 1.

(Detection of Agent Produced by Culturing Chondrocytes Capable of Hypertrophication Derived from Sternal Cartilage in MEM Differentiation Agent Producing Medium)

An MEM differentiation agent producing medium (containing a minimum essential medium (MEM), 15% FBS (fetal bovine serum), 10 nM dexamethasone, 10 mM β-glycerophosphate, 50 μg/mL ascorbic acid, 100 U/mL penicillin, 0.1 mg/mL streptomycin and 0.25 μg/mL amphotericin B) is added to the chondrocytes capable of hypertrophication derived from sternal cartilage so that they are diluted so as to become a density of 4×10⁴ cells/cm². The chondrocytes capable of hypertrophication are cultured, and then a supernatant of the medium is collected on a time course (4 days, 7 days, 11 days, 14 days, 18 days, 21 days) to obtain fractional supernatants.

Mouse C3H10T1/2 cells (“CCL-226” produced by Dainippon Sumitomo Pharma Co. Ltd.) are inoculated in a 24-well plate. Eighteen hours after the inoculation, 1 mL of each of the fractional supernatants and the medium is added to the plate, and then the mouse C3H10T1/2 cells are cultured in a 5% CO₂ incubator at 37° C. After 72 hours, an alkaline phosphatase activity of the C3H10T1/2 cells is measured in the same manner as Example 1.

A value of the alkaline phosphatase activity of the mouse C3H10T1/2 cells cultured by adding the supernatant of the MEM differentiation agent producing medium in which the chondrocytes capable of hypertrophication are cultured (culture of cells) increases, as compared with a value of the alkaline phosphatase activity thereof cultured by adding only the MEM differentiation agent producing medium.

(Identification of Induced Osteoblasts)

In the same method and criteria as Example 1, it is confirmed that the supernatant of the medium (culture of cells) obtained using the above-described manipulation expresses induced osteoblast markers in the mouse C3H10T1/2 cells.

### Comparative Example 2

### Preparation of Agent Produced by Culturing Chondrocytes Capable of Hypertrophication Derived from Sternal Cartilage in MEM Growth Medium

Chondrocytes capable of hypertrophication are collected from sternal cartilages in the same manner as Example 2. An MEM growth medium (containing a minimum essential medium (MEM), 15% FBS, 100 U/mL penicillin, 0.1 mg/mL streptomycin and 0.25 μg/mL amphotericin B) is added to the chondrocytes capable of hypertrophication so that they are diluted so as to become a density of 4×10⁴ cells/cm². The chondrocytes capable of hypertrophication are cultured, and then a supernatant of the medium (culture supernatant) is collected on a time course to obtain fractional supernatants.

Mouse C3H10T1/2 cells (“CCL-226” produced by Dainippon Sumitomo Pharma Co. Ltd.) are inoculated in a 24-well plate. Eighteen hours after the inoculation, 1 mL of each of the fractional supernatants and the medium was added to the plate, and then the mouse C3H10T1/2 cells are cultured in a 5% CO₂ incubator at 37° C. After 72 hours, an alkaline phosphatase activity of the mouse C3H10T1/2 cells is measured in the same manner as Example 1.

There is little difference between a value of the alkaline phosphatase activity of the mouse C3H10T1/2 cells cultured by adding the supernatant of the MEM growth medium in which the chondrocytes capable of hypertrophication are cultured (culture of cells) and a value of the alkaline phosphatase activity thereof cultured by adding only the MEM growth medium.

In the same method and criteria as Example 1, it can be determined whether the supernatant of the medium (culture of cells) obtained using the above-described manipulation expresses induced osteoblast markers in the mouse C3H10T1/2 cells.

### Conclusion of Example 2 and Comparative Example 2

In the case where a value of the alkaline phosphatase activity of the mouse C3H10T1/2 cells cultured by adding the supernatant of the MEM differentiation agent producing medium, in which the chondrocytes capable of hypertrophication are cultured, increases, it is determined that there is an agent capable of inducing differentiation into induced osteoblasts in the supernatant.

On the other hand, in the case where a value of the alkaline phosphatase activity of the mouse C3H10T1/2 cells cultured by adding the supernatant of the MEM growth medium, in which the chondrocytes capable of hypertrophication are cultured, does not increase, it is determined that there is not the above agent in the supernatant.

In this case, it is determined that the chondrocytes capable of hypertrophication cultured in the MEM differentiation agent producing medium produce the agent capable of inducing differentiation of undifferentiated cells into induced osteoblasts.

### Example 3

### Preparation and Detection of Cellular Function Regulating Agent Produced by Culturing Chondrocytes Capable of Hypertrophication Derived from Costa/Costal Cartilage in HAM Differentiation Agent Producing Medium

(Detection of Agent Produced by Chondrocytes Capable of Hypertrophication Collected from Costa/Costal Cartilage)

Chondrocytes capable of hypertrophication were collected from costa/costal cartilages in the same manner as Example 1. A HAM differentiation agent producing medium (containing a HAM medium, 10% FBS (fetal bovine serum), 10 nM dexamethasone, 10 mM β-glycerophosphate, 50 μg/mL ascorbic acid, 100 U/mL penicillin, 0.1 mg/mL streptomycin and 0.25 μg/mL amphotericin B) was added to the chondrocytes capable of hypertrophication so that they were diluted so as to become a density of 4×10⁴ cells/cm². The chondrocytes capable of hypertrophication were cultured, and then a supernatant of the medium (culture supernatant) was collected on a time course (4 days, 7 days, 11 days, 14 days, 18 days, 21 days) to obtain fractional supernatants.

Mouse C3H10T1/2 cells (“CCL-226” produced by Dainippon Sumitomo Pharma CO. Ltd.) were inoculated in a 24-well plate. Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

In the case where the alkaline phosphatase activity was evaluated using a relative active value, when a value of the alkaline phosphatase activity of the mouse C3H10T1/2 cells cultured by adding only the HAM differentiation agent producing medium was defined as “1”, the relative active value thereof was about 1.2 times by adding the fractural supernatant collected 4 days after the culture; about 2.3 times by adding the fractural supernatant collected 1 week after the culture; about 3.1 times by adding the fractural supernatant collected 2 weeks after the culture; and about 2.2 times by adding the fractural supernatant collected 3 weeks after the culture (see upper column in Table 3-2, and FIG. 5B).

It was shown that the alkaline phosphatase (ALP) activity, which was one of the induced osteoblast markers, of the mouse C3H10T1/2 cells increased by an agent capable of inducing differentiation into induced osteoblasts (see Table 3-2, and FIG. 5B). Furthermore, expression of alkaline phosphatase was also indicated using an alkaline phosphatase staining method. As a result, it was confirmed that the mouse C3H10T1/2 cells were differentiated into induced osteoblasts.

TABLE 3-2
(alkaline phosphatase activity in the
case of addition of supernatant of HAM differentiation agent
producing medium or HAM growth medium in which chondrocytes
capable of hypertrophication were cultured)

| | | | | |
|---|---|---|---|---|
| 0 day | 4 days | 1 week | 2 weeks | 3 weeks |

HAM differentiation agent producing medium (mean value)

| | | | | | |
|---|---|---|---|---|---|
| Relative value | 1 | 1.2 | 2.3 | 3.1 | 2.2 |
| Absolute value | 0.015 | 0.018 | 0.033 | 0.047 | 0.037 |
| (addition of supernatant) | | | | | |
| Absolute value | 0.015 | | 0.014 | 0.015 | 0.017 |
| (only addition of medium) | | | | | |

Three experiments were performed. Three trials were
performed per 1 experiment.

HAM growth medium (mean value)

| | | | | | |
|---|---|---|---|---|---|
| Relative value | 1 | 1.0 | 0.9 | 1.2 | 1.2 |
| Absolute value | 0.026 | 0.025 | 0.023 | 0.020 | 0.024 |
| (addition of supernatant) | | | | | |
| Absolute value | 0.026 | | 0.024 | 0.021 | 0.023 |
| (only addition of medium) | | | | | |

Five experiments were performed. Three trials in the first experiment,
three trials in the second experiment, three trials in the third experiment,
three trials in the fourth experiment, and two trials in the fifth experiment
were performed.

Five experiments were performed. Three trials in the first experiment, three trials in the second experiment, three trials in the third experiment, three trials in the fourth experiment, and two trials in the fifth experiment were performed.

### Comparative Example 3A

### Preparation and Detection of Agent Produced by Culturing Chondrocytes Capable of Hypertrophication Derived from Costa/Costal Cartilage in HAM Growth Medium

Chondrocytes capable of hypertrophication were collected from costa/costal cartilages in the same manner as Example 1. A HAM growth medium (containing a HAM medium, 10% FBS, 100 U/mL penicillin, 0.1 mg/mL streptomycin and 0.25 μg/mL amphotericin B) was added to the chondrocytes capable of hypertrophication so that they were diluted so as to become a density of 4×10⁴ cells/cm². The chondrocytes capable of hypertrophication were cultured, and then a supernatant of the medium was collected on a time course to obtain fractional supernatants.

Mouse C3H10T1/2 cells (“CCL-226” produced by Dainippon Sumitomo Pharma Co. Ltd.) were inoculated in a 24-well plate. Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

In the case where the alkaline phosphatase activity was evaluated using a relative active value, when a value of the alkaline phosphatase activity of the mouse C3H10T1/2 cells cultured by adding only the HAM growth medium was defined as “1”, the relative active value thereof was about 1.0 time by adding the fractural supernatant collected 4 days after the culture; about 0.9 time by adding the fractural supernatant collected 1 week after the culture; about 1.2 times by adding the fractural supernatant collected 2 weeks after the culture; and about 1.2 times by adding the fractural supernatant collected 3 weeks after the culture (see lower column in Table 3-2, and FIG. 5C).

There was little difference between a value of the alkaline phosphatase activity of the mouse C3H10T1/2 cells cultured by adding the supernatant of the MEM growth medium in which the chondrocytes capable of hypertrophication were cultured (cell curture) and a value of the alkaline phosphatase activity thereof cultured by adding only the HAM growth medium (see lower column in Table 3-2, and FIG. 5C).

It was confirmed that the supernatant of the medium (culture of cells) obtained using the above-described manipulation did not express induced osteoblast markers in the mouse C3H10T1/2 cells.

### Conclusion of Example 3 and Comparative Example 3A

In the case where the chondrocytes capable of hypertrophication were cultured in the HAM differentiation agent producing medium, it was confirmed that there was an agent capable of increasing the alkaline phosphatase activity of the mouse C3H10T1/2 cells which were undifferentiated cells, and capable of inducing differentiation thereof into induced osteoblasts in the supernatant of the medium.

On the other hand, in the case where the chondrocytes capable of hypertrophication were cultured in the HAM growth medium, it was confirmed that there was not the agent in the supernatant of the madium. Therefore, it was found that the chondrocytes capable of hypertrophication cultured in the HAM differentiation agent producing medium produced the agent capable of inducing differentiation of undifferentiated cells into induced osteoblasts.

### Comparative Example 3B

### Preparation and Detection of Agent Produced by Culturing Resting Cartilage Cells Derived from Costal Cartilage in HAM Differentiation Agent Producing Medium

Resting cartilage cells are collected from costal cartilages in the same manner as Comparative Example 1B. A HAM differentiation agent producing medium (containing a HAM medium, 10% FBS (fetal bovine serum), 10 nM dexamethasone, 10 mM β-glycerophosphate, 50 μg/mL ascorbic acid, 100 U/mL penicillin, 0.1 mg/mL streptomycin and 0.25 μg/mL amphotericin B) is added to the resting cartilage cells so that they are diluted so as to become a density of 4×10⁴ cells/cm². The resting cartilage cells are cultured, and then a supernatant of the medium is collected on a time course to obtain fractional supernatants.

Mouse C3H10T1/2 cells (“CCL-226” produced by Dainippon Sumitomo Pharma Co. Ltd.) are inoculated evenly in a 24-well plate. Eighteen hours after the inoculation, 1 mL of each of the fractional supernatants is added to the plate, and then the mouse C3H10T1/2 cells are cultured. After 72 hours, an alkaline phosphatase activity of the mouse C3H10T1/2 cells is measured in the same manner as Example 1.

In the case where both conditions described below are satisfied, it is determined that the mouse C3H10T1/2 cells are not differentiated into induced osteoblasts. Namely, first, there is little difference between a value of the alkaline phosphatase activity thereof cultured by adding the supernatant of the HAM differentiation agent producing medium in which the resting cartilage cells are cultured (culture of cells) and a value of the alkaline phosphatase activity thereof cultured by adding only the HAM differentiation agent producing medium or the HAM growth medium. Second, the supernatant of the medium (culture of cells) obtained using the above-described manipulation does not express induced osteoblast markers in the mouse C3H10T1/2 cells.

In this case, it is determined that the resting cartilage cells derived from costal cartilage cultured in the HAM differentiation agent producing medium do not produce an agent capable of inducing differentiation of undifferentiated cells into induced osteoblasts.

### Comparative Example 3C

### Preparation and Detection of Agent Produced by Culturing Resting Cartilage Cells Derived from Costal Cartilage in HAM Growth Medium

Resting cartilage cells are collected from costal cartilages in the same manner as Comparative Example 1B. A HAM growth medium (containing a HAM medium, 10% FBS (fetal bovine serum), 100 U/mL penicillin, 0.1 mg/mL streptomycin and 0.25 μg/mL amphotericin B) is added to the resting cartilage cells so that they are diluted so as to become a density of 4×10⁴ cells/cm². The resting cartilage cells are cultured, and then a supernatant of the medium is collected on a time course to obtain fractional supernatants.

Mouse C3H10T1/2 cells (“CCL-226” produced by Dainippon Sumitomo Pharma Co. Ltd.) are inoculated evenly in a 24-well plate. Eighteen hours after the inoculation, 1 mL of each of the fractional supernatants is added to the plate, and then the mouse C3H10T1/2 cells are cultured. After 72 hours, an alkaline phosphatase activity of the mouse C3H10T1/2 cells is measured in the same manner as Example 1.

In the case where both conditions described below are satisfied, it is determined that the mouse C3H10T1/2 cells are not differentiated into induced osteoblasts. Namely, first, there is little difference between a value of the alkaline phosphatase activity thereof cultured by adding the supernatant of the HAM growth medium in which the resting cartilage cells are cultured (culture of cells) and a value of the alkaline phosphatase activity thereof cultured by adding only the HAM differentiation agent producing medium or the HAM growth medium. Second, the supernatant of the medium (culture of cells) obtained using the above-described manipulation does not express induced osteoblast markers in the mouse C3H10T1/2 cells.

In this case, it is determined that the resting cartilage cells derived from costal cartilage cultured in the HAM growth medium do not produce an agent capable of inducing differentiation of undifferentiated cells into induced osteoblasts.

### Conclusion of Example 1, Example 3, Comparative Examples 1A to 1E and 3A to 3C

According to Examples described above, the chondrocytes capable of hypertrophication cultured in the differentiation agent producing medium produce the agent capable of inducing differentiation of undifferentiated cells into induced osteoblasts, in spite of the type of the basal medium contained in the medium. Even in the case where the chondrocytes capable of hypertrophication are cultured in any growth medium, they do not produce the agent capable of inducing differentiation of undifferentiated cells into induced osteoblasts.

Furthermore, even in the case where the resting cartilage cells incapable of hypertrophication and the articular cartilage cells incapable of hypertrophication are cultured in any medium, they do not produce the agent capable of inducing differentiation of undifferentiated cells into induced osteoblasts.

For these reasons, it is suggested that the agent capable of inducing differentiation of undifferentiated cells into induced osteoblasts is produced only by culturing the chondrocytes capable of hypertrophication in the differentiation agent producing medium. Furthermore, since the basal medium contained in the medium is unlikely to affect the production of the induced osteoblast differentiation inducing agent as long as it can be normally used in a cell culture, it is believed that any basal medium can be used in the present method.

### Example 4

### Preparation and Detection of Cellular Function Regulating Agent Produced by Culturing Chondrocytes Capable of Hypertrophication Derived from Human in MEM Differentiation Agent Producing Medium

(Detection of Agent Produced by Chondrocytes Capable of Hypertrophication Derived from Human

Chondrocytes capable of hypertrophication derived from human tissue (e.g., polydactyly, tumor, provided cartilaginous tissue) are obtained from human tissue resource exploitation organizations such as domestic organizations (e.g., Health Science Research Resources Bank; Cell Bank, RIKEN BioResource Center; Cell Bank, National Institute of Health Sciences; Institute of Development, Aging and Cancer at Tohoku University) and foreign organizations (e.g., IIAM, ATCC), and cell providing companies such as Osiris.

An MEM differentiation agent producing medium (containing a minimum essential medium (MEM), 15% FBS (fetal bovine serum), 10 nM dexamethasone, 10 mM β-glycerophosphate, 50 μg/mL ascorbic acid, 100 U/mL penicillin, 0.1 mg/mL streptomycin and 0.25 μg/mL amphotericin B) is added to the chondrocytes capable of hypertrophication obtained so that they are diluted so as to become a density of 4×10⁴ cells/cm². The chondrocytes capable of hypertrophication are cultured, and then a supernatant of the medium is collected on a time course to obtain fractional supernatants.

Investigational human mesenchymal stem cells are obtained from the above-described organizations. The human mesenchymal stem cells are inoculated evenly in a 24-well plate. Eighteen hours after the inoculation, 1 mL of each of the fractional supernatants was added to the plate, and then the human mesenchymal stem cells are cultured. After 72 hours, an alkaline phosphatase activity of the human mesenchymal stem cells is measured in the same manner as Example 1.

In the case where the alkaline phosphatase (ALP) activity, which is one of the induced osteoblast markers, of the investigational human undifferentiated cells increases by an agent capable of inducing differentiation into induced osteoblasts, it is determined that the undifferentiated cells are differentiated into induced osteoblasts. Furthermore, in the case where expression of the alkaline phosphatase is detected with alkaline phosphatase staining, it is also determined that the undifferentiated cells are differentiated into the induced osteoblasts.

### Comparative Example 4A

### Preparation and Detection of Agent Produced by Culturing Chondrocytes Capable of Hypertrophication Derived from Human in MEM Growth Medium

Chondrocytes capable of hypertrophication are obtained in the same manner as Example 4. An MEM growth medium (containing a minimum essential medium (MEM), 15% FBS, 100 U/mL penicillin, 0.1 mg/mL streptomycin and 0.25 μg/mL amphotericin B) is added to the chondrocytes capable of hypertrophication so that they are diluted so as to become a density of 4×10⁴ cells/cm². The chondrocytes capable of hypertrophication are cultured, and then a supernatant of the medium is collected on a time course to obtain fractional supernatants.

Investigational human undifferentiated cells are inoculated in a 24-well plate. Eighteen hours after the inoculation, 1 mL of each of the fractional supernatants is added to the plate, and then the human undifferentiated cells are cultured. After 72 hours, an alkaline phosphatase activity of the human undifferentiated cells is measured in the same manner as Example 1.

In the case where there is little difference between a value of the alkaline phosphatase activity of the human undifferentiated cells cultured by adding the supernatant of the MEM growth medium in which the chondrocytes capable of hypertrophication are cultured (culture of cells) and a value of the alkaline phosphatase activity thereof cultured by adding only the MEM growth medium, it is determined that the chondrocytes capable of hypertrophication do not produce an agent capable of inducing differentiation of undifferentiated cells into induced osteoblasts.

In the case where the chondrocytes capable of hypertrophication derived from human are cultured in the MEM differentiation agent producing medium, it is predicted that they produce the agent capable of inducing differentiation of undifferentiated cells into induced osteoblasts. On the other hand, in the case where the chondrocytes capable of hypertrophication derived from human are cultured in the MEM growth medium, it is predicted that they do not produce the agent capable of inducing differentiation of undifferentiated cells into induced osteoblasts.

### Comparative Example 4B

### Preparation and Detection of Agent Produced by Culturing Chondrocytes Incapable of Hypertrophication Derived from Human in MEM Differentiation Agent Producing Medium or MEM Growth Medium

Chondrocytes incapable of hypertrophication derived from human are obtained from the above-described organization. Each of an MEM differentiation agent producing medium and an MEM growth medium is added to the chondrocytes incapable of hypertrophication so that they are diluted so as to become a density of 4×10⁴ cells/cm². The chondrocytes incapable of hypertrophication are cultured, and then a supernatant of the medium is collected on a time course to obtain fractional supernatants.

Investigational human undifferentiated cells are inoculated in a 24-well plate. Eighteen hours after the inoculation, 1 mL of each of the fractional supernatants is added to the plate, and then the human undifferentiated cells are cultured. After 72 hours, an alkaline phosphatase activity of the human undifferentiated cells is measured in the same manner as Example 1.

In the case where a value of the alkaline phosphatase activity of the human undifferentiated cells cultured by adding the supernatant of the MEM differentiation agent producing medium, in which the chondrocytes incapable of hypertrophication derived from human are cultured, is hardly changed, it is determined that the chondrocytes incapable of hypertrophication do not produce an agent capable of inducing differentiation of undifferentiated cells into induced osteoblasts.

Further, in the case where the alkaline phosphatase activity of the human undifferentiated cells cultured by adding the supernatant of the MEM growth medium, in which the chondrocytes incapable of hypertrophication are cultured, is hardly changed, it is also determined that the chondrocytes incapable of hypertrophication do not produce the agent capable of inducing differentiation of undifferentiated cells into induced osteoblasts.

Even in the case where the chondrocytes incapable of hypertrophication derived from human are cultured in the MEM differentiation agent producing medium or the MEM growth medium, it is predicted they do not produce the agent capable of inducing differentiation of undifferentiated cells into induced osteoblasts.

### Example 5

### Preparation and Detection of Cellular Function Regulating Agent Produced by Culturing Chondrocytes Capable of Hypertrophication Derived from Human in HAM Differentiation Agent Producing Medium

Chondrocytes capable of hypertrophication are obtained in the same manner as Example 4. A HAM differentiation agent producing medium is added to the chondrocytes capable of hypertrophication so that they are diluted so as to become a density of 4×10⁴ cells/cm². The chondrocytes capable of hypertrophication are cultured, and then a supernatant of the medium is collected on a time course to obtain fractional supernatants.

Investigational human undifferentiated cells are inoculated in a 24-well plate. Eighteen hours after the inoculation, 1 mL of each of the fractional supernatants is added to the plate, and then the human undifferentiated cells are cultured. After 72 hours, an alkaline phosphatase activity of the human undifferentiated cells is measured in the same manner as Example 1.

In the case where the chondrocytes capable of hypertrophication derived from human are cultured in the HAM differentiation agent producing medium, it can be confirmed that they produce an agent capable of inducing differentiation of undifferentiated cells into induced osteoblasts in the same method and criteria as Example 1.

### Comparative Example 5A

### Preparation and Detection of Agent Produced by Culturing Chondrocytes Capable of Hypertrophication Derived from Human in HAM growth Medium

A HAM growth medium is added to chondrocytes capable of hypertrophication derived from human so that they are diluted so as to become a density of 4×10⁴ cells/cm². The chondrocytes capable of hypertrophication are cultured, and a supernatant of the medium is collected on a time course to obtain fractional supernatants.

Investigational human undifferentiated cells are inoculated in a 24-well plate. Eighteen hours after the inoculation, 1 mL of each of the fractional supernatants is added to the plat, and then the human undifferentiated cells are cultured. After 72 hours, an alkaline phosphatase activity of the human undifferentiated cells is measured in the same manner as Example 1.

In the case where the chondrocytes capable of hypertrophication derived from human are cultured in the HAM growth medium, it can be confirmed that they do not produce an agent capable of inducing differentiation of undifferentiated cells into induced osteoblasts in the same method and criteria as Example 1.

### Comparative Example 5B

### Preparation and Detection of Agent Produced by Culturing chondrocytes Incapable of Hypertrophication Derived from Human in HAM differentiation Agent Producing Medium or HAM Growth Medium

Chondrocytes incapable of hypertrophication derived from human are obtained in the same manner as Comparative Example 4B. Each of a HAM differentiation agent producing medium and a HAM growth medium is added to the chondrocytes incapable of hypertrophication so that they are diluted so as to become a density of 4×10⁴ cells/cm². The chondrocytes incapable of hypertrophication are cultured, and then a supernatant of the medium is collected on a time course to obtain fractional supernatants.

Investigational human undifferentiated cells are inoculated in a 24-well plate. Eighteen hours after the inoculation, 1 mL of each of the fractional supernatants is added to the plate, and then the human undifferentiated cells are cultured. After 72 hours, an alkaline phosphatase activity of the human undifferentiated cells is measured in the same method as Example 1.

It can be confirmed whether the human undifferentiated cells cultured by adding the supernatant of the HAM differentiation agent producing medium or the HAM growth medium in which the chondrocytes incapable of hypertrophication derived from human are cultured (culture supernatant) express induced osteoblast markers in the same method and criteria as Example 1.

### Conclusion of Examples 4 and 5 and Comparative Examples 4A to 5B

According to Examples described above, it can be examined whether the chondrocytes capable of hypertrophication derived from human produce the agent capable of inducing differentiation of undifferentiated cells into induced osteoblasts, in spite of the kind of the basal medium contained in the differentiation agent producing medium.

According to Examples 1 and 3, and Comparative Examples 1A to 1E and 3A to 3C, even in the case where the chondrocytes capable of hypertrophication derived from rat are cultured in any growth medium, it is substantiated that they do not produce the agent capable of inducing differentiation of undifferentiated cells into induced osteoblasts. Furthermore, even in the case where the chondrocytes incapable of hypertrophication derived from rat are cultured in any medium, it is substantiated that they do not produce the agent capable of inducing differentiation of undifferentiated cells into induced osteoblasts.

For these reasons, it is suggested that the agent capable of inducing differentiation of undifferentiated cells into induced osteoblasts is produced only by culturing the chondrocytes capable of hypertrophication in the differentiation agent producing medium. Therefore, since the basal medium contained in the medium is also unlikely to affect the production of the induced osteoblast differentiation inducing agent by the chondrocytes capable of hypertrophication derived from human as long as it can be normally used in a cell culture, it is presumed that any basal medium can be used in the present method.

### Example 6

### Study on Whether Agent Produced by Chondrocytes Capable of Hypertrophication has Ability of Inducing Differentiation of Undifferentiated cells other than Mouse C3H10T1/2 Cells into Induced Osteoblasts

A supernatant of an MEM differentiation agent producing medium or an MEM growth medium in which chondrocytes capable of hypertrophication were cultured (culture supernatant) was obtained in the same manner as Example 1. BALB/3T3 cells, 3T3-Swiss albino cells and NIH3T3 cells were used as undifferentiated cells. Each kind of the cells was inoculated in a 24-well plate. Eighteen hours after the inoculation, 1 mL of each of the supernatant and the medium was added to the plate, and then the cells were cultured in a 5% CO₂ incubator at 37° C. After 72 hours, an alkaline phosphatase activity of the cells was measured in the same manner as Example 1.

In the case where the supernatant of the MEM differentiation agent producing medium, in which the chondrocytes capable of hypertrophication were cultured, was added to each kind of the cells, when a value of the alkaline phosphatase activity thereof cultured by adding only the MEM differentiation agent producing medium was defined as “1”, a value of the alkaline phosphatase activity of the BALB/3T3 cells was about 5.9 times (see left in Table 4, and FIG. 6A), that of the 3T3-Swiss albino cells was about 13.8 times (see center in Table 4, and FIG. 6A), and that of the NIH3T3 cells was about 5.4 times (see right in Table 4, and FIG. 6A).

In the case where the supernatant of the MEM growth medium, in which the chondrocytes capable of hypertrophication were cultured, was added to each kind of the cells, when a value of the alkaline phosphatase activity thereof cultured by adding only the MEM growth medium was defined as “1”, a value of the alkaline phosphatase activity of the BALB/3T3 cells was about 1.3 times (see left in Table 4, and FIG. 6A), that of the 3T3-Swiss albino cells was about 1.1 times (see center in Table 4, and FIG. 6A), and that of the NIH3T3 cells was about 0.9 time (see right in Table 4, and FIG. 6A).

TABLE 4
(ability of inducing differentiation of
BALB/3T3 cells, 3T3-Swiss albino cells or NIH-3T3 cells into osteoblasts)

| | BALB/3T3 | | 3T3-Swiss albino | | NIH-3T3 | |
|---|---|---|---|---|---|---|
| | Relative | Absolute | Relative | Absolute | Relative | Absolute |
| | value | value | value | value | value | value |
| GC differentiation | 5.9 | 0.107 | 13.8 | 0.174 | 5.4 | 0.097 |
| supernatant | | | | | | |
| Only differentiation | 1 | 0.018 | 1 | 0.013 | 1 | 0.018 |
| medium | | | | | | |
| GC growth supernatant | 1.3 | 0.021 | 1.1 | 0.013 | 0.9 | 0.016 |
| Only growth medium | 1 | 0.016 | 1 | 0.013 | 1 | 0.018 |

GC (4 week-old): one experiment was performed. Three trials were performed.
GC differentiation supernatant: supernatant of MEM differentiation agent producing medium in which chondrocytes capable of hypertrophication were cultured
GC growth supernatant: supernatant of MEM growth medium in which chondrocytes capable of hypertrophication were cultured
Only differentiation medium: MEM differentiation agent producing medium alone
Only growth medium: MEM growth medium alone

In the case where the chondrocytes capable of hypertrophication were cultured in the MEM differentiation agent producing medium, it was confirmed that there was an agent capable of increasing the alkaline phosphatase activity of each kind of the 3T3-Swiss albino cells, the BALB/3T3 cells and the NIH3T3 cells, and capable of inducing differentiation thereof into induced osteoblasts in the supernatant of the medium (culture supernatant). On the other hand, in the case where the chondrocytes capable of hypertrophication were cultured in the MEM growth medium, it was confirmed that there was not the agent in the supernatant of the medium.

### Comparative Example 6

### Study on Whether Component Existing in Supernatant of Medium, in which Resting Cartilage Cells Incapable of Hypertrophication are Cultured, has Ability of Inducing Differentiation of Undifferentiated Cells other than Mouse C3H10T1/2 Cells into Induced Osteoblasts

A supernatant of an MEM differentiation agent producing medium or an MEM growth medium, in which resting cartilage cells incapable of hypertrophication were cultured, was obtained in the same manner as Comparative Example 1B. BALB/3T3 cells, 3T3-Swiss albino cells and NIH3T3 cells were used as undifferentiated cells. Each kind of the cells was inoculated in a 24-well plate. Eighteen hours after the inoculation, 1 mL of each of the supernatant and the medium was added to the plate, and then the cells were cultured in a 5% CO₂ incubator at 37° C. After 72 hours, an alkaline phosphatase activity of the cells was measured in the same manner as Example 1.

In the case where the supernatant of the MEM differentiation agent producing medium, in which the resting cartilage cells incapable of hypertrophication were cultured, was added to each kind of the cells, when a value of the alkaline phosphatase activity thereof cultured by adding only the MEM differentiation agent producing medium was defined as “1”, a value of the alkaline phosphatase activity of the BALB/3T3 cells was about 1.0 time (see left in Table 5, and FIG. 6A), that of the 3T3-Swiss albino cells was about 1.1 times (see center in Table 5, and FIG. 6A), and that of the NIH3T3 cells was about 1.0 time (see right in Table 5, and FIG. 6A).

In the case where the supernatant of the MEM growth medium, in which the resting cartilage cells incapable of hypertrophication were cultured, was added to each kind of the cells, when a value of the alkaline phosphatase activity thereof cultured by adding only the MEM growth medium was defined as “1”, a value of the alkaline phosphatase activity of the BALB/3T3 cells was about 1.3 times (see left in Table 5, and FIG. 6A), that of the 3T3-Swiss albino cells was about 0.9 time (see center in Table 5, and FIG. 6A), and that of the NIH3T3 cells was about 1.0 time (see right in Table 5, and FIG. 6A).

TABLE 5
(ability of inducing differentiation of
BALB/3T3 cells, 3T3-Swiss albino cells or NIH-3T3 cells into osteoblast)

| | BALB/3T3 | | 3T3-Swiss albino | | NIH-3T3 | |
|---|---|---|---|---|---|---|
| | Relative | Absolute | Relative | Absolute | Relative | Absolute |
| | value | value | value | value | value | value |
| RC differentiation | 1.0 | 0.018 | 1.1 | 0.014 | 1.0 | 0.018 |
| supernatant | | | | | | |
| Only differentiation | 1 | 0.018 | 1 | 0.013 | 1 | 0.018 |
| medium | | | | | | |
| RC growth supernatant | 1.3 | 0.020 | 0.9 | 0.012 | 1.0 | 0.019 |
| Only growth medium | 1 | 0.016 | 1 | 0.013 | 1 | 0.018 |

RC (8 week-old): one experiment was performed. Three trials were performed.
RC differentiation supernatant: supernatant of MEM differentiation agent producing medium in which resting cartilage cells were cultured
RC growth supernatant: supernatant of MEM growth medium in which resting cartilage cells were cultured
Only differentiation medium: MEM differentiation medium alone
Only growth medium: MEM growth medium alone.

There was little difference between a value of the alkaline phosphatase activity of each kind of the 3T3-Swiss albino cells, the BALB/3T3 cells and the NIH3T3 cells cultured by adding the supernatant of the MEM differentiation agent producing medium in which the resting cartilage cells incapable of hypertrophication were cultured and a value of the alkaline phosphatase activity thereof cultured by adding only the MEM differentiation agent producing medium.

Therefore, it was confirmed that there was not an agent capable of inducing differentiation of each kind of these undifferentiated cells into induced osteoblasts in the supernatant of the medium (culture supernatant). It was also confirmed that there was not the agent in the supernatant of the MEM growth medium in which the resting cartilage cells incapable of hypertrophication were cultured.

### Example 7

### Preparation and Detection of Cellular Function Regulating Agent Produced by Culturing Chondrocytes Capable of Hypertrophication Derived from Costal Cartilage in Medium Containing Various Kinds of Conventional Osteoblast Differentiation Inducing Components)

Chondrocytes capable of hypertrophication derived from costal cartilage were obtained in the same manner as Example 1. An MEM growth medium (containing a minimum essential medium (MEM), 15% FBS, 100 U/mL penicillin, 0.1 mg/mL streptomycin and 0.25 μg/mL amphotericin B) was added to the chondrocytes capable of hypertrophication so that they were diluted so as to become a density of 4×10⁴ cells/cm², and then dexamethasone, β-glycerophosphate, ascorbic acid or a combination thereof as a conventional osteoblast differentiation component was further added to the medium. The chondrocytes capable of hypertrophication were cultured, and then a supernatant of the medium was collected on a time course to obtain fractional supernatants.

| | Concentration of each osteoblast |
|---|---|
| | differentiation inducing component |
| Component added | added |
| Differentiation agent | Dex: 10 nM, βGP: 10 mM, Asc: 50 μg/mL |
| producing medium | |
| Dex | Dex: 10 nM |
| βGP | βGP: 10 mM |
| Asc | Asc: 50 μg/mL |
| Dex + βGP | Dex: 10 nM, βGP: 10 mM |
| Dex + Asc | Dex: 10 nM, Asc: 50 μg/mL |
| βGP + Asc | βGP: 10 mM, Asc: 50 μg/mL |
| Growth medium | No osteoblast differentiation |
| | inducing component |

Dex: dexamethasone,
βGP: β-glycerophosphate,
Asc: ascorbic acid

1 mL of each of the fractional supernatants was added to mouse C3H10T1/2 cells (1.25×10⁴ cells/cm²), they were cultured in a 5% CO₂ incubator at 37° C., and then an alkaline phosphatase activity of the mouse C3H10T1/2 cells was measured in the same manner as Example 1. As shown in the following Table and FIG. 6B, a value of the alkaline phosphatase activity thereof cultured by adding the supernatant of the MEM growth medium containing Dex, βGP and Asc (MEM differentiation agent producing medium) was 0.041, and a value of the alkaline phosphatase activity thereof cultured by adding the supernatant of the MEM growth medium containing βGP and Asc was 0.044.

Furthermore, a value of the alkaline phosphatase activity thereof cultured by adding the supernatant of the MEM growth medium containing only Dex was 0.016, a value of the alkaline phosphatase activity thereof cultured by adding the supernatant of the MEM growth medium containing only BGP was 0.015, and a value of the alkaline phosphatase activity thereof cultured by adding the supernatant of the MEM growth medium containing only Asc was 0.016. A value of the alkaline phosphatase activity thereof cultured by adding the supernatant of the MEM growth medium containing Dex and BGP was 0.022, and a value of the alkaline phosphatase activity thereof cultured by adding the supernatant of the MEM growth medium containing Dex and Asc was 0.017.

In the case where a supernatant of the growth medium, in which the chondrocytes capable of hypertrophication were cultured, was added to the mouse C3H10T1/2 cells as a control, a value of the alkaline phosphatase activity thereof was 0.014. In the case where only the MEM differentiation agent producing medium and the MEM growth medium were added to the mouse C3H10T1/2 cells, values of the alkaline phosphatase activities thereof were 0.016 and 0.014, respectively.

(Effect of Conventional Osteoblast Differentiation Inducing Components on Production of Agent Capable of Inducing Differentiation into Induced Osteoblasts)

| | Mean | SD |
|---|---|---|
| Dex + βGP + Asc | 0.041 | 0.008 |
| Dex | 0.016 | 0.004 |
| βGP | 0.015 | 0.004 |
| Asc | 0.016 | 0.001 |
| Dex + βGP | 0.022 | 0.004 |
| Dex + Asc | 0.017 | 0.002 |
| βGP + Asc | 0.044 | 0.016 |
| Growth medium | 0.014 | 0.002 |
| Only differentiation medium | 0.016 | 0.002 |
| Only growth medium | 0.014 | 0.001 |

Dex: dexamethasone
βGP: β-glycerophosphate
Asc: ascorbic acid
Only differentiation medium: MEM differentiation producing medium alone (in which chondrocytes capable of hypertrophication were not cultured)
Only growth medium: MEM growth medium alone (in which chondrocytes capable of hypertrophication were not cultured)

In the case where each of the conventional osteoblast differentiation inducing components was independently added to the MEM growth medium in which the chondrocytes capable of hypertrophication were cultured, the agent capable of inducing differentiation of undifferentiated cells into induced osteoblasts was not produced. In the case where the β-glycerophosphate and the ascorbic acid were added to the MEM growth medium, the agent capable of inducing differentiation of undifferentiated cells into induced osteoblasts was produced.

In the case where all of the dexamethasone, the β-glycerophosphate and the ascorbic acid were added to the MEM growth medium (i.e., the MEM differentiation agent producing medium was used), it was confirmed that the production of the agent capable of inducing differentiation of undifferentiated cells into induced osteoblasts was promoted.

### Example 8

### Study on Agent Contained in Supernatant of MEM Differentiation Agent Producing Medium in which Chondrocytes Capable of Hypertrophication were Cultured

Chondrocytes capable of hypertrophication were cultured in an MEM differentiation agent producing medium in the same manner as Example 1, and a supernatant of the medium was collected on a time course ranging from 4 days to 3 weeks to obtain fractional supernatants. Each of the fractional supernatants was placed in a centrifugal filter, and then subjected to centrifugal ultrafiltration at 4,000×g and at 4° C. for 30 minutes under such a condition that a high molecular fraction and a low molecular fraction were separated from each other. In this way, the fractional supernatant was separated into a high molecular weight fraction with a molecular weight of 50,000 or higher and a low molecular weight fraction with a molecular weight of 50,000 or lower.

Next, mouse C3H10T1/2 cells (in a BME medium) were inoculated in a 24-well plate at a density of 1.25×10⁴ cells/cm² and on hydroxyapatite at a density of 1×10⁶ cells/mL. Eighteen hours after the inoculation, 1 mL of each of the high and low molecular weight fractions was added to the plate and the hydroxyapatite, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

In the case where the high molecular weight fraction with the molecular weight of 50,000 or higher separated from each of the fractional supernatants was added to the mouse C3H10T1/2 cells inoculated both in the 24-well plate and on the hydroxyapatite, they were stained red (see FIGS. 7A and 7B). It was indicated that an agent capable of increasing the alkaline phosphatase activity of the mouse C3H10T1/2 cells was present in the high molecular weight fraction.

On the other hand, in the case where the low molecular weight fraction with the molecular weight of 50,000 or lower separated from each of the fractional supernatants was added to the mouse C3H10T1/2 cells inoculated both in the 24-well plate and on the hydroxyapatite, they were not stained. Namely, the alkaline phosphatase activity of the mouse C3H10T1/2 cells was not observed (see FIGS. 7C and 7D).

According to the above results, it was found that an agent capable of inducing differentiation of the mouse C3H10T1/2 cells into induced osteoblasts was present in the high molecular weight fraction with the molecular weight of 50,000 or higher separated from the supernatant of the MEM differentiation agent producing medium in which the chondrocytes capable of hypertrophication were cultured.

### Example 9

### Preparation and Detection of Cellular Function Regulating Agent Produced by Culturing Chondrocytes Capable of Hypertrophication Derived from Mouse Costa/Costal Cartilage in MEM Differentiation Agent Producing Medium

(Preparation of Chondrocytes Capable of Hypertrophication from Mouse Costa/Costal Cartilages)

Eight week-old male mice (Balb/cA) were examined in this Example. The mice were sacrificed using chloroform. The mice's chests were shaved using a razor and their whole bodies were immersed into a Hibitane solution (10-fold dilution) to be disinfected. The mice's chests were incised and costa/costal cartilages were collected aseptically.

Translucent growth cartilage regions were collected from boundary regions of the costa/costal cartilages. The growth cartilage regions were sectioned and stirred in a 0.25% trypsin-EDTA/dulbecco's phosphate buffered saline (D-PBS) at 37° C. for 1 hour. Next, the sections were rinsed by centrifugation (at 170×g for 3 minutes), and then stirred together with a 0.2% collagenase (produced by Invitrogen Corporation)/D-PBS at 37° C. for 2.5 hours.

Thereafter, the sections were rinsed by centrifugation (at 170×g for 3 minutes), and then stirred together with a 0.2% dispase (produced by Invitrogen Corporation)/(HAM+10% FBS) in a stirring flask overnight at 37° C. In the following day, cells were filtered and rinsed by centrifugation (at 170×g for 3 minutes). The cells were stained with trypan blue and the number thereof was counted under a microscope.

The cells evaluated as cells not stained were considered to be living cells, and those stained blue were considered to be dead cells.

(Identification of Chondrocytes Capable of Hypertrophication)

Since the cells obtained in Example 9 were impaired by the enzymes used in the separation thereof (e.g. the trypsin, the collagenase, and the dispase), they were cultured to recover. Chondrocytes capable of hypertrophication are identified using localization or expression of chondrocyte markers and their morphological hypertrophies under a microscope.

(Localization or Expression of Specific Markers for Chondrocytes Capable of Hypertrophication)

A lysate prepared using the method as described above is treated with sodium dodecyl sulfate (SDS) to obtain a SDS-treated solution. The SDS-treated solution is subjected to SDS polyacrylamide gel electrophoresis. Thereafter, a gel used in the SDS polyacrylamide gel electrophoresis is blotted onto a transfer membrane (Western blotting), reacted with a primary antibody to a chondrocyte marker, and then detected with a secondary antibody labeled with an enzyme such as peroxidase, alkaline phosphatase or glucosidase, or a fluorescent tag such as fluorescein isothiocyanate (FITC), phycoerythrin (PE), Texas Red, 7-amino-4-methyl coumarin-3-acetate (AMCA) or rhodamine.

A culture (culture of cells) obtained using the above-described manipulation is fixed with a 10% neutral formalin buffer, reacted with a primary antibody to a chondrocyte marker, and then detected with a secondary antibody labeled with an enzyme such as peroxidase, alkaline phosphatase or glucosidase, or a fluorescent tag such as FITC, PE, Texas Red, AMCA or rhodamine.

An alkaline phosphatase also can be detected using a staining method. A culture obtained using the above-described manipulation is stained by fixing it with a 60% acetone/citric acid buffer, rinsing it with distilled water, and then immersing it into a mixture of First Violet B and Naphthol AS-MX at room temperature in the dark for 30 minutes to react with each other.

(Morphological Assessment on Ability of Hypertrophication in Chondrocytes)

A HAM's F12 medium containing 5×10⁵ cells is centrifuged to form a pellet of the cells. The pellet (cell pellet) is cultured for a predetermined period. Cell sizes before and after the culture are compared under a microscope. In the case where a significant increase in size is observed, the cells are determined to be capable of hypertrophication.

It can be confirmed whether the cells obtained in Example 9 are the chondrocytes capable of hypertrophication by determining whether these cells express a chondrocyte marker or morphologically hypertrophy.

(Detection of Agent Produced by Chondrocytes Capable of Hypertrophication Collected from Mouse Costa/Costal Cartilage)

Chondrocytes capable of hypertrophication were obtained in the same manner as Example 9. An MEM differentiation agent producing medium (containing a minimum essential medium (MEM), 15% FBS (fetal bovine serum), 10 nM dexamethasone, 10 mM β-glycerophosphate, 50 μg/mL ascorbic acid, 100 U/mL penicillin, 0.1 mg/mL streptomycin and 0.25 μg/mL amphotericin B) was added to the chondrocytes capable of hypertrophication so that they were diluted so as to become a density of 4×10⁴ cells/cm² to prepare a cell suspension.

The cell suspension was inoculated evenly on a dish (produced by Becton, Dickinson and Company), the chondrocytes capable of hypertrophication were cultured in a 5% CO₂ incubator at 37° C., and then a supernatant of the medium (culture supernatant) was collected on a time course (4 days, 7 days, 11 days, 14 days, 18 days and 21 days) to obtain fractional supernatants.

(Study on Whether Supernatant Collected has Ability of Inducing Differentiation of Undifferentiated Cells into Induced Osteoblasts)

Mouse C3H10T1/2 cells (“CCL-226” produced by Dainippon Sumitomo Pharma Co. Ltd.) were inoculated evenly in a 24-well plate (produced by Becton, Dickinson and Company) at a density of 1.25×10⁴ cells/cm² (i.e., 2.5×10⁴ cells/well). Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

In this Example, in the case where a value of the alkaline phosphatase (ALP) activity of the mouse C3H10T1/2 cells (whole the cells) cultured by adding the supernatant containing the present agent increased by more than about 1.5 times that of the mouse C3H10T1/2 cells cultured by adding the medium containing no present agent, the present agent was determined to have an ability of increasing the alkaline phosphatase activity.

When a value of the alkaline phosphatase activity of the mouse C3H10T1/2 cells cultured by adding only the MEM differentiation agent producing medium was defined as “1”, a value of the alkaline phosphatase activity thereof cultured by adding the supernatant increased to about 3.1 times (see upper column in Table 6, and FIG. 8).

(Identification of Induced Osteoblasts)

As described above, it was shown that the alkaline phosphatase (ALP) activity, which was one of the induced osteoblast markers, of the mouse C3H10T1/2 cells increased by an agent capable of inducing differentiation into induced osteoblasts. Furthermore, the mouse C3H10T1/2 cells cultured by adding the agent capable of inducing differentiation into induced osteoblasts for 72 hours were significantly stained red with alkaline phosphatase staining.

For this reason, expression of the alkaline phosphatase was also indicated using the staining method. As a result, it was confirmed that the mouse C3H10T1/2 cells were differentiated into the induced osteoblasts.

### Comparative Example 9A

### Preparation and Detection of Agent Produced by Culturing Chondrocytes Capable of Hypertrophication Derived from Mouse Costa/Costal Cartilage in MEM Growth Medium)

Chondrocytes capable of hypertrophication were collected from mouse costa/costal cartilages in the same manner as Example 9. An MEM growth medium (containing a minimum essential medium (MEM), 15% FBS, 100 U/mL penicillin, 0.1 mg/mL streptomycin and 0.25 μg/mL amphotericin B) was added to the chondrocytes capable of hypertrophication so that they were diluted so as to become a density of 4×10⁴ cells/cm². The chondrocytes capable of hypertrophication were cultured, and then a supernatant of the medium was collected on a time course (4 days, 7 days, 11 days, 14 days, 18 days, 21 days) to obtain fractional supernatants.

Mouse C3H10T1/2 cells (“CCL-226” produced by Dainippon Sumitomo Pharma Co. Ltd.) were inoculated evenly in a 24-well plate (produced by Becton, Dickinson and Company) at a density of 1.25×10⁴ cells/cm² (i.e., 2.5×10⁴ cells/well). Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

When a value of the alkaline phosphatase activity of the mouse C3H10T1/2 cells cultured by adding only the MEM growth medium was defined as “1”, a value of the alkaline phosphatase activity thereof cultured by adding the supernatant was about 1.6 times (see lower column in Table 6, and FIG. 8).

There was little difference between a value of the alkaline phosphatase activity of the mouse C3H10T1/2 cells cultured by adding the supernatant of the MEM growth medium in which the chondrocytes capable of hypertrophication were cultured (culture of cells) and a value of the alkaline phosphatase activity thereof cultured by adding only the MEM growth medium (see FIG. 8).

(Identification of Induced Osteoblast)

In the same method as Example 9, it was confirmed that the supernatant of the medium (culture of cells) obtained by the above-described manipulation did not express induced osteoblast markers in the mouse C3H10T1/2 cells.

### Comparative Example 9B

### Preparation and Detection of Agent Produced by Culturing Resting Cartilage Cells Derived from Mouse Costal Cartilage in MEM Differentiation Agent Producing Medium)

(Preparation of Resting Cartilage Cells from Costal Cartilages)

Eight week-old male mice (Balb/cA) were sacrificed using chloroform. The mice's chests were shaved using a razor and their whole bodies were immersed into a Hibitane solution (10-fold dilution) to be disinfected. The mice's chests were incised and costal cartilages were collected aseptically.

Opaque resting cartilage regions were collected from the costal cartilages. The resting cartilage regions were sectioned and stirred in a 0.25% trypsin-EDTA/D-PBS (dulbecco's phosphate buffered saline) at 37° C. for 1 hour. Next, the sections were rinsed by centrifugation (at 170×g for 3 minutes), and then stirred together with a 0.2% collagenase (produced by Invitrogen Corporation)/D-PBS at 37° C. for 2.5 hours.

Thereafter, the sections were rinsed by centrifugation (at 170×g for 3minutes), and then stirred together with a 0.2% dispase (produced by Invitrogen Corporation)/(HAM+10% FBS) in a stirring flask overnight at 37° C. Optionally, the overnight treatment with the 0.2% dispase was omitted. In the following day, cells were filtered and rinsed by centrifugation (at 170×g for 3 minutes). The cells were stained with trypan blue and the number thereof was counted under a microscope.

The cells evaluated as cells not stained were considered to be living cells, and those stained blue were considered to be dead cells.

(Identification of Chondrocytes Incapable of Hypertrophication Derived from Costal Cartilage)

In the same method as Example 9, localization or expression of chondrocyte markers can be detected. Further, it can be confirmed whether the cells obtained are chondrocytes capable of hypertrophication by assessing them morphologically.

(Detection of Agent Produced by Culturing Resting Cartilage Cells Collected from Costal Cartilage in MEM Differentiation Agent Producing Medium)

An MEM differentiation agent producing medium (comprising a minimum essential medium (MEM), 15% FBS (fetal bovine serum), 10 nM dexamethasone, 10 mM β-glycerophosphate, μg/mL ascorbic acid, 100 U/mL penicillin, 0.1 mg/mL streptomycin and 0.25 μg/mL amphotericin B) was added to resting cartilage cells collected from costal cartilage so that they were diluted so as to become a density of 4×10⁴ cells/cm². The resting cartilage cells were cultured, and then a supernatant of the medium was collected on a time course (4 days, 7 days, 11 days, 14 days, 18 days, 21 days) to obtain fractional supernatants.

Mouse C3H10T1/2 cells (“CCL-226” produced by Dainippon Sumitomo Pharma Co. Ltd.) were inoculated in a 24-well plate. Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

When a value of the alkaline phosphatase activity of the mouse C3H10T1/2 cells cultured by adding only the MEM differentiation agent producing medium was defined as “1”, a value of the alkaline phosphatase activity thereof cultured by adding the supernatant was about 0.8 time (see upper column in Table 6, and FIG. 8).

There was little difference between a value of the alkaline phosphatase activity of the mouse C3H10T1/2 cells cultured by adding the supernatant of the MEM differentiation agent producing medium in which the resting cartilage cells were cultured (culture of cells) and a value of the alkaline phosphatase activity thereof cultured by adding only the MEM differentiation agent producing medium (see upper column in Table 6, and FIG. 8).

In the same method and criteria as Example 1, it can be confirmed whether the supernatant of the medium (culture of cells) obtained by the above-described manipulation expresses induced osteoblast markers in the mouse C3H10T1/2 cells.

### Comparative Example 9C

### Preparation and Detection of Agent Produced by Culturing Resting Cartilage Cells Collected from Mouse Costal Cartilage in MEM Growth Medium

Resting cartilage cells were collected from costal cartilages in the same manner as Comparative Example 9B. An MEM growth medium (containing a minimum essential medium (MEM), 15% FBS, 100 U/mL penicillin, 0.1 mg/mL streptomycin and 0.25 μg/mL amphotericin B) was added to the resting cartilage cells so that they were diluted so as to become a density of 4×10⁴ cells/cm². The resting cartilage cells were cultured, and then a supernatant of the medium on a time course (4 days, 7 days, 11 days, 14 day, 18 days, 21 days) to obtain fractional supernatants.

Mouse C3H10T1/2 cells (“CCL-226” produced by Dainippon Sumitomo Pharma Co. Ltd.) were inoculated in a 24-well plate. Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

When a value of the alkaline phosphatase activity of the mouse C3H10T1/2 cells cultured by adding only the MEM growth medium was defined as “1”, a value of the alkaline phosphatase activity thereof cultured by adding the supernatant was about 1.0 time (see lower column in Table 6, and FIG. 8).

There was little difference between a value of the alkaline phosphatase activity of the mouse C3H10T1/2 cells cultured by adding the supernatant of the MEM growth medium in which the resting cartilage cells were cultured and a value of the alkaline phosphatase activity thereof cultured by adding only the MEM growth medium (see lower column in Table 6, and FIG. 8).

It was confirmed that the supernatant of the medium (culture of cells) obtained by the above-described manipulation did not express induced osteoblast markers in the mouse C3H10T1/2 cells.

TABLE 6
(comparison of alkaline phosphatase
activities of Example 9 and Comparative Examples 9A to 9C in
each of which supernatant of MEM differentiation agent
producing medium or MEM growth medium in which chondrocytes
derived from mouse were cultured was added to mouse C3H10T1/2
cells)

| | |
|---|---|
| Relative value | Absolute value |

MEM differentiation agent producing medium (mean value)

| | | |
|---|---|---|
| GC supernatant | 3.1 | 0.038 |
| RC supernatant | 0.8 | 0.011 |
| Only differentiation medium | 1 | 0.012 |

MEM growth medium (mean value)

| | | |
|---|---|---|
| GC supernatant | 1.6 | 0.021 |
| RC supernatant | 1.0 | 0.013 |
| Only growth medium | 1 | 0.014 |

8 week-old: one experiment was performed. Two trials were carried out.
GC supernatant: supernatant of each of mediums in which chondrocytes capable of hypertrophication were cultured RC supernatant: supernatant of each of mediums in which resting cartilage cells were cultured
Only differentiation medium: MEM differentiation agent producing medium alone
Only growth medium: MEM growth medium alone

### Conclusion of Example 9 and Comparative Examples 9A to 9C

In the case where the chondrocytes capable of hypertrophication collected from mouse costa/costal cartilage were cultured in the MEM differentiation agent producing medium, it was confirmed that there was an agent capable of increasing the alkaline phosphatase activity of the mouse C3H10T1/2 cells, and capable of inducing differentiation thereof into induced osteoblasts in the supernatant of the medium (culture supernatant).

On the other hand, in the case where the chondrocytes capable of hypertrophication were cultured in the MEM growth medium, it was confirmed that there was not the agent in the supernatant of the medium (culture supernatant). Therefore, it was found that the chondrocytes capable of hypertrophication produced an agent capable of inducing differentiation of undifferentiated cells into induced osteoblasts by culturing them in the MEM differentiation agent producing medium.

Even in the case where the resting cartilage cells derived from mouse costal cartilage were cultured in the MEM differentiation agent producing medium or the MEM growth medium, it was confirmed that they did not produce the agent capable of inducing differentiation of undifferentiated cells into induced osteoblasts.

### Example 10

### Preparation and Detection of Cellular Function Regulating Agent Produced by Culturing Chondrocytes Capable of Hypertrophication Derived from Rabbit Costa/Costal Cartilage in MEM Differentiation Agent Producing Medium

(Preparation of Chondrocytes Capable of Hypertrophication from Rabbit Costa/Costal Cartilages)

Eight week-old male rabbits (Japanese White) were examined in this Example. The rabbits were sacrificed using chloroform. The rabbits' chests were shaved using a razor and their whole bodies were immersed into a Hibitane solution (10-fold dilution) to be disinfected. The rabbits' chests were incised and costa/costal cartilages were collected aseptically.

Translucent growth cartilage regions were collected from boundary regions of the costa/costal cartilages. The growth cartilage regions were sectioned and stirred in a 0.25% trypsin-EDTA/D-PBS (dulbecco's phosphate buffered saline) at 37° C. for 1 hour. Next, the sections were rinsed by centrifugation (at 170×g for 3 minutes), and then stirred together with a 0.2% collagenase (produced by Invitrogen Corporation)/D-PBS at 37° C. for 2.5 hours.

Thereafter, the sections were rinsed by centrifugation (at 170×g for 3minutes), and then stirred together with a 0.2% dispase (produced by Invitrogen Corporation)/(HAM+10% FBS) in a stirring flask overnight at 37° C. In the following day, cells were filtered and rinsed by centrifugation (at 170×g for 3 minutes). The cells were stained with trypan blue and the number thereof was counted under a microscope.

The cells evaluated as cells not stained were considered to be living cells, and those stained blue were considered to be dead cells.

(Identification of Chondrocytes Capable of Hypertrophication)

Since the cells obtained in Example 10 were impaired by the enzymes used in the separation thereof (e.g. the trypsin, the collagenase, and the dispase), they were cultured to recover. Chondrocytes capable of hypertrophication are identified using localization or expression of chondrocyte markers and their morphological hypertrophies under a microscope.

(Localization or Expression of Specific Markers for Chondrocytes Capable of Hypertrophication)

A lysate prepared using the method as described above is treated with sodium dodecyl sulfate (SDS) to obtain a SDS-treated solution. The SDS-treated solution is subjected to SDS polyacrylamide gel electrophoresis. Thereafter, a gel used is the SDS polyacrylamide gel electrophoresis is blotted onto a transfer membrane (Western blotting), reacted with a primary antibody to a chondrocyte marker, and then detected with a secondary antibody labeled with an enzyme such as peroxidase, alkaline phosphatase or glucosidase, or a fluorescent tag such as fluorescein isothiocyanate (FITC), phycoerythrin (PE), Texas Red, 7-amino-4-methyl coumarin-3-acetate (AMCA) or rhodamine.

A culture (culture of cells) obtained using the above-described manipulation is fixed with a 10% neutral formalin buffer, reacted with a primary antibody to a chondrocyte marker, and then detected with a secondary antibody labeled with an enzyme such as peroxidase, alkaline phosphatase or glucosidase, or a fluorescent tag such as FITC, PE, Texas Red, AMCA or rhodamine.

An alkaline phosphatase also can be detected using a staining method. A culture obtained using the above-described manipulation is stained by fixing it with a 60% acetone/citric acid buffer, rinsing it with distilled water, and them immersing it into a mixture of First Violet B and Naphthol AS-MX at room temperature in the dark for 30 minutes to react with each other.

(Morphological Assessment on Ability of Hypertrophication in Chondrocytes)

A HAM's F12 medium containing 5×10⁵ cells is centrifuged to form a pellet of the cells. The pellet (cell pellet) is cultured for a pre-determined period. Cell sizes before and after the culture are compared under a microscope. In the case where a significant increase in size is observed, the cells are determined to be capable of hypertrophication.

(Results)

It can be confirmed whether the cells obtained in Example are the chondrocytes capable of hypertrophication by determining whether these cells express a chondrocyte marker or morphologically hypertrophy.

(Detection of Agent Produced by Chondrocytes Capable of Hypertrophication Collected from Rabbit Costa/Costal Cartilage)

Chondrocytes capable of hypertrophication were obtained in the same manner as Example 10. An MEM differentiation agent producing medium (containing a minimum essential medium (MEM), 15% FBS (fetal bovine serum), 10 nM dexamethasone, 10 mM β-glycerophosphate, 50 μg/mL ascorbic acid, 100 U/mL penicillin, 0.1 mg/mL streptomycin and 0.25 μg/mL amphotericin B) was added to the chondrocytes capable of hypertrophication so that they were diluted so as to become a density of 4×10⁴ cells/cm² to obtain a cell suspension.

The cell suspension was inoculated evenly on a dish (produced by Becton, Dickinson and Company), the chondrocytes capable of hypertrophication were cultured in a 5% CO₂ incubator at 37° C., and then a supernatant of the medium (culture supernatant) was collected on a time course (4 days, 7 days, 11 days, 14 days, 18 days, 21 days) to obtain fractional supernatants.

(Study on whether Supernatant Collected has Ability of Inducing Differentiation of Undifferentiated Cells into Induced Osteoblasts)

Mouse C3H10T1/2 cells (“CCL-226” produced by Dainippon Sumitomo Pharma Co. Ltd.) were inoculated evenly in a 24-well plate (produced by Becton, Dickinson and Company) at a density of 1.25×10⁴ cells/cm² (i.e., 2.5×10⁴ cells/well). Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

In this Example, in the case where a value of the alkaline phosphatase (ALP) activity of the mouse C3H10T1/2 cells (whole the cells) cultured by adding the supernatant containing the present agent increased by more than about 1.5 times that of the mouse C3H10T1/2 cells cultured by adding the medium containing no present agent, the present agent was determined to have an ability of increasing the alkaline phosphatase activity.

When a value of the alkaline phosphatase activity of the mouse C3H10T1/2 cells cultured by adding only the MEM differentiation agent producing medium was defined as “1”, a value of the alkaline phosphatase activity thereof cultured by adding the supernatant increases.

(Identification of Induced Osteoblasts)

As described above, it was shown that the alkaline phosphatase (ALP) activity, which was one of the induced osteoblast markers, of the mouse C3H10T1/2 cells increased by an agent capable of inducing differentiation into induced osteoblasts. Furthermore, the mouse C3H10T1/2 cells cultured by adding the agent capable of inducing differentiation into induced osteoblasts for 72 hours were significantly stained red with alkaline phosphatase staining.

For this reason, expression of the alkaline phosphatase was also indicated using the staining method. As a result, it was confirmed that the mouse C3H10T1/2 cells were differentiated into the induced osteoblasts.

### Comparative Example 10A

### Preparation and Detection of Agent Produced by Culturing Chondrocytes Capable of Hypertrophication Derived from rabbit Costa/Costal cartilage in MEM Growth Medium)

Chondrocytes capable of hypertrophication were collected from rabbit costa/costal cartilages in the same manner as Example 10. An MEM growth medium (containing a minimum essential medium (MEM), 15% FBS, 100 U/mL penicillin, 0.1 mg/mL streptomycin and 0.25 μg/mL amphotericin B) was added to the chondrocytes capable of hypertrophication so that they were diluted so as to become a density of 4×10⁴ cells/cm². The chondrocytes capable of hypertrophication were cultured, and then a supernatant of the medium was collected on a time course (4 days, 7 days, 11 days, 14 days, 18 days, 21 days) to obtain fractional supernatants.

Mouse C3H10T1/2 cells (“CCL-226” produced by Dainippon Sumitomo Pharma Co. Ltd.) were inoculated evenly in a 24-well plate (produced by Becton, Dickinson and Company) at a density of 1.25×10⁴ cells/cm² (i.e., 2.5×10⁴ cells/well). Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

There was little difference between a value of the alkaline phosphatase activity of the mouse C3H10T1/2 cells cultured by adding the supernatant of the MEM growth medium in which the chondrocytes capable of hypertrophication were cultured (culture of cells) and a value of the alkaline phosphatase activity thereof cultured by adding only the MEM growth medium.

(Identification of Induced Osteoblasts)

In the same method and criteria as Example 10, it can be confirmed whether the supernatant of the medium (culture of cells) obtained by the above-described manipulation expresses induced osteoblast markers in the mouse C3H10T1/2 cells.

### Comparative Example 10B

### Preparation and Detection of Agent Produced by Culturing Resting Cartilage Cells Derived from rabbit Costal Cartilage in MEM Differentiation Agent Producing Medium

(Preparation of Resting Cartilage Cells from Costal Cartilages)

Eight week-old male rabbits (Japanese White) were sacrificed using chloroform. The rabbits' chests were shaved using a razor and their whole bodies were immersed into a Hibitane solution (10-fold dilution) to be disinfected. The rabbits' chests were incised and costal cartilages were collected aseptically.

Opaque resting cartilage regions were collected from the costal cartilages. The resting cartilage regions were sectioned and stirred in a 0.25% trypsin-EDTA/D-PBS (dulbecco's phosphate buffered saline) at 37° C. for 1 hour. Next, the sections were rinsed by centrifugation (at 170×g for 3 minutes), and then stirred together with a 0.2% collagenase (produced by Invitrogen Corporation)/D-PBS at 37° C. for 2.5 hours.

Thereafter, the sections were rinsed by centrifugation (at 170×g for 3minutes), and then stirred together with a 0.2% dispase (produced by Invitrogen Corporation)/(HAM +10% FBS) in a stirring flask overnight at 37° C. Optionally, the overnight treatment with the 0.2% dispase was omitted. In the following day, cells were filtered and rinsed by centrifugation (at 170×g for 3 minutes). The cells were stained with trypan blue and the number thereof was counted under a microscope.

The cells evaluated as cells not stained were considered to be living cells, and those stained blue were considered to be dead cells.

(Identification of Chondrocytes Incapable of Hypertrophication Derived from Costal Cartilage)

In the same method and criteria as Example 10, it can be confirmed whether the cells obtained are chondrocytes incapable of hypertrophication by detecting localization or expression of chondrocyte markers and assessing them morphologically.

(Detection of Agent Produced by Culturing Resting Cartilage Cells Collected from Costal Cartilage in MEM Differentiation Agent Producing Medium)

An MEM differentiation agent producing medium (containing a minimum essential medium (MEM), 15% FBS (fetal bovine serum), 10 nM dexamethasone, 10 mM β-glycerophosphate, μg/mL ascorbic acid, 100 U/mL penicillin, 0.1 mg/mL streptomycin and 0.25 μg/mL amphotericin B) was added to resting cartilage cells collected from costal cartilage so that they were diluted so as to become a density of 4×10⁴ cells/cm². The resting cartilage cells were cultured, and then a culture supernatant of the medium was collected on a time course (4 days, 7 days, 11 days, 14 days, 18 days, 21 days) to obtain fractional supernatants.

Mouse C3H10T1/2 cells (“CCL-226” produced by Dainippon Sumitomo Pharma Co. Ltd.) were inoculated in a 24-well plate. Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

There was little difference between a value of the alkaline phosphatase activity of the mouse C3H10T1/2 cells cultured by adding the supernatant of the MEM differentiation agent producing medium in which the resting cartilage cells were cultured (culture of cells) and a value of the alkaline phosphatase activity thereof cultured by adding only the MEM differentiation agent producing medium.

In the same method and criteria as Example 1, it can be confirmed whether the supernatant of the medium (culture of cells) obtained by the above-described manipulation expresses induced osteoblast markers in the mouse C3H10T1/2 cells.

### Comparative Example 10C

### Preparation and Detection of Agent Produced by Culturing Resting Cartilage Cells Collected from Costal Cartilage in MEM Growth Medium

Resting cartilage cells were collected from costal cartilages in the same manner as Comparative Example 10B. An MEM growth medium (containing a minimum essential medium (MEM), 15% FBS, 100 U/mL penicillin, 0.1 mg/mL streptomycin and 0.25 μg/mL amphotericin B) was added to the resting cartilage cells so that they were diluted so as to become a density of 4×10⁴ cells/cm². The resting cartilage cells were cultured, and then a supernatant of the medium was collected on a time course (4 days, 7 days, 11 days, 14 days, 18 days, 21 days) to obtain fractional supernatants.

Mouse C3H10T1/2 cells (“CCL-226” produced by Dainippon Sumitomo Pharma Co. Ltd.) were inoculated in a 24-well plate. Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

There was little difference between a value of the alkaline phosphatase activity of the mouse C3H10T1/2 cells cultured by adding the supernatant of the MEM growth medium in which the resting cartilage cells derived from costal cartilage were cultured and a value of the alkaline phosphatase activity thereof cultured by adding only the MEM growth medium.

In the same method and criteria as Example 1, it can be confirmed whether the supernatant of the medium (culture of cells) obtained by the above-described manipulation expresses induced osteoblast markers in the mouse C3H10T1/2 cells.

### Conclusion of Example 10 and Comparative Examples 10A to 10C

In the case where the chondrocytes capable of hypertrophication collected from rabbit costa/costal cartilage were cultured in the MEM differentiation agent producing medium, it was confirmed that there was an agent capable of increasing the alkaline phosphatase activity of mouse C3H10T1/2 cells, and capable of inducing differentiation thereof into induced osteoblasts in the supernatant of the medium (culture supernatant).

On the other hand, in the case where the chondrocytes capable of hypertrophication were cultured in the MEM growth medium, it was confirmed that there was not the agent in the supernatant of the medium (culture supernatant). Therefore, it was found that the chondrocytes capable of hypertrophication produced an agent capable of inducing differentiation of undifferentiated cells into induced osteoblasts by culturing them in the MEM differentiation agent producing medium.

Even in the case where the chondrocytes incapable of hypertrophication derived from rabbit costal cartilage were cultured in the MEM differentiation agent producing medium or the MEM growth medium, it was confirmed that they did not produce the agent capable of inducing differentiation of undifferentiated cells into induced osteoblasts.

### Example 11

### Study on Effect of Medium for Culturing Undifferentiated Cells (Undifferentiated Cell Culture Medium) on Induction of Differentiation of Undifferentiated Cells into Induced Osteoblasts

Chondrocytes capable of hypertrophication, resting cartilage cells incapable of hypertrophication, and articular cartilage cells incapable of hypertrophication were collected in the same manners as Example 1, Comparative Example 1B and Comparative Example 1D, respectively. Each of an MEM differentiation agent producing medium and an MEM growth medium was added to each kind of the cells so that they were diluted so as to become a density of 4×10⁴ cells/cm². The cells were cultured in a 5% CO₂ incubator at 37° C., and then a supernatant of the medium (culture supernatant) was collected on a time course (4 days, 7 days, 11 days, 14 days, 18 days, 21 days) to obtain fractional supernatants.

Mouse C3H10T1/2 cells were used as undifferentiated cells. The mouse C3H10T1/2 cells were inoculated in a 24-well plate reserving a HAM medium or an MEM medium at a density of 1.25×10⁴ cells/cm². Eighteen hours after the inoculation, 1 mL of each of the fractional supernatants and the medium was added to the plate, and then the mouse C3H10T1/2 cells were cultured in a 5% CO₂ incubator at 37° C. After 72 hours, an alkaline phosphatase activity of the mouse C3H10T1/2 cells was measured in the same manner as Example 1.

It was found that a value of the alkaline phosphatase activity of the mouse C3H10T1/2 cells cultured in the MEM medium, to which the supernatant of the MEM differentiation agent producing medium in which the chondrocytes capable of hypertrophication were cultured was added, increased by about 10.8 times that of the mouse C3H10T1/2 cells cultured in the MEM medium to which only the MEM differentiation agent producing medium was added.

It was found that the alkaline phosphatase activity of the mouse C3H10T1/2 cells cultured in the MEM medium, to which the supernatant of the MEM growth medium in which the chondrocytes capable of hypertrophication were cultured (culture supernatant) was added, did not increase. It was also found that the alkaline phosphatase activity of the mouse C3H10T1/2 cells cultured in the MEM medium, to which the supernatant of each of the MEM differentiation agent producing medium and the MEM growth medium in which the resting cartilage cells incapable of hypertrophication or the articular cartilage chondrocytes incapable of hypertrophication were cultured (culture supernatant) was added, did not increase.

Therefore, it was proved that the basal medium used for culturing the mouse C3H1T01/2 cells did not affect induction of differentiation thereof into induced osteoblasts (see Table 7, and FIG. 9).

TABLE 7
(effect of basal medium used for culturing
undifferentiated cells on induction of differentiation thereof
into induced osteoblasts)

| | |
|---|---|
| Relative value | Absolute value |

HAM medium (mean value)

| | | |
|---|---|---|
| GC differentiation supernatant | 6.7 | 0.058 |
| GC growth supernatant | 1.1 | 0.010 |
| RC differentiation supernatant | 1.2 | 0.011 |
| RC growth supernatant | 1.1 | 0.010 |
| AC differentiation supernatant | 1.2 | 0.010 |
| AC growth supernatant | 1.2 | 0.011 |
| Only differentiation medium | 1 | 0.009 |
| Only growth medium | 1 | 0.009 |

MEM medium (mean value)

| | | |
|---|---|---|
| GC differentiation supernatant | 10.8 | 0.085 |
| GC growth supernatant | 1.3 | 0.015 |
| RC differentiation supernatant | 1.5 | 0.012 |
| RC growth supernatant | 0.7 | 0.008 |
| AC differentiation supernatant | 1.3 | 0.010 |
| AC growth supernatant | 0.6 | 0.007 |
| Only differentiation medium | 1 | 0.008 |
| Only growth medium | 1 | 0.011 |

GC (4 week-old): one experiment was performed. Three trials were performed.
RC (8 week-old): one experiment was performed. Three trials were performed.
AC (8 week-old): one experiment was performed. Three trials were performed.
GC differentiation supernatant: supernatant of MEM differentiation agent producing medium in which chondrocytes capable of hypertrophication were cultured
GC growth supernatant: supernatant of MEM growth medium in which chondrocytes capable of hypertrophication were cultured
RC differentiation supernatant: supernatant of MEM differentiation agent producing medium in which resting cartilage cells were cultured
RC growth supernatant: supernatant of MEM growth medium in which resting cartilage cells were cultured
AC differentiation supernatant: supernatant of MEM differentiation agent producing medium in which articular cartilage cells were cultured
AC growth supernatant: supernatant of MEM growth medium in which articular cartilage cells were cultured
Only differentiation medium: MEM differentiation agent producing medium alone
Only growth medium: MEM growth medium alone

It was found that a value of the alkaline phosphatase activity of the mouse C3H10T1/2 cells cultured in the HAM medium, to which the supernatant of the MEM differentiation agent producing medium in which the chondrocytes capable of hypertrophication were cultured, was added, increased by about 6.7 times that of the mouse C3H10T1/2 cells cultured in the HAM medium to which only the MEM differentiation agent producing medium was added.

It was found that the alkaline phosphatase activity of the mouse C3H10T1/2 cells cultured in the HAM medium, to which the supernatant of the MEM growth medium in which the chondrocytes capable of hypertrophication were cultured (culture supernatant) was added, did not increase. It was also found that the alkaline phosphatase activity of the mouse C3H10T1/2 cells cultured in the HAM medium, to which the supernatant of each of the MEM differentiation agent producing medium and the MEM growth medium in which the resting cartilage cells incapable of hypertrophication or the articular cartilage chondrocytes incapable of hypertrophication were cultured (culture supernatant) was added, did not increase (see Table 7, and FIG. 9).

### Example 12

### Heat Degeneration of Agent Capable of Inducing Differentiation of Undifferentiated Cells into Induced Osteoblasts Produced by Chondrocytes Capable of Hypertrophication

Chondrocytes capable of hypertrophication were collected in the same manner as Example 1. An MEM differentiation agent producing medium (containing a minimum essential medium (MEM), 15% FBS (fetal bovine serum), 10 nM dexamethasone, 10 mM β-glycerophosphate, 50 μg/mL ascorbic acid, 100 U/mL penicillin, 0.1 mg/mL streptomycin and 0.25 μg/mL amphotericin B) was added to the chondrocytes capable of hypertrophication so that they were diluted so as to become a density of 4×10⁴ cells/cm². The chondrocytes capable of hypertrophication were cultured, and then a supernatant of the medium (culture supernatant) was collected on a time course (4 days, 7 days, 11 days, 14 days, 18 days, 21 days) to obtain fractional supernatants. A part of each of the fractional supernatants was heated for 3 minutes in boiling water.

Mouse C3H10T1/2 cells were cultured in a BME medium at a density of 1.25×10⁴ cells/cm². After 18 hours, 1 mL of each of the non-heated fractional supernatants, the heated fractional supernatants and the MEM differentiation agent producing medium alone was added to the medium. After 72 hours, an alkaline phosphatase activity of the mouse C3H10T1/2 cells was measured in the same manner as Example 1.

When a value of the alkaline phosphatase activity of the mouse C3H10T1/2 cells cultured by adding only the MEM differentiation agent producing medium was defined as “1”, a value of the alkaline phosphatase activity thereof cultured by adding the non-heated supernatant of the MEM differentiation agent producing medium, in which the chondrocytes capable of hypertrophication were cultured, was about 12.8 times (see Table 8, and FIG. 10).

On the other hand, a value of the alkaline phosphatase activity of the mouse C3H10T1/2 cells cultured by adding the heated supernatant was about 1.6 times (see Table 8, and FIG. 10). According to the results, it was confirmed that an agent capable of inducing differentiation of undifferentiated cells into induced osteoblasts, which existed in the supernatant of the MEM differentiation agent producing medium in which the chondrocytes capable of hypertrophication were cultured, was degenerated (inactivated) by the heat treatment.

TABLE 8
(heat degerenation of agent capable of
inducing differentiation of undifferentiated cells into
osteoblasts)
ALP activity (mean value)

| | Relative value | Absolute value |
|---|---|---|
| Heated | 1.6 | 0.014 |
| Non-treated | 12.8 | 0.111 |
| Only differentiation medium | 1 | 0.009 |

4 week-old: one experiment was performed. Three trials were performed.
Heated: heat-treated supernatant of MEM differentiation agent producing medium in which chondrocytes capable of hypertrophication were cultured
Non-treated: supernatant of MEM differentiation agent producing medium in which chondrocytes capable of hypertrophication were cultured
Only differentiation medium: MEM differentiation agent producing medium alone

### Example 13

### Effect of Implantation of Composite Material Produced Using Chondrocytes Capable of Hypertrophication having Ability of Producing Agent Capable of Inducing Differentiation into Induced Osteoblasts and Biocompatible Scaffold Under the Skin

Chondrocytes capable of hypertrophication derived from costa/costal cartilage were prepared in the same manner as Example 1. An MEM differentiation agent producing medium was added to the prepared chondrocytes capable of hypertrophication so that they were diluted so as to become a density of 1×10⁶ cells/mL to obtain a cell suspension. The cell suspension was inoculated evenly on collagen gel, alginic acid and Matrigel™ (produced by Becton, Dickinson and Company), respectively, and then the chondrocytes capable of hypertrophication were cultured in a 5% CO₂ incubator at 37° C. for 1 week, to produce composite materials. In the culture, an MEM differentiation agent producing medium was used.

These composite materials were implanted under the dorsal skins of syngenic animals. Four weeks after the implantation, the syngenic animals were sacrificed, and then implanted regions were surgically removed and fixed with a 10% neutral buffered formalin. After the implanted regions were subjected to a roentgenography and a micro-computerized tomography, they were embedded into paraffin. Thin slice samples of the implanted regions were produced based on a routine procedure, and then stained with hematoxylin-eosin (HE) staining, toluidine blue (TB) staining, alcian blue (AB) staining and safranin O (SO) staining. Thereafter, conditions of the implanted regions were evaluated.

Each of the following experiments was performed.

Roentgenography: the implanted region was roentgenographed from a vertical direction thereof at 100 KV using a micro-computerized tomography apparatus (“High Resolution X-ray micro-CT scanner SKYSCAN1172” produced by TOYO Corporation).

Micro-computerized tomography: the implanted region was roentgenographed at 100 KV using the same apparatus as the above micro-computerized tomography apparatus while spinning it every 4 degrees to obtain roentgenographic data, and then the roentgenographic data were restructured using NRecon which was an bundled software and a three-dimensional image was obtained using VGStudio Max which was a three-dimensional volume rendering software.

HE staining: the thin slice sample was deparaffinized and immersed into a hematoxylin solution for 5 to 10 minutes, and then rinsed. After producing a color of the thin slice sample, it was immersed into an eosin solution for 3 to 5 minutes.

TB staining: the thin slice sample was deparaffinized and immersed into a 0.05% toluidine blue solution for 15 to 30 minutes.

AB staining: the thin slice sample was deparaffinized and immersed into a 3% acetic acid solution for 3 to 5 minutes. Next, it was immersed into an alcian blue solution for 20 to 30 minutes, rinsed, and then immersed into a kernechtrot (nuclear fast red) solution for 10 to 15 minutes.

SO staining: the thin slice sample was deparaffinized, immersed into an iron hematoxylin solution for to 15 minutes, rinsed, and then fractionated (with hydrochloric acid alcohol). After producing a color of the thin slice sample, it was immersed into a 1% acetic acid, into a fast green solution for 1 to 5 minutes, into a 1% acetic acid, and then into a safranin O solution for 3 to 5 minutes.

In all of the composite materials containing the chondrocytes capable of hypertrophication, each of which obtained the ability of producing the agent capable of inducing differentiation into induced osteoblasts by culturing it in the MEM differentiation agent producing medium, osteogenesis was observed (see FIGS. 13 to 24).

Controls, which were a part of composite materials to be used for implantation, were fixed with a 10% neutral buffered formalin, and then embedded into paraffin. Thereafter, a thin slice sample of each of the controls was produced, and then stained.

In the same manner as this Example, composite materials are produced using the chondrocytes capable of hypertrophication prepared in Examples 2 and 3 (rat), Examples 4 and 5 (human), Example 7 (rat), Example 9 (mouse) and Example 10 (rabbit), respectively, instead of the above chondrocytes capable of hypertrophication, and then implanted under the skins of syngenic animals or immunodeficient animals. In this way, effect of the implantation of each of the composite materials under the skins thereof can be evaluated.

Further, in the same manner as this Example, other composite materials are produced using, for example, hydroxyapatite, PuraMatrix™ (“catalog number 354250: BD PuraMatrix peptide hydrogel” produced by Becton, Dickinson and Company), collagen (sponge), gelatin (sponge) and agarose, respectively, as biocompatible scaffolds instead of the above collagen gel, alginic acid and Matrigel™, and then implanted under the skins of syngenic animals or immunodeficient animals. In this way, effect of the implantation of each of the composite materials under the skins thereof can be evaluated.

### Comparative Example 13A

### Effect of Implantation of Composite Material Produced Using Chondrocytes Incapable of Hypertrophication and Biocompatible Scaffold Under the Skin

Chondrocytes incapable of hypertrophication prepared in the same manner as Comparative Example 1B (rat) were used. The chondrocytes incapable of hypertrophication were diluted in an MEM differentiation agent producing medium or an MEM growth medium, and then composite materials were produced in the same manner as Example 13. As biocompatible scaffolds, collagen gel, Matrigel™ (produced by Becton, Dickinson and Company) and alginic acid were used, respectively.

These composite materials were implanted under the dorsal skins of syngenic animals. Four weeks after the implantation, the syngenic animals were sacrificed, and then implanted regions were surgically removed and fixed with a 10% neutral buffered formalin. After the implanted regions were subjected to a roentgenography and a micro-computerized tomography, they were embedded into paraffin. Thin slice samples were produced, and then stained with hematoxylin-eosin (HE) staining, toluidine blue (TB) staining, alcian blue (AB) staining and safranin O (SO) staining. Thereafter, conditions of the implanted regions were evaluated.

In the case where the chondrocytes incapable of hypertrophication were used, osteogenesis was not observed in the composite material produced using any biocompatible scaffold. The results obtained in the case of the use of the MEM differentiation agent producing medium are shown in FIGS. 25 to 33.

In the same manner as this Comparative Example, composite materials are produced using the chondrocytes incapable of hypertrophication prepared in Comparative Example 1D (rat), Comparative Example 3B (rat), Comparative Example 4B (human), Comparative Example 5B (human), Comparative Example 9B (mouse) and Comparative Example 10B (rabbit), respectively, instead of the above chondrocytes incapable of hypertrophication, and then implanted under the skins of syngenic animals or immunodeficient animals. In this way, an effect of the implantation of each of the composite materials under the skins thereof can be evaluated.

Further, in the same manner as this Comparative Example, other composite materials are produced using, for example, hydroxyapatite, PuraMatrix™ (“catalog number 354250: BD PuraMatrix peptide hydrogel” produced by Becton, Dickinson and Company), collagen (sponge), gelatin (sponge) and agarose, respectively, as biocompatible scaffolds instead of the above collagen gel, Matrigel™ and alginic acid, and then implanted under the skins of syngenic animals or immunodeficient animals. In this way, an effect of the implantation of each of the composite materials under the skins thereof can be evaluated.

### Comparative Example 13B

### Effect of Independent Implantation of Scaffold Under the Skin

This Comparative Example was performed in the same manner as Example 13, except that the scaffolds were independently implanted. Hydroxyapatite, collagen gel, alginic acid and Matrigel™ (produced by Becton, Dickinson and Company), which were the scaffolds, were independently implanted under the skins of syngenic animals, respectively. As a result, osteogenesis was not observed (see FIG. 34).

In the same manner as this Comparative Example, for example, PuraMatrix™ (“catalog number 354250: BD PuraMatrix peptide hydrogel” produced by Becton, Dickinson and Company), collagen (sponge) and gelatin (sponge) are independently implanted under the skins of syngenic animals or immunodeficient animals, respectively, instead of the above hydroxyapatite, collagen gel, alginic acid and Matrigel™. In this way, an effect of each of the scaffolds is evaluated.

### Example 14

### Effect of Implantation of Pellet of Chondrocytes Capable of Hypertrophication having Ability of Producing Agent Capable of Inducing Differentiation into Induced Osteoblasts Under the Skin

(Preparation of Pellet of Chondrocytes Capable of Hypertrophication having Ability of Producing Agent Capable of Inducing Differentiation into Induced Osteoblasts)

Chondrocytes capable of hypertrophication derived from costa/costal cartilage were prepared in the same manner as Example 1. An MEM differentiation agent producing medium was added to 5×10⁵ chondrocytes capable of hypertrophication so that they were diluted so as to become a density of 5×10⁵ cells/0.5 mL to obtain a cell suspension. The cell suspension was centrifuged (at 1000 rpm (170×g) for 5 minutes) to form pellets of the chondrocytes capable of hypertrophication having an ability of producing an agent capable of inducing differentiation into induced osteoblasts. These pellets were cultured at 37° C. for 1 week (see FIG. 35A). In this culture, the MEM differentiation agent producing medium was used.

Thereafter, these pellets were implanted under the dorsal skins of syngenic animals. Four weeks after the implantation, the syngenic animals were sacrificed, and then implanted regions were surgically removed and fixed with a 10% neutral buffered formalin. After the implanted regions were subjected to a roentgenography and a micro-computerized tomography, they were embedded into paraffin.

Thin slice samples of the implanted regions were produced based on a routine procedure, and then stained with hematoxylin-eosin (HE) staining, toluidine blue (TB) staining, alcian blue (AB) staining and safranin O (SO) staining in the same manner as Example 13. Thereafter, conditions of the implanted regions were evaluated. As a result, in the case where the pellets of the chondrocytes capable of hypertrophication having the activity of producing the agent capable of inducing differentiation into induced osteoblasts were implanted, osteogenesis was observed in the implanted regions (see FIGS. 35C, 35D, 36 and 37).

In the same manner as this Example, pellets are produced using the chondrocytes capable of hypertrophication prepared in Examples 2 and 3 (rat), Examples 4 and 5 (human), Example 7 (rat), Example 9 (mouse) and Example 10 (rabbit), respectively, instead of the above chondrocytes capable of hypertrophication, and then implanted under the skins of syngenic animals or immunodeficient animals. In this way, an effect of the implantation of each of the pellets under the skins thereof can be evaluated.

### Comparative Example 14A

### Effect of Implantation of Pellet of Chondrocytes Incapable of Hypertrophication Under the Skin

Chondrocytes incapable of hypertrophication prepared in the same manner as Comparative Example 1B (rat) were used. An MEM differentiation agent producing medium was added to 5×10⁵ chondrocytes incapable of hypertrophication so that they were diluted so as to become a density of 5×10⁵cells/0.5 mL to obtain a cell suspension. The cell suspension was centrifuged (at 1000 rpm (170×g) for 5 minutes) to prepare pellets of the chondrocytes incapable of hypertrophication. These pellets were cultured at 37° C. for 1 week (see FIG. 35B).

Thereafter, these pellets were implanted under the dorsal skins of syngenic animals. Further, an effect of the implantation of the pellet of the chondrocytes incapable of hypertrophication in each of implanted regions was observed in the same manner as Example 14. As a result, osteogenesis was not observed in the implanted regions (FIGS. 35E, 35F and 38).

In the same manner as this Comparative Example, pellets (cell pellets) were produced using the chondrocytes incapable of hypertrophication prepared in Comparative Example 1D (rat), Comparative Example 3B (rat), Comparative Example 4B (human), Comparative Example 5B (human), Comparative Example 9B (mouse) and Comparative Example 10B (rabbit), respectively, instead of the above chondrocytes incapable of hypertrophication. Next, these pellets were implanted under the skins of syngenic animals or immunodeficient animals. After the implantation, an effect of the implantation of the pellet of the chondrocytes incapable of hypertrophication in each of implanted regions can be observed in the same manner as Example 14.

### Example 15

### Relation Between Agent Capable of Inducing Differentiation into Induced Osteoblasts Produced by Chondrocytes Capable of Hypertrophication, and BMP or TGFβ

Chondrocytes capable of hypertrophication were collected from rat costa/costal cartilages in the same manner as Example 1. An MEM differentiation agent producing medium (containing a minimum essential medium (MEM), 15% FBS (fetal bovine serum), nM dexamethasone, 10 mM β-glycerophosphate, 50 μg/mL ascorbic acid, 100 U/mL penicillin, 0.1 mg/mL streptomycin and 0.25 μg/mL amphotericin B) was added to the chondrocytes capable of hypertrophication so that they were diluted so as to become a density of 4×10⁴ cells/cm².

The chondrocytes capable of hypertrophication were cultured, and then a supernatant of the medium was collected on a time course. TGFβ and BMP assays of the supernatant were performed as follows, and TGFβ and BMP activities were detected by measuring an alkaline phosphatase activity.

(TGFβ Assay)

TGFβ assay of the supernatant was performed using a method described in Nagano, T. et al.: Effect of heat treatment on bioactivities of enamel matrix derivatives in human periodontal ligament (HPDL) cells. J. Periodont. Res., 39: 249-256, 2004. HPDL cells were inoculated in a 96-well plate at a density of 5×10⁴ cells/well and cultured for 24 hours. Next, the culture medium was substituted with a medium containing 10 nM 1α, 25-dihydroxyvitamin D3, and the supernatant as a test sample.

Thereafter, the HPDL cells were cultured for 96 hours, rinsed with PBS, and then the alkaline phosphatase activity thereof was measured. Specifically, the HPDL cells were reacted with 10 mM p-nitrophenyl phosphate as a substrate in a 100 mM 2-amino-2-methyl-1,3 propanediol hydrochloric acid buffer (pH 10.0) containing 5 mM MgCl₂ at 37° C. for 10 minutes. Thereafter, NaOH was added to the buffer, and then absorbance thereof at 405 nm was measured.

In the case where the supernatant of the MEM differentiation agent producing medium in which the chondrocytes capable of hypertrophication were cultured (culture supernatant) was added to the 96-well plate as the test sample, the absorbance was about 0.1515, about0.2545, and about 0.1242 (see Table 9, and FIG. 11A).

TABLE 9
(TGFβ activity)

| | 405 nm (OD) | SD |
|---|---|---|
| 1 | 0.1515 | 0.01818 |
| 2 | 0.2545 | 0.00303 |
| 3 | 0.1242 | 0.03030 |

(BMP Assay)

BMP assay of the supernatant was performed using a method described in Iwata, T. et al.: Noggin Blocks Osteoinductive Activity of Porcine Enamel Extracts. J. Dent. Res., 81: 387-391, 2002. ST2 cells were inoculated in a 96-well plate at a density of 5×10⁴ cells/well and cultured for 24 hours. Next, the culture medium was substituted with a medium containing 200 nM all-trans retinoic acid, and the supernatant as a test sample.

Thereafter, the ST2 cells were cultured for 72 hours, rinsed with PBS, and then the alkaline phosphatase activity thereof was measured. Specifically, the ST2 cells were reacted with 10 mM p-nitrophenyl phosphate as a substrate in a 100 mM 2-amino-2-methyl-1,3 propanediol hydrochloric acid buffer (pH 10.0) containing 5 mM MgCl₂ at 37° C. for 8 minutes. Thereafter, NaOH was added to the buffer, and then absorbance thereof at 405 nm was measured.

In the case where the supernatant of the MEM differentiation agent producing medium in which the chondrocytes capable of hypertrophication were cultured was added to the 96-well plate as the test sample, the absorbance was about 0.05, about 0.075, and about 0.075 (see Table 10, and FIG. 11B).

TABLE 10
(BMP activity)

| | 405 nm (OD) | SD |
|---|---|---|
| 1 | 0.0500 | 0.0188 |
| 2 | 0.0750 | 0.0125 |
| 3 | 0.0750 | 0.0063 |

The TGFβ activity was observed in the supernatant of the MEM differentiation agent producing medium containing an induced osteoblast differentiation inducing agent. Namely, it was proved that the TGFβ was present in this supernatant of the differentiation agent producing medium (see FIG. 11A). On the other hand, the BMP activity was also slightly observed (see FIG. 11B).

Although a BMP pathway is suppressed by the presence of the TGFβ, the alkaline phosphatase activity of the cells increased even in the case where the supernatant of the differentiation agent producing medium containing the TGFβ was added to the cells. According to the result, it is believed that the increase of the alkaline phosphatase activity of the cells was induced by the induced osteoblast differentiation inducing agent, which was not the BMP.

### Example 16

### Effect of Agent Produced by Chondrocytes Capable of Hypertrophication on Pellet Formed from Undifferentiated Cells

A supernatant of an MEM differentiation agent producing medium, in which the chondrocytes capable of hypertrophication were cultured, was obtained in the same manner as Example 1. 5×10⁵ mouse C3H10T1/2 cells (“CCL-226” produced by Dainippon Sumitomo Pharma Co. Ltd.) were centrifuged at 1,000 rpm (170×g) for 3 to 5 minutes at room temperature to form them into pellets, and then the pellets were cultured for 1 week.

In this culture, a BME medium, to which the supernatant of the differentiation agent producing medium in which the chondrocytes capable of hypertrophication were cultured was added, was prepared in the same manner as Example 1 and used.

(Method of Forming Subcutaneous Pocket)

Each of syngenic animals or immunodeficient animals to be used for implantation is anesthetized, and then a skin thereof is aseptically incised to form an open wound portion. A round nose scissors is subcutaneously inserted through the open wound portion to separate the skin from a subcutaneous tissue. In this way, a subcutaneous pocket is formed in each of the animals.

(Formation of Bone Defective Region)

Each of syngenic animals or immunodeficient animals to be used for implantation is anesthetized. Thereafter, a skin thereof outside femur or tibia is aseptically incised, and then a cartilaginous tissue is bent to expose a bone defective region to be formed, or a skin thereof outside skull is incised to expose a bone defective region to be formed. A perforative or disjunctive bone defective region is formed in each of the animals using a trephine bar or a disc attached to a dental drill.

(Implantation of Pellet)

(Implantation Under the Skin)

Subcutaneous pockets each having a diameter of 1 to 2 cm were formed under the dorsal skin of each of three 8 week-old male C3H mice (produced by CLEA Japan, Inc.) using the above-described method. The pellets (cell pellets) produced in this Example were implanted under the dorsal skin of each of the C3H mice. Next, 4 weeks after the implantation, implanted regions and the periphery thereof were surgically removed. Thereafter, an osteogenic ability of the mouse C3H10T1/2 cells forming each of the pellets was evaluated using micro-computerized tomography measurement and preparation production.

Osteogenesis in the three C3H mice (individual numbers 1 to 3) was induced on one level or another. Namely, the mouse C3H10T1/2 cells cultured in the medium, to which the supernatant containing the agent was added, had an ectopic osteogenic ability (ability of forming bone under the skin) (see FIGS. 12A to 12C).

(Implantation into Bone Defective Region)

The pellets produced in this Example are implanted into the bone defective regions of the syngenic animals or the immunodeficient animals, and then an osteogenic ability of the mouse C3H10T1/2 cells forming each of the pellets is evaluated.

Further, the same experiment is performed for an induced osteoblast differentiation inducing agent produced by the chondrocytes capable of hypertrophication prepared in each of Examples 2 and 3 (rat), Examples 4 and 5 (human), Example 7 (rat), Example 9 (mouse) and Example 10 (rabbit). Thereafter, an osteogenic ability of the mouse C3H10T1/2 cells forming each of pellets is evaluated using micro-computerized tomography measurement and preparation production.

### Comparative Example 16A

This Comparative Example is performed in the same manner as Example 16, except that pellets formed from mouse C3H10T1/2 cells are cultured in a medium to which a supernatant of a growth medium in which the chondrocytes capable of hypertrophication are cultured (supernatant containing no induced osteoblast differentiation inducing agent) is added, instead of the medium to which the supernatant of the differentiation agent producing medium in which the chondrocytes capable of hypertrophication are cultured (supernatant containing the induced osteoblast differentiation inducing agent) is added.

(Formation of Subcutaneous Pocket)

Subcutaneous pockets are formed in the same manner as Example 16.

(Formation of Bone Defective Region)

Bone defective regions are formed in the same manner as Example 16.

(Implantation of Pellet)

The pellets (cell pellets) produced in this Comparative Example are implanted under the skins and into the bone defective regions. Thereafter, an osteogenic ability of the mouse C3H10T1/2 cells forming each of the pellets cultured in the medium, to which the supernatant containing no induced osteoblast differentiation inducing agent is added, is evaluated.

### Comparative Example 16B

This Comparative Example is performed in the same manner as Example 16, except that pellets formed from mouse C3H10T1/2 cells are cultured in a medium to which a supernatant of a differentiation agent producing medium in which chondrocytes incapable of hypertrophication are cultured (supernatant containing no induced osteoblast differentiation inducing agent) is added, instead of the medium to which the supernatant of the differentiation agent producing medium in which the chondrocytes capable of hypertrophication are cultured (supernatant containing the induced osteoblast differentiation inducing agent) is added.

(Formation of Subcutaneous Pocket)

Subcutaneous pockets are formed in the same manner as Example 16.

(Formation of Bone Defective Region)

Bone defective regions are formed in the same manner as Example 16.

5×10⁵ mouse C3H10T1/2 cells (“CCL-226” produced by Dainippon Sumitomo Pharma Co. Ltd.) were formed into pellets, and then the pellets were cultured in a medium, to which a supernatant of a differentiation agent producing medium in which the chondrocytes incapable of hypertrophication were cultured was added, for 1 week. These pellets were implanted under the dorsal skin of each of C3H mice (individual numbers 1 to 3) used in Example 16.

Four weeks after the implantation, the pellets were surgically removed, and then the pellets removed were roentgenographed. Since a shadow had been observed in each of roentgenograms of the pellets removed from the C3H mouse of the individual number 1, the pellets were subjected to a micro-computerized tomography (see FIG. 12D). On the other hand, since a shadow had not been observed in each of roentgenograms of the pellets removed from the C3H mice of the individual numbers 2 and 3 (see FIG. 12E), the pellets were not subjected to the micro-computerized tomography.

It was confirmed that the mouse C3H10T1/2 cells cultured in the medium, to which the supernatant containing no induced osteoblast differentiation inducing agent was added, did not have an ectopic osteogenic ability.

The pellets produced in this Comparative Example are implanted into the bone defective regions, and then an osteogenic ability of the mouse C3H10T1/2 cells forming each of the pellets is evaluated.

### Comparative Example 16C

This Comparative Example is performed in the same manner as Example 16, except that pellets formed from mouse C3H10T1/2 cells are cultured in a medium to which only a differentiation agent producing medium or a growth medium is added, instead of the medium to which the supernatant of the differentiation agent producing medium in which the chondrocytes capable of hypertrophication are cultured (supernatant containing the induced osteoblast differentiation inducing agent) is added.

(Formation of Subcutaneous Pocket)

Subcutaneous pockets are formed in the same manner as Example 16.

(Formation of Bone Defective Region)

Bone defective regions are formed in the same manner as Example 16.

(Implantation of Pellet)

The pellets (cell pellets) produced in this Comparative Example are implanted under the skins and into the bone defective regions. Thereafter, an osteogenic ability of the mouse C3H10T1/2 cells forming each of the pellets cultured in the medium, to which only the differentiation agent producing medium or the growth medium is added, is evaluated.

### Example 17

### Effect of Implantation of Composite Material Produced Using Induced Osteoblasts Induced by Induced Osteoblast Differentiation Inducing Agent and Biocompatible Scaffold Under the Skin and into Bone Defective region

(Production of Composite Material)

In this Example, a BEM medium, to which a supernatant of a differentiation agent producing medium in which the chondrocytes capable of hypertrophication prepared in each of Examples 1 to 3 (rat), Examples 4 and 5 (human), Example 7 (rat), Example 9 (mouse) and Example 10 (rabbit) are cultured (supernatant containing an induced osteoblast differentiation inducing agent) is added, is used.

Composite materials are produced by inoculating mouse C3H10T1/2 cells (“CCL-226” produced by Dainippon Sumitomo Pharma Co. Ltd.) on scaffolds recited in Table 11. The composite materials are cultured in a 5% CO₂ incubator at 37° C. for 1 week. In this culture, as a culture solution, the BEM medium to which the supernatant containing the present agent is added is used as described above. In the same manner and criteria as Example 1, it can be confirmed whether the mouse C3H10T1/2 cells on each of the scaffolds are induced into induced osteoblasts.

(Formation of Subcutaneous Pocket)

Each of syngenic animals or immunodeficient animals to be used for implantation is anesthetized, and then a skin thereof is aseptically incised to form an open wound portion. A round nose scissors is subcutaneously inserted through the open wound portion to separate the skin from a subcutaneous tissue. In this way, a subcutaneous pocket is formed in each of the animals.

(Formation of Bone Defective Region)

Each of syngenic animals or immunodeficient animals to be used for implantation is anesthetized. Thereafter, a skin thereof outside femur or tibia is aseptically incised, and then a cartilaginous tissue is bent to expose a bone defective region to be formed, or a skin thereof outside skull is incised to expose a bone defective region to be formed. A perforative or disjunctive bone defective region is formed in each of the animals using a trephine bar or a disc attached to a dental drill.

(Implantation of Composite Material)

The composite materials are implanted under the skins and into the bone defective regions of the syngenic animals or the immunodeficient animals. Four weeks after the implantation, the syngenic animals or the immunodeficient animals are sacrificed. Thereafter, implanted regions are surgically removed, fixed with a 10% neutral buffered formalin, and then embedded into paraffin.

Thin slice samples of the implanted regions were produced, and then stained with HE staining. Thereafter, conditions of the implanted regions are evaluated. In each of the composite materials containing the induced chondrocytes induced by the induced osteoblast differentiation inducing agent and implanted under the skins and into the bone defective regions, an osteogenic ability of the mouse C3H10T1/2 cells is evaluated.

TABLE 11

| Scaffold | Composition |
|---|---|
| Porous hydroxyapatite | “APACERAM porosity of 50%” produced by HOYA |
| | CORPORATION |
| Super porous | “APACERAM porosity of 85%” produced by HOYA |
| hydroxyapatite | CORPORATION |
| Super porous | “3D Scaffold” produced by BD Corporation |
| hydroxyapatite | |
| Apatite-collagen mixture | “APACERAM GRANULE” produced by HOYA CORPORATION + |
| | “Collagen Gel” produced by Nitta Gelatin Inc. |
| Apatite-collagen complex | “APACOLLA” produced by HOYA CORPORATION |
| Collagen gel | “Collagen Gel” produced by Nitta Gelatin Inc. |
| Collagen sponge | “Collagen Sponge” produced by Nitta Gelatin |
| | Inc. |
| Gelatin sponge | “Hemostatic Gelatin Sponge” produced by |
| | Yamanouchi Pharmaceutical Co., Ltd. |
| Fibrin gel | “Beriplast P” produced by Nipro |
| Synthetic peptide | “Pramax” produced by 3D Matrix Corporation |
| Extracellular matrix | “Matrigel” produced by BD Corporation |
| mixture | |
| Alginate | “Kelton LVCR” produced by Kelco Corporation |
| Agarose | “Agarose” produced by Wako Pure Chemical |
| | Industries, Ltd. |
| Polyglycolic acid | “Polyglycolic acid” produced by COREFRONT |
| | Corporation |
| Polylactic acid | “Polylactic acid” produced by COREFRONT |
| | Corporation |
| Polyglycolic acid- | “Polyglycolic acid-polylactic acid copolymer” |
| polylactic acid copolymer | produced by COREFRONT Corporation |

### Comparative Example 17A

### Effect of Implantation of Composite Material Produced using Undifferentiated Cells Cultured in Medium Containing No Induced Osteoblast Differentiation Inducing Agent and Biocompatible Scaffold Under the Skin and into Bone Defective Region

(Production of Composite Material)

In this Comparative Example, a BME medium, to which a supernatant of a growth medium in which the chondrocytes capable of hypertrophication prepared in each of Examples 1 to 3 (rat), Examples 4 and 5 (human), Example 7 (rat), Example 9 (mouse) and Example 10 (rabbit) are cultured (supernatant containing no induced osteoblast differentiation inducing agent) is added, is used.

Composite materials are produced by inoculating mouse C3H10T1/2 cells (“CCL-226” produced by Dainippon Sumitomo Pharma Co. Ltd.) on scaffolds recited in Table 11 in the same manner as Example 17. The composite materials are cultured in a 5% CO₂ incubator at 37° C. for 1 week. In this culture, as a culture solution, the BEM medium to which the supernatant containing no present agent is added is used as described above. In the same manner and criteria as Example 1, it can be confirmed whether the mouse C3H10T1/2 cells on each of the scaffolds are induced into induced osteoblasts.

(Formation of Subcutaneous Pocket)

Subcutaneous pockets are formed in the same manner as Example 17.

(Formation of Bone Defective Region)

Bone defective regions are formed in the same manner as Example 17.

(Implantation of Composite Material)

The composite materials are implanted under the skins and into the bone defective regions of the syngenic animals or the immunodeficient animals. Four weeks after the implantation, the syngenic animals or the immunodeficient animals are sacrificed. Thereafter, implanted regions are surgically removed, fixed with a 10% neutral buffered formalin, and then embedded into paraffin.

Thin slice samples of the implanted regions were produced, and then stained with HE staining. Thereafter, conditions of the implanted regions are evaluated. In each of the composite materials implanted under the skins and into the bone defective regions, an osteogenic ability of the mouse C3H10T1/2 cells is evaluated.

### Comparative Example 17B

### Effect of Implantation of Composite Material Produced using Undifferentiated Cells Cultured in Medium Containing No Induced Osteoblast Differentiation Inducing Agent and Biocompatible Scaffold Under the Skin and into Bone Defective Region

(Production of Composite Material)

In this Comparative Example, a BEM medium, to which a supernatant of a differentiation agent producing medium in which the chondrocytes incapable of hypertrophication prepared in each of Comparative Example 1B (rat), Comparative Example 1D (rat), Comparative Example 3B (rat), Comparative Example 4B (human), Comparative Example 5B (human), Comparative Example 9B (mouse) and Comparative Example 10B (rabbit) are cultured (supernatant containing no induced osteoblast differentiation inducing agent) is added, is used.

Composite materials are produced by inoculating mouse C3H10T1/2 cells (“CCL-226” produced by Dainippon Sumitomo Pharma Co. Ltd.) on scaffolds recited in Table 11 in the same manner as Example 17. The composite materials are cultured in a 5% CO₂ incubator at 37° C. for 1 week. In this culture, as a culture solution, the BEM medium to which the supernatant containing no present agent is added is used as described above. In the same manner and criteria as Example 1, it can be confirmed whether the mouse C3H10T1/2 cells on each of the scaffolds are induced into induced osteoblasts.

(Formation of Subcutaneous Pocket)

Subcutaneous pockets are formed in the same manner as Example 17.

(Formation of Bone Defective Region)

Bone defective regions are formed in the same manner as Example 17.

(Implantation of Composite Material)

The composite materials are implanted under the skins and into the bone defective regions of the syngenic animals or the immunodeficient animals. Four weeks after the implantation, the syngenic animals or the immunodeficient animals are sacrificed. Thereafter, implanted regions are surgically removed, fixed with a 10% neutral buffered formalin, and then embedded into paraffin.

Thin slice samples of the implanted regions were produced, and then stained with HE staining. Thereafter, conditions of the implanted regions are evaluated. In each of the composite materials implanted under the skins and into the bone defective regions, an osteogenic ability of the mouse C3H10T1/2 cells is evaluated.

### Comparative Example 17C

### Effect of Implantation of Composite Material Produced using Undifferentiated Cells Cultured in Medium Containing No Induced Osteoblast Differentiation Inducing Agent and Biocompatible Scaffold Under the Skin and into Bone Defective Region

(Production of Composite Material)

Composite materials are produced by inoculating mouse C3H10T1/2 cells (“CCL-226” produced by Dainippon Sumitomo Pharma Co. Ltd.) in scaffolds recited in Table 11 in the same manner as Example 17. The composite materials are cultured in a 5% CO₂ incubator at 37° C. for 1 week. In this culture, as a culture solution, a medium to which only the differentiation agent producing medium or the growth medium is added is used. In the same manner and criteria as Example 1, it can be confirmed whether the mouse C3H10T1/2 cells on each of the scaffolds are induced into induced osteoblasts.

(Formation of Subcutaneous Pocket)

Subcutaneous pockets are formed in the same manner as Example 17.

(Formation of Bone Defective Region)

Bone defective regions are formed in the same manner as Example 17.

(Implantation of Composite Material)

The composite materials are implanted under the skins and into the bone defective regions of the syngenic animals or the immunodeficient animals. Four weeks after the implantation, the syngenic animals or the immunodeficient animals are sacrificed. Thereafter, implanted regions are surgically removed, fixed with a 10% neutral buffered formalin, and then embedded into paraffin.

Thin slice samples of the implanted regions were produced, and then stained with HE staining. Thereafter, conditions of the implanted regions are evaluated. In each of the composite materials implanted under the skins and into the bone defective regions, an osteogenic ability thereof is evaluated.

### Comparative Example 17D

### Effect of Independent Implantation of Scaffold Under the Skin and into Bone Defective Region

Scaffolds recited in Table 11 are independently implanted under the skins and into the bone defective regions of syngenic animals or immunodeficient animals in the same manner as Example 17. In each of the scaffolds implanted under the skins and into the bone defective regions, an osteogenic ability of the mouse C3H10T1/2 cells is evaluated.

### Example 18

### Comparison Between Induced Osteoblasts Induced by Induced Osteoblast Differentiation Inducing Agent and Natural Osteoblasts

(Pellet Formed from Induced Osteoblasts Induced by Induced Osteoblast Differentiation Inducing Agent)

This Example is performed in the same manner as Example 16. Mouse C3H10T1/2 cells were formed into pellets, and then the pellets were cultured in a medium, to which a supernatant containing an induced osteoblast differentiation inducing agent produced by chondrocytes capable of hypertrophication was added, for 1 week. In this way, the mouse C3H10T1/2 cells were induced into induced osteoblasts.

In order to form the pellets (cell pellets), the induced osteoblasts are centrifuged in the same manner as Example 16. As a result, the mouse C3H10T1/2 cells were formed into the pellets.

(Composite Material Produced using Induced Chondrocytes Induced by Induced Osteoblast Differentiation Inducing Agent and Biocompatible Scaffold)

Composite materials are produced in the same manner as Example 17, and then implanted under the skins and into the bone defective regions. In each of the composite materials implanted under the skins and into the bone defective regions, an osteogenic ability of the mouse C3H10T1/2 cells is evaluated.

### Comparative Example 18A

### Pellet of Natural Osteoblasts

(Method of Collecting and Culturing Osteoblasts)

Calvariae of rat newborns are collected. The calvariae are rinsed with DPBSA (dulbecco's buffer solution containing phosphates) after removing connective tissues therefrom. These calvariae are sectioned, and then placed in a vessel with a stirrer. Thereafter, a 0.1% collagenase/DPBS after filter sterilization is introduced into the vessel, and then stirred at 37° C. for 90 minutes to culture the sectioned calvariae.

Next, cells are filtered using a cell straightener, rinsed with a medium (MEM), inoculated in a culture vessel, and then cultured in a 5% CO₂ incubator.

(Production of Pellet)

In order to form pellets (cell pellets), osteoblasts are centrifuged in the same manner as Example 16. Thereafter, it is evaluated whether the pellets are formed.

### Comparative Example 18B

### Composite Material Produced using Natural Osteoblasts and Biocompatible Scaffold

Composite materials are produced using the natural osteoblasts obtained in this Comparative Example in the same manner as Example 17. These composite materials are independently implanted under the skins and into bone defective regions of syngenic animals or immunodeficient animals. In each of the composite materials implanted under the skins and into the bone defective regions, an osteogenic ability of the natural osteoblasts is evaluated.

### Example 19

### Effect of Induced Osteoblast Differentiation Inducing Agent on Undifferentiated Cells Derived from Rat Bone Marrow

(Preparation of Induced Osteoblast Differentiation Inducing Agent Produced by Chondrocytes Capable of Hypertrophication)

Induced osteoblast differentiation inducing agents are prepared in the same manner as Examples 1 to 3 (rat), Examples 4 and 5 (human), Example 7 (rat), Example 9 (mouse) and Example 10 (rabbit).

(Preparation of Undifferentiated Cells Derived from Rat Bone Marrow)

Femurs are collected from rats, soft tissues are removed therefrom, and then both epiphyseal regions thereof are removed. A medium is introduced into a syringe, and then infused into each of the femurs from both ends thereof via a needle of the syringe so that a bone marrow thereinside is flushed out with the medium. In this way, a cell mixed solution is obtained. As the medium, an MEM medium containing 15% FBS is used.

The obtained cell mixed solution is subjected to a pipetting treatment, inoculated in T-75 flasks in an amount thereof corresponding to one femur per one T-75 flask, and then cultured in a CO₂ incubator at 37° C. A half of each of the medium is exchanged by a new medium three times per 1 week. Seven to ten days after the culture, cells adhering to the T-75 flasks are separated therefrom with a 0.05% trypsin-EDTA solution, to obtain a culture.

A supernatant containing each of the induced osteoblast differentiation inducing agents prepared in this Example is added to a medium containing the culture of the undifferentiated cells derived from rat bone marrow, and then the culture is further cultured. Thereafter, in each of the induced osteoblast differentiation inducing agents, an ability of inducing the undifferentiated cells derived from rat bone marrow into induced osteoblasts is evaluated in the same manner as Example 1.

Undifferentiated cells derived from bone marrow were induced into osteoblasts using a conventional method, and the induced osteoblasts were used. Specifically, the undifferentiated stem cells were collected from rat femur marrows according to a method described in Maniatopoulos et al., Cell Tissue Res., 254: 317-330, 1988. Thereafter, these undifferentiated stem cells were centrifuged at 170 to 200×g for 3 to 5 minutes to form them into pellets, and then the pellets were cultured in a medium proposed by Maniatopoulos (herein, referred to as a “differentiation agent producing medium”) in a 5% CO₂ incubator at 37° C. for 2 weeks to induce the osteoblasts (Bp). These osteoblasts (Bp) were used.

A real-time PCR is performed in the same manner as Example 1, and each of cell markers was measured using a real-time PCR apparatus (“PRISM 7900HT” produced by Applied Biosystems, Inc.). After complication of the PCR, setting of threshold values and calculation of attainment cycles were performed using an analysis software incorporated in the apparatus (“PRISM 7900HT”).

Expression amounts of each cell marker were divided by an expression amount of the GAPDH to calculate correction values thereof, and then an average expression amount thereof was obtained by averaging the correction values. As a result, chondrocytes incapable of hypertrophication expressed type II collagen and aglycan, but did not express alkaline phosphatase and osteocalcin (see Comparative Example 1B, and Table II).

On the other hand, the osteoblasts induced from the undifferentiated cells derived from bone marrow using the conventional method expressed the alkaline phosphatase and the osteocalcin, but did not express the type II collagen and the aglycan (Table III).

TABLE III

Amount (correction value by GAPDH)
average value

| | | | | |
|---|---|---|---|---|
| Sample | 1 | 2 | 3 | Average |

Alkaline Phosphatase

| | | | | |
|---|---|---|---|---|
| Bp | 0.0815 | 0.0776 | 0.0839 | 0.0810 |

Type II Collagen

| | | | | |
|---|---|---|---|---|
| Bp | 0.0000 | 0.0000 | 0.0000 | 0.0000 |

Aglycan

| | | | | |
|---|---|---|---|---|
| Bp | 0.0010 | 0.0009 | 0.0013 | 0.0011 |

Osteocalcin

| | | | | |
|---|---|---|---|---|
| Bp | 0.7282 | 0.7136 | 1.1064 | 0.8494 |

Bp: pellet of undifferentiated stem cells collected from femur marrow and cultured in osteoblast differentiation medium

### Comparative Example 19A

### Effect of Supernatant Containing No Induced Osteoblast Differentiation Inducing Agent on Undifferentiated Cells Derived from Rat Bone Marrow

This Comparative Example is preformed in the same manner as Example 19, except that a supernatant of a growth medium, in which chondrocytes capable of hypertrophication are cultured (supernatant containing no induced osteoblast differentiation inducing agent), is added to the medium, instead of the supernatant containing the induced osteoblast differentiation inducing agent. The supernatant containing no induced osteoblast differentiation inducing agent is added to the medium containing the culture of the undifferentiated cells derived from rat bone marrow, and then the culture is further cultured.

Thereafter, an effect of the supernatant of the growth medium in which the chondrocytes capable of hypertrophication are cultured (supernatant containing no induced osteoblast differentiation inducing agent) on the undifferentiated cells derived from rat bone marrow is evaluated in the same manner as Example 1.

### Comparative Example 19B

### Effect of Supernatant Containing No Induced Osteoblast Differentiation Inducing Agent on Undifferentiated Cells Derived from Rat Bone Marrow

Chondrocytes incapable of hypertrophication prepared in each of Comparative Example 1B (rat), Comparative Example 1D (rat), Comparative Example 3B (rat), Comparative Example 4B (human), Comparative Example 5B (human), Comparative Example 9B (mouse) and Comparative Example 10B (rabbit) are used. A supernatant of a differentiation agent producing medium in which the cells prepared in each of the Comparative Examples are cultured (supernatant containing no induced osteoblast differentiation inducing agent) is added to the medium containing the culture of the undifferentiated cells derived from rat bone marrow, and then the culture is further cultured.

Thereafter, an effect of the supernatant of the differentiation agent producing medium in which the chondrocytes incapable of hypertrophication are cultured (supernatant containing no induced osteoblast differentiation inducing agent) on the undifferentiated cells derived from rat bone marrow is evaluated in the same manner as Example 1.

### Comparative Example 19C

### Effect of Differentiation Agent Producing Medium or Growth Medium on Undifferentiated Cells Derived from Rat Bone Marrow

Undifferentiated cells derived from rat bone marrow are cultured in a medium to which a supernatant containing an induced osteoblast differentiation inducing agent is not added, but only a differentiation agent producing medium or a growth medium is added. Thereafter, an effect of the differentiation agent producing medium or the growth medium on the undifferentiated cells derived from rat bone marrow is evaluated in the same manner as Example 1.

### Example 20

### Effect of Agent Produced by Chondrocytes Capable of Hypertrophication on Pellet Formed from Undifferentiated Cells Derived from Rat Bone Marrow

Rat bone marrow cells are collected in the same manner as Example 19. These rat bone marrow cells are centrifuged to form them into pellets, and then the pellets are cultured for 1 week in the same manner as Example 16. In this culture, a supernatant of a differentiation agent producing medium in which the chondrocytes capable of hypertrophication are cultured (supernatant containing induced osteoblast differentiation inducing agent) is added to a medium. In this regard, the supernatant is prepared in the same manner as each of Examples 1 to 3 (rat), Examples 4 and 5 (human), Example 7 (rat), Example 9 (mouse) and Example 10 (rabbit).

(Formation of Subcutaneous Pocket)

Subcutaneous pockets are formed in the same manner as Example 16.

(Formation of Bone Defective Region)

Bone defective regions are formed in the same manner as Example 16.

(Implantation of Pellet)

The pellets (cell pellets) are implanted under the dorsal skins and into the bone defective regions of syngenic animals. Four weeks after the implantation, implanted regions and the periphery thereof are surgically removed. In each of the pellets implanted under the skins and into the bone defective regions, an osteogenic ability of the rat bone marrow cells is evaluated.

### Comparative Example 20A

### Effect of Supernatant Containing No Induced Osteoblast Differentiation Inducing Agent on Pellet Formed from Undifferentiated Cells Derived from Rat Bone Marrow

Rat bone marrow cells are collected in the same manner as Example 19. This Comparative Example is preformed in the same manner as Example 20, except that a supernatant of a growth medium in which chondrocytes capable of hypertrophication are cultured (supernatant containing no induced osteoblast differentiation inducing agent) is added to the medium, instead of the supernatant of the differentiation agent producing medium in which the chondrocytes capable of hypertrophication are cultured (supernatant containing induced osteoblast differentiation inducing agent).

(Formation of Subcutaneous Pocket)

Subcutaneous pockets are formed in the same manner as Example 16.

(Formation of Bone Defective Region)

Bone defective regions are formed in the same manner as Example 16.

(Implantation of Pellet)

The pellets (cell pellets) produced in this Comparative Example are implanted under the skins and into the bone defective regions. In each of the pellets implanted under the skins and into the bone defective regions, an osteogenic ability of the rat bone marrow cells is evaluated.

### Comparative Example 20B

### Effect of Supernatant Containing No Induced Osteoblast Differentiation Inducing Agent on Pellet Formed from Undifferentiated Cells Derived from Rat Bone Marrow

Rat bone marrow cells are collected in the same manner as Example 19. Chondrocytes incapable of hypertrophication prepared in each of Comparative Example 1B (rat), Comparative Example 1D (rat), Comparative Example 3B (rat), Comparative Example 4B (human), Comparative Example 5B (human), Comparative Example 9B (mouse) and Comparative Example 10B (rabbit) are used.

A supernatant of a differentiation agent producing medium in which the cells prepared in each of the Comparative Examples are cultured (supernatant containing no induced osteoblast differentiation inducing agent) is added to the medium containing the culture of pellets formed from the bone marrow cells, and then the culture is further cultured.

(Formation of Subcutaneous Pocket)

Subcutaneous pockets are formed in the same manner as Example 16.

(Formation of Bone Defective Region)

Bone defective regions are formed in the same manner as Example 16.

(Implantation of Pellet)

The pellets (cell pellets) produced in this Comparative Example are implanted under the skins and into the bone defective regions. In each of the pellets implanted under the skins and into the bone defective regions, an osteogenic ability of the rat bone marrow cells is evaluated.

### Comparative Example 20C

### Effect of Differentiation Agent Producing Medium or Growth Medium on Undifferentiated Cells Derived from Rat Bone Marrow

Rat bone marrow cells are collected in the same manner as Example 19. Pellets formed from the rat bone marrow cells are cultured using only a differentiation agent producing medium or a growth medium.

(Formation of Subcutaneous Pocket)

Subcutaneous pockets are formed in the same manner as Example 16.

(Formation of Bone Defective Region)

Bone defective regions are formed in the same manner as Example 16.

(Implantation of Pellet)

The pellets (cell pellets) produced in this Comparative Example are implanted under the skins and into the bone defective regions. In each of the pellets implanted under the skins and into the bone defective regions, an osteogenic ability of the rat bone marrow cells is evaluated.

### Example 21

### Effect of Implantation of Composite Material Produced using Induced Chondrocytes Induced by Induced Osteoblast Differentiation Inducing Agent and Biocompatible Scaffold Under the Skin and into Bone Defective Region

(Production of Composite Material)

This Example is performed in the same manner as Example 17, except that undifferentiated cells derived from rat bone marrow are used instead of the mouse C3H10T1/2 cells.

Undifferentiated cells derived from rat bone marrow are collected in the same manner as Example 19. Composite materials are produced by inoculating these undifferentiated cells derived from rat bone marrow on scaffolds recited in Table 11. The composite materials are cultured in a 5% CO₂ incubator at 37° C. for 1 week. Thereafter, the cells on each of the scaffolds are observed.

(Formation of Subcutaneous Pocket)

Subcutaneous pockets are formed in the same manner as Example 17.

(Formation of Bone Defective Region)

Bone defective regions are formed in the same manner as Example 17.

(Implantation of Composite Material)

The composite materials are implanted under the skins and into the bone defective regions of the syngenic animals or the immunodeficient animals. Four weeks after the implantation, the syngenic animals or the immunodeficient animals are sacrificed. Thereafter, implanted regions are surgically removed, fixed with a 10% neutral buffered formalin, and then embedded into paraffin.

Thin slice samples of the implanted regions were produced, and then stained with HE staining. Thereafter, conditions of the implanted regions are evaluated. In each of the composite materials implanted under the skins and into the bone defective regions, an osteogenic ability of the undifferentiated cells is evaluated.

### Comparative Example 21A

### Effect of Implantation of Composite Material Produced using Undifferentiated Cells Cultured in Medium Containing No Induced Osteoblast Differentiation Inducing Agent and Biocompatible Scaffold Under the Skin and into Bone Defective Region

(Production of Composite Material)

As a medium solution, a medium to which a supernatant of a growth medium in which chondrocytes capable of hypertrophication are cultured (supernatant containing no induced osteoblast differentiation inducing agent) is added is used. Composite materials are produced by inoculating undifferentiated cells derived from rat bone marrow on scaffolds recited in Table 11 in the same manner as Example 21. The composite materials are cultured in a 5% CO₂ incubator at 37° C. for 1 week. Thereafter, the cells on each of the scaffolds are observed.

(Formation of Subcutaneous Pocket)

Subcutaneous pockets are formed in the same manner as Example 17.

(Formation of Bone Defective Region)

Bone defective regions are formed in the same manner as Example 17.

(Implantation of Composite Material)

The composite materials are implanted under the skins and into the bone defective regions of the syngenic animals or the immunodeficient animals. Four weeks after the implantation, the syngenic animals or the immunodeficient animals are sacrificed. Thereafter, implanted regions are surgically removed, fixed with a 10% neutral buffered formalin, and then embedded into paraffin.

Thin slice samples of the implanted regions were produced, and then stained with HE staining. Thereafter, conditions of the implanted regions are evaluated. In each of the composite materials implanted under the skins and into the bone defective regions, an osteogenic ability of the undifferentiated cells is evaluated.

### Comparative Example 21B

### Composite Material Cultured using Supernatant Containing No Induced Osteoblast Differentiation Inducing Agent

The chondrocytes incapable of hypertrophication prepared in each of Comparative Example 1B (rat), Comparative Example 1D (rat), Comparative Example 3B (rat), Comparative Example 4B (human), Comparative Example 5B (human), Comparative Example 9B (mouse) and Comparative Example 10B (rabbit) are used. Composite materials are produced in the same manner as Example 21, except that a medium, to which a supernatant of a differentiation agent producing medium in which the cells prepared in each of Comparative Examples are cultured (supernatant containing no induced osteoblast differentiation inducing agent) is added, is used. Thereafter, the cells on each of the scaffolds are observed.

(Formation of Subcutaneous Pocket)

Subcutaneous pockets are formed in the same manner as Example 17.

(Formation of Bone Defective Region)

Bone defective regions are formed in the same manner as Example 17.

(Implantation of Composite Material)

The composite materials are implanted under the skins and into the bone defective regions of the syngenic animals or the immunodeficient animals. Four weeks after the implantation, the syngenic animals or the immunodeficient animals are sacrificed. Thereafter, implanted regions are surgically removed, fixed with a 10% neutral buffered formalin, and then embedded into paraffin.

Thin slice samples of the implanted regions were produced, and then stained with HE staining. Thereafter, conditions of the implanted regions are evaluated. In each of the composite materials implanted under the skins and into the bone defective regions, an osteogenic ability of the undifferentiated cells is evaluated.

### Comparative Example 21C

### Composite Material Cultured using Only Differentiation Agent Producing Medium or Growth Medium

Composite materials are produced in the same manner as Example 21, except that a medium to which only a differentiation agent producing medium or a growth medium is added is used. Thereafter, cells on each of the composite materials are observed.

(Formation of Subcutaneous Pocket)

Subcutaneous pockets are formed in the same manner as Example 17.

(Formation of Bone Defective Region)

Bone defective regions are formed in the same manner as Example 17.

(Implantation of Composite Material)

The composite materials are implanted under the skins and into the bone defective regions of the syngenic animals or the immunodeficient animals. Four weeks after the implantation, the syngenic animals or the immunodeficient animals are sacrificed. Thereafter, implanted regions are surgically removed, fixed with a 10% neutral buffered formalin, and then embedded into paraffin.

Thin slice samples of the implanted regions were produced, and then stained with HE staining. Thereafter, conditions of the implanted regions are evaluated. In each of the composite materials implanted under the skins and into the bone defective regions, an osteogenic ability of the undifferentiated cells is evaluated.

### Example 22

### Effect of Induced Osteoblast Differentiation Inducing Agent on Undifferentiated Cell Strain Derived from Human

(Preparation of Induced Osteoblast Differentiation Inducing Agent Produced by Chondrocytes Capable of Hypertrophication)

Induced osteoblast differentiation inducing agents are prepared in the same manner as Examples 1 to 3 (rat), Examples 4 and 5 (human), Example 7 (rat), Example 9 (mouse) and Example 10 (rabbit).

(Preparation of Undifferentiated Cell Strain Derived from Human)

As undifferentiated cells derived from human, human mesenchymal stem cells derived from bone marrow (hMSC: “PT-2501” produced by Cambrex Corporation) are bought.

These human mesenchymal stem cells are cultured according to a routine procedure. A supernatant containing each of the induced osteoblast differentiation inducing agents prepared in this Example is added to a medium containing a culture of the human mesenchymal stem cells, and then the culture is further cultured in the same manner as Example 1. Thereafter, an effect of each of the induced osteoblast differentiation inducing agents on the undifferentiated cell strain derived from human is evaluated in the same manner as Example 1.

### Comparative Example 22A

### Effect of Supernatant Containing No Induced Osteoblast Differentiation Inducing Agent on Human Mesenchymal Stem Cells

This Comparative Example is preformed in the same manner as Example 22, except that a supernatant of a growth medium, in which chondrocytes capable of hypertrophication are cultured (supernatant containing no induced osteoblast differentiation inducing agent), is added to the medium, instead of the supernatant containing the induced osteoblast differentiation inducing agent. The supernatant containing no induced osteoblast differentiation inducing agent is added to the medium containing a culture of human mesenchymal stem cells, and then the culture is further cultured.

Thereafter, an effect of the supernatant of the growth medium in which the chondrocytes capable of hypertrophication are cultured (supernatant containing no induced osteoblast differentiation inducing agent) on the human mesenchymal stem cells is evaluated.

### Comparative Example 22B

### Effect of Supernatant Containing No Induced Osteoblast Differentiation Inducing Agent on Human Mesenchymal Stem Cells

Chondrocytes incapable of hypertrophication prepared in each of Comparative Example 1B (rat), Comparative Example 1D (rat), Comparative Example 3B (rat), Comparative Example 4B (human), Comparative Example 5B (human), Comparative Example 9B (mouse) and Comparative Example 10B (rabbit) are used. A supernatant of a differentiation agent producing medium in which the cells prepared in each of the Comparative Examples are cultured (supernatant containing no induced osteoblast differentiation inducing agent) is added to the medium containing a culture of human mesenchymal stem cells, and then the culture is further cultured.

Thereafter, an effect of the supernatant of the differentiation agent producing medium in which the chondrocytes incapable of hypertrophication are cultured (supernatant containing no induced osteoblast differentiation inducing agent) on the human mesenchymal stem cells is evaluated.

### Comparative Example 22C

### Effect of Differentiation Agent Producing Medium or Growth Medium on Human Mesenchymal Stem Cells

Human mesenchymal stem cells are cultured in a medium to which only a differentiation agent producing medium or a growth medium is added. Thereafter, an effect of the differentiation agent producing medium or the growth medium on the human mesenchymal stem cells is evaluated.

### Example 23

### Effect of Agent Produced by Chondrocytes Capable of Hypertrophication on Pellet Formed from Human Undifferentiated Cells

Human mesenchymal stem cells derived from bone marrow (hMSC: “PT-2501” produced by Cambrex Corporation) are centrifuged to form them into pellets, and then the pellets are cultured for 1 week. In this culture, a supernatant of a differentiation agent producing medium in which the chondrocytes capable of hypertrophication are cultured (supernatant containing induced osteoblast differentiation inducing agent) is added to a medium. In this regard, the supernatant is prepared in the same manner as each of Examples 1 to 3 (rat), Examples 4 and 5 (human), Example 7 (rat), Example 9 (mouse) and Example 10 (rabbit).

(Formation of Subcutaneous Pocket)

Subcutaneous pockets are formed in the same manner as Example 16.

(Formation of Bone Defective Region)

Bone defective regions are formed in the same manner as Example 16.

(Implantation of Pellet)

The pellets (cell pellets) prepared in this Example are implanted under the dorsal skins of the immunodeficient animals. Four weeks after the implantation, implanted regions and the periphery thereof are surgically removed. In each of the pellets implanted under the skins, an osteogenic ability of the mesenchymal stem cells is evaluated.

### Comparative Example 23A

### Effect of Supernatant Containing No Induced Osteoblast Differentiation Inducing Agent on Pellet Formed from Human Mesenchymal Stem Cells

Human mesenchymal stem cells are prepared in the same manner as Example 19. This Comparative Example is preformed in the same manner as Example 23, except that a supernatant of a growth medium in which chondrocytes capable of hypertrophication are cultured (supernatant containing no induced osteoblast differentiation inducing agent) is added to the medium, instead of the supernatant of the differentiation agent producing medium in which the chondrocytes capable of hypertrophication are cultured (supernatant containing induced osteoblast differentiation inducing agent).

(Formation of Subcutaneous Pocket)

Subcutaneous pockets are formed in the same manner as Example 16.

(Formation of Bone Defective Region)

Bone defective regions are formed in the same manner as Example 16.

(Implantation of Pellet)

The pellets (cell pellets) produced in this Comparative Example are implanted under the skins and into the bone defective regions. In each of the pellets implanted under the skins and into the bone defective regions, an osteogenic ability of the mesenchymal stem cells is evaluated.

### Comparative Example 23B

### Effect of Supernatant Containing No Induced Osteoblast Differentiation Inducing Agent on Pellet Formed from Human Mesenchymal Stem Cells

Human mesenchymal stem cells are prepared in the same manner as Example 19. Chondrocytes incapable of hypertrophication prepared in each of Comparative Example 1B (rat), Comparative Example 1D (rat), Comparative Example 3B (rat), Comparative Example 4B (human), Comparative Example 5B (human), Comparative Example 9B (mouse) and Comparative Example 10B (rabbit) are used.

A supernatant of a differentiation agent producing medium in which the cells prepared in each of the Comparative Examples are cultured (supernatant containing no induced osteoblast differentiation inducing agent) is added to a medium containing pellets formed form the human mesenchymal stem cells, and then the pellets are further cultured.

(Formation of Subcutaneous Pocket)

Subcutaneous pockets are formed in the same manner as Example 16.

(Formation of Bone Defective Region)

Bone defective regions are formed in the same manner as Example 16.

(Implantation of Pellet)

The pellets (cell pellets) produced in this Comparative Example are implanted under the skins and into the bone defective regions. In each of the pellets implanted under the skins and into the bone defective regions, an osteogenic ability of the mesenchymal stem cells is evaluated.

### Comparative Example 23C

### Effect of Differentiation Agent Producing Medium or Growth Medium on Human Mesenchymal Stem Cells

Human mesenchymal stem cells are prepared in the same manner as Example 19. Pellets formed from the human mesenchymal stem cells are cultured in a medium to which only a differentiation agent producing medium or a growth medium is added.

(Formation of Subcutaneous Pocket)

Subcutaneous pockets are formed in the same manner as Example 16.

(Formation of Bone Defective Region)

Bone defective regions are formed in the same manner as Example 16.

(Implementation of Pellet)

The pellets (cell pellets) produced in this Comparative Example are implanted under the skins and into the bone defective regions. In each of the pellets implanted under the skins and into the bone defective regions, an osteogenic ability of the mesenchymal stem cells is evaluated.

### Example 24

### Effect of Implantation of Composite Material Produced using Induced Chondrocytes Induced by Induced Osteoblast Differentiation Inducing Agent and Biocompatible Scaffold Under the Skin and into Bone Defective Region

(Production of Composite Material)

This Example is performed in the same manner as Example 17, except that human mesenchymal stem cells derived from bone marrow (hMSC: “PT-2501” produced by Cambrex Corporation) are used instead of the mouse C3H10T1/2 cells, and immunodeficient animals are used as experimental animals.

Composite materials are produced by inoculating these human mesenchymal stem cells on scaffolds recited in Table 11. These composite materials are cultured in a 5% CO₂ incubator at 37° C. for 1 week. Thereafter, the cells on each of the scaffolds are observed.

(Formation of Subcutaneous Pocket)

Each of the immunodeficient animals to be used for implantation is anesthetized, and then a skin thereof is aseptically incised to form an open wound portion. A round nose scissors is subcutaneously inserted through the open wound portion to separate the skin from a subcutaneous tissue. In this way, a subcutaneous pocket is formed in each of the animals.

(Formation of Bone Defective Region)

Bone defective regions are formed in the same manner as Example 17.

(Implantation of Composite Material)

The composite materials are implanted under the skins and into the bone defective regions of the immunodeficient animals. Four weeks after the implantation, the immunodeficient animals are sacrificed. Thereafter, implanted regions are surgically removed, fixed with a 10% neutral buffered formalin, and then embedded into paraffin.

Thin slice samples of the implanted regions were produced, and then stained with HE staining. Thereafter, conditions of the implanted regions are evaluated. In each of the composite materials implanted under the skins and into the bone defective regions, an osteogenic ability of the mesenchymal stem cells is evaluated.

### Comparative Example 24A

### Effect of Implantation of Composite Material Produced using Undifferentiated Cells Cultured in Medium Containing No Induced Osteoblast Differentiation Inducing Agent and Biocompatible Scaffold Under the Skin and into Bone Defective Region

(Production of Composite Material)

A medium, to which a supernatant of a growth medium in which chondrocytes capable of hypertrophication are cultured (supernatant containing no induced osteoblast differentiation inducing agent) is added, is used. Composite materials are produced by inoculating the human mesenchymal stem cells on scaffolds recited in Table 11 in the same manner as Example 24. These composite materials are cultured in a 5% CO₂ incubator at 37° C. for 1 week. Thereafter, the cells on each of the scaffolds are observed.

(Formation of Subcutaneous Pocket)

Each of immunodeficient animals to be used for implantation is anesthetized, and then a skin thereof is aseptically incised to form an open wound portion. A round nose scissors is subcutaneously inserted through the open wound portion to separate the skin from a subcutaneous tissue. In this way, a subcutaneous pocket is formed in each of the animals.

(Formation of Bone Defective Region)

Bone defective regions are formed in the same manner as Example 17.

(Implantation of Composite Material)

The composite materials are implanted under the skins and into the bone defective regions of the immunodeficient animals. Four weeks after the implantation, the immunodeficient animals are sacrificed. Thereafter, implanted regions are surgically removed, fixed with a 10% neutral buffered formalin, and then embedded into paraffin.

Thin slice samples of the implanted regions were produced, and then stained with HE staining. Thereafter, conditions of the implanted regions are evaluated. In each of the composite materials implanted under the skins and into the bone defective regions, an osteogenic ability of the mesenchymal stem cells is evaluated.

### Comparative Example 24B

### Composite Material Cultured using Supernatant Containing No Induced Osteoblast Differentiation Inducing Agent

Chondrocytes incapable of hypertrophication prepared in each of Comparative Example 1B (rat), Comparative Example 1D (rat), Comparative Example 3B (rat), Comparative Example 4B (human), Comparative Example 5B (human), Comparative Example 9B (mouse) and Comparative Example 10B (rabbit) are used.

Composite materials are produced in the same manner as Example 24, except that a medium, to which a supernatant of a differentiation agent producing medium in which the cells prepared in each of Comparative Examples are cultured (supernatant containing no induced osteoblast differentiation inducing agent) is added, is used. Thereafter, the cells on each of the scaffolds are observed.

(Formation of Subcutaneous Pocket)

Subcutaneous pockets are formed in the same manner as Example 24.

(Formation of Bone Defective Region)

Bone defective regions are formed in the same manner as Example 24.

(Implantation of Composite Material)

The composite materials are implanted under the skins and into the bone defective regions of the immunodeficient animals. Four weeks after the implantation, the immunodeficient animals are sacrificed. Thereafter, implanted regions are surgically removed, fixed with a 10% neutral buffered formalin, and then embedded into paraffin.

Thin slice samples of the implanted regions were produced, and then stained with HE staining. Thereafter, conditions of the implanted regions are evaluated. In each of the composite materials implanted under the skins and into the bone defective regions, an osteogenic ability of mesenchymal stem cells is evaluated.

### Comparative Example 24C

### Composite Material Cultured using Only Differentiation Agent Producing Medium or Growth Medium

Composite materials are produced in the same manner as Example 21, except that a medium to which only a differentiation agent producing medium or a growth medium is added is used. Thereafter, cells on each of the composite materials are observed.

(Formation of Subcutaneous Pocket)

Subcutaneous pockets are formed in the same manner as Example 24.

(Formation of Bone Defective Region)

Bone defective regions are formed in the same manner as Example 24.

(Implantation of Composite Material)

The composite materials are implanted under the skins and into the bone defective regions of the immunodeficient animals. Four weeks after the implantation, the immunodeficient animals are sacrificed. Thereafter, implanted regions are surgically removed, fixed with a 10% neutral buffered formalin, and then embedded into paraffin.

Thin slice samples of the implanted regions were produced, and then stained with HE staining. Thereafter, conditions of the implanted regions are evaluated. In each of the composite materials implanted under the skins and into the bone defective regions, an osteogenic ability of mesenchymal stem cells is evaluated.

### Example 25

### Osteogenic Rate and Osteogenic Amount in Implanting Composite Material Produced using Induced Chondrocytes Induced by Agent Produced by Chondrocytes Capable of Hypertrophication and Biocompatible Scaffold into Bone Defective Region

(Formation of Bone Defective Region, and Measurement of Osteogenic Rate and Osteogenic Amount

The composite materials of Examples 17, 21 and 24 which have induced osteogenesis are implanted into bone defective regions, respectively. Thereafter, an osteogenic rate and an osteogenic amount in each of the bone defective regions are measured using MZ-500S (produced by MARUTO INSTRUMENT CO., LTD.). This measurement is performed using a columnar bar-shaped punching jig at test conditions in which a speed thereof is 2 mm/min. As each of the biocompatible scaffolds, hydroxyapatite is used.

Composite materials (disc shape having a diameter of 3 mm) each produced using chondrocytes capable of hypertrophication obtained in the same manner as Example 1 and the hydroxyapatite are implanted into the bone defective regions (punching aperture having a diameter of 3 mm), respectively.

In this regard, the bone defective regions are formed in the same manner as Example 17. Four and twelve weeks after the implantation, implanted regions are surgically removed, and then μCT data thereof are measured.

### Comparative Example 25A

The composite materials of Comparative Examples 17A to 17A, 21A to 21C and 24A to 24C, composite materials each containing natural osteoblasts and a scaffold, and scaffolds alone are implanted into bone defective regions, respectively. Thereafter, osteogenic rate and osteogenic amount in each of the bone defective regions are measured in the same manner as Example 25.

### Example 26

### Induction of Induced Osteoblasts using Induced Osteoblast Differentiation Inducing Agent Produced by Chondrocytes Capable of Hypertrophication

(Preparation of Induced Osteoblast Differentiation Inducing Agent Produced by Chondrocytes Capable of Hypertrophication)

In this Example, chondrocytes capable of hypertrophication were cultured in an MEM differentiation agent producing medium, and then a supernatant thereof was collected on a time course of 4 day to 3 weeks in the same manner as Example 1 to obtain fractional supernatants. Next, each of the fractional supernatants was put into a centrifugal filter, and then centrifuged at 4,000×g and at 4° C. for 30 minutes under such a condition that a high molecular fraction and a low molecular fraction were separated from each other.

In this way, each fractional supernatant was separated into a high molecular weight fraction with a molecular weight of 50,000 or higher and a low molecular weight fraction with a molecular weight of 50,000 or lower, and at the same time, the high molecular weight fraction was concentrated to 10-fold.

Thereafter, this concentrated high molecular weight fraction (medium supernatant) was diluted to 5-fold using a differentiation agent producing medium. In the centrifugation, a 50K film (“Amicon Ultra 15, 50,000NMWL, catalog number: UFC905024” produced by Millipore Corporation) was used.

(Preparation of Undifferentiated Mesenchymal Stem Cells Derived from Bone Marrow)

Four week-old Wistar male rats were sacrificed using chloroform. The rats' femoral regions were shaved using a razor and their whole bodies were immersed into a Hibitane solution (10-fold dilution) to be disinfected. The femoral regions were incised and femurs were surgically removed aseptically. Thereafter, both epiphyseal regions of each of the femurs are removed to collect diaphyses.

Ten to fifteen mL of an MEM growth medium (containing a minimum essential medium (MEM), 15% FBS, 100 U/mL penicillin, 0.1 mg/mL streptomycin and 0.25 μg/mL amphotericin B) was introduced into a syringe to which a needle attached, and then infused into the collected diaphyses via the needle so that a bone marrow thereinside was flushed out with the medium to obtain a bone marrow solution.

The bone marrow suspension was inoculated in a T-75 flask (produced by Becton, Dickinson and Company) to adjust a final amount to 30 mL. This bone marrow suspension was cultured at 37° C. for 1 week. As a medium, an MEM differentiation agent producing medium was used. A half of the medium is exchanged by a new medium two times per 1 week. One week after the culture, cells adhering to a bottom surface of the T-75 flask are determined as undifferentiated mesenchymal stem cells.

These cells were rinsed with a dulbecco's phosphate buffered saline (“D-PBS, catalog number: 14190” produced by Invitrogen Corporation), separated from the T-75 flask with a 0.05% trypsin-EDTA solution (produced by Invitrogen Corporation). Thereafter, the cells were collected and rinsed by centrifugation at 170×g for 3 minutes, and then used.

(I: Direct Addition of Induced Osteoblast Differentiation Inducing Agent Produced by Chondrocytes Capable of Hypertrophication)

The undifferentiated mesenchymal stem cells derived from bone marrow prepared in this Example were inoculated in a well at a density of 1×10⁻⁵ cells/mL/well, and then cultured in an MEM growth medium (containing a minimum essential medium (MEM), 15% FBS, 100 U/mL penicillin, 0.1 mg/mL streptomycin and 0.25 μg/mL amphotericin B) overnight (for 18 hours).

After the culture, 1 mL of an MEM differentiation agent producing medium (containing a minimum essential medium (MEM), 15% FBS (fetal bovine serum), 10 nM dexamethasone, 10 mM β-glycerophosphate, 50 μg/mL ascorbic acid, 100 U/mL penicillin, 0.1 mg/mL streptomycin and 0.25 μg/mL amphotericin B) containing an agent or an MEM differentiation agent producing medium containing no agent (differentiation agent producing medium alone) was added to the well, and then the mesenchymal stem cells were further cultured for 72 hours.

Thereafter, an alkaline phosphatase activity, which was one of osteoblast markers, of the mesenchymal stem cells was measured. The measurement of the alkaline phosphatase activity was performed in the same manner as Example 1. Results thereof are shown in the following Table 12.

TABLE 12

| | | | | Mean | Relative |
|---|---|---|---|---|---|
| | 1 | 2 | 3 | value | value |
| Agent (+) | 0.208 | 0.226 | 0.299 | 0.244 | 2.17 |
| Agent (−) | 0.120 | 0.111 | 0.107 | 0.113 | 1 |

Agent (+): differentiation agent producing medium containing agent-adding group
Agent (−): differentiation agent producing medium containing no agent (differentiation agent producing medium alone)-adding group

It was confirmed that an induced osteoblast differentiation inducing agent produced by chondrocytes capable of hypertrophication could increase an alkaline phosphatase activity of the undifferentiated mesenchymal stem cells derived from primary rat bone marrow.

(II: Addition of Induced Osteoblast Differentiation Inducing Agent Produced by Chondrocytes Capable of Hypertrophication Impregnated into Hydroxyapatite)

(Preparation of Hydroxyapatite)

As hydroxyapatite, APACERAM AX (produced by HOYA CORPORATION, Artificial bone AB-01, GA-3) was used. A dilute solution having such an amount that the APACERAM AX is fully immersed thereinto (e.g., 1 mL of the dilute solution per 10 hydroxyapatite particles) and the APACERAM AX were introduced into a syringe. In this state, a plunger of the syringe was pulled so that the APACERAM AX was degassed. At this time, about 0.3 mL of the dilute solution was impregnated into the hydroxyapatite particles.

The undifferentiated mesenchymal stem cells derived from bone marrow prepared in this Example were inoculated in a well at a density of 1×10⁻⁵ cells/mL/well, and then cultured in an MEM growth medium overnight (for 18 hours). Next, an MEM differentiation agent producing medium containing an agent or an MEM differentiation agent producing medium containing no agent (differentiation agent producing medium alone) impregnated into the APACERAM AX was added to the well in which the mesenchymal stem cells cultured in this Example were inoculated, and then they were further cultured for 72 hours.

Thereafter, an alkaline phosphatase activity, which was one of osteoblast markers, of the mesenchymal stem cells was measured. The measurement of the alkaline phosphatase activity was performed in the same manner as Example 1. Results thereof are shown in the following Table 13.

TABLE 13

| | | | | Mean | Relative |
|---|---|---|---|---|---|
| | 1 | 2 | 3 | value | value |
| Agent (+) | 0.169 | 0.153 | 0.191 | 0.171 | 2.15 |
| Agent (−) | 0.085 | 0.077 | 0.077 | 0.080 | 1 |

Agent (+): differentiation agent producing medium containing agent impregnated into APACERAM AX-adding group
Agent (−): differentiation agent producing medium containing no agent (differentiation agent producing medium alone) impregnated into APACERAM AX-adding group

It was confirmed that an induced osteoblast differentiation inducing agent produced by chondrocytes capable of hypertrophication could increase the alkaline phosphatase activity of the undifferentiated mesenchymal stem cells derived from primary rat bone marrow.

### Example 27A

### Effect of Agent on Induction of Differentiation of Undifferentiated Mesenchymal Stem Cells Derived from Rat Bone Marrow into Osteoblasts

(Detection of Agent Produced by Chondrocytes Capable of Hypertrophication Derived from Rat Bone Marrow)

In this Example, a cellular function regulating agent was prepared by culturing chondrocytes capable of hypertrophication derived from costa/costal cartilage in an MEM differentiation agent producing medium in the same manner as Example 1.

(Collection of Undifferentiated Mesenchymal Stem Cells Derived from Rat Bone Marrow)

Cells were collected from bone marrows of rat's femurs, and then were cultured for 1 week in the same manner as Example 26 to obtain a cell solution. One mL of a 2.5×10⁻⁴ cells/mL cell solution was inoculated in a 24-well plate, and then the cells were cultured in an MEM growth medium. In this way, mesenchymal stem cells derived from primary rat bone marrow were prepared.

(Addition of Sample and Measurement of Alkaline Phosphatase Activity)

After the mesenchymal stem cells derived from primary rat bone marrow were cultured in the MEM growth medium for 18 hours, 1 mL of each of the following sample solutions was added to the mesenchymal stem cells derived from primary rat bone marrow. Further, the mesenchymal stem cells derived from primary rat bone marrow were cultured for 72 hours, and then an alkaline phosphatase activity thereof was measured in the same manner as Example 1.

(Sample Solution Added)

(1) Supernatant of differentiation agent producing medium in which chondrocytes capable of hypertrophication were cultured; differentiation agent producing medium containing agent.

(2) Only MEM differentiation agent producing medium; medium containing no agent according to the present invention, but containing dexamethasone; Maniatopoorus's osteoblast differentiation medium

(3) Only MEM growth medium; MEM growth medium containing no agent and no dexamethasone

Results thereof are shown in the following Table.

A value of the alkaline phosphatase activity of the mesenchymal stem cells derived from primary rat bone marrow cultured by adding the supernatant containing the induced osteoblast differentiation inducing agent increased by more than 2 times that of the mesenchymal stem cells cultured by adding the MEM growth medium containing no agent and no dexamethasone.

On the other hand, even in the case where the mesenchymal stem cells derived from primary rat bone marrow were cultured by adding only the differentiation agent producing medium containing the dexamethasone, but no induced osteoblast differentiation inducing agent, the alkaline phosphatase activity thereof increased. However, the increase of the alkaline phosphatase activity was little as compared with that of the mesenchymal stem cells cultured by adding the supernatant containing the induced osteoblast differentiation inducing agent.

From the results of FIG. 8 and the like, conventional low molecular components (including the dexamethasone) are unlikely to be contained in the supernatant containing the induced osteoblast differentiation inducing agent. For this reason, it is believed that the effect resulting from the addition of the supernatant containing the induced osteoblast differentiation inducing agent is obtained by the induced osteoblast differentiation inducing agent itself.

Therefore, from the results in this Example, it is believed that the induced osteoblast differentiation inducing agent has higher ability of inducing differentiation into osteoblasts than that of the dexamethasone which is one of conventional osteoblast differentiation inducing components.

TABLE 14
ALP (Abs 405), 72 hours (3 days) after addition of supernatant

| | Sample | | | | Mean | Relative |
|---|---|---|---|---|---|---|
| | added | 1 | 2 | 3 | value | value |
| Addition of | (1) | 0.688 | 0.665 | 0.686 | 0.680 | 2.1 |
| supernatant | | | | | | |
| Only medium | (2) | 0.426 | 0.420 | 0.490 | 0.445 | 1.4 |
| | (3) | 0.324 | 0.270 | 0.364 | 0.321 | 1 |

### Example 27B

### Effect of Supernatant of MEM Growth Medium in which Chondrocytes Capable of Hypertrophication were Cultured on Undifferentiated Mesenchymal Stem Cells Derived from Primary Rat Bone Marrow

In this Example, a supernatant of an MEM growth medium in which chondrocytes capable of hypertrophication were cultured was collected in the same manner as Comparative Example 1A.

Mesenchymal stem cells derived from primary rat bone marrow were prepared in the same manner as Example 27A.

(Addition of Sample and Measurement of Alkaline Phosphatase Activity)

After the mesenchymal stem cells derived from primary rat bone marrow were cultured in the MEM growth medium for 18 hours, 1 mL of each of the following sample solutions was added to the mesenchymal stem cells derived from primary rat bone marrow. Further, the mesenchymal stem cells derived from primary rat bone marrow were cultured for 72 hours, and then an alkaline phosphatase activity thereof was measured in the same manner as Example 1.

(Sample Solution Added)

GC/differentiation: a sample solution added is a supernatant of an MEM differentiation agent producing medium in which chondrocytes capable of hypertrophication were cultured.

GC/growth: a sample solution added is a supernatant of an MEM growth medium in which chondrocytes capable of hypertrophication were cultured.

Growth: a sample solution added is an MEM growth medium.

Hereinafter, results thereof are shown.

It was confirmed that an agent capable of increasing the alkaline phosphatase activity of the mesenchymal stem cells derived from primary rat bone marrow was present in the supernatant of the MEM differentiation agent producing medium in which the chondrocytes capable of hypertrophication were cultured, but the agent capable of increasing the alkaline phosphatase activity of the mesenchymal stem cells derived from primary rat bone marrow was not present in the supernatant of the MEM growth medium in which the chondrocytes capable of hypertrophication were cultured.

TABLE 15
Cells: rMSC (rat)

| | | | | Mean | Relative |
|---|---|---|---|---|---|
| | 1 | 2 | 3 | value | value |
| GC/ | 0.688 | 0.665 | 0.686 | 0.680 | 2.120 |
| differentiation | | | | | |
| GC/growth | 0.192 | 0.184 | 0.151 | 0.176 | 0.548 |
| Only growth | 0.324 | 0.274 | 0.364 | 0.321 | 1.000 |

It is also possible to confirm an effect of a supernatant of a HAM differentiation agent producing medium in which the chondrocytes capable of hypertrophication derived from rat were cultured on the mesenchymal stem cells derived from primary rat bone marrow in the same manner as this Example.

### Example 28A

### Effect of Agent on Induction of Differentiation of Human Undifferentiated Mesenchymal Stem Cells into Osteoblasts

(Detection of Agent Produced by Chondrocytes Capable of Hypertrophication Derived from Rat)

In this Example, a cellular function regulating agent was prepared by culturing chondrocytes capable of hypertrophication derived from costa/costal cartilage in an MEM differentiation agent producing medium in the same manner as Example 1.

(Preparation of Human Mesenchymal Stem Cells)

A human mesenchymal stem cell strain (hMSC: “PT-2501”) were bought from Cambrex Corporation, and then cultured for 1 week to obtain a cell solution. One mL of a 2.5×10⁻⁴ cells/mL cell solution was inoculated in a 24-well plate, and then the cells were cultured in a MSCGM medium (growth medium).

(Addition of Sample and Measurement of Alkaline Phosphatase Activity)

After the human mesenchymal stem cells were cultured in the MSCGM medium for 18 hours, 1 mL of each of the following sample solutions was added to the human mesenchymal stem cells. Further, the mesenchymal stem cells were cultured for 72 hours, and then an alkaline phosphatase activity thereof was measured in the same manner as Example 1.

(Sample Solution Added)

(1) Supernatant of differentiation agent producing medium in which chondrocytes capable of hypertrophication were cultured; differentiation agent producing medium containing agent.

(2) Only MEM differentiation agent producing medium; medium containing no agent according to the present invention, but containing dexamethasone; Maniatopoorus's osteoblast differentiation medium

(3) Only MEM growth medium; MEM growth medium containing no agent and no dexamethasone

Results thereof are shown in the following Table.

A value of the alkaline phosphatase activity of the human mesenchymal stem cells cultured by adding the supernatant containing the induced osteoblast differentiation inducing agent increased by more than 5 times that of the human mesenchymal stem cells cultured by adding the MEM growth medium containing no agent and no dexamethasone.

On the other hand, even in the case where the human mesenchymal stem cells were cultured by adding only the differentiation agent producing medium containing the dexamethasone, but no induced osteoblast differentiation inducing agent, the alkaline phosphatase activity thereof increased. However, the increase of the alkaline phosphatase activity was little as compared with that of the human mesenchymal stem cells cultured by adding the supernatant containing the induced osteoblast differentiation inducing agent.

From the results of FIG. 8 and the like, conventional low molecular components (including the dexamethasone) are unlikely to be contained in the supernatant containing the induced osteoblast differentiation inducing agent. For this reason, it is believed that the effect resulting from the addition of the supernatant containing the induced osteoblast differentiation inducing agent is obtained by the induced osteoblast differentiation inducing agent itself.

Therefore, from the results in this Example, it is believed that the induced osteoblast differentiation inducing agent has higher ability of inducing differentiation into osteoblasts than that of the dexamethasone which is one of conventional osteoblast differentiation inducing components.

TABLE 16
ALP (Abs 405), 72 hours (3 days) after addition of supernatant

| Sample | | | | Mean | Relative |
|---|---|---|---|---|---|
| added | 1 | 2 | 3 | value | value |
| (1) | 0.83 | 0.73 | 0.84 | 0.80 | 5.2 |
| (2) | 0.20 | 0.35 | 0.26 | 0.27 | 1.8 |
| (3) | 0.13 | 0.14 | 0.18 | 0.15 | 1 |

(Alkaline Phosphatase Staining)

After the human mesenchymal stem cells were cultured in the MSCGM medium (growth medium) for 18 hours, 1 mL of each of the following sample solutions was added to the human mesenchymal stem cells. Further, the human mesenchymal stem cells were cultured for 72 hours, and then an alkaline phosphatase activity staining thereof was performed in the same manner as Example 1.

(Sample Solution Added)

(1) Supernatant of differentiation agent producing medium in which chondrocytes capable of hypertrophication were cultured; differentiation agent producing medium containing agent.

(2) Only MEM differentiation agent producing medium; medium containing no agent according to the present invention, but containing dexamethasone; Maniatopoorus's osteoblast differentiation medium

(3) Only MEM growth medium; MEM growth medium containing no agent and no dexamethasone

Results thereof are shown in FIG. 39.

The human mesenchymal stem cells cultured by adding the supernatant containing the induced osteoblast differentiation inducing agent strongly expressed the alkaline phosphatase as compared with those cultured by adding the MEM growth medium containing no agent and no dexamethasone.

On the other hand, even in the case where the human mesenchymal stem cells were cultured by adding only the differentiation agent producing medium containing the dexamethasone, but no induced osteoblast differentiation inducing agent, they expressed the alkaline phosphatase. However, an expression level of the alkaline phosphatase was little as compared with that of the human mesenchymal stem cells cultured by adding the supernatant containing the induced osteoblast differentiation inducing agent.

From the results of FIG. 8 and the like, conventional low molecular components (including the dexamethasone) are unlikely to be contained in the supernatant containing the induced osteoblast differentiation inducing agent. For this reason, it is believed that the effect resulting from the addition of the supernatant containing the induced osteoblast differentiation inducing agent is obtained by the induced osteoblast differentiation inducing agent itself.

Therefore, from the results in this Example, it is believed that the induced osteoblast differentiation inducing agent has higher ability of inducing differentiation into osteoblasts than that of the dexamethasone which is one of conventional osteoblast differentiation inducing components.

### Example 28B

### Effect of Supernatant of MEM Growth Medium in which Chondrocytes Capable of Hypertrophication were Cultured on Human Undifferentiated Mesenchymal Stem Cells

In this Example, a supernatant of an MEM growth medium in which chondrocytes capable of hypertrophication were cultured was collected in the same manner as Comparative Example 1A.

Human undifferentiated mesenchymal stem cells (hMSC: “PT-2501”) were bought from Cambrex Corporation, and then cultured in a MSCGM medium (growth medium) in the same manner as Example 28A.

(Addition of Sample and Measurement of Alkaline Phosphatase Activity)

After the human mesenchymal stem cells were cultured in the MSCGM medium (growth medium) for 18 hours, 1 mL of each of the following sample solutions was added to the human mesenchymal stem cells. Further, the human mesenchymal stem cells were cultured for 72 hours, and then an alkaline phosphatase activity thereof was measured in the same manner as Example 1.

(Sample Solution Added)

GC/differentiation: a sample solution added is a supernatant of an MEM differentiation agent producing medium in which chondrocytes capable of hypertrophication were cultured.

GC/growth: a sample solution added is a supernatant of an MEM growth medium in which chondrocytes capable of hypertrophication were cultured.

Growth: a sample solution added is an MEM growth medium.

Hereinafter, results thereof are shown.

It was confirmed that an agent capable of increasing the alkaline phosphatase activity of the human mesenchymal stem cells was present in the supernatant of the MEM differentiation agent producing medium in which the chondrocytes capable of hypertrophication were cultured, but the agent capable of increasing the alkaline phosphatase activity of the human mesenchymal stem cells was not present in the supernatant of the MEM growth medium in which the chondrocytes capable of hypertrophication were cultured.

TABLE 17
Cells: hMSC (human)

| | | | | Mean | Relative |
|---|---|---|---|---|---|
| | 1 | 2 | 3 | value | value |
| GC/ | 1.504 | 2.315 | 1.773 | 1.864 | 4.618 |
| differentiation | | | | | |
| GC/growth | 0.560 | 0.395 | 0.523 | 0.493 | 1.220 |
| Only growth | 0.435 | 0.322 | 0.454 | 0.404 | 1.000 |

It is also possible to confirm an effect of a supernatant of a HAM differentiation agent producing medium in which the chondrocytes capable of hypertrophication derived from rat were cultured on the human mesenchymal stem cells in the same manner as this Example.

As discussed above, the present invention has been illustrated by preferred embodiments of the present invention. However, the scope of the present invention should not be limited by such embodiments. It is appreciated that the present invention should be limited only by the scope of the claims.

It is understood that those skilled in the art can perform equivalents of the present invention according to the description of the present invention or the common technical knowledge within the art. It is also understood that the contents of patents, patent application and documents cited herein should be incorporated as references, as specifically described herein.

### INDUSTRIAL APPLICABILITY

The present invention successfully produces an agent capable of inducing differentiation into induced osteoblasts from a wide rang of cells, including conventional cell lines and/or non-conventional cells, by providing a cellular function regulating agent produced by chondrocytes capable of hypertrophication. The chondrocytes capable of hypertrophication are capable of inducing differentiation of undifferentiated cells into the induced osteoblasts. Since such an agent is not known, the existence of the agent itself is industrially applicable.

What is claimed is:

1. A method of inducing undifferentiated cells into induced osteoblasts, comprising:
A) a step of providing an induced osteoblast differentiation inducing agent obtained by culturing chondrocytes capable of hypertrophication in a differentiation agent producing medium containing dexamethasone, β-glycerophosphate, ascorbic acid and a serum component; and
B) a step of culturing the undifferentiated cells in an undifferentiated cell culture medium containing the induced osteoblast differentiation inducing agent and a medium component, to differentiate the undifferentiated cells into the induced osteoblasts.

2. The method as claimed in claim 1, wherein the induced osteoblast differentiation inducing agent exists (1) in the medium in which the chondrocytes capable of hypertrophication are cultured, or (2) in a fraction with a molecular weight of 50,000 or higher obtained by subjecting the medium in which the chondrocytes capable of hypertrophication are cultured to ultrafiltration using a filter having a molecular cutoff of 50,000.

3. The method as claimed in claim 1, wherein the step A) includes:
culturing the chondrocytes capable of hypertrophication in the differentiation agent producing medium containing the dexamethasone, the β-glycerophosphate, the ascorbic acid and the serum component; and
collecting a supernatant of the differentiation agent producing medium after the culture.

4. The method as claimed in claim 1, wherein the step A) includes subjecting a supernatant of the differentiation agent producing medium in which the chondrocytes capable of hypertrophication are cultured to ultrafiltration to separate it into a fraction with a molecular weight of 50,000 or higher.

5. The method as claimed in claim 1, wherein the undifferentiated cells comprise mesenchymal stem cells.

6. The method as claimed in claim 5, wherein the undifferentiated cells comprise mesenchymal stem cells derived from bone marrow.

7. The method as claimed in claim 1, wherein the undifferentiated cells comprise cells selected from the group comprising C3H10T1/2 cells, 3T3-Swiss albino cells, BALB/3T3 cells, NIH3T3 cells, PT-2501 cells and stem cells derived from primary rat bone marrow.

8. The method as claimed in claim 7, wherein the undifferentiated cells comprise the C3H10T1/2 cells.

9. The method as claimed in claim 1, wherein the undifferentiated cell culture medium contains an eagle's basal medium (BME), a minimum essential medium (MEM), a Ham's F12 medium (HAM) or a dulbecco's modified eagle medium (DMEM).

10. The method as claimed in claim 1, wherein the undifferentiated cells comprise mesenchymal stem cells derived from bone marrow, and
wherein the step A) includes (1) culturing the chondrocytes capable of hypertrophication in the differentiation agent producing medium containing the dexamethasone, the β-glycerophosphate, the ascorbic acid and the serum component, and then collecting a supernatant of the differentiation agent producing medium after the culture, and (2) subjecting the supernatant to ultrafiltration to separate it into a fraction with a molecular weight of 50,000 or higher.

11. The method as claimed in claim 1, wherein the undifferentiated cells comprise C3H10T1/2 cells, PT-2501 cells or stem cells derived from primary rat bone marrow, and
wherein the step A) includes (1) culturing the chondrocytes capable of hypertrophication in the differentiation agent producing medium containing the dexamethasone, the β-glycerophosphate, the ascorbic acid and the serum component, and then collecting a supernatant of the differentiation agent producing medium after the culture, and (2) subjecting the supernatant to ultrafiltration to separate it into a fraction with a molecular weight of 50,000 or higher.

12. Induced osteoblasts produced using the method defined by claim 1.

13. A medicine or medical material for promoting or inducing osteogenesis in a biological organism, comprising:
The induced osteoblasts produced using the method defined by claim 1.

14. The medicine or medical material as claimed in claim 13, wherein the undifferentiated cells comprise C3H10T1/2 cells, PT-2501 cells or stem cells derived from primary rat bone marrow.

15. The medicine or medical material as claimed in claim 14, wherein the C3H10T1/2 cells are formed into a pellet.

16. A method of producing a composite material for promoting or inducing osteogenesis in a biological organism, comprising:
A) a step of providing the induced osteoblasts produced using the method defined by claim 1;
B) a step of culturing the induced osteoblasts on a biocompatible scaffold.

17. The method as claimed in claim 16, wherein the induced osteoblasts are induced from the undifferentiated cells on the biocompatible scaffold.

18. A method of promoting or inducing osteogenesis in a biological organism, comprising:
a step of implanting the induced osteoblasts produced using the method defined by claim 1, into a region where the osteogenesis is required to be promoted or induced in the biological organism.

19. Use of induced osteoblasts for producing a medicine or medical material for promoting or inducing osteogenesis in a biological organism, wherein the induced osteoblasts are produced using the method defined by claim 1.
